(19) United States
(12) Patent Application Publication
(10) Pub. No.: US 2025/0215096 A1
(43) Pub. Date: Jul. 3, 2025

(54) CHIMERIC ANTIGEN RECEPTOR THERAPY IN COMBINATION WITH IL-15R AND IL15

(71) Applicants: Novartis AG, Basel (CH); The Trustees of the University of Pennsylvania, Philadelphia, PA (US)

(72) Inventors: John Scholler, Narberth, PA (US); Carl H. June, Merion Station, PA (US)

(21) Appl. No.: 18/795,560

(22) Filed: Aug. 6, 2024

Related U.S. Application Data
(63) Continuation of application No. 16/969,381, filed on Aug. 12, 2020, now abandoned.
(60) Provisional application No. 62/630,109, filed on Feb. 13, 2018.

(51) Int. Cl. C07K 16/28; A61K 40/11; A61K 40/31; A61K 40/35; A61K 40/42; A61P 35/00; C07K 16/24; C12N 15/86

(52) U.S. Cl. CPC C07K 16/2866; A61K 40/11; A61K 40/31; A61K 40/35; A61K 40/4211; A61K 40/4255; A61P 35/00; C07K 16/244; C07K 16/2878; C12N 15/86; A61K 2239/31; A61K 2239/38; A61K 2239/48; A61K 2239/54

(57) ABSTRACT

The invention provides compositions and methods for treating diseases such as cancer. The invention also relates to a method of administering a therapy comprising a chimeric antigen receptor, an IL-15R molecule and an IL-15 molecule.

This application is a continuation of U.S. application Ser. No. 16/969,381, filed Aug. 12, 2020, which is a U.S. National Phase Application under 35 U.S.C. § 371 of International Application No. PCT/US2019/017853, filed Feb. 13, 2019, which claims priority to U.S. Ser. No. 62/630,109 filed Feb. 13, 2018, the entire contents of which are herein incorporated by reference in its entirety.

### SEQUENCE LISTING

The instant application contains a Sequence Listing which has been submitted electronically in XML format and is hereby incorporated by reference in its entirety. Said XML copy, created on Aug. 5, 2024, is named N2067-715120_SL.xml and is 1,309,354 bytes in size.

### FIELD OF THE INVENTION

The present invention relates generally to the use of cells engineered to express a chimeric antigen receptor, in combination with an IL-15R molecule and an IL-15 molecule, to treat a disease such as cancer.

### BACKGROUND OF THE INVENTION

Recent developments using chimeric antigen receptor (CAR) modified T cell (CART) therapy, which relies on redirecting T cells to a suitable cell-surface molecule on cancer cells, show promising results in harnessing the power of the immune system to treat cancers (see, e.g., Sadelain et al., Cancer Discovery 3:388-398 (2013)).

Given the ongoing need for improved strategies for targeting diseases such as cancer, new compositions and methods for improving CART therapies are highly desirable.

### SUMMARY OF THE INVENTION

This disclosure features, at least in part, compositions and methods of treating disorders such as cancer using immune effector cells (e.g., T cells or NK cells) that express a chimeric antigen receptor (CAR) molecule, e.g., a CAR molecule that binds to a tumor antigen, e.g., an antigen expressed on the surface of a solid tumor or a hematological tumor. In one aspect, the invention features use of the CAR-expressing cell therapy in combination with an Interleukin 15 receptor (IL-15R) molecule, and/or an Interleukin 15 (IL-15) cytokine molecule. In some embodiments, disclosed herein, inter alia, is a composition comprising a CAR molecule co-expressing an IL-15R molecule and an IL-15 molecule, and uses thereof. In some embodiments, the CAR molecule, the IL-15R molecule and the IL-15 molecule are disposed on a single nucleic acid molecule. In some embodiments, the CAR molecule, the IL-15R molecule and the IL-15 molecule are disposed on separate nucleic acid molecules. Without wishing to be bound by theory, it is believed that in some embodiments, a CAR cell co-expressing an IL-15R molecule and an IL-15 molecule (“CAR IL-15R/IL-15 expressing cell”), has enhanced expansion and proliferative capacity. In some embodiments, a CAR IL-15R/IL-15 expressing cell has improved efficacy.

In one aspect, disclosed herein is a nucleic acid molecule, e.g., an isolated nucleic acid molecule, comprising:
(i) a first nucleic acid sequence encoding a chimeric antigen receptor (CAR) molecule that binds to an antigen (e.g., an antigen described herein, e.g., CD19, Mesothelin or BCMA);
(ii) a second nucleic acid sequence comprising an IL-15 receptor (IL-15R) molecule; and
(iii) a third nucleic acid sequence comprising an IL-15 molecule.

In some embodiments, the first nucleic acid sequence, second nucleic acid sequence and third nucleic acid sequence are disposed on a single nucleic acid molecule, e.g., a vector, e.g., a viral vector, e.g., a lentivirus vector.

In some embodiments, the first nucleic acid sequence, second nucleic acid sequence and third nucleic acid sequence are disposed on separate nucleic acid molecules, e.g., separate vectors, e.g., separate viral vectors, e.g., separate lentivirus vectors. In some embodiments, the first nucleic acid sequence is disposed on a first nucleic acid molecule, e.g., a first vector, e.g., a first viral vector, e.g., a first lentivirus vector. In some embodiments, the second nucleic acid sequence is disposed on a second nucleic acid molecule, e.g., a second vector, e.g., a second viral vector, e.g., a second lentivirus vector. In some embodiments, the third nucleic acid sequence is disposed on a third nucleic acid molecule, e.g., a third vector, e.g., a third viral vector, e.g., a third lentivirus vector.

In some embodiments, the first nucleic acid sequence and the second nucleic acid sequence are disposed on a first nucleic acid molecule, e.g., a first vector, e.g., a first viral vector, e.g., a first lentivirus vector. In some embodiments, the third nucleic acid sequence is disposed on a second nucleic acid molecule, e.g., a second vector, e.g., a second viral vector, e.g., a second lentivirus vector.

In some embodiments, the first nucleic acid sequence and the third nucleic acid sequence are disposed on a first nucleic acid molecule, e.g., a first vector, e.g., a first viral vector, e.g., a first lentivirus vector. In some embodiments, the second nucleic acid sequence is disposed on a second nucleic acid molecule, e.g., a second vector, e.g., a second viral vector, e.g., a second lentivirus vector.

In some embodiments, the first nucleic acid sequence is disposed on a first nucleic acid molecule, e.g., a first vector, e.g., a first viral vector, e.g., a first lentivirus vector. In some embodiments, the second nucleic acid sequence and the third nucleic acid sequence are disposed on a second nucleic acid molecule, e.g., a second vector, e.g., a second viral vector, e.g., a second lentivirus vector.

In some embodiments, the nucleic acid molecule comprises a multicistronic lentivirus vector.

In some embodiments, the single nucleic acid molecule has the following arrangement in an N- to C-terminal orientation:
(i) the first nucleic acid sequence-a first linker-the second nucleic acid sequence-a second linker-the third nucleic acid sequence; or
(ii) the first nucleic acid sequence-a first linker-the third nucleic acid sequence-a second linker-the second nucleic acid sequence,
wherein the first and second linkers are different.

In some embodiments, the single nucleic acid molecule has the following arrangement in an N- to C-terminal orientation:
(iii) the second nucleic acid sequence-a first linker-the third nucleic acid sequence-a second linker-the first nucleic acid sequence; or
(iv) the third nucleic acid sequence-a first linker-the second nucleic acid sequence-a second linker-the first nucleic acid sequence,
wherein the first and second linkers are different.

In some embodiments, the linker encodes a self-cleavage site, e.g., a P2A site, a T2A site, an E2A site, or an F2A site.

In some embodiments, the first linker encodes a P2A site and the second linker encodes a T2A site, wherein:
(i) the first linker comprises the nucleotide sequence of SEQ ID NO: 23 (or a sequence at least about 85%, 90%, 95%, 99% or more identical thereto, and/or having one, two, three or more substitutions, insertions, deletions, or modifications), and/or
(ii) the second linker comprises a nucleotide sequence encoding SEQ ID NO: 1478 (or a sequence at least about 85%, 90%, 95%, 99% or more identical thereto, and/or having one, two, three or more substitutions, insertions, deletions, or modifications).

In some embodiments, the first nucleic acid sequence, second nucleic acid sequence and third nucleic acid sequence are disposed on separate nucleic acid molecules, e.g., the first nucleic acid sequence is on one nucleic acid molecule and the second and third nucleic acid sequences are one a second nucleic acid molecule; or the first, second and third nucleic acid sequences are on three separate nucleic acid molecules.

In some embodiments, the third nucleic acid sequence encodes an amino acid comprising the sequence of SEQ ID NO: 1002, a sequence at least about 85%, 90%, 95%, 99% or more identical to SEQ ID NO: 1002, or a sequence having one, two, three, four, five or more substitutions, insertions, deletions, or modifications compared to SEQ ID NO: 1002.

In some embodiments, the second nucleic acid sequence encodes an amino acid comprising the sequence of SEQ ID NO: 1001, a sequence at least about 85%, 90%, 95%, 99% or more identical to SEQ ID NO:1001, or a sequence having one, two, three, four, five or more substitutions, insertions, deletions, or modifications compared to SEQ ID NO: 1001.

In some embodiments, the single nucleic acid molecule does not comprise a suicide gene, e.g., an inducible suicide gene.

In some embodiments, the CAR molecule comprises, in an N- to C-terminal orientation, an antigen binding domain that binds to the antigen, a transmembrane domain, and an intracellular signaling domain. In some embodiments, the antigen binding domain is connected to the transmembrane domain by a hinge domain. In some embodiments, the CAR molecule further comprises a leader sequence. In some embodiments, the antigen is a solid tumor antigen.

In some embodiments, the antigen is chosen from: CD19; CD123; CD22; CD30; CD171; CS-1; C-type lectin-like molecule-1, CD33; epidermal growth factor receptor variant III (EGFRvIII); ganglioside G2 (GD2); ganglioside GD3; TNF receptor family member; B-cell maturation antigen; Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2; Mesothelin; Interleukin 11 receptor alpha (IL-11Ra); prostate stem cell antigen (PSCA); Protease Serine 21; vascular endothelial growth factor receptor 2 (VEGFR2); Lewis (Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene polypeptide consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3; transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1, melanoma antigen recognized by T cells 1; Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like, Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); or immunoglobulin lambda-like polypeptide 1 (IGLL1).

In some embodiments, the antigen is selected from mesothelin, EGFRvIII, GD2, Tn antigen, sTn antigen, Tn-O-Glycopeptides, sTn-O-Glycopeptides, PSMA, CD97, TAG72, CD44v6, CEA, EPCAM, KIT, IL-13Ra2, leguman, GD3, CD171, IL-11Ra, PSCA, MAD-CT-1, MAD-CT-2, VEGFR2, LewisY, CD24, PDGFR-beta, SSEA-4, folate receptor alpha, ERBBs (e.g., ERBB2), Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, NY-ESO-1, GPR20, Ly6k, OR51E2, TARP, or GFRα4.

In some embodiments, the antigen is chosen from CD19, CD22, BCMA, CD20, CD123, EGFRvIII, or mesothelin.

In some embodiments, the antigen comprises mesothelin.

In some embodiments, the antigen comprises CD19.

In some embodiments, the antigen comprises BCMA.

In some embodiments, the transmembrane domain of the CAR molecule comprises a transmembrane domain of a protein chosen from the alpha, beta or zeta chain of the T-cell receptor, CD28, CD3 epsilon, CD45, CD4, CD5, CD8, CD9, CD16, CD22, CD33, CD37, CD64, CD80, CD86, CD123, CD134, CD137 or CD154. In some embodiments, the transmembrane domain comprises a transmembrane domain of CD8. In some embodiments, the transmembrane domain comprises the amino acid sequence of SEQ ID NO: 1026 (or a sequence at least about 85%, 90%, 95%, 99% or more identical thereto, and/or having one, two, three or more substitutions, insertions or deletions, e.g., conserved substitutions).

In some embodiments, the intracellular signaling domain of the CAR molecule comprises a primary signaling domain. In some embodiments, the primary signaling domain comprises a functional signaling domain derived from CD3 zeta, TCR zeta, FcR gamma, FcR beta, CD3 gamma, CD3 delta, CD3 epsilon, CD5, CD22, CD79a, CD79b, CD278 (ICOS), FcεRI, DAP10, DAP12, or CD66d. In some embodiments, the primary signaling domain comprises a functional signaling domain derived from CD3 zeta. In some embodiments, the primary signaling domain comprises the amino acid sequence of SEQ ID NO: 1034 or 1037 (or a sequence at least about 85%, 90%, 95%, 99% or more identical thereto, and/or having one, two, three or more substitutions, insertions or deletions, e.g., conserved substitutions).

In some embodiments, the intracellular signaling domain of the CAR molecule comprises a costimulatory domain. In some embodiments, the costimulatory domain comprises a functional signaling domain derived from a MHC class I molecule, TNF receptor protein, Immunoglobulin-like protein, cytokine receptor, integrin, signalling lymphocytic activation molecule (SLAM), activating NK cell receptor, BTLA, a Toll ligand receptor, OX40, CD2, CD7, CD27, CD28, CD30, CD40, ICAM-1, 4-1BB (CD137), B7-H3, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD28-OX40, CD28-4-1BB, or a ligand that specifically binds with CD83. In some embodiments, the costimulatory domain comprises a functional signaling domain derived from 4-1BB. In some embodiments, the costimulatory domain comprises the amino acid sequence of SEQ ID NO: 1029 (or a sequence at least about 85%, 90%, 95%, 99% or more identical thereto, and/or having one, two, three or more substitutions, insertions or deletions, e.g., conserved substitutions).

In another aspect, disclosed herein is a CAR IL-15/IL15R polypeptide comprising:
(i) a chimeric antigen receptor (CAR) molecule that binds to an antigen (e.g., an antigen described herein, e.g., CD19, mesothelin, or BCMA);
(ii) an IL-15 receptor (IL-15R) molecule; and
(iii) an IL-15 molecule.

In some embodiments, (i)-(iii) are expressed in the same frame on a single polypeptide chain.

In some embodiments, the polypeptide has the following arrangement in an N- to C-terminal orientation: CAR molecule-a first linker-IL-15R molecule-a second linker-IL-15 molecule; or CAR molecule-a first linker-IL-15 molecule-a second linker-IL-15R molecule, wherein the first and second linkers are different.

In some embodiments, the polypeptide has the following arrangement in an N- to C-terminal orientation: IL-15R molecule-a first linker-IL-15 molecule-a second linker-CAR molecule; or IL-15 molecule-a first linker-IL-15R molecule-a second linker-CAR molecule, e.g., wherein the first and second linkers are different.

In some embodiments, the first linker comprises a P2A site and the second linker comprises a T2A site, wherein:
(i) the first linker comprises the amino acid sequence of SEQ ID NO: 1479 (or a sequence at least about 85%, 90%, 95%, 99% or more identical thereto, and/or having one, two, three or more substitutions, insertions, deletions, or modifications), and/or
(ii) the second linker comprises the amino acid sequence of SEQ ID NO: 1478 (or a sequence at least about 85%, 90%, 95%, 99% or more identical thereto, and/or having one, two, three or more substitutions, insertions, deletions, or modifications).

In some embodiments, (i)-(iii) are expressed as separate polypeptides.

In some embodiments, (i)-(iii) are all expressed as three separate polypeptides.

In some embodiments, (i) and (ii) are expressed in the same frame on a single polypeptide chain. In some embodiments, (iii) is expressed on a separate polypeptide.

In some embodiments, (i) and (iii) are expressed in the same frame on a single polypeptide chain. In some embodiments, (ii) is expressed on a separate polypeptide.

In some embodiments, (ii) and (iii) are expressed in the same frame on a single polypeptide chain. In some embodiments, (i) is expressed on a separate polypeptide.

In some embodiments, the polypeptide comprises the amino acid sequence of SEQ ID NO: 1002, a sequence at least about 85%, 90%, 95%, 99% or more identical to SEQ ID NO: 1002, or a sequence having one, two, three, four, five or more substitutions, insertions, deletions, or modifications compared to SEQ ID NO: 1002.

In some embodiments, the polypeptide comprises comprising the amino acid sequence of SEQ ID NO: 1001, a sequence at least about 85%, 90%, 95%, 99% or more identical to SEQ ID NO:1001, or a sequence having one, two, three, four, five or more substitutions, insertions, deletions, or modifications compared to SEQ ID NO: 1001.

In some embodiments, the polypeptide comprising the CAR molecule comprises, in an N- to C-terminal orientation, an antigen binding domain that binds to the antigen, a transmembrane domain, and an intracellular signaling domain, optionally wherein the antigen binding domain is connected to the transmembrane domain by a hinge domain.

In some embodiments, (i)-(iii) are in a single composition.

In one aspect, disclosed herein is a cell, e.g., an immune effector cell, comprising a nucleic acid molecule disclosed herein or a polypeptide disclosed herein.

In another aspect, disclosed herein is a vector, e.g., a lentiviral vector, comprising a comprising a nucleic acid molecule disclosed herein.

In yet another aspect, the disclosure provides a method of making a population of immune effector cells expressing Chimeric Antigen Receptor (CAR) molecule, IL-15R molecule and IL-15 molecule (“CAR IL-15/IL-15R expressing cells”), comprising:
a) providing a population of immune effector cells, e.g., T cells or NK cells;
b) contacting the population of immune effector cells with a nucleic acid molecule disclosed herein, or a vector disclosed herein; and
c) maintaining the cells under conditions that allow expression of the CAR polypeptide, thereby making a population of CAR IL-15/IL-15R expressing immune effector cells.

In some embodiments, the nucleic acid is DNA or RNA.

In some embodiments, (b) comprises performing lentiviral transduction to deliver the nucleic acid to the immune effector cells.

In some embodiments, the CAR IL-15/IL-15R expressing cells comprise a single composition.

In some embodiments, the method further comprises contacting the population of cells with a ligand, e.g., with an extracellular ligand, that binds to the CAR molecule, thereby stimulating the population of cells. In some embodiments, the ligand comprises a cognate antigen molecule or an antibody molecule that binds to the CAR molecule. In some embodiments, the ligand, e.g., cognate antigen molecule, is immobilized, e.g., on a substrate, e.g., a bead or a cell, or is soluble. In some embodiments, the population of cells is contacted, e.g., stimulated, with the cognate antigen molecule at least 1 time, 2 times, 3 times, 4 times, 5 times, 6 times, 7 times or 8 times, e.g., 4 times, wherein each contact period, e.g., stimulation, lasts for about 1 week. In some embodiments, the method further comprises contacting the population of cells with an IL-21 molecule. In some embodiments, the IL-21 molecule is provided at an amount of at least 5, 10, 15, 20, 30, 40, 50 or 100 μg/ml, e.g., 10 μg/ml. In some embodiments, the IL-21 molecule promotes a naïve T cell phenotype, e.g., CD45RO− CCR7+.

In some embodiments, following contacting, e.g., stimulating, with the cognate antigen molecule, the population of cells is not contacted with an exogenous cytokine or cognate antigen molecule.

In some embodiments, the population of cells is maintained for at least 1, 2, 3, 4, 5, 6, 7, 8, 9, 10, 11, 12, 13, 14, 15 or 20 weeks, e.g., 10 weeks.

In some embodiments, the population of cells has one, two, three, four or all of the following characteristics:
(i) no or minimal loss in viability as measured by an assay of Example 1;
(ii) an antigen specific response, e.g., maintenance of an antigen specific response, as measured by an assay of Example 1;
(iii) ability to induce degranulation, e.g., maintenance of an ability to induce degranulation, e.g., as measured by CD107a expression, as measured by an assay of Example 1;
(iv) ability to induce IFN-g release, e.g., maintenance of an ability to induce IFN-g release, e.g., as measured by IFN-g expression in an assay of Example 1; or
(v) mitochondrial activity, e.g., maintenance of mitochondrial activity, as measured by an assay of Example 1,
compared to an otherwise similar CAR IL-15/IL-15R expressing population prior to culturing without cytokine or antigen, e.g., at four weeks after contacting, e.g., stimulation, with a cognate antigen molecule.

In some embodiments, any of the methods disclosed herein results in an increase in the population of cells expressing CD45RO-CCR7+, e.g., by about at least 10%, 20%, 30%, 40%, 50%, 60%, 70%, 80%, 90%, 95%, 100% or greater, compared to a population of immune effector cells contacted with a nucleic acid molecule encoding a CAR molecule without IL-15R and/or IL-15.

In some embodiments, any of the methods of making disclosed herein comprises
(i) expanding the population of cells, e.g., for at least 1, 2, 3, 4, 5, 6, 7, 8, 9, 10, 11, 12, 13, 14, 15, 16, 17, 18, 19, 20, 21, 22, 23, 24, 25, 26, 27, or 28 days or for 1-7, 7-14, 14-21, or 14-28 days; or
(ii) expanding the population of cells, e.g., by at least, 5, 10, 20, 30, 40, 50, 60, 70, 80, 90, or 100-fold change in cell number or more, e.g., up to about 40 or 50-fold, e.g., under growth conditions of Example 1.

In some embodiments, the population of CAR IL-15/IL-15R expressing cell exhibits enhanced anti-tumor efficacy compared to a population of cells expressing a CAR molecule, as measured by an assay of Example 1.

In one aspect, disclosed herein is a method of evaluating a population of CAR IL-15/IL-15R expressing cells, comprising measuring the level, e.g., activity or expression level, of CD45RO and CCR7 in the population of cells, wherein:
a low level of CD45RO expression (e.g., CD45RO−) and a high level of CCR7 expression (e.g., CCR7+) is indicative that the sample is suitable for treatment; and
a high level of CD45RO expression (e.g., CD45RO+) and a low level of CCR7 expression (e.g., CCR7−) is indicative that the sample is not suitable for treatment
   thereby evaluating the CAR-IL-15 complex expressing cell.

In another aspect, the disclosure provides, a cell, e.g., an immune effector cell, e.g., a T cell or NK cell, comprising:
(i) a first nucleic acid sequence encoding a chimeric antigen receptor (CAR) molecule that binds to an antigen (e.g., an antigen described herein, e.g., CD19, mesothelin or BCMA);
(ii) a second nucleic acid sequence encoding an IL-15 receptor (IL-15R) molecule; and
(iii) a third nucleic acid sequence encoding an IL-15 molecule.

In some embodiments, the IL-15R molecule and IL-15 molecule are expressed in the same cell as the CAR molecule.

In some embodiments, the first nucleic acid sequence, the second nucleic acid sequence, and the third nucleic acid sequence are disposed on a single nucleic acid molecule, e.g., a vector, e.g., a viral vector, e.g., a lentivirus vector.

In some embodiments, the single nucleic acid molecule has the following arrangement in an N- to C-terminal orientation:
(i) the first nucleic acid sequence-a first linker-the second nucleic acid sequence-a second linker-the third nucleic acid sequence; or
(ii) the first nucleic acid sequence-a first linker-the third nucleic acid sequence-a second linker-the second nucleic acid sequence,
wherein the first and second linkers are different.

In some embodiments, the single nucleic acid molecule has the following arrangement in an N- to C-terminal orientation:
(iii) the second nucleic acid sequence-a first linker-the third nucleic acid sequence-a second linker-the first nucleic acid sequence; or
(iv) the third nucleic acid sequence-a first linker-the second nucleic acid sequence-a second linker-the first nucleic acid sequence,
wherein the first and second linkers are different.

In some embodiments, the linker encodes a self-cleavage site, e.g., a P2A site, a T2A site, an E2A site, or an F2A site.

In some embodiments, the first nucleic acid sequence, second nucleic acid sequence and third nucleic acid sequence are disposed on separate nucleic acid molecules, e.g., separate vectors, e.g., separate viral vectors, e.g., separate lentivirus vectors.

In some embodiments, the first nucleic acid sequence is disposed on a first nucleic acid molecule, e.g., a first vector, e.g., a first viral vector, e.g., a first lentivirus vector. In some embodiments, the second nucleic acid sequence is disposed on a second nucleic acid molecule, e.g., a second vector, e.g., a second viral vector, e.g., a second lentivirus vector. In some embodiments, the third nucleic acid sequence is disposed on a third nucleic acid molecule, e.g., a third vector, e.g., a third viral vector, e.g., a third lentivirus vector.

In some embodiments, the first nucleic acid sequence and the second nucleic acid sequence are disposed on a first nucleic acid molecule, e.g., a first vector, e.g., a first viral vector, e.g., a first lentivirus vector. In some embodiments, the third nucleic acid sequence is disposed on a second nucleic acid molecule, e.g., a second vector, e.g., a second viral vector, e.g., a second lentivirus vector.

In some embodiments, the first nucleic acid sequence and the third nucleic acid sequence are disposed on a first nucleic acid molecule, e.g., a first vector, e.g., a first viral vector, e.g., a first lentivirus vector. In some embodiments, the second nucleic acid sequence is disposed on a second nucleic acid molecule, e.g., a second vector, e.g., a second viral vector, e.g., a second lentivirus vector.

In some embodiments, the first nucleic acid sequence is disposed on a first nucleic acid molecule, e.g., a first vector, e.g., a first viral vector, e.g., a first lentivirus vector. In some embodiments, the second nucleic acid sequence and the third nucleic acid sequence are disposed on a second nucleic acid molecule, e.g., a second vector, e.g., a second viral vector, e.g., a second lentivirus vector.

In some embodiments, the CAR molecule comprises, in an N- to C-terminal orientation, an antigen binding domain that binds to the antigen, a transmembrane domain, and an intracellular signaling domain. In some embodiments, the antigen binding domain is connected to the transmembrane domain by a hinge domain.

In yet another aspect, disclosed herein is a method of treating a subject having a disease associated with expression of an antigen, e.g., a tumor antigen described herein, e.g., CD19, mesothelin or BCMA, comprising administering to the subject an effective number of a population of cells disclosed herein, or a population of cells comprising a nucleic acid molecule disclosed herein (e.g., a population of cells comprising one or more nucleic acid molecules disclosed herein), or a population of cells comprising a polypeptide disclosed herein (e.g., a population of cells comprising one or more polypeptides disclosed herein).

In a further aspect, disclosed herein, is a method of providing an anti-cancer immune response in a subject having a disease associated with expression of an antigen, e.g., a tumor antigen described herein, e.g., CD19, mesothelin or BCMA, comprising administering to the subject an effective number of a population of cells disclosed herein, or a population of cells comprising a nucleic acid molecule disclosed herein (e.g., a population of cells comprising one or more nucleic acid molecules disclosed herein), or a population of cells comprising a polypeptide disclosed herein (e.g., a population of cells comprising one or more polypeptides disclosed herein).

In some embodiments, the disease associated with expression of an antigen, e.g., a tumor antigen described herein, e.g., CD19, mesothelin or BCMA, is a cancer.

In some embodiments, cancer is a solid tumor. In some embodiments, the cancer is chosen from mesothelioma (e.g., malignant pleural mesothelioma); lung cancer (e.g., non-small cell lung cancer, small cell lung cancer, squamous cell lung cancer, or large cell lung cancer); pancreatic cancer (e.g., pancreatic ductal adenocarcinoma, or metastatic pancreatic ductal adenocarcinoma (PDA)); esophageal cancer (e.g., esophageal adenocarcinoma), ovarian cancer (e.g., serous epithelial ovarian cancer), breast cancer, colorectal cancer, bladder cancer, glioblastoma, prostate cancer, cervical cancer, skin cancer, melanoma, renal cancer, liver cancer, brain cancer, thymoma, sarcoma, carcinoma, uterine cancer, kidney cancer, gastrointestinal cancer, urothelial cancer, pharynx cancer, head and neck cancer, rectal cancer, or any combination thereof.

In some embodiments, the cancer is a hematological cancer, e.g., a hematological cancer chosen from a leukemia or lymphoma, e.g., the cancer is chosen from chronic lymphocytic leukemia (CLL), mantle cell lymphoma (MCL), multiple myeloma, acute lymphoblastic leukemia (ALL), Hodgkin lymphoma, B-cell acute lymphoblastic leukemia (BALL), T-cell acute lymphoblastic leukemia (TALL), small lymphocytic lymphoma (SLL), B cell prolymphocytic leukemia, blastic plasmacytoid dendritic cell neoplasm, Burkitt's lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoblastic tissue), Marginal zone lymphoma, myelodysplasia, myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+ large B-cell lymphoma, large B-cell lymphoma arising in HHV8-associated multicentric Castleman disease, primary effusion lymphoma, B-cell lymphoma, acute myeloid leukemia (AML), or unclassifiable lymphoma.

In some embodiments, a cell described herein is administered systemically or locally.

In some embodiments, the subject has a tumor, e.g., a solid tumor and the cell, is administered through intratumoral administration.

In some embodiments, the method further comprises administering a third therapeutic agent, e.g., as described herein. In some embodiments, the third therapeutic agent is a checkpoint modulator. In some embodiments, the third therapeutic agent is an anti-PD-1 antibody molecule, an anti-PD-L1 antibody molecule, an anti-CTLA-4 antibody molecule, an anti-TIM-3 antibody molecule, or an anti-LAG-3 molecule.

In one aspect, disclosed herein, is method of evaluating or predicting a subject's responsiveness to a CAR-expressing cell therapy, comprising acquiring a value for the level, e.g., activity or expression level, of CD45RO and CCR7 in the population of cells, wherein:
a low level of CD45RO expression (e.g., CD45RO−) and a high level of CCR7 expression (e.g., CCR7+) is indicative or predictive of increased responsiveness of the subject to the CAR IL-15/IL-15R-expressing cell therapy; and
a high level of CD45RO expression (e.g., CD45RO+) and a low level of CCR7 expression (e.g., CCR7−) is indicative or predictive of decreased responsiveness of the subject to the CAR IL-15/IL-15R-expressing cell therapy,
thereby evaluating or predicting the subject's responsiveness to the CAR IL-15/IL-15R expressing cell therapy.

Although methods and materials similar or equivalent to those described herein can be used in the practice or testing of the present invention, suitable methods and materials are described below. All publications, patent applications, patents, and other references (e.g., sequence database reference numbers) mentioned herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Feb. 13, 2019. When one gene or protein references a plurality of sequence accession numbers, all of the sequence variants are encompassed.

In addition, the materials, methods, and examples are illustrative only and not intended to be limiting. Headings, sub-headings or numbered or lettered elements, e.g., (a), (b), (i) etc., are presented merely for ease of reading. The use of headings or numbered or lettered elements in this document does not require the steps or elements be performed in alphabetical order or that the steps or elements are necessarily discrete from one another. Other features, objects, and advantages of the invention will be apparent from the description and drawings, and from the claims.

### BRIEF DESCRIPTION OF THE DRAWINGS

FIG. 1 shows schematics of lentiviral constructs used in Example 1. The CAR19 construct comprised a chimeric antigen receptor (CAR) targeting huCD19 with a CD8 leader (L), CD8 hinge (H), transmembrane domain (M), and intracellular domains (ICD) CD137 (4-1BB) and CD3 zeta (CD3-z). The CAR19+IL-15R/-IL15 construct comprised the CAR19 construct and a P2A system, IL-15R, a T2A system and IL-15. The IL-15R/-IL15 construct comprised IL-15R, a T2A system and IL-15. The CARmeso+IL-15R/-IL15 construct comprised the CARmeso construct and a P2A system, IL-15R, a T2A system and IL-15. The CARmeso construct comprised a chimeric antigen receptor (CAR) targeting human mesothelin, with a CD8 leader (L), CD8 hinge (H), transmembrane domain (M), and intracellular domains (ICD) CD137 (4-1BB) and CD3 zeta (CD3-z). Lentiviral constructs used to express chimeric antigen receptors (CAR) targeting huCD19 (CAR19) or hu mesothelin (CARmeso) with a CD8 leader (L), CD8 hinge (H), transmembrane domain (M), and intracellular domains (ICD) CD137 (4-1BB) and CD3 zeta (CD3-z). IL-15Ra and IL-15 were co-expressed using the T2A system (T) which was co-expressed with the CAR using the P2A system (P). Lentiviral constructs were pseudotyped with VSV/G.

FIGS. 2A, 2B, 2C and 2D show CAR modified T cell expansions. FIG. 2A shows CAR19 T cells, and FIG. 2B shows CARmeso T cells, normalized for transductions efficiency with similar mean fluorescent expression. The CAR T cells were expanded through four rounds of re-stimulation with Nalm6 (N6) or K562-meso cells. Some groups were cultured with no antigen (No N6, No K-meso, or No Ag), while others were supplemented with 100 U/ml IL-2 and/or 10 ug/ml IL-21 as indicated in the figure. FIGS. 2C and 2D show results after 4 weeks of re-stimulation. After re-stimulation, T cells were cultured alone, e.g., without cytokines, re-placing half media volume every 3-4 days without any cytokine supplements for up to an additional 13 weeks. T cell growth expansions monitored by number and size using a Coulter Multisizer IV.

FIG. 3 shows flow cytometry plots depicting phenotypes of CAR T cells after expansion. CAR19 T cells cultures at the end of weeks 1, 3 and 4 were stained for CCR7 and CD45 expression and the T cell subsets were defined as follows: naïve (CCR7+/CD45RO−), Central memory (CCR7+/CD45RO+), Effector memory (CCR7−/CD45RO+) and terminal effectors (CCR7−/CD45RO−). T cell cultures at end of week 4 was >90% CD8+.

FIG. 4 shows flow cytometry plots depicting effector function of CAR T cells. T cell populations at week 4 and week 13 were co-cultured with or without either K562 cells expressing CD19 (K562-CD19) or mesothelin (K562-Meso), or PMA and ionomycin and incubated for 6 hours with CD107a, monesin and Brefeldin A. The resulting T cell populations were fluorescently stained for life/dead, surface expression of CD3, CD4, CD8 and followed by intracellular staining detecting IFN-γ.

FIGS. 5A, 5B, 5C, and 5D are graphs depicting long term tumor control in CARmeso T cells co-expressing IL-15R/IL-15 compared to CARmeso T cells. One million were injected subcutaneously into the right flanks of NSG mice to establish tumors. Tumor growth was monitored by caliper measurements. On day 38 (tumors averaged 250 mm³) either 1×10⁵ or 3×10⁵ CAR+ T cells were injected intratumorally (IT) and tumors were monitored twice weekly.

### DESCRIPTION

### Definitions

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of +20% or in some instances ±10%, or in some instances ±5%, or in some instances ±1%, or in some instances ±0.1% from the specified value, as such variations are appropriate to perform the disclosed methods.

The term “Chimeric Antigen Receptor” or alternatively a “CAR” refers to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule as defined below. In some embodiments, the domains in the CAR polypeptide construct are in the same polypeptide chain, e.g., comprise a chimeric fusion protein. In some embodiments, the domains in the CAR polypeptide construct are not contiguous with each other, e.g., are in different polypeptide chains, e.g., as provided in an RCAR as described herein.

In one aspect, the cytoplasmic signaling domain comprises a primary signaling domain (e.g., a primary signaling domain of CD3-zeta). In one aspect, the cytoplasmic signaling domain further comprises one or more functional signaling domains derived from at least one costimulatory molecule as defined below. In one aspect, the costimulatory molecule is chosen from 41BB (i.e., CD137), CD27, ICOS, and/or CD28. In one aspect, the CAR comprises a chimeric fusion protein comprising an extracellular antigen recognition domain, a transmembrane domain and an intracellular signaling domain comprising a functional signaling domain derived from a stimulatory molecule. In one aspect, the CAR comprises a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule. In one aspect, the CAR comprises a chimeric fusion protein comprising an extracellular antigen recognition domain, a transmembrane domain and an intracellular signaling domain comprising two functional signaling domains derived from one or more co-stimulatory molecule(s) and a functional signaling domain derived from a stimulatory molecule. In one aspect, the CAR comprises a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule. In one aspect the CAR comprises an optional leader sequence at the amino-terminus (N-ter) of the CAR fusion protein. In one aspect, the CAR further comprises a leader sequence at the N-terminus of the extracellular antigen recognition domain, wherein the leader sequence is optionally cleaved from the antigen recognition domain (e.g., an scFv) during cellular processing and localization of the CAR to the cellular membrane.

A CAR that comprises an antigen binding domain (e.g., an scFv, a single domain antibody, or TCR (e.g., a TCR alpha binding domain or TCR beta binding domain)) that targets a specific tumor marker X, wherein X can be a tumor marker as described herein, is also referred to as XCAR. For example, a CAR that comprises an antigen binding domain that targets CD19 is referred to as CD19CAR. The CAR can be expressed in any cell, e.g., an immune effector cell as described herein (e.g., a T cell or an NK cell).

The term “signaling domain” refers to the functional portion of a protein which acts by transmitting information within the cell to regulate cellular activity via defined signaling pathways by generating second messengers or functioning as effectors by responding to such messengers.

The term “antibody,” as used herein, refers to a protein, or polypeptide sequence derived from an immunoglobulin molecule, which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

The term “antibody fragment” refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)2, and Fv fragments, scFv antibody fragments, linear antibodies, single domain antibodies such as sdAb (either VL or VH), camelid VHH domains, and multi-specific molecules formed from antibody fragments such as a bivalent fragment comprising two or more, e.g., two, Fab fragments linked by a disulfide brudge at the hinge region, or two or more, e.g., two isolated CDR or other epitope binding fragments of an antibody linked. An antibody fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antibody fragments can also be grafted into scaffolds based on polypeptides such as a fibronectin type III (Fn3) (see U.S. Pat. No. 6,703,199, which describes fibronectin polypeptide minibodies).

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The terms “complementarity determining region” or “CDR,” as used herein, refer to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme), or a combination thereof. Under the Kabat numbering scheme, in some embodiments, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under the Chothia numbering scheme, in some embodiments, the CDR amino acids in the VH are numbered 26-32 (HCDR1), 52-56 (HCDR2), and 95-102 (HCDR3); and the CDR amino acid residues in the VL are numbered 26-32 (LCDR1), 50-52 (LCDR2), and 91-96 (LCDR3). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-102 (HCDR3) in a VH, e.g., a mammalian VH, e.g., a human VH; and amino acid residues 24-34 (LCDR1), 50-56 (LCDR2), and 89-97 (LCDR3) in a VL, e.g., a mammalian VL, e.g., a human VL.

The portion of the CAR composition of the invention comprising an antibody or antibody fragment thereof may exist in a variety of forms, for example, where the antigen binding domain is expressed as part of a polypeptide chain including, for example, a single domain antibody fragment (sdAb), a single chain antibody (scFv), or e.g., a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426). In one aspect, the antigen binding domain of a CAR composition of the invention comprises an antibody fragment. In a further aspect, the CAR comprises an antibody fragment that comprises an scFv.

As used herein, the term “binding domain” or “antibody molecule” (also referred to herein as “anti-target binding domain”) refers to a protein, e.g., an immunoglobulin chain or fragment thereof, comprising at least one immunoglobulin variable domain sequence. The term “binding domain” or “antibody molecule” encompasses antibodies and antibody fragments. In an embodiment, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (K) and lambda (2) light chains refer to the two major antibody light chain isotypes.

The term “recombinant antibody” refers to an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

The term “anti-tumor effect” refers to a biological effect which can be manifested by various means, including but not limited to, e.g., a decrease in tumor volume, a decrease in the number of tumor cells, a decrease in the number of metastases, an increase in life expectancy, decrease in tumor cell proliferation, decrease in tumor cell survival, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

The term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the peptides, polynucleotides, cells and antibodies in prevention of the occurrence of cancer in the first place. The term “anti-tumor effect” refers to a biological effect which can be manifested by various means, including but not limited to, e.g., a decrease in tumor volume, a decrease in the number of tumor cells, a decrease in tumor cell proliferation, or a decrease in tumor cell survival. The term “autologous” refers to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “xenogeneic” refers to a graft derived from an animal of a different species.

The term “apheresis” as used herein refers to the art-recognized extracorporeal process by which the blood of a donor or patient is removed from the donor or patient and passed through an apparatus that separates out selected particular constituent(s) and returns the remainder to the circulation of the donor or patient, e.g., by retransfusion. Thus, in the context of “an apheresis sample” refers to a sample obtained using apheresis.

The term “combination” refers to either a fixed combination in one dosage unit form, or a combined administration where a compound of the present invention and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g. a compound of the present invention and a combination partner, are both administered to a patient simultaneously in the form of a single entity or dosage. The term “non-fixed combination” means that the active ingredients, e.g. a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. Preferred cancers treated by the methods described herein include multiple myeloma, Hodgkin's lymphoma or non-Hodgkin's lymphoma.

The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

“Derived from” as that term is used herein, indicates a relationship between a first and a second molecule. It generally refers to structural similarity between the first molecule and a second molecule and does not connotate or include a process or source limitation on a first molecule that is derived from a second molecule. For example, in the case of an intracellular signaling domain that is derived from a CD3zeta molecule, the intracellular signaling domain retains sufficient CD3zeta structure such that is has the required function, namely, the ability to generate a signal under the appropriate conditions. It does not connotate or include a limitation to a particular process of producing the intracellular signaling domain, e.g., it does not mean that, to provide the intracellular signaling domain, one must start with a CD3zeta sequence and delete unwanted sequence, or impose mutations, to arrive at the intracellular signaling domain.

The phrase “disease associated with expression of an antigen, e.g., a tumor antigen” includes, but is not limited to, a disease associated with a cell which expresses the antigen (e.g., wild-type or mutant antigen) or condition associated with a cell which expresses the antigen (e.g., wild-type or mutant antigen) including, e.g., proliferative diseases such as a cancer or malignancy or a precancerous condition such as a myelodysplasia, a myelodysplastic syndrome or a preleukemia; or a noncancer related indication associated with a cell which expresses the antigen (e.g., wild-type or mutant antigen). For the avoidance of doubt, a disease associated with expression of the antigen may include a condition associated with a cell which does not presently express the antigen, e.g., because expression of the antigen has been downregulated, e.g., due to treatment with a molecule targeting the antigen, but which at one time expressed the antigen. In some embodiments, the disease associated with expression of an antigen, e.g., a tumor antigen is a cancer (e.g., a solid cancer or a hematological cancer), a viral infection (e.g., HIV, a fungal infection, e.g., C. neoformans), an autoimmune disease (e.g. rheumatoid arthritis, system lupus erythematosus (SLE or lupus), pemphigus vulgaris, and Sjogren's syndrome; inflammatory bowel disease, ulcerative colitis; transplant-related allospecific immunity disorders related to mucosal immunity; and unwanted immune responses towards biologics (e.g., Factor VIII) where humoral immunity is important).

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a CAR of the invention can be replaced with other amino acid residues from the same side chain family and the altered CAR can be tested using the functional assays described herein.

The term “stimulation,” refers to a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-β, and/or reorganization of cytoskeletal structures, and the like.

The term “stimulatory molecule,” refers to a molecule expressed by a T cell that provides the primary cytoplasmic signaling sequence(s) that regulate primary activation of the TCR complex in a stimulatory way for at least some aspect of the T cell signaling pathway. In some embodiments, the ITAM-containing domain within the CAR recapitulates the signaling of the primary TCR independently of endogenous TCR complexes. In one aspect, the primary signal is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM. Examples of an ITAM containing primary cytoplasmic signaling sequence that is of particular use in the invention includes, but is not limited to, those derived from TCR zeta, FcR gamma, FcR beta, CD3 gamma, CD3 delta, CD3 epsilon, CD5, CD22, CD79a, CD79b, CD278 (also known as “ICOS”), FcεRI and CD66d, DAP10 and DAP12. In a specific CAR of the invention, the intracellular signaling domain in any one or more CARS of the invention comprises an intracellular signaling sequence, e.g., a primary signaling sequence of CD3-zeta. The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T-cells may recognize these complexes using their T-cell receptors (TCRs). APCs process antigens and present them to T-cells.

An “intracellular signaling domain,” as the term is used herein, refers to an intracellular portion of a molecule. In embodiments, the intracellular signal domain transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

The intracellular signaling domain generates a signal that promotes an immune effector function of the CAR containing cell, e.g., a CART cell. Examples of immune effector function, e.g., in a CART cell, include cytolytic activity and helper activity, including the secretion of cytokines.

In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, CD278 (also known as “ICOS”), FcεRI, CD66d, DAP10 and DAP12.

The term “zeta” or alternatively “zeta chain”, “CD3-zeta” or “TCR-zeta” refers to CD247. Swiss-Prot accession number P20963 provides exemplary human CD3 zeta amino acid sequences. A “zeta stimulatory domain” or alternatively a “CD3-zeta stimulatory domain” or a “TCR-zeta stimulatory domain” refers to a stimulatory domain of CD3-zeta or a variant thereof (e.g., a molecule having mutations, e.g., point mutations, fragments, insertions, or deletions). In one embodiment, the cytoplasmic domain of zeta comprises residues 52 through 164 of GenBank Acc. No. BAG36664.1 or a variant thereof (e.g., a molecule having mutations, e.g., point mutations, fragments, insertions, or deletions). In one embodiment, the “zeta stimulatory domain” or a “CD3-zeta stimulatory domain” is the sequence provided as SEQ ID NO: 1034 or 1037 or a variant thereof (e.g., a molecule having mutations, e.g., point mutations, fragments, insertions, or deletions).

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to an MHC class I molecule, TNF receptor proteins, Immunoglobulin-like proteins, cytokine receptors, integrins, signaling lymphocytic activation molecules (SLAM proteins), activating NK cell receptors, BTLA, Toll ligand receptor, OX40, CD2, CD7, CD27, CD28, CD30, CD40, ICAM-1, LFA-1 (CD11a/CD18), 4-1BB (CD137), B7-H3, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD28-OX40, CD28-4-1BB, and a ligand that specifically binds with CD83.

A costimulatory intracellular signaling domain refers to the intracellular portion of a costimulatory molecule.

The intracellular signaling domain can comprise the entire intracellular portion, or the entire native intracellular signaling domain, of the molecule from which it is derived, or a functional fragment thereof.

The term “4-1BB” refers to CD137 or Tumor necrosis factor receptor superfamily member 9. Swiss-Prot accession number P20963 provides exemplary human 4-1BB amino acid sequences. A “4-1BB costimulatory domain” refers to a costimulatory domain of 4-1BB, or a variant thereof (e.g., a molecule having mutations, e.g., point mutations, fragments, insertions, or deletions). In one embodiment, the “4-1BB costimulatory domain” is the sequence provided as SEQ ID NO: 1029 or a variant thereof (e.g., a molecule having mutations, e.g., point mutations, fragments, insertions, or deletions).

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloic-derived phagocytes.

“Immune effector function or immune effector response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

The term “effector function” refers to a specialized function of a cell. Effector function of a T cell, for example, may be cytolytic activity or helper activity including the secretion of cytokines.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “effective amount” or “therapeutically effective amount” are used interchangeably herein, and refer to an amount of a compound, formulation, material, or composition, as described herein effective to achieve a particular biological result.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence. In some embodiments, expression comprises translation of an mRNA introduced into a cell.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementarity-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321:522-525, 1986; Reichmann et al., Nature, 332:323-329, 1988; Presta, Curr. Op. Struct. Biol., 2:593-596, 1992.

“Fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “parenteral” administration of an immunogenic composition includes, e.g., subcutaneous (s.c.), intravenous (i.v.), intramuscular (i.m.), or intrasternal injection, intratumoral, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions, e.g., conservative substitutions), alleles, orthologs, SNPs, and complementary sequences as well as the sequence explicitly indicated. Specifically, degenerate codon substitutions, e.g., conservative substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

The terms “cancer associated antigen” or “tumor antigen” interchangeably refers to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells, e.g., a lineage marker, e.g., CD19 on B cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell. In some embodiments, the CARs of the present invention includes CARs comprising an antigen binding domain (e.g., antibody or antibody fragment) that binds to a MHC presented peptide. Normally, peptides derived from endogenous proteins fill the pockets of Major histocompatibility complex (MHC) class I molecules, and are recognized by T cell receptors (TCRs) on CD8+T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, e.g., Sastry et al., J Virol. 2011 85(5):1935-1942; Sergeeva et al., Blood, 2011 117(16):4262-4272; Verma et al., J Immunol 2010 184(4):2156-2165; Willemsen et al., Gene Ther 2001 8(21):1601-1608; Dao et al., Sci Transl Med 2013 5(176):176ra33; Tassev et al., Cancer Gene Ther 2012 19(2):84-100). For example, TCR-like antibody can be identified from screening a library, such as a human scFv phage displayed library.

The term “tumor-supporting antigen” or “cancer-supporting antigen” interchangeably refer to a molecule (typically a protein, carbohydrate or lipid) that is expressed on the surface of a cell that is, itself, not cancerous, but supports the cancer cells, e.g., by promoting their growth or survival e.g., resistance to immune cells. Exemplary cells of this type include stromal cells and myeloid-derived suppressor cells (MDSCs). The tumor-supporting antigen itself need not play a role in supporting the tumor cells so long as the antigen is present on a cell that supports cancer cells.

The term “flexible polypeptide linker” or “linker” as used in the context of an scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)n, where n is a positive integer equal to or greater than 1. For example, n=1, n=2, n=3, n=4, n=5 and n=6, n=7, n=8, n=9 and n=10 (SEQ ID NO: 1009). In one embodiment, the flexible polypeptide linkers include, but are not limited to, (Gly4 Ser)4 (SEQ ID NO: 1010) or (Gly4 Ser)3 (SEQ ID NO: 1011). In another embodiment, the linkers include multiple repeats of (Gly2Ser), (GlySer) or (Gly3Ser) (SEQ ID NO: 1012). Also included within the scope of the invention are linkers described in WO2012/138475, incorporated herein by reference.

As used herein, a 5′ cap (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA, preferably mRNA, that has been synthesized in vitro. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In the preferred embodiment of a construct for transient expression, the polyA is between 50 and 5000 (SEQ ID NO: 1013), preferably greater than 64, more preferably greater than 100, most preferably greater than 300 or 400. poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more therapies (e.g., one or more therapeutic agents such as a CAR of the invention). In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating”-refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human).

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

In the context of the present invention, “tumor antigen” or “hyperproliferative disorder antigen” or “antigen associated with a hyperproliferative disorder” refers to antigens that are common to specific hyperproliferative disorders. In certain aspects, the hyperproliferative disorder antigens of the present invention are derived from, cancers including but not limited to primary or metastatic melanoma, thymoma, lymphoma, sarcoma, lung cancer, liver cancer, non-Hodgkin lymphoma, Hodgkin lymphoma, leukemias, uterine cancer, cervical cancer, bladder cancer, kidney cancer and adenocarcinomas such as breast cancer, prostate cancer (e.g., castrate-resistant or therapy-resistant prostate cancer, or metastatic prostate cancer), ovarian cancer, pancreatic cancer, and the like, or a plasma cell proliferative disorder, e.g., asymptomatic myeloma (smoldering multiple myeloma or indolent myeloma), monoclonal gammapathy of undetermined significance (MGUS), Waldenstrom's macroglobulinemia, plasmacytomas (e.g., plasma cell dyscrasia, solitary myeloma, solitary plasmacytoma, extramedullary plasmacytoma, and multiple plasmacytoma), systemic amyloid light chain amyloidosis, and POEMS syndrome (also known as Crow-Fukase syndrome, Takatsuki disease, and PEP syndrome).

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, or a ligand, which recognizes and binds with a cognate binding partner (e.g., a stimulatory and/or costimulatory molecule present on a T cell) protein present in a sample, but which antibody or ligand does not substantially recognize or bind other molecules in the sample.

“Regulatable chimeric antigen receptor (RCAR),” as used herein, refers to a set of polypeptides, typically two in the simplest embodiments, which when in an immune effector cell, provides the cell with specificity for a target cell, typically a cancer cell, and with intracellular signal generation. In some embodiments, an RCAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined herein in the context of a CAR molecule. In some embodiments, the set of polypeptides in the RCAR are not contiguous with each other, e.g., are in different polypeptide chains. In some embodiments, the RCAR includes a dimerization switch that, upon the presence of a dimerization molecule, can couple the polypeptides to one another, e.g., can couple an antigen binding domain to an intracellular signaling domain. In some embodiments, the RCAR is expressed in a cell (e.g., an immune effector cell) as described herein, e.g., an RCAR-expressing cell (also referred to herein as “RCARX cell”). In an embodiment the RCARX cell is a T cell, and is referred to as a RCART cell. In an embodiment the RCARX cell is an NK cell, and is referred to as a RCARN cell. The RCAR can provide the RCAR-expressing cell with specificity for a target cell, typically a cancer cell, and with regulatable intracellular signal generation or proliferation, which can optimize an immune effector property of the RCAR-expressing cell. In embodiments, an RCAR cell relies at least in part, on an antigen binding domain to provide specificity to a target cell that comprises the antigen bound by the antigen binding domain.

“Membrane anchor” or “membrane tethering domain”, as that term is used herein, refers to a polypeptide or moiety, e.g., a myristoyl group, sufficient to anchor an extracellular or intracellular domain to the plasma membrane.

“Switch domain,” as that term is used herein, e.g., when referring to an RCAR, refers to an entity, typically a polypeptide-based entity, that, in the presence of a dimerization molecule, associates with another switch domain. The association results in a functional coupling of a first entity linked to, e.g., fused to, a first switch domain, and a second entity linked to, e.g., fused to, a second switch domain. A first and second switch domain are collectively referred to as a dimerization switch. In embodiments, the first and second switch domains are the same as one another, e.g., they are polypeptides having the same primary amino acid sequence, and are referred to collectively as a homodimerization switch. In embodiments, the first and second switch domains are different from one another, e.g., they are polypeptides having different primary amino acid sequences, and are referred to collectively as a heterodimerization switch. In embodiments, the switch is intracellular. In embodiments, the switch is extracellular. In embodiments, the switch domain is a polypeptide-based entity, e.g., FKBP or FRB-based, and the dimerization molecule is small molecule, e.g., a rapalogue. In embodiments, the switch domain is a polypeptide-based entity, e.g., an scFv that binds a myc peptide, and the dimerization molecule is a polypeptide, a fragment thereof, or a multimer of a polypeptide, e.g., a myc ligand or multimers of a myc ligand that bind to one or more myc scFvs. In embodiments, the switch domain is a polypeptide-based entity, e.g., myc receptor, and the dimerization molecule is an antibody or fragments thereof, e.g., myc antibody.

“Dimerization molecule,” as that term is used herein, e.g., when referring to an RCAR, refers to a molecule that promotes the association of a first switch domain with a second switch domain. In embodiments, the dimerization molecule does not naturally occur in the subject, or does not occur in concentrations that would result in significant dimerization. In embodiments, the dimerization molecule is a small molecule, e.g., rapamycin or a rapalogue, e.g, RAD001.

The term “bioequivalent” refers to an amount of an agent other than the reference compound (e.g., RAD001), required to produce an effect equivalent to the effect produced by the reference dose or reference amount of the reference compound (e.g., RAD001). In an embodiment the effect is the level of mTOR inhibition, e.g., as measured by P70 S6 kinase inhibition, e.g., as evaluated in an in vivo or in vitro assay, e.g., as measured by an assay described herein, e.g., the Boulay assay, or measurement of phosphorylated S6 levels by western blot. In an embodiment, the effect is alteration of the ratio of PD-1 positive/PD-1 negative T cells, as measured by cell sorting. In an embodiment a bioequivalent amount or dose of an mTOR inhibitor is the amount or dose that achieves the same level of P70 S6 kinase inhibition as does the reference dose or reference amount of a reference compound. In an embodiment, a bioequivalent amount or dose of an mTOR inhibitor is the amount or dose that achieves the same level of alteration in the ratio of PD-1 positive/PD-1 negative T cells as does the reference dose or reference amount of a reference compound.

The term “low, immune enhancing, dose” when used in conjunction with an mTOR inhibitor, e.g., an allosteric mTOR inhibitor, e.g., RAD001 or rapamycin, or a catalytic mTOR inhibitor, refers to a dose of mTOR inhibitor that partially, but not fully, inhibits mTOR activity, e.g., as measured by the inhibition of P70 S6 kinase activity. Methods for evaluating mTOR activity, e.g., by inhibition of P70 S6 kinase, are discussed herein. The dose is insufficient to result in complete immune suppression but is sufficient to enhance the immune response. In an embodiment, the low, immune enhancing, dose of mTOR inhibitor results in a decrease in the number of PD-1 positive immune effector cells, e.g., T cells or NK cells, and/or an increase in the number of PD-1 negative immune effector cells, e.g., T cells or NK cells, or an increase in the ratio of PD-1 negative immune effector cells (e.g., T cells or NK cells)/PD-1 positive immune effector cells (e.g., T cells or NK cells).

In an embodiment, the low, immune enhancing, dose of mTOR inhibitor results in an increase in the number of naive T cells. In an embodiment, the low, immune enhancing, dose of mTOR inhibitor results in one or more of the following:
an increase in the expression of one or more of the following markers: CD62Lhigh, CD127high, CD27+, and BCL2, e.g., on memory T cells, e.g., memory T cell precursors;
a decrease in the expression of KLRG1, e.g., on memory T cells, e.g., memory T cell precursors; and
an increase in the number of memory T cell precursors, e.g., cells with any one or combination of the following characteristics: increased CD62Lhigh, increased CD127high, increased CD27+, decreased KLRG1, and increased BCL2;
wherein any of the changes described above occurs, e.g., at least transiently, e.g., as compared to a non-treated subject.

“Refractory” as used herein refers to a disease, e.g., cancer, that does not respond to a treatment. In embodiments, a refractory cancer can be resistant to a treatment before or at the beginning of the treatment. In other embodiments, the refractory cancer can become resistant during a treatment. A refractory cancer is also called a resistant cancer.

“Relapsed” or a “relapse” as used herein refers to the reappearance of a disease (e.g., cancer) or the signs and symptoms of a disease such as cancer after a period of improvement or responsiveness, e.g., after prior treatment of a therapy, e.g., cancer therapy. For example, the period of responsiveness may involve the level of cancer cells falling below a certain threshold, e.g., below 20%, 15%, 10%, 5%, 4%, 3%, 2%, or 1%. The reappearance may involve the level of cancer cells rising above a certain threshold, e.g., above 20%, 15%, 10%, 5%, 4%, 3%, 2%, or 1%.

In one aspect, a “responder” of a therapy can be a subject having complete response, very good partial response, or partial response after receiving the therapy. In one aspect, a “non-responder” of a therapy can be a subject having minor response, stable disease, or progressive disease after receiving the therapy. In some embodiments, the subject has multiple myeloma and the response of the subject to a multiple myeloma therapy is determined based on IMWG 2016 criteria, e.g., as disclosed in Kumar, et al., Lancet Oncol. 17, e328-346 (2016), hereby incorporated herein by reference in its entirety, e.g., as described in Table 8.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

A “gene editing system” as the term is used herein, refers to a system, e.g., one or more molecules, that direct and effect an alteration, e.g., a deletion, of one or more nucleic acids at or near a site of genomic DNA targeted by said system. Gene editing systems are known in the art, and are described more fully below.

The term “cognate antigen molecule” refers to any antigen described herein. In some embodiments, it refers to an antigen bound, e.g., recognized or targeted, by a CAR polypeptide, e.g., any target CAR described herein. In some embodiments, it refers to a cancer associated antigen described herein. In some embodiments, the cognate antigen molecule is a recombinant molecule.

The term “IL-15 receptor molecule” as used herein refers to a full-length naturally-occurring IL-15 receptor alpha (IL-15Ra) (e.g., a mammalian IL-15Ra, e.g., human IL-15Ra, e.g., GenBank Accession Number AAI21141.1), an active fragment of IL-15Ra, or an active variant having at least 80%, 85%, 90%, 95%, 96%, 97%, 98%, or 99% sequence identity to a naturally-occurring wild type polypeptide of IL-15Ra or fragment thereof. In some embodiments, the variant is a derivative, e.g., a mutant, of a wild type polypeptide or nucleic acid encoding the same. In some embodiments, the IL-15Ra variant, e.g., active variant of IL-15Ra, has at least 50%, 60%, 70%, 80%, 85%, 90%, 95%, 96%, 97%, 98%, 99% or 100% activity of the wild type IL-15Ra polypeptide. In some embodiments, the IL-15Ra molecule comprises one or more post-translational modifications. As used herein, the terms IL-15R and IL-15Ra are interchangeable.

The term “IL-15 molecule” as used herein refers to a full-length naturally-occurring IL-15 (e.g., a mammalian IL-15, e.g., human IL-15, e.g., GenBank Accession Number AAI00963.1), an active fragment of IL-15, or an active variant having at least 80%, 85%, 90%, 95%, 96%, 97%, 98%, or 99% sequence identity to a naturally-occurring wild type polypeptide of IL-15 or fragment thereof. In some embodiments, the variant is a derivative, e.g., a mutant, of a wild type polypeptide or nucleic acid encoding the same. In some embodiments, the IL-15 variant, e.g., active variant of IL-15, has at least 50%, 60%, 70%, 80%, 85%, 90%, 95%, 96%, 97%, 98%, 99% or 100% activity of the wild type IL-15 polypeptide. In some embodiments, the IL-15 molecule comprises one or more post-translational modifications.

As used herein, an “active variant” of a cytokine molecule refers to a cytokine variant having at least 50%, 60%, 70%, 80%, 85%, 90%, 95%, 96%, 97%, 98%, 99% or 100% activity of wild type cytokine, e.g., as measured by an art-recognized assay.

Various aspects of the compositions and methods herein are described in further detail below. Additional definitions are set out throughout the specification.

### DETAILED DESCRIPTION

The present invention provides, at least in part, a method of treating a subject, e.g., a subject having a cancer, comprising administering to the subject an effective number of a cell (e.g., a population of cells) that expresses a CAR molecule, in combination with an IL-15R molecule and/or and IL-15 molecule.

### Chimeric Antigen Receptor (CAR)

In one aspect, disclosed herein are methods of using a cell (e.g., a population of cells) that expresses a CAR molecule, an IL-15R molecule and an IL-15 molecule (“CAR IL-15R/IL-15 expressing cell”). In one aspect, an exemplary CAR construct comprises an optional leader sequence (e.g., a leader sequence described herein), an antigen binding domain (e.g., an antigen binding domain described herein), a hinge (e.g., a hinge region described herein), a transmembrane domain (e.g., a transmembrane domain described herein), and an intracellular stimulatory domain (e.g., an intracellular stimulatory domain described herein). In one aspect, an exemplary CAR construct comprises an optional leader sequence (e.g., a leader sequence described herein), an extracellular antigen binding domain (e.g., an antigen binding domain described herein), a hinge (e.g., a hinge region described herein), a transmembrane domain (e.g., a transmembrane domain described herein), an intracellular costimulatory signaling domain (e.g., a costimulatory signaling domain described herein) and/or an intracellular primary signaling domain (e.g., a primary signaling domain described herein).

Sequences of non-limiting examples of various components that can be part of a CAR molecule described herein, are listed in Table 1 and Table 2, where “aa” stands for amino acids, and “na” stands for nucleic acids that encode the corresponding peptide.

TABLE 1
Sequences for various components of CAR

| | | |
|---|---|---|
| SEQ | pGK | AGCTTATGGTGCCCCAACCCCAACGCGGAAAAGGTTCCGTCGGGAC |
| ID | promoter | CCAAACGCGTCCCTGCGCCGACGAGACCCGCACCAAGGCCCTTTGC |
| NO: | | GTCGCCGCGGCTGGGACCCAGAGCGTGTAAGAAGTGCAGGCAAGC |
| 13 | | GTCGCAGTGGGCCTAGAAGCGGCGATGGGAACACCCGGGGGGCCG |
| | | CTGCGAAGGACGAGGCGGGGATTCAGCCCTTCCAAGGAACGCCAAG |
| | | CGCCGCACGGCCTGCACTATTTGCCTTCGGCGTGCAGAGTGATCATG |
| | | GGAGCGTCTGCCTGTCGCGGTCCCTCGTTACCGTCGCGCGGCTGGCG |
| | | CTACCCGACACCGGTTATCGCCGACGAGTCGTCCCGCGCGGCTCTCG |
| | | TCGCCGGCCCTTCCCCGCCACGCCCTCCGCCCCACACCCCGCCATCA |
| | | CACCCGGGACAAGGACGGGCGCGCCACAAGGCGTAAGACGTTCGG |
| | | AGGCCTCGCGTGCAGCCGTCAGCCGAGGGAGCAACTGGCTTAGTGG |
| | | CTGGAGAGAGGGGT |
| SEQ | CTL019 | GACATCCAGATGACACAGACTACATCCTCCCTGTCTGCCTCTCTGGG |
| ID | scFv | AGACAGAGTCACCATCAGTTGCAGGGCAAGTCAGGACATTAGTAAA |
| NO: | nucleotide | TATTTAAATTGGTATCAGCAGAAACCAGATGGAACTGTTAAACTCCT |
| 14 | sequence | GATCTACCATACATCAAGATTACACTCAGGAGTCCCATCAAGGTTC |
| | | AGTGGCAGTGGGTCTGGAACAGATTATTCTCTCACCATTAGCAACCT |
| | | GGAGCAAGAAGATATTGCCACTTACTTTTGCCAACAGGGTAATACG |
| | | CTTCCGTACACGTTCGGAGGGGGGACCAAGCTGGAGATCACAGGTG |
| | | GCGGTGGCTCGGGCGGTGGTGGGTCGGGTGGCGGCGGATCTGAGGT |
| | | GAAACTGCAGGAGTCAGGACCTGGCCTGGTGGCGCCCTCACAGAGC |
| | | CTGTCCGTCACATGCACTGTCTCAGGGGTCTCATTACCCGACTATGG |
| | | TGTAAGCTGGATTCGCCAGCCTCCACGAAAGGGTCTGGAGTGGCTG |
| | | GGAGTAATATGGGGTAGTGAAACCACATACTATAATTCAGCTCTCA |
| | | AATCCAGACTGACCATCATCAAGGACAACTCCAAGAGCCAAGTTTT |
| | | CTTAAAAATGAACAGTCTGCAAACTGATGACACAGCCATTTACTAC |
| | | TGTGCCAAACATTATTACTACGGTGGTAGCTATGCTATGGACTACTG |
| | | GGGCCAAGGAACCTCAGTCACCGTCTCCTCA |
| SEQ | CTL019 | DIQMTQTTSSLSASLGDRVTISCRASQDISKYLNWYQQKPDGTVKLLIY |
| ID | scFv amino | HTSRLHSGVPSRFSGSGSGTDYSLTISNLEQEDIATYFCQQGNTLPYTFG |
| NO: | acid | GGTKLEITGGGGSGGGGSGGGGSEVKLQESGPGLVAPSQSLSVTCTVS |
| 15 | sequence | GVSLPDYGVSWIRQPPRKGLEWLGVIWGSETTYYNSALKSRLTIIKDNS |
| | | KSQVFLKMNSLQTDDTAIYYCAKHYYYGGSYAMDYWGQGTSVTVSS |
| SEQ | P2A | GGAAGCGGAGCTACTAACTTCAGCCTGCTGAAGCAGGCTGGAGACG |
| ID | nucleotide | TGGAGGAGAACCCTGGACCT |
| NO: | sequence | |
| 23 | | |
| SEQ | P2A amino | GSGATNFSLLKQAGDVEENPGP |
| ID | acid | |
| NO: | sequence | |
| 24 | | |
| SEQ | CTL019 | GACATCCAGATGACACAGACTACATCCTCCCTGTCTGCCTCTCTGGG |
| ID | full-length | AGACAGAGTCACCATCAGTTGCAGGGCAAGTCAGGACATTAGTAAA |
| NO: | nucleotide | TATTTAAATTGGTATCAGCAGAAACCAGATGGAACTGTTAAACTCCT |
| 25 | sequence | GATCTACCATACATCAAGATTACACTCAGGAGTCCCATCAAGGTTC |
| | | AGTGGCAGTGGGTCTGGAACAGATTATTCTCTCACCATTAGCAACCT |
| | | GGAGCAAGAAGATATTGCCACTTACTTTTGCCAACAGGGTAATACG |
| | | CTTCCGTACACGTTCGGAGGGGGGACCAAGCTGGAGATCACAGGTG |
| | | GCGGTGGCTCGGGCGGTGGTGGGTCGGGTGGCGGCGGATCTGAGGT |
| | | GAAACTGCAGGAGTCAGGACCTGGCCTGGTGGCGCCCTCACAGAGC |
| | | CTGTCCGTCACATGCACTGTCTCAGGGGTCTCATTACCCGACTATGG |
| | | TGTAAGCTGGATTCGCCAGCCTCCACGAAAGGGTCTGGAGTGGCTG |
| | | GGAGTAATATGGGGTAGTGAAACCACATACTATAATTCAGCTCTCA |
| | | AATCCAGACTGACCATCATCAAGGACAACTCCAAGAGCCAAGTTTT |
| | | CTTAAAAATGAACAGTCTGCAAACTGATGACACAGCCATTTACTAC |
| | | TGTGCCAAACATTATTACTACGGTGGTAGCTATGCTATGGACTACTG |
| | | GGGCCAAGGAACCTCAGTCACCGTCTCCTCAACCACGACGCCAGCG |
| | | CCGCGACCACCAACACCGGCGCCCACCATCGCGTCGCAGCCCCTGT |
| | | CCCTGCGCCCAGAGGCGTGCCGGCCAGCGGCGGGGGGCGCAGTGCA |
| | | CACGAGGGGGCTGGACTTCGCCTGTGATATCTACATCTGGGCGCCCT |
| | | TGGCCGGGACTTGTGGGGTCCTTCTCCTGTCACTGGTTATCACCCTT |
| | | TACTGCAAACGGGGCAGAAAGAAACTCCTGTATATATTCAAACAAC |
| | | CATTTATGAGACCAGTACAAACTACTCAAGAGGAAGATGGCTGTAG |
| | | CTGCCGATTTCCAGAAGAAGAAGAAGGAGGATGTGAACTGAGAGT |
| | | GAAGTTCAGCAGGAGCGCAGACGCCCCCGCGTACAAGCAGGGCCA |
| | | GAACCAGCTCTATAACGAGCTCAATCTAGGACGAAGAGAGGAGTAC |
| | | GATGTTTTGGACAAGAGACGTGGCCGGGACCCTGAGATGGGGGGAA |
| | | AGCCGAGAAGGAAGAACCCTCAGGAAGGCCTGTACAATGAACTGC |
| | | AGAAAGATAAGATGGCGGAGGCCTACAGTGAGATTGGGATGAAAG |
| | | GCGAGCGCCGGAGGGGCAAGGGGCACGATGGCCTTTACCAGGGTCT |
| | | CAGTACAGCCACCAAGGACACCTACGACGCCCTTCACATGCAGGCC |
| | | CTGCCCCCTCGC |
| SEQ | CTL019 | DIQMTQTTSSLSASLGDRVTISCRASQDISKYLNWYQQKPDGTVKLLIY |
| ID | full-length | HTSRLHSGVPSRFSGSGSGTDYSLTISNLEQEDIATYFCQQGNTLPYTFG |
| NO: | amino acid | GGTKLEITGGGGSGGGGSGGGGSEVKLQESGPGLVAPSQSLSVTCTVS |
| 26 | sequence | GVSLPDYGVSWIRQPPRKGLEWLGVIWGSETTYYNSALKSRLTIIKDNS |
| | | KSQVFLKMNSLQTDDTAIYYCAKHYYYGGSYAMDYWGQGTSVTVSS |
| | | TTTPAPRPPTPAPTIASQPLSLRPEACRPAAGGAVHTRGLDFACDIYIWA |
| | | PLAGTCGVLLLSLVITLYCKRGRKKLLYIFKQPFMRPVQTTQEEDGCSC |
| | | RFPEEEEGGCELRVKFSRSADAPAYKQGQNQLYNELNLGRREEYDVLD |
| | | KRRGRDPEMGGKPRRKNPQEGLYNELQKDKMAEAYSEIGMKGERRR |
| | | GKGHDGLYQGLSTATKDTYDALHMQALPPR |

TABLE 10
Sequences of various components of CAR (aa-amino acid sequence, na-nucleic acid
sequence).

| SEQ ID | | |
|---|---|---|
| NO: | description | Sequence |
| SEQ ID | EF-1 | CGTGAGGCTCCGGTGCCCGTCAGTGGGCAGAGCGCACATCGCCC |
| NO: 1014 | promoter | ACAGTCCCCGAGAAGTTGGGGGGAGGGGTCGGCAATTGAACCG |
| | | GTGCCTAGAGAAGGTGGCGCGGGGTAAACTGGGAAAGTGATGT |
| | | CGTGTACTGGCTCCGCCTTTTTCCCGAGGGTGGGGGAGAACCGT |
| | | ATATAAGTGCAGTAGTCGCCGTGAACGTTCTTTTTCGCAACGGGT |
| | | TTGCCGCCAGAACACAGGTAAGTGCCGTGTGTGGTTCCCGCGGG |
| | | CCTGGCCTCTTTACGGGTTATGGCCCTTGCGTGCCTTGAATTACT |
| | | TCCACCTGGCTGCAGTACGTGATTCTTGATCCCGAGCTTCGGGTT |
| | | GGAAGTGGGTGGGAGAGTTCGAGGCCTTGCGCTTAAGGAGCCCC |
| | | TTCGCCTCGTGCTTGAGTTGAGGCCTGGCCTGGGCGCTGGGGCC |
| | | GCCGCGTGCGAATCTGGTGGCACCTTCGCGCCTGTCTCGCTGCTT |
| | | TCGATAAGTCTCTAGCCATTTAAAATTTTTGATGACCTGCTGCGA |
| | | CGCTTTTTTTCTGGCAAGATAGTCTTGTAAATGCGGGCCAAGATC |
| | | TGCACACTGGTATTTCGGTTTTTGGGGCCGCGGGCGGCGACGGG |
| | | GCCCGTGCGTCCCAGCGCACATGTTCGGCGAGGCGGGGCCTGCG |
| | | AGCGCGGCCACCGAGAATCGGACGGGGGTAGTCTCAAGCTGGCC |
| | | GGCCTGCTCTGGTGCCTGGCCTCGCGCCGCCGTGTATCGCCCCGC |
| | | CCTGGGCGGCAAGGCTGGCCCGGTCGGCACCAGTTGCGTGAGCG |
| | | GAAAGATGGCCGCTTCCCGGCCCTGCTGCAGGGAGCTCAAAATG |
| | | GAGGACGCGGCGCTCGGGAGAGCGGGCGGGTGAGTCACCCACA |
| | | CAAAGGAAAAGGGCCTTTCCGTCCTCAGCCGTCGCTTCATGTGA |
| | | CTCCACGGAGTACCGGGCGCCGTCCAGGCACCTCGATTAGTTCT |
| | | CGAGCTTTTGGAGTACGTCGTCTTTAGGTTGGGGGGAGGGGTTTT |
| | | ATGCGATGGAGTTTCCCCACACTGAGTGGGTGGAGACTGAAGTT |
| | | AGGCCAGCTTGGCACTTGATGTAATTCTCCTTGGAATTTGCCCTT |
| | | TTTGAGTTTGGATCTTGGTTCATTCTCAAGCCTCAGACAGTGGTT |
| | | CAAAGTTTTTTTCTTCCATTTCAGGTGTCGTGA |
| SEQ ID | Leader (aa) | MALPVTALLLPLALLLHAARP |
| NO: 1015 | | |
| SEQ ID | Leader (na) | ATGGCCCTGCCTGTGACAGCCCTGCTGCTGCCTCTGGCTCTGCTG |
| NO: 1016 | | CTGCATGCCGCTAGACCC |
| SEQ ID | Leader (na) | ATGGCCCTCCCTGTCACCGCCCTGCTGCTTCCGCTGGCTCTTCTG |
| NO: 1017 | | CTCCACGCCGCTCGGCCC |
| SEQ ID | CD8 hinge | TTTPAPRPPTPAPTIASQPLSLRPEACRPAAGGAVHTRGLDFACD |
| NO: 1018 | (aa) | |
| SEQ ID | CD8 hinge | ACCACGACGCCAGCGCCGCGACCACCAACACCGGCGCCCACCAT |
| NO: 1019 | (na) | CGCGTCGCAGCCCCTGTCCCTGCGCCCAGAGGCGTGCCGGCCAG |
| | | CGGCGGGGGGCGCAGTGCACACGAGGGGGCTGGACTTCGCCTGT |
| | | GAT |
| SEQ ID | Ig4 hinge | ESKYGPPCPPCPAPEFLGGPSVFLFPPKPKDTLMISRTPEVTCVVVDV |
| NO: 1020 | (aa) | SQEDPEVQFNWYVDGVEVHNAKTKPREEQFNSTYRVVSVLTVLHQ |
| | | DWLNGKEYKCKVSNKGLPSSIEKTISKAKGQPREPQVYTLPPSQEE |
| | | MTKNQVSLTCLVKGFYPSDIAVEWESNGQPENNYKTTPPVLDSDGS |
| | | FFLYSRLTVDKSRWQEGNVFSCSVMHEALHNHYTQKSLSLSLGKM |
| SEQ ID | Ig4 hinge | GAGAGCAAGTACGGCCCTCCCTGCCCCCCTTGCCCTGCCCCCGA |
| NO: 1021 | (na) | GTTCCTGGGCGGACCCAGCGTGTTCCTGTTCCCCCCCAAGCCCAA |
| | | GGACACCCTGATGATCAGCCGGACCCCCGAGGTGACCTGTGTGG |
| | | TGGTGGACGTGTCCCAGGAGGACCCCGAGGTCCAGTTCAACTGG |
| | | TACGTGGACGGCGTGGAGGTGCACAACGCCAAGACCAAGCCCC |
| | | GGGAGGAGCAGTTCAATAGCACCTACCGGGTGGTGTCCGTGCTG |
| | | ACCGTGCTGCACCAGGACTGGCTGAACGGCAAGGAATACAAGTG |
| | | TAAGGTGTCCAACAAGGGCCTGCCCAGCAGCATCGAGAAAACCA |
| | | TCAGCAAGGCCAAGGGCCAGCCTCGGGAGCCCCAGGTGTACACC |
| | | CTGCCCCCTAGCCAAGAGGAGATGACCAAGAACCAGGTGTCCCT |
| | | GACCTGCCTGGTGAAGGGCTTCTACCCCAGCGACATCGCCGTGG |
| | | AGTGGGAGAGCAACGGCCAGCCCGAGAACAACTACAAGACCAC |
| | | CCCCCCTGTGCTGGACAGCGACGGCAGCTTCTTCCTGTACAGCCG |
| | | GCTGACCGTGGACAAGAGCCGGTGGCAGGAGGGCAACGTCTTTA |
| | | GCTGCTCCGTGATGCACGAGGCCCTGCACAACCACTACACCCAG |
| | | AAGAGCCTGAGCCTGTCCCTGGGCAAGATG |
| SEQ ID | IgD hinge | RWPESPKAQASSVPTAQPQAEGSLAKATTAPATTRNTGRGGEEKK |
| NO: 1022 | (aa) | KEKEKEEQEERETKTPECPSHTQPLGVYLLTPAVQDLWLRDKATFT |
| | | CFVVGSDLKDAHLTWEVAGKVPTGGVEEGLLERHSNGSQSQHSRL |
| | | TLPRSLWNAGTSVTCTLNHPSLPPQRLMALREPAAQAPVKLSLNLL |
| | | ASSDPPEAASWLLCEVSGFSPPNILLMWLEDQREVNTSGFAPARPPP |
| | | QPGSTTFWAWSVLRVPAPPSPQPATYTCVVSHEDSRTLLNASRSLE |
| | | VSYVTDH |
| SEQ ID | IgD hinge | AGGTGGCCCGAAAGTCCCAAGGCCCAGGCATCTAGTGTTCCTAC |
| NO: 1023 | (na) | TGCACAGCCCCAGGCAGAAGGCAGCCTAGCCAAAGCTACTACTG |
| | | CACCTGCCACTACGCGCAATACTGGCCGTGGCGGGGAGGAGAAG |
| | | AAAAAGGAGAAAGAGAAAGAAGAACAGGAAGAGAGGGAGACC |
| | | AAGACCCCTGAATGTCCATCCCATACCCAGCCGCTGGGCGTCTA |
| | | TCTCTTGACTCCCGCAGTACAGGACTTGTGGCTTAGAGATAAGG |
| | | CCACCTTTACATGTTTCGTCGTGGGCTCTGACCTGAAGGATGCCC |
| | | ATTTGACTTGGGAGGTTGCCGGAAAGGTACCCACAGGGGGGGTT |
| | | GAGGAAGGGTTGCTGGAGCGCCATTCCAATGGCTCTCAGAGCCA |
| | | GCACTCAAGACTCACCCTTCCGAGATCCCTGTGGAACGCCGGGA |
| | | CCTCTGTCACATGTACTCTAAATCATCCTAGCCTGCCCCCACAGC |
| | | GTCTGATGGCCCTTAGAGAGCCAGCCGCCCAGGCACCAGTTAAG |
| | | CTTAGCCTGAATCTGCTCGCCAGTAGTGATCCCCCAGAGGCCGC |
| | | CAGCTGGCTCTTATGCGAAGTGTCCGGCTTTAGCCCGCCCAACAT |
| | | CTTGCTCATGTGGCTGGAGGACCAGCGAGAAGTGAACACCAGCG |
| | | GCTTCGCTCCAGCCCGGCCCCCACCCCAGCCGGGTTCTACCACAT |
| | | TCTGGGCCTGGAGTGTCTTAAGGGTCCCAGCACCACCTAGCCCC |
| | | CAGCCAGCCACATACACCTGTGTTGTGTCCCATGAAGATAGCAG |
| | | GACCCTGCTAAATGCTTCTAGGAGTCTGGAGGTTTCCTACGTGAC |
| | | TGACCATT |
| SEQ ID | GS | GGGGSGGGGS |
| NO: 1024 | hinge/linker | |
| | (aa) | |
| SEQ ID | GS | GGTGGCGGAGGTTCTGGAGGTGGAGGTTCC |
| NO: 1025 | hinge/linker | |
| | (na) | |
| SEQ ID | CD8 | IYIWAPLAGTCGVLLLSLVITLYC |
| NO: 1026 | transmembrane | |
| | (TM) (aa) | |
| SEQ ID | CD8 | ATCTACATCTGGGCGCCCTTGGCCGGGACTTGTGGGGTCCTTCTC |
| NO: 1027 | transmembrane | CTGTCACTGGTTATCACCCTTTACTGC |
| | (TM) (na) | |
| SEQ ID | CD8 TM (na) | ATCTACATTTGGGCCCCTCTGGCTGGTACTTGCGGGGTCCTGCTG |
| NO: 1028 | | CTTTCACTCGTGATCACTCTTTACTGT |
| SEQ ID | 4-1BB | KRGRKKLLYIFKQPFMRPVQTTQEEDGCSCRFPEEEEGGCEL |
| NO: 1029 | intracellular | |
| | domain (aa) | |
| SEQ ID | 4-1BB | AAACGGGGCAGAAAGAAACTCCTGTATATATTCAAACAACCATT |
| NO: 1030 | intracellular | TATGAGACCAGTACAAACTACTCAAGAGGAAGATGGCTGTAGCT |
| | domain (na) | GCCGATTTCCAGAAGAAGAAGAAGGAGGATGTGAACTG |
| SEQ ID | 4-1BB | AAGCGCGGTCGGAAGAAGCTGCTGTACATCTTTAAGCAACCCTT |
| NO: 1031 | intracellular | CATGAGGCCTGTGCAGACTACTCAAGAGGAGGACGGCTGTTCAT |
| | domain (na) | GCCGGTTCCCAGAGGAGGAGGAAGGCGGCTGCGAACTG |
| SEQ ID | CD27 (aa) | QRRKYRSNKGESPVEPAEPCRYSCPREEEGSTIPIQEDYRKPEPACSP |
| NO: 1032 | | |
| SEQ ID | CD27 (na) | AGGAGTAAGAGGAGCAGGCTCCTGCACAGTGACTACATGAACAT |
| NO: 1033 | | GACTCCCCGCCGCCCCGGGCCCACCCGCAAGCATTACCAGCCCT |
| | | ATGCCCCACCACGCGACTTCGCAGCCTATCGCTCC |
| SEQ ID | CD3-zeta | RVKFSRSADAPAYKQGQNQLYNELNLGRREEYDVLDKRRGRDPE |
| NO: 1034 | (aa) | MGGKPRRKNPQEGLYNELQKDKMAEAYSEIGMKGERRRGKGHDG |
| | | LYQGLSTATKDTYDALHMQALPPR |
| SEQ ID | CD3-zeta | AGAGTGAAGTTCAGCAGGAGCGCAGACGCCCCCGCGTACAAGC |
| NO: 1035 | (na) | AGGGCCAGAACCAGCTCTATAACGAGCTCAATCTAGGACGAAGA |
| | | GAGGAGTACGATGTTTTGGACAAGAGACGTGGCCGGGACCCTGA |
| | | GATGGGGGGAAAGCCGAGAAGGAAGAACCCTCAGGAAGGCCTG |
| | | TACAATGAACTGCAGAAAGATAAGATGGCGGAGGCCTACAGTG |
| | | AGATTGGGATGAAAGGCGAGCGCCGGAGGGGCAAGGGGCACGA |
| | | TGGCCTTTACCAGGGTCTCAGTACAGCCACCAAGGACACCTACG |
| | | ACGCCCTTCACATGCAGGCCCTGCCCCCTCGC |
| SEQ ID | CD3-zeta | CGCGTGAAATTCAGCCGCAGCGCAGATGCTCCAGCCTACAAGCA |
| NO: 1036 | (na) | GGGGCAGAACCAGCTCTACAACGAACTCAATCTTGGTCGGAGAG |
| | | AGGAGTACGACGTGCTGGACAAGCGGAGAGGACGGGACCCAGA |
| | | AATGGGCGGGAAGCCGCGCAGAAAGAATCCCCAAGAGGGCCTG |
| | | TACAACGAGCTCCAAAAGGATAAGATGGCAGAAGCCTATAGCG |
| | | AGATTGGTATGAAAGGGGAACGCAGAAGAGGCAAAGGCCACGA |
| | | CGGACTGTACCAGGGACTCAGCACCGCCACCAAGGACACCTATG |
| | | ACGCTCTTCACATGCAGGCCCTGCCGCCTCGG |
| SEQ ID | CD3-zeta | RVKFSRSADAPAYQQGQNQLYNELNLGRREEYDVLDKRRGRDPE |
| NO: 1037 | (aa) | MGGKPRRKNPQEGLYNELQKDKMAEAYSEIGMKGERRRGKGHDG |
| | | LYQGLSTATKDTYDALHMQALPPR |
| SEQ ID | CD3-zeta | AGAGTGAAGTTCAGCAGGAGCGCAGACGCCCCCGCGTACCAGC |
| NO: 1038 | (na) | AGGGCCAGAACCAGCTCTATAACGAGCTCAATCTAGGACGAAGA |
| | | GAGGAGTACGATGTTTTGGACAAGAGACGTGGCCGGGACCCTGA |
| | | GATGGGGGGAAAGCCGAGAAGGAAGAACCCTCAGGAAGGCCTG |
| | | TACAATGAACTGCAGAAAGATAAGATGGCGGAGGCCTACAGTG |
| | | AGATTGGGATGAAAGGCGAGCGCCGGAGGGGCAAGGGGCACGA |
| | | TGGCCTTTACCAGGGTCTCAGTACAGCCACCAAGGACACCTACG |
| | | ACGCCCTTCACATGCAGGCCCTGCCCCCTCGC |
| SEQ ID | linker | GGGGS |
| NO: 1039 | | |
| SEQ ID | linker | GGTGGCGGAGGTTCTGGAGGTGGAGGTTCC |
| NO: 1040 | | |
| SEQ ID | PD-1 | Pgwfldspdrpwnpptfspallvvtegdnatftcsfsntsesfvlnwyrmspsnqtdklaafpedrsqp |
| NO: 1041 | extracellular | gqdcrfrvtqlpngrdfhmsvvrarrndsgtylcgaislapkaqikeslraelrvterraevptahpspspr |
| | domain (aa) | pagqfqtlv |
| SEQ ID | PD-1 | Cccggatggtttctggactctccggatcgcccgtggaatcccccaaccttctcaccggcactcttggttgtga |
| NO: 1042 | extracellular | ctgagggcgataatgcgaccttcacgtgctcgttctccaacacctccgaatcattcgtgctgaactggtaccg |
| | domain (na) | catgagcccgtcaaaccagaccgacaagctcgccgcgtttccggaagatcggtcgcaaccgggacagga |
| | | ttgtcggttccgcgtgactcaactgccgaatggcagagacttccacatgagcgtggtccgcgctaggcgaa |
| | | acgactccgggacctacctgtgcggagccatctcgctggcgcctaaggcccaaatcaaagagagcttgag |
| | | ggccgaactgagagtgaccgagcgcagagctgaggtgccaactgcacatccatccccatcgcctcggcc |
| | | tgcggggcagtttcagaccctggtc |
| SEQ ID | PD-1 CAR | Malpvtalllplalllhaarppgwfldspdrpwnpptfspallvvtegdnatftcsfsntsesvlnwyrm |
| NO: 1043 | (aa) with | spsnqtdklaafpedrsqpgqdcrfrvtqlpngrdfhmsvvrarrndsgtylcgaislapkaqikeslrae |
| | signal | lrvterraevptahpspsprpagqfqtlvtttpaprpptpaptiasqplslrpeacrpaaggavhtrgldfac |
| | | diyiwaplagtcgvlllslvitlyckrgrkkllyifkqpfmrpvqttqeedgcscrfpeeeeggcelrvkfsr |
| | | sadapaykqgqnqlynelnlgrreeydvldkagrdpemggkprrknpqeglynelqkdkmaeays |
| | | eigmkgerrrgkghdglyqglstatkdtydalhmqalppr |
| SEQ ID | PD-1 CAR | Atggccctccctgtcactgccctgcttctccccctcgcactcctgctccacgccgctagaccacccggatg |
| NO: 1044 | (na) | gtttctggactctccggatcgcccgtggaatcccccaaccttctcaccggcactcttggttgtgactgagggc |
| | | gataatgcgaccttcacgtgctcgttctccaacacctccgaatcattcgtgctgaactggtaccgcatgagcc |
| | | cgtcaaaccagaccgacaagctcgccgcgtttccggaagatcggtcgcaaccgggacaggattgtcggtt |
| | | ccgcgtgactcaactgccgaatggcagagacttccacatgagcgtggtccgcgctaggcgaaacgactcc |
| | | gggacctacctgtgcggagccatctcgctggcgcctaaggcccaaatcaaagagagcttgagggccgaa |
| | | ctgagagtgaccgagcgcagagctgaggtgccaactgcacatccatccccatcgcctcggcctgcgggg |
| | | cagtttcagaccctggtcacgaccactccggcgccgcgcccaccgactccggccccaactatcgcgagcc |
| | | agcccctgtcgctgaggccggaagcatgccgccctgccgccggaggtgctgtgcatacccggggattgg |
| | | acttcgcatgcgacatctacatttgggctcctctcgccggaacttgtggcgtgctccttctgtccctggtcatca |
| | | ccctgtactgcaagcggggtcggaaaaagcttctgtacattttcaagcagcccttcatgaggcccgtgcaaa |
| | | ccacccaggaggaggacggttgctcctgccggttccccgaagaggaagaaggaggttgcgagctgcgc |
| | | gtgaagttctcccggagcgccgacgcccccgcctataagcagggccagaaccagctgtacaacgaactg |
| | | aacctgggacggcgggaagagtacgatgtgctggacaagcggcgcggccgggaccccgaaatgggcg |
| | | ggaagcctagaagaaagaaccctcaggaaggcctgtataacgagctgcagaaggacaagatggccgag |
| | | gcctactccgaaattgggatgaagggagagcggcggaggggaaaggggcacgacggcctgtaccaag |
| | | gactgtccaccgccaccaaggacacatacgatgccctgcacatgcaggcccttccccctcgc |
| SEQ ID | linker | (Gly-Gly-Gly-Ser)n, where n = 1-10 |
| NO: 1009 | | |
| SEQ ID | linker | (Gly4 Ser)4 |
| NO: 1010 | | |
| SEQ ID | linker | (Gly4 Ser)3 |
| NO: 1011 | | |
| SEQ ID | linker | (Gly3Ser) |
| NO: 1012 | | |
| SEQ ID | linker | ASGGGGSGGRASGGGGS |
| NO: 1045 | | |
| SEQ ID | polyA | [a]₅₀₋₅₀₀₀ |
| NO: 1013 | | |
| SEQ ID | PD1 CAR | Pgwfldspdrpwnpptfspallvvtegdnatftcsfsntsesfvlnwyrmspsnqtdklaafpedrsqp |
| NO: 1046 | (aa) | gqdcrfrvtqlpngrdfhmsvvrarrndsgtylcgaislapkaqikeslraelrvterraevptahpspspr |
| | | pagqfqtlvtttpaprpptpaptiasqplslrpeacrpaaggavhtrgldfacdiyiwaplagtcgvlllslvi |
| | | tlyckrgrkkllyiflcqpfmrpvqttqeedgcscrfpeeeeggcelrvkfsrsadapaykqgqnqlynel |
| | | nlgrreeydvldlargrdpemggkprrknpqeglynelqkdkmaeayseigmkgeragkghdgly |
| | | qglstatkdtydalhmqalppr |
| SEQ ID | ICOS | TKKKYSSSVHDPNGEYMFMRAVNTAKKSRLTDVTL |
| NO: 1047 | intracellular | |
| | domain (aa) | |
| SEQ ID | ICOS | ACAAAAAAGAAGTATTCATCCAGTGTGCACGACCCTAACGGTGA |
| NO: 1048 | intracellular | ATACATGTTCATGAGAGCAGTGAACACAGCCAAAAAATCCAGAC |
| | domain (na) | TCACAGATGTGACCCTA |
| SEQ ID | ICOS TM | TTTPAPRPPTPAPTIASQPLSLRPEACRPAAGGAVHTRGLDFACDFW |
| NO: 1049 | domain (aa) | LPIGCAAFVVVCILGCILICWL |
| SEQ ID | ICOS TM | ACCACGACGCCAGCGCCGCGACCACCAACACCGGCGCCCACCAT |
| NO: 1050 | domain (na) | CGCGTCGCAGCCCCTGTCCCTGCGCCCAGAGGCGTGCCGGCCAG |
| | | CGGCGGGGGGCGCAGTGCACACGAGGGGGCTGGACTTCGCCTGT |
| | | GATTTCTGGTTACCCATAGGATGTGCAGCCTTTGTTGTAGTCTGC |
| | | ATTTTGGGATGCATACTTATTTGTTGGCTT |
| SEQ ID | CD28 | RSKRSRLLHSDYMNMTPRRPGPTRKHYQPYAPPRDFAAYRS |
| NO: 1051 | intracellular | |
| | domain (aa) | |
| SEQ ID | CD28 | AGGAGTAAGAGGAGCAGGCTCCTGCACAGTGACTACATGAACAT |
| NO: 1052 | intracellular | GACTCCCCGCCGCCCCGGGCCCACCCGCAAGCATTACCAGCCCT |
| | domain (na) | ATGCCCCACCACGCGACTTCGCAGCCTATCGCTCC |

### CAR Antigen Binding Domain

In one aspect, the portion of the CAR comprising the antigen binding domain comprises an antigen binding domain that targets a tumor antigen, e.g., a tumor antigen described herein. In some embodiments, the antigen binding domain binds to: CD19; CD123; CD22; CD30; CD171; CS-1; C-type lectin-like molecule-1, CD33; epidermal growth factor receptor variant III (EGFRvIII); ganglioside G2 (GD2); ganglioside GD3; TNF receptor family member; B-cell maturation antigen (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2; Mesothelin; Interleukin 11 receptor alpha (IL-11Ra); prostate stem cell antigen (PSCA); Protease Serine 21; vascular endothelial growth factor receptor 2 (VEGFR2); Lewis (Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3; transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1, melanoma antigen recognized by T cells 1; Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like, Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); or immunoglobulin lambda-like polypeptide 1 (IGLL1).

The antigen binding domain can be any domain that binds to an antigen, including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, a T cell receptor (TCR), or a fragment there of, e.g., single chain TCR, and the like. In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment.

### CAR Transmembrane Domain

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In one aspect, the transmembrane domain is one that is associated with one of the other domains of the CAR. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another CAR on the cell surface of a CAR-expressing cell. In a different aspect, the amino acid sequence of the transmembrane domain may be modified or substituted so as to minimize interactions with the binding domains of the native binding partner present in the same CART.

The transmembrane domain may be derived either from a natural or from a recombinant source. Where the source is natural, the domain may be derived from any membrane-bound or transmembrane protein. In one aspect the transmembrane domain is capable of signaling to the intracellular domain(s) whenever the CAR has bound to a target. A transmembrane domain of particular use in this invention may include at least the transmembrane region(s) of e.g., the alpha, beta or zeta chain of the T-cell receptor, CD28, CD27, CD3 epsilon, CD45, CD4, CD5, CD8, CD9, CD16, CD22, CD33, CD37, CD64, CD80, CD86, CD134, CD137, CD154. In some embodiments, a transmembrane domain may include at least the transmembrane region(s) of, e.g., KIR2DS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, NKG2C.

In some instances, the transmembrane domain can be attached to the extracellular region of the CAR, e.g., the antigen binding domain of the CAR, via a hinge, e.g., a hinge from a human protein. For example, in one embodiment, the hinge can be a human Ig (immunoglobulin) hinge, e.g., an IgG4 hinge, or a CD8a hinge. In one embodiment, the hinge or spacer comprises (e.g., consists of) the amino acid sequence of SEQ ID NO: 1018. In one aspect, the transmembrane domain comprises (e.g., consists of) a transmembrane domain of SEQ ID NO: 1026.

In one aspect, the hinge or spacer comprises an IgG4 hinge. For example, in one embodiment, the hinge or spacer comprises a hinge of the amino acid sequence of SEQ ID NO: 1020. In some embodiments, the hinge or spacer comprises a hinge encoded by a nucleotide sequence of SEQ ID NO: 1021.

In one aspect, the hinge or spacer comprises an IgD hinge. For example, in one embodiment, the hinge or spacer comprises a hinge of the amino acid sequence of SEQ ID NO: 1022. In some embodiments, the hinge or spacer comprises a hinge encoded by a nucleotide sequence of SEQ ID NO: 1023.

In one aspect, the transmembrane domain may be recombinant, in which case it will comprise predominantly hydrophobic residues such as leucine and valine. In one aspect a triplet of phenylalanine, tryptophan and valine can be found at each end of a recombinant transmembrane domain.

Optionally, a short oligo- or polypeptide linker, between 2 and 10 amino acids in length may form the linkage between the transmembrane domain and the cytoplasmic region of the CAR. A glycine-serine doublet provides a particularly suitable linker. For example, in one aspect, the linker comprises the amino acid sequence of SEQ ID NO: 1024. In some embodiments, the linker is encoded by a nucleotide sequence of SEQ ID NO: 1025.

In one aspect, the hinge or spacer comprises a KIR2DS2 hinge.

### Cytoplasmic Domain

The cytoplasmic domain or region of the CAR includes an intracellular signaling domain. An intracellular signaling domain is generally responsible for activation of at least one of the normal effector functions of the immune cell in which the CAR has been introduced.

Examples of intracellular signaling domains for use in a CAR described herein include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAM containing primary intracellular signaling domains that are of particular use in the invention include those of TCR zeta, FcR gamma, FcR beta, CD3 gamma, CD3 delta, CD3 epsilon, CD5, CD22, CD79a, CD79b, CD278 (also known as “ICOS”), FcεRI, DAP10, DAP12, and CD66d. In one embodiment, a CAR of the invention comprises an intracellular signaling domain, e.g., a primary signaling domain of CD3-zeta, e.g., a CD3-zeta sequence described herein.

In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

### Costimulatory Signaling Domain

The intracellular signalling domain of the CAR can comprise the CD3-zeta signaling domain by itself or it can be combined with any other desired intracellular signaling domain(s) useful in the context of a CAR of the invention. For example, the intracellular signaling domain of the CAR can comprise a CD3 zeta chain portion and a costimulatory signaling domain. The costimulatory signaling domain refers to a portion of the CAR comprising the intracellular domain of a costimulatory molecule. In one embodiment, the intracellular domain is designed to comprise the signaling domain of CD3-zeta and the signaling domain of CD28. In one aspect, the intracellular domain is designed to comprise the signaling domain of CD3-zeta and the signaling domain of ICOS.

A costimulatory molecule can be a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. For example, CD27 costimulation has been demonstrated to enhance expansion, effector function, and survival of human CART cells in vitro and augments human T cell persistence and antitumor activity in vivo (Song et al. Blood. 2012; 119(3):696-706). Further examples of such costimulatory molecules include ICAM-1, GITR, BAFFR, HVEM (LIGHTR), SLAMF7, NKp80 (KLRF1), NKp30, NKp44, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, NKG2D, NKG2C and PAG/Cbp.

The intracellular signaling sequences within the cytoplasmic portion of the CAR may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequence. In one embodiment, a glycine-serine doublet can be used as a suitable linker. In one embodiment, a single amino acid, e.g., an alanine, a glycine, can be used as a suitable linker.

In one aspect, the intracellular signaling domain is designed to comprise two or more, e.g., 2, 3, 4, 5, or more, costimulatory signaling domains. In an embodiment, the two or more, e.g., 2, 3, 4, 5, or more, costimulatory signaling domains, are separated by a linker molecule, e.g., a linker molecule described herein. In one embodiment, the intracellular signaling domain comprises two costimulatory signaling domains. In some embodiments, the linker molecule is a glycine residue. In some embodiments, the linker is an alanine residue.

In one aspect, the intracellular signaling domain is designed to comprise the signaling domain of CD3-zeta and the signaling domain of CD28. In one aspect, the intracellular signaling domain is designed to comprise the signaling domain of CD3-zeta and the signaling domain of 4-1BB. In one aspect, the signaling domain of 4-1BB is a signaling domain of SEQ ID NO: 1029. In one aspect, the signaling domain of CD3-zeta is a signaling domain of SEQ ID NO: 1034.

In one aspect, the intracellular signaling domain is designed to comprise the signaling domain of CD3-zeta and the signaling domain of CD27. In one aspect, the signaling domain of CD27 comprises an amino acid sequence of SEQ ID NO: 1032. In one aspect, the signalling domain of CD27 is encoded by a nucleic acid sequence of SEQ ID NO: 1033.

In one aspect, the CAR-expressing cell described herein can further comprise a second CAR, e.g., a second CAR that includes a different antigen binding domain, e.g., to the same target or a different target (e.g., a target other than a cancer associated antigen described herein or a different cancer associated antigen described herein, e.g., CD19, CD33, CLL-1, CD34, FLT3, or folate receptor beta). In one embodiment, the second CAR includes an antigen binding domain to a target expressed the same cancer cell type as the cancer associated antigen. In one embodiment, the CAR-expressing cell comprises a first CAR that targets a first antigen and includes an intracellular signaling domain having a costimulatory signaling domain but not a primary signaling domain, and a second CAR that targets a second, different, antigen and includes an intracellular signaling domain having a primary signaling domain but not a costimulatory signaling domain. While not wishing to be bound by theory, placement of a costimulatory signaling domain, e.g., 4-1BB, CD28, ICOS, CD27 or OX-40, onto the first CAR, and the primary signaling domain, e.g., CD3 zeta, on the second CAR can limit the CAR activity to cells where both targets are expressed. In one embodiment, the CAR expressing cell comprises a first cancer associated antigen CAR that includes an antigen binding domain that binds a target antigen described herein, a transmembrane domain and a costimulatory domain and a second CAR that targets a different target antigen (e.g., an antigen expressed on that same cancer cell type as the first target antigen) and includes an antigen binding domain, a transmembrane domain and a primary signaling domain. In another embodiment, the CAR expressing cell comprises a first CAR that includes an antigen binding domain that binds a target antigen described herein, a transmembrane domain and a primary signaling domain and a second CAR that targets an antigen other than the first target antigen (e.g., an antigen expressed on the same cancer cell type as the first target antigen) and includes an antigen binding domain to the antigen, a transmembrane domain and a costimulatory signaling domain.

In another aspect, the disclosure features a population of CAR-expressing cells, e.g., CART cells. In some embodiments, the population of CAR-expressing cells comprises a mixture of cells expressing different CARs. For example, in one embodiment, the population of CART cells can include a first cell expressing a CAR having an antigen binding domain to a cancer associated antigen described herein, and a second cell expressing a CAR having a different antigen binding domain, e.g., an antigen binding domain to a different a cancer associated antigen described herein, e.g., an antigen binding domain to a cancer associated antigen described herein that differs from the cancer associate antigen bound by the antigen binding domain of the CAR expressed by the first cell. As another example, the population of CAR-expressing cells can include a first cell expressing a CAR that includes an antigen binding domain to a cancer associated antigen described herein, and a second cell expressing a CAR that includes an antigen binding domain to a target other than a cancer associate antigen as described herein. In one embodiment, the population of CAR-expressing cells includes, e.g., a first cell expressing a CAR that includes a primary intracellular signaling domain, and a second cell expressing a CAR that includes a secondary signaling domain.

In another aspect, the disclosure features a population of cells wherein at least one cell in the population expresses a CAR having an antigen binding domain to a cancer associated antigen described herein, and a second cell expressing another agent, e.g., an agent which enhances the activity of a CAR-expressing cell. For example, in one embodiment, the agent can be an agent which inhibits an inhibitory molecule. Inhibitory molecules, e.g., PD-1, can, in some embodiments, decrease the ability of a CAR-expressing cell to mount an immune effector response. Examples of inhibitory molecules include PD-1, PD-L1, CTLA4, TIM3, CEACAM (CEACAM-1, CEACAM-3, and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF (e.g., TGFbeta). In one embodiment, the agent which inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD-1, PD-L1, CTLA4, TIM3, CEACAM (CEACAM-1, CEACAM-3, and/or CEACAM-5), LAG3, VISTA, BTLA, TIGIT, LAIR1, CD160, 2B4 and TGF beta, or a fragment of any of these, and a second polypeptide which is an intracellular signaling domain described herein (e.g., comprising a costimulatory domain (e.g., 41BB, CD27, OX40 or CD28, e.g., as described herein) and/or a primary signaling domain (e.g., a CD3 zeta signaling domain described herein). In one embodiment, the agent comprises a first polypeptide of PD-1 or a fragment thereof, and a second polypeptide of an intracellular signaling domain described herein (e.g., a CD28 signaling domain described herein and/or a CD3 zeta signaling domain described herein).

### CD19 CAR and CD19-Binding Sequences

In some embodiments, the CAR IL-15R/IL-15-expressing cell described herein is a CD19 CAR-expressing cell (e.g., a cell expressing a CAR that binds to human CD19).

In one embodiment, the antigen binding domain of the CD19 CAR has the same or a similar binding specificity as the FMC63 scFv fragment described in Nicholson et al. Mol. Immun. 34 (16-17): 1157-1165 (1997). In one embodiment, the antigen binding domain of the CD19 CAR includes the scFv fragment described in Nicholson et al. Mol. Immun. 34 (16-17): 1157-1165 (1997).

In some embodiments, the CD19 CAR includes an antigen binding domain (e.g., a humanized antigen binding domain) according to Table 3 of WO2014/153270, incorporated herein by reference. WO2014/153270 also describes methods of assaying the binding and efficacy of various CAR constructs.

In one aspect, the parental murine scFv sequence is the CAR19 construct provided in PCT publication WO2012/079000 (incorporated herein by reference). In one embodiment, the anti-CD19 binding domain is a scFv described in WO2012/079000.

In one embodiment, the CAR molecule comprises the fusion polypeptide sequence provided as SEQ ID NO: 12 in PCT publication WO2012/079000, which provides an scFv fragment of murine origin that specifically binds to human CD19.

In one embodiment, the CD19 CAR comprises an amino acid sequence provided as SEQ ID NO: 12 in PCT publication WO2012/079000. In embodiment, the amino acid sequence is

(MALPVTALLLPLALLLHAARP)diqmtqttsslsaslgdrvtiscrasqdiskylnwyqqkpdgtvklliyhtsrlhsg vpsrfsgsgsgtdysltisnleqediatyfcqqgntlpytfgggtkleitggggggggggggsevklqesgpglvapsqslsvtctvsgvslpdyg vswirqpprkglewlgviwgsettyynsalksrltiikdnsksqvflkmnslqtddtaiyycakhyyyggsyamdywgqgtsvtvsstttpaprp ptpaptiasqplslrpeacrpaaggavhtrgldfacdiyiwaplagtcgvlllslvitlyckrgrkkllyifkqpfmrpvqttqeedgccrfpeeeegg celrvkfsrsadapaykqgqnqlynelnlgrreeydvldkrrgrdpemggkprrknpqeglynelqkdkmaeayseigmkgerrrgkghdgl yqglstatkdtydalhmqalppr (SEQ ID NO: 1053), or a sequence substantially homologous thereto. The optional sequence of the signal peptide is shown in capital letters and parenthesis.

In one embodiment, the amino acid sequence is:

(SEQ ID NO: 1054)
diqmtqttsslsaslgdrvtiscrasqdiskylnwyqqkpdgtvklliyh
tsrlhsgvpsrfsgsgsgtdysltisnleqediatyfcqqgntlpytfgg
gtkleitggggsggggsggggsevklqesgpglvapsqslsvtctvsgvs
lpdygvswirqpprkglewlgviwgsettyynsalksrltiikdnsksqv
flkmnslqtddtaiyycakhyyyggsyamdywgqgtsvtvsstttpaprp
ptpaptiasqplslrpeacrpaaggavhtrgldfacdiyiwaplagtcgv
lllslvitlyckrgrkkllyifkqpfmrpvqttqeedgescrfpeeeegg
celrvkfsrsadapaykqgqnqlynelnlgrreeydvldkrrgrdpemgg
kprrknpqeglynelqkdkmaeayseigmkgerrrgkghdglyqglstat
kdtydalhmqalppr,
or
a sequence substantially homologous thereto.

In one embodiment, the CD19 CAR has the USAN designation TISAGENLECLEUCEL-T. In embodiments, CTL019 is made by a gene modification of T cells is mediated by stable insertion via transduction with a self-inactivating, replication deficient Lentiviral (LV) vector containing the CTL019 transgene under the control of the EF-1 alpha promoter. CTL019 can be a mixture of transgene positive and negative T cells that are delivered to the subject on the basis of percent transgene positive T cells.

In other embodiments, the CD19 CAR comprises an antigen binding domain (e.g., a humanized antigen binding domain) according to Table 3 of WO2014/153270, incorporated herein by reference.

Humanization of murine CD19 antibody is desired for the clinical setting, where the mouse-specific residues may induce a human-anti-mouse antigen (HAMA) response in patients who receive CART19 treatment, i.e., treatment with T cells transduced with the CAR19 construct. The production, characterization, and efficacy of humanized CD19 CAR sequences is described in International Application WO2014/153270 which is herein incorporated by reference in its entirety, including Examples 1-5 (p. 115-159).

In some embodiments, CD19 CAR constructs are described in PCT publication WO 2012/079000, incorporated herein by reference, and the amino acid sequence of the murine CD19 CAR and scFv constructs are shown in Table 3 below, or a sequence substantially identical to any of the aforesaid sequences (e.g., at least 85%, 90%, 95% or more identical to any of the sequences described herein).

TABLE 11
CD19 CAR Constructs

| SEQ ID | | |
|---|---|---|
| NO | Region | Sequence |
| CTL019 | | |
| SEQ ID NO: | CTL019 | MALPVTALLLPLALLLHAARPDIQMTQTTSSLSASLGDRVTISCRASQDIS |
| 1055 | Full amino | KYLNWYQQKPDGTVKLLIYHTSRLHSGVPSRFSGSGSGTDYSLTISNLEQE |
| | acid | DIATYFCQQGNTLPYTFGGGTKLEITGGGGSGGGGSGGGGSEVKLQESGP |
| | sequence | GLVAPSQSLSVTCTVSGVSLPDYGVSWIRQPPRKGLEWLGVIWGSETTYY |
| | | NSALKSRLTIIKDNSKSQVFLKMNSLQTDDTAIYYCAKHYYYGGSYAMD |
| | | YWGQGTSVTVSSTTTPAPRPPTPAPTIASQPLSLRPEACRPAAGGAVHTRG |
| | | LDFACDIYIWAPLAGTCGVLLLSLVITLYCKRGRKKLLYIFKQPFMRPVQT |
| | | TQEEDGCSCRFPEEEEGGCELRVKFSRSADAPAYKQGQNQLYNELNLGRR |
| | | EEYDVLDKRRGRDPEMGGKPRRKNPQEGLYNELQKDKMAEAYSEIGMK |
| | | GERRRGKGHDGLYQGLSTATKDTYDALHMQALPPR |
| SEQ ID NO: | CTL019 | ATGGCCTTACCAGTGACCGCCTTGCTCCTGCCGCTGGCCTTGCTGCTCC |
| 1056 | Full | ACGCCGCCAGGCCGGACATCCAGATGACACAGACTACATCCTCCCTGT |
| | nucleotide | CTGCCTCTCTGGGAGACAGAGTCACCATCAGTTGCAGGGCAAGTCAGG |
| | sequence | ACATTAGTAAATATTTAAATTGGTATCAGCAGAAACCAGATGGAACTG |
| | | TTAAACTCCTGATCTACCATACATCAAGATTACACTCAGGAGTCCCATC |
| | | AAGGTTCAGTGGCAGTGGGTCTGGAACAGATTATTCTCTCACCATTAG |
| | | CAACCTGGAGCAAGAAGATATTGCCACTTACTTTTGCCAACAGGGTAA |
| | | TACGCTTCCGTACACGTTCGGAGGGGGGACCAAGCTGGAGATCACAGG |
| | | TGGCGGTGGCTCGGGCGGTGGTGGGTCGGGTGGCGGCGGATCTGAGGT |
| | | GAAACTGCAGGAGTCAGGACCTGGCCTGGTGGCGCCCTCACAGAGCCT |
| | | GTCCGTCACATGCACTGTCTCAGGGGTCTCATTACCCGACTATGGTGTA |
| | | AGCTGGATTCGCCAGCCTCCACGAAAGGGTCTGGAGTGGCTGGGAGTA |
| | | ATATGGGGTAGTGAAACCACATACTATAATTCAGCTCTCAAATCCAGA |
| | | CTGACCATCATCAAGGACAACTCCAAGAGCCAAGTTTTCTTAAAAATG |
| | | AACAGTCTGCAAACTGATGACACAGCCATTTACTACTGTGCCAAACAT |
| | | TATTACTACGGTGGTAGCTATGCTATGGACTACTGGGGCCAAGGAACC |
| | | TCAGTCACCGTCTCCTCAACCACGACGCCAGCGCCGCGACCACCAACA |
| | | CCGGCGCCCACCATCGCGTCGCAGCCCCTGTCCCTGCGCCCAGAGGCG |
| | | TGCCGGCCAGCGGCGGGGGGCGCAGTGCACACGAGGGGGCTGGACTT |
| | | CGCCTGTGATATCTACATCTGGGCGCCCTTGGCCGGGACTTGTGGGGT |
| | | CCTTCTCCTGTCACTGGTTATCACCCTTTACTGCAAACGGGGCAGAAAG |
| | | AAACTCCTGTATATATTCAAACAACCATTTATGAGACCAGTACAAACT |
| | | ACTCAAGAGGAAGATGGCTGTAGCTGCCGATTTCCAGAAGAAGAAGA |
| | | AGGAGGATGTGAACTGAGAGTGAAGTTCAGCAGGAGCGCAGACGCCC |
| | | CCGCGTACAAGCAGGGCCAGAACCAGCTCTATAACGAGCTCAATCTAG |
| | | GACGAAGAGAGGAGTACGATGTTTTGGACAAGAGACGTGGCCGGGAC |
| | | CCTGAGATGGGGGGAAAGCCGAGAAGGAAGAACCCTCAGGAAGGCCT |
| | | GTACAATGAACTGCAGAAAGATAAGATGGCGGAGGCCTACAGTGAGA |
| | | TTGGGATGAAAGGCGAGCGCCGGAGGGGCAAGGGGCACGATGGCCTT |
| | | TACCAGGGTCTCAGTACAGCCACCAAGGACACCTACGACGCCCTTCAC |
| | | ATGCAGGCCCTGCCCCCTCGC |
| SEQ ID NO: | CTL019 | DIQMTQTTSSLSASLGDRVTISCRASQDISKYLNWYQQKPDGTVKLLIYHT |
| 1057 | scFv domain | SRLHSGVPSRFSGSGSGTDYSLTISNLEQEDIATYFCQQGNTLPYTFGGGTK |
| | | LEITGGGGSGGGGSGGGGSEVKLQESGPGLVAPSQSLSVTCTVSGVSLPD |
| | | YGVSWIRQPPRKGLEWLGVIWGSETTYYNSALKSRLTIIKDNSKSQVFLK |
| | | MNSLQTDDTAIYYCAKHYYYGGSYAMDYWGQGTSVTVSS |
| mCAR1 | | |
| SEQ ID NO: | mCAR1 | QVQLLESGAELVRPGSSVKISCKASGYAFSSYWMNWVKQRPGQGLEWIG |
| 1058 | scFv | QIYPGDGDTNYNGKFKGQATLTADKSSSTAYMQLSGLTSEDSAVYSCAR |
| | | KTISSVVDFYFDYWGQGTTVTGGGSGGGSGGGSGGGSELVLTQSPKFMST |
| | | SVGDRVSVTCKASQNVGTNVAWYQQKPGQSPKPLIYSATYRNSGVPDRF |
| | | TGSGSGTDFTLTITNVQSKDLADYFCQYNRYPYTSFFFTKLEIKRRS |
| SEQ ID NO: | mCAR1 Full | QVQLLESGAELVRPGSSVKISCKASGYAFSSYWMNWVKQRPGQGLEWIG |
| 1059 | amino acid | QIYPGDGDTNYNGKFKGQATLTADKSSSTAYMQLSGLTSEDSAVYSCAR |
| | sequence | KTISSVVDFYFDYWGQGTTVTGGGSGGGSGGGSGGGSELVLTQSPKFMST |
| | | SVGDRVSVTCKASQNVGTNVAWYQQKPGQSPKPLIYSATYRNSGVPDRF |
| | | TGSGSGTDFTLTITNVQSKDLADYFCQYNRYPYTSFFFTKLEIKRRSKIEV |
| | | MYPPPYLDNEKSNGTIIHVKGKHLCPSPLFPGPSKPFWVLVVVGGVLACY |
| | | SLLVTVAFIIFWVRSKRSRLLHSDYMNMTPRRPGPTRKHYQPYAPPRDFA |
| | | AYRSRVKFSRSADAPAYQQGQNQLYNELNLGRREEYDVLDKRRGRDPE |
| | | MGGKPRRKNPQEGLYNELQKDKMAEAYSEIGMKGERRRGKGHDGLYQG |
| | | LSTATKDTYDALHMQALPPR |
| mCAR2 | | |
| SEQ ID NO: | mCAR2 | DIQMTQTTSSLSASLGDRVTISCRASQDISKYLNWYQQKPDGTVKLLIYHT |
| 1060 | scFv | SRLHSGVPSRFSGSGSGTDYSLTISNLEQEDIATYFCQQGNTLPYTFGGGTK |
| | | LEITGSTSGSGKPGSGEGSTKGEVKLQESGPGLVAPSQSLSVTCTVSGVSLP |
| | | DYGVSWIRQPPRKGLEWLGVIWGSETTYYNSALKSRLTIIKDNSKSQVFL |
| | | KNINSLQTDDTAIYYCAKHYYYGGSYAMDYWGQGTSVTVSSE |
| SEQ ID NO: | mCAR2 | DIQMTQTTSSLSASLGDRVTISCRASQDISKYLNWYQQKPDGTVKLLIYHT |
| 1061 | amino acid | SRLHSGVPSRFSGSGSGTDYSLTISNLEQEDIATYFCQQGNTLPYTFGGGTK |
| | sequence | LEITGSTSGSGKPGSGEGSTKGEVKLQESGPGLVAPSQSLSVTCTVSGVSLP |
| | | DYGVSWIRQPPRKGLEWLGVIWGSETTYYNSALKSRLTIIKDNSKSQVFL |
| | | KMNSLQTDDTAIYYCAKHYYYGGSYAMDYWGQGTSVTVSSESKYGPPC |
| | | PPCPMFWVLVVVGGVLACYSLLVTVAFIIFWVKRGRKKLLYIFKQPFMRP |
| | | VQTTQEEDGCSCRFEEEEGGCELRVKFSRSADAPAYQQGQNQLYNELNL |
| | | GRREEYDVLDKRRGRDPEMGGKPRRKNPQEGLYNELQKDKMAEAYSEI |
| | | GMKGERRRGKGHDGLYQGLSTATKDTYDALHMQALPPRL |
| SEQ ID NO: | mCAR2 full | DIQMTQTTSSLSASLGDRVTISCRASQDISKYLNWYQQKPDGTVKLLIYHT |
| 1062 | amino acid | SRLHSGVPSRFSGSGSGTDYSLTISNLEQEDIATYFCQQGNTLPYTFGGGTK |
| | sequence | LEITGSTSGSGKPGSGEGSTKGEVKLQESGPGLVAPSQSLSVTCTVSGVSLP |
| | | DYGVSWIRQPPRKGLEWLGVIWGSETTYYNSALKSRLTIIKDNSKSQVFL |
| | | KMNSLQTDDTAIYYCAKHYYYGGSYAMDYWGQGTSVTVSSESKYGPPC |
| | | PPCPMFWVLVVVGGVLACYSLLVTVAFIIFWVKRGRKKLLYIFKQPFMRP |
| | | VQTTQEEDGCSCRFEEEEGGCELRVKFSRSADAPAYQQGQNQLYNELNL |
| | | GRREEYDVLDKRRGRDPEMGGKPRRKNPQEGLYNELQKDKMAEAYSEI |
| | | GMKGERRRGKGHDGLYQGLSTATKDTYDALHMQALPPRLEGGGEGRGS |
| | | LLTCGDVEENPGPRMLLLVTSLLLCELPHPAFLLIPRKVCNGIGIGEFKDSL |
| | | SINATNIKHFKNCTSISGDLHILPVAFRGDSFTHTPPLDPQELDILKTVKEIT |
| | | GFLLIQAWPENRTDLHAFENLEIIRGRTKQHGQFSLAVVSLNITSLGLRSLK |
| | | EISDGDVIISGNKNLCYANTINWKKLFGTSGQKTKIISNRGENSCKATGQV |
| | | CHALCSPEGCWGPEPRDCVSCRNVSRGRECVDKCNLLEGEPREFVENSEC |
| | | IQCHPECLPQAMNITCTGRGPDNCIQCAHYIDGPHCVKTCPAGVMGENNT |
| | | LVWKYADAGHVCHLCHPNCTYGCTGPGLEGCPTNGPKIPSIATGMVGAL |
| | | LLLLVVALGIGLFM |
| mCAR3 | | |
| SEQ ID NO: | mCAR3 | DIQMTQTTSSLSASLGDRVTISCRASQDISKYLNWYQQKPDGTVKLLIYHT |
| 1063 | scFv | SRLHSGVPSRFSGSGSGTDYSLTISNLEQEDIATYFCQQGNTLPYTFGGGTK |
| | | LEITGSTSGSGKPGSGEGSTKGEVKLQESGPGLVAPSQSLSVTCTVSGVSLP |
| | | DYGVSWIRQPPRKGLEWLGVIWGSETTYYNSALKSRLTIIKDNSKSQVFL |
| | | KNINSLQTDDTAIYYCAKHYYYGGSYAMDYWGQGTSVTVSS |
| SEQ ID NO: | mCAR3 full | DIQMTQTTSSLSASLGDRVTISCRASQDISKYLNWYQQKPDGTVKLLIYHT |
| 1064 | amino acid | SRLHSGVPSRFSGSGSGTDYSLTISNLEQEDIATYFCQQGNTLPYTFGGGTK |
| | sequence | LEITGSTSGSGKPGSGEGSTKGEVKLQESGPGLVAPSQSLSVTCTVSGVSLP |
| | | DYGVSWIRQPPRKGLEWLGVIWGSETTYYNSALKSRLTIIKDNSKSQVFL |
| | | KMNSLQTDDTAIYYCAKHYYYGGSYAMDYWGQGTSVTVSSAAAIEVMY |
| | | PPPYLDNEKSNGTIIHVKGKHLCPSPLFPGPSKPFWVLVVVGGVLACYSLL |
| | | VTVAFIIFWVRSKRSRLLHSDYMNMTPRRPGPTRKHYQPYAPPRDFAAYR |
| | | SRVKFSRSADAPAYQQGQNQLYNELNLGRREEYDVLDKRRGRDPEMGG |
| | | KPRRKNPQEGLYNELQKDKMAEAYSEIGMKGERRRGKGHDGLYQGLST |
| | | ATKDTYDALHMQALPPR |
| SSJ25-C1 | | |
| SEQ ID NO: | SSJ25-C1 | QVQLLESGAELVRPGSSVKISCKASGYAFSSYWMNWVKQRPGQGLEWIG |
| 1065 | VH | QIYPGDGDTNYNGKFKGQATLTADKSSSTAYMQLSGLTSEDSAVYSCAR |
| | sequence | KTISSVVDFYFDYWGQGTTVT |
| SEQ ID NO: | SSJ25-C1 | ELVLTQSPKFMSTSVGDRVSVTCKASQNVGTNVAWYQQKPGQSPKPLIYS |
| 1066 | VL | ATYRNSGVPDRFTGSGSGTDFTLTITNVQSKDLADYFYFCQYNRYPYTSG |
| | | GGTKLEIKRRS |
| Humanized | | |
| CAR1 | | |
| SEQ ID NO: | CAR1 scFv | EIVMTQSPATLSLSPGERATLSCRASQDISKYLNWYQQKPGQAPRLLIYHT |
| 1067 | domain | SRLHSGIPARFSGSGSGTDYTLTISSLQPEDFAVYFCQQGNTLPYTFGQGTK |
| | | LEIKGGGGSGGGGSGGGGSQVQLQESGPGLVKPSETLSLTCTVSGVSLPD |
| | | YGVSWIRQPPGKGLEWIGVIWGSETTYYSSSLKSRVTISKDNSKNQVSLKL |
| | | SSVTAADTAVYYCAKHYYYGGSYAMDYWGQGTLVTVSS |
| SEQ ID NO: | CAR 1- | MALPVTALLLPLALLLHAARPEIVMTQSPATLSLSPGERATLSCRASQDIS |
| 1068 | Full-aa | KYLNWYQQKPGQAPRLLIYHTSRLHSGIPARFSGSGSGTDYTLTISSLQPE |
| | | DFAVYFCQQGNTLPYTFGQGTKLEIKGGGGSGGGGSGGGGSQVQLQESG |
| | | PGLVKPSETLSLTCTVSGVSLPDYGVSWIRQPPGKGLEWIGVIWGSETTYY |
| | | SSSLKSRVTISKDNSKNQVSLKLSSVTAADTAVYYCAKHYYYGGSYAMD |
| | | YWGQGTLVTVSSTTTPAPRPPTPAPTIASQPLSLRPEACRPAAGGAVHTRG |
| | | LDFACDIYIWAPLAGTCGVLLLSLVITLYCKRGRKKLLYIFKQPFMRPVQT |
| | | TQEEDGCSCRFPEEEEGGCELRVKFSRSADAPAYKQGQNQLYNELNLGRR |
| | | EEYDVLDKRRGRDPEMGGKPRRKNPQEGLYNELQKDKMAEAYSEIGMK |
| | | GERRRGKGHDGLYQGLSTATKDTYDALHMQALPPR |
| Humanized | | |
| CAR2 | | |
| SEQ ID NO: | CAR2 scFv | EIVMTQSPATLSLSPGERATLSCRASQDISKYLNWYQQKPGQAPRLLIYHT |
| 1069 | domain-aa | SRLHSGIPARFSGSGSGTDYTLTISSLQPEDFAVYFCQQGNTLPYTFGQGTK |
| | (Linker is | LEIKGGGGSGGGGSGGGGSQVQLQESGPGLVKPSETLSLTCTVSGVSLPD |
| | underlined) | YGVSWIRQPPGKGLEWIGVIWGSETTYYQSSLKSRVTISKDNSKNQVSLK |
| | | LSSVTAADTAVYYCAKHYYYGGSYAMDYWGQGTLVTVSS |
| SEQ ID NO: | CAR2 scFv | atggccctccctgtcaccgccctgctgcttccgctggctcttctgctccacgccgctcggcccgaaattgtgatgaccc |
| 1070 | domain-nt | agtcacccgccactcttagcctttcacccggtgagcgcgcaaccctgtcttgcagagcctcccaagacatctcaaaat |
| | | accttaattggtatcaacagaagcccggacaggctcctcgccttctgatctaccacaccagccggctccattctggaat |
| | | ccctgccaggttcagcggtagcggatctgggaccgactacaccctcactatcagctcactgcagccagaggacttcg |
| | | ctgtctatttctgtcagcaagggaacaccctgccctacacctttggacagggcaccaagctcgagattaaaggtggag |
| | | gtggcagcggaggaggtgggtccggcggtggaggaagccaggtccaactccaagaaagcggaccgggtcttgtg |
| | | aagccatcagaaactctttcactgacttgtactgtgagcggagtgtctctccccgattacggggtgtcttggatcagaca |
| | | gccaccggggaagggtctggaatggattggagtgatttggggctctgagactacttactaccaatcatccctcaagtc |
| | | acgcgtcaccatctcaaaggacaactctaagaatcaggtgtcactgaaactgtcatctgtgaccgcagccgacaccg |
| | | ccgtgtactattgcgctaagcattactattatggcgggagctacgcaatggattactggggacagggtactctggtcac |
| | | cgtgtccagccaccaccatcatcaccatcaccat |
| SEQ ID NO: | CAR 2- | MALPVTALLLPLALLLHAARPEIVMTQSPATLSLSPGERATLSCRASQDIS |
| 1071 | Full-aa | KYLNWYQQKPGQAPRLLIYHTSRLHSGIPARFSGSGSGTDYTLTISSLQPE |
| | | DFAVYFCQQGNTLPYTFGQGTKLEIKGGGGSGGGGSGGGGSQVQLQESG |
| | | PGLVKPSETLSLTCTVSGVSLPDYGVSWIRQPPGKGLEWIGVIWGSETTYY |
| | | QSSLKSRVTISKDNSKNQVSLKLSSVTAADTAVYYCAKHYYYGGSYAMD |
| | | YWGQGTLVTVSSTTTPAPRPPTPAPTIASQPLSLRPEACRPAAGGAVHTRG |
| | | LDFACDIYIWAPLAGTCGVLLLSLVITLYCKRGRKKLLYIFKQPFMRPVQT |
| | | TQEEDGCSCRFPEEEEGGCELRVKFSRSADAPAYKQGQNQLYNELNLGRR |
| | | EEYDVLDKRRGRDPEMGGKPRRKNPQEGLYNELQKDKMAEAYSEIGMK |
| | | GERRRGKGHDGLYQGLSTATKDTYDALHMQALPPR |
| SEQ ID NO: | CAR 2- | atggccctccctgtcaccgccctgctgcttccgctggctcttctgctccacgccgctcggcccgaaattgtgatgaccc |
| 1072 | Full-nt | agtcacccgccactcttagcctttcacccggtgagcgcgcaaccctgtcttgcagagcctcccaagacatctcaaaat |
| | | accttaattggtatcaacagaagcccggacaggctcctcgccttctgatctaccacaccagccggctccattctggaat |
| | | ccctgccaggttcagcggtagcggatctgggaccgactacaccctcactatcagctcactgcagccagaggacttcg |
| | | ctgtctatttctgtcagcaagggaacaccctgccctacacctttggacagggcaccaagctcgagattaaaggtggag |
| | | gtggcagcggaggaggtgggtccggcggtggaggaagccaggtccaactccaagaaagcggaccgggtcttgtg |
| | | aagccatcagaaactctttcactgacttgtactgtgagcggagtgtctctccccgattacggggtgtcttggatcagaca |
| | | gccaccggggaagggtctggaatggattggagtgatttggggctctgagactacttactaccaatcatccctcaagtc |
| | | acgcgtcaccatctcaaaggacaactctaagaatcaggtgtcactgaaactgtcatctgtgaccgcagccgacaccg |
| | | ccgtgtactattgcgctaagcattactattatggcgggagctacgcaatggattactggggacagggtactctggtcac |
| | | cgtgtccagcaccactaccccagcaccgaggccacccaccccggctcctaccatcgcctcccagcctctgtccctgc |
| | | gtccggaggcatgtagacccgcagctggtggggccgtgcatacccggggtcttgacttcgcctgcgatatctacattt |
| | | gggcccctctggctggtacttgcggggtcctgctgctttcactcgtgatcactctttactgtaagcgcggtcggaagaa |
| | | gctgctgtacatctttaagcaacccttcatgaggcctgtgcagactactcaagaggaggacggctgttcatgccggttc |
| | | ccagaggaggaggaaggcggctgcgaactgcgcgtgaaattcagccgcagcgcagatgctccagcctacaagca |
| | | ggggcagaaccagctctacaacgaactcaatcttggtcggagagaggagtacgacgtgctggacaagcggagag |
| | | gacgggacccagaaatgggcgggaagccgcgcagaaagaatccccaagagggcctgtacaacgagctccaaaa |
| | | ggataagatggcagaagcctatagcgagattggtatgaaaggggaacgcagaagaggcaaaggccacgacggac |
| | | tgtaccagggactcagcaccgccaccaaggacacctatgacgctcttcacatgcaggccctgccgcctcgg |
| SEQ ID NO: | CAR2- | MALPVTALLLPLALLLHAARPeivmtqspatlslspgeratlscrasqdiskylnwyqqkpgq |
| 1073 | Soluble | aprlliyhtsrlhsgiparfsgsgsgtdytltisslqpedfavyfcqqgntlpytfgqgtkleikggggsggggsgggg |
| | scFv-aa | sqvqlqesgpglvkpsetlsltctvsgvslpdygvswirqppgkglewigviwgsettyyqsslksrvtiskdnsk |
| | | nqvslklssvtaadtavyycakhyyyggsyamdywgqgtlvtvsshhhhhhhh |
| Humanized | | |
| CAR3 | | |
| SEQ ID NO: | CAR3 scFv | QVQLQESGPGLVKPSETLSLTCTVSGVSLPDYGVSWIRQPPGKGLEWIGVI |
| 1074 | domain | WGSETTYYSSSLKSRVTISKDNSKNQVSLKLSSVTAADTAVYYCAKHYY |
| | | YGGSYAMDYWGQGTLVTVSSGGGGSGGGGSGGGGSEIVMTQSPATLSLS |
| | | PGERATLSCRASQDISKYLNWYQQKPGQAPRLLIYHTSRLHSGIPARFSGS |
| | | GSGTDYTLTISSLQPEDFAVYFCQQGNTLPYTFGQGTKLEIK |
| SEQ ID NO: | CAR 3- | MALPVTALLLPLALLLHAARPQVQLQESGPGLVKPSETLSLTCTVSGVSLP |
| 1075 | Full-aa | DYGVSWIRQPPGKGLEWIGVIWGSETTYYSSSLKSRVTISKDNSKNQVSLK |
| | | LSSVTAADTAVYYCAKHYYYGGSYAMDYWGQGTLVTVSSGGGGSGGG |
| | | GSGGGGSEIVMTQSPATLSLSPGERATLSCRASQDISKYLNWYQQKPGQA |
| | | PRLLIYHTSRLHSGIPARFSGSGSGTDYTLTISSLQPEDFAVYFCQQGNTLP |
| | | YTFGQGTKLEIKTTTPAPRPPTPAPTIASQPLSLRPEACRPAAGGAVHTRGL |
| | | DFACDIYIWAPLAGTCGVLLLSLVITLYCKRGRKKLLYIFKQPFMRPVQTT |
| | | QEEDGCSCRFPEEEEGGCELRVKFSRSADAPAYKQGQNQLYNELNLGRRE |
| | | EYDVLDKRRGRDPEMGGKPRRKNPQEGLYNELQKDKMAEAYSEIGMKG |
| | | ERRRGKGHDGLYQGLSTATKDTYDALHMQALPPR |
| Humanized | | |
| CAR4 | | |
| SEQ ID NO: | CAR4 scFv | QVQLQESGPGLVKPSETLSLTCTVSGVSLPDYGVSWIRQPPGKGLEWIGVI |
| 1076 | domain | WGSETTYYQSSLKSRVTISKDNSKNQVSLKLSSVTAADTAVYYCAKHYY |
| | | YGGSYAMDYWGQGTLVTVSSGGGGSGGGGSGGGGSEIVMTQSPATLSLS |
| | | PGERATLSCRASQDISKYLNWYQQKPGQAPRLLIYHTSRLHSGIPARFSGS |
| | | GSGTDYTLTISSLQPEDFAVYFCQQGNTLPYTFGQGTKLEIK |
| SEQ ID NO: | CAR 4- | MALPVTALLLPLALLLHAARPQVQLQESGPGLVKPSETLSLTCTVSGVSLP |
| 1077 | Full-aa | DYGVSWIRQPPGKGLEWIGVIWGSETTYYQSSLKSRVTISKDNSKNQVSL |
| | | KLSSVTAADTAVYYCAKHYYYGGSYAMDYWGQGTLVTVSSGGGGSGG |
| | | GGSGGGGSEIVMTQSPATLSLSPGERATLSCRASQDISKYLNWYQQKPGQ |
| | | APRLLIYHTSRLHSGIPARFSGSGSGTDYTLTISSLQPEDFAVYFCQQGNTL |
| | | PYTFGQGTKLEIKTTTPAPRPPTPAPTIASQPLSLRPEACRPAAGGAVHTRG |
| | | LDFACDIYIWAPLAGTCGVLLLSLVITLYCKRGRKKLLYIFKQPFMRPVQT |
| | | TQEEDGCSCRFPEEEEGGCELRVKFSRSADAPAYKQGQNQLYNELNLGRR |
| | | EEYDVLDKRRGRDPEMGGKPRRKNPQEGLYNELQKDKMAEAYSEIGMK |
| | | GERRRGKGHDGLYQGLSTATKDTYDALHMQALPPR |
| Humanized | | |
| CAR5 | | |
| SEQ ID NO: | CARS scFv | EIVMTQSPATLSLSPGERATLSCRASQDISKYLNWYQQKPGQAPRLLIYHT |
| 1078 | domain | SRLHSGIPARFSGSGSGTDYTLTISSLQPEDFAVYFCQQGNTLPYTFGQGTK |
| | | LEIKGGGGSGGGGSGGGGSGGGGSQVQLQESGPGLVKPSETLSLTCTVSG |
| | | VSLPDYGVSWIRQPPGKGLEWIGVIWGSETTYYSSSLKSRVTISKDNSKNQ |
| | | VSLKLSSVTAADTAVYYCAKHYYYGGSYAMDYWGQGTLVTVSS |
| SEQ ID NO: | CAR 5- | MALPVTALLLPLALLLHAARPEIVMTQSPATLSLSPGERATLSCRASQDIS |
| 1079 | Full-aa | KYLNWYQQKPGQAPRLLIYHTSRLHSGIPARFSGSGSGTDYTLTISSLQPE |
| | | DFAVYFCQQGNTLPYTFGQGTKLEIKGGGGSGGGGSGGGGSGGGGSQVQ |
| | | LQESGPGLVKPSETLSLTCTVSGVSLPDYGVSWIRQPPGKGLEWIGVIWGS |
| | | ETTYYSSSLKSRVTISKDNSKNQVSLKLSSVTAADTAVYYCAKHYYYGGS |
| | | YAMDYWGQGTLVTVSSTTTPAPRPPTPAPTIASQPLSLRPEACRPAAGGA |
| | | VHTRGLDFACDIYIWAPLAGTCGVLLLSLVITLYCKRGRKKLLYIFKQPFM |
| | | RPVQTTQEEDGCSCRFPEEEEGGCELRVKFSRSADAPAYKQGQNQLYNEL |
| | | NLGRREEYDVLDKRRGRDPEMGGKPRRKNPQEGLYNELQKDKMAEAYS |
| | | EIGMKGERRRGKGHDGLYQGLSTATKDTYDALHMQALPPR |
| Humanized | | |
| CAR6 | | |
| SEQ ID NO: | CAR6 | EIVMTQSPATLSLSPGERATLSCRASQDISKYLNWYQQKPGQAPRLLIYHT |
| 1080 | scFv domain | SRLHSGIPARFSGSGSGTDYTLTISSLQPEDFAVYFCQQGNTLPYTFGQGTK |
| | | LEIKGGGGSGGGGSGGGGSGGGGSQVQLQESGPGLVKPSETLSLTCTVSG |
| | | VSLPDYGVSWIRQPPGKGLEWIGVIWGSETTYYQSSLKSRVTISKDNSKNQ |
| | | VSLKLSSVTAADTAVYYCAKHYYYGGSYAMDYWGQGTLVTVSS |
| SEQ ID NO: | CAR6- | MALPVTALLLPLALLLHAARPEIVMTQSPATLSLSPGERATLSCRASQDIS |
| 1081 | Full-aa | KYLNWYQQKPGQAPRLLIYHTSRLHSGIPARFSGSGSGTDYTLTISSLQPE |
| | | DFAVYFCQQGNTLPYTFGQGTKLEIKGGGGSGGGGSGGGGSGGGGSQVQ |
| | | LQESGPGLVKPSETLSLTCTVSGVSLPDYGVSWIRQPPGKGLEWIGVIWGS |
| | | ETTYYQSSLKSRVTISKDNSKNQVSLKLSSVTAADTAVYYCAKHYYYGGS |
| | | YAMDYWGQGTLVTVSSTTTPAPRPPTPAPTIASQPLSLRPEACRPAAGGA |
| | | VHTRGLDFACDIYIWAPLAGTCGVLLLSLVITLYCKRGRKKLLYIFKQPFM |
| | | RPVQTTQEEDGCSCRFPEEEEGGCELRVKFSRSADAPAYKQGQNQLYNEL |
| | | NLGRREEYDVLDKRRGRDPEMGGKPRRKNPQEGLYNELQKDKMAEAYS |
| | | EIGMKGERRRGKGHDGLYQGLSTATKDTYDALHMQALPPR |
| Humanized | | |
| CAR7 | | |
| SEQ ID NO: | CAR7 scFv | QVQLQESGPGLVKPSETLSLTCTVSGVSLPDYGVSWIRQPPGKGLEWIGVI |
| 1082 | domain | WGSETTYYSSSLKSRVTISKDNSKNQVSLKLSSVTAADTAVYYCAKHYY |
| | | YGGSYAMDYWGQGTLVTVSSGGGGSGGGGSGGGGSGGGGSEIVMTQSP |
| | | ATLSLSPGERATLSCRASQDISKYLNWYQQKPGQAPRLLIYHTSRLHSGIP |
| | | ARFSGSGSGTDYTLTISSLQPEDFAVYFCQQGNTLPYTFGQGTKLEIK |
| SEQ ID NO: | CAR 7 | MALPVTALLLPLALLLHAARPQVQLQESGPGLVKPSETLSLTCTVSGVSLP |
| 1083 | Full-aa | DYGVSWIRQPPGKGLEWIGVIWGSETTYYSSSLKSRVTISKDNSKNQVSLK |
| | | LSSVTAADTAVYYCAKHYYYGGSYAMDYWGQGTLVTVSSGGGGSGGG |
| | | GSGGGGSGGGGSEIVMTQSPATLSLSPGERATLSCRASQDISKYLNWYQQ |
| | | KPGQAPRLLIYHTSRLHSGIPARFSGSGSGTDYTLTISSLQPEDFAVYFCQQ |
| | | GNTLPYTFGQGTKLEIKTTTPAPRPPTPAPTIASQPLSLRPEACRPAAGGAV |
| | | HTRGLDFACDIYIWAPLAGTCGVLLLSLVITLYCKRGRKKLLYIFKQPFMR |
| | | PVQTTQEEDGCSCRFPEEEEGGCELRVKFSRSADAPAYKQGQNQLYNELN |
| | | LGRREEYDVLDKRRGRDPEMGGKPRRKNPQEGLYNELQKDKMAEAYSEI |
| | | GMKGERRRGKGHDGLYQGLSTATKDTYDALHMQALPPR |
| Humanized | | |
| CAR8 | | |
| SEQ ID NO: | CAR8 scFv | QVQLQESGPGLVKPSETLSLTCTVSGVSLPDYGVSWIRQPPGKGLEWIGVI |
| 1084 | domain | WGSETTYYQSSLKSRVTISKDNSKNQVSLKLSSVTAADTAVYYCAKHYY |
| | | YGGSYAMDYWGQGTLVTVSSGGGGSGGGGSGGGGSGGGGSEIVMTQSP |
| | | ATLSLSPGERATLSCRASQDISKYLNWYQQKPGQAPRLLIYHTSRLHSGIP |
| | | ARFSGSGSGTDYTLTISSLQPEDFAVYFCQQGNTLPYTFGQGTKLEIK |
| SEQ ID NO: | CAR 8- | MALPVTALLLPLALLLHAARPQVQLQESGPGLVKPSETLSLTCTVSGVSLP |
| 1085 | Full-aa | DYGVSWIRQPPGKGLEWIGVIWGSETTYYQSSLKSRVTISKDNSKNQVSL |
| | | KLSSVTAADTAVYYCAKHYYYGGSYAMDYWGQGTLVTVSSGGGGSGG |
| | | GGSGGGGSGGGGSEIVMTQSPATLSLSPGERATLSCRASQDISKYLNWYQ |
| | | QKPGQAPRLLIYHTSRLHSGIPARFSGSGSGTDYTLTISSLQPEDFAVYFCQ |
| | | QGNTLPYTFGQGTKLEIKTTTPAPRPPTPAPTIASQPLSLRPEACRPAAGGA |
| | | VHTRGLDFACDIYIWAPLAGTCGVLLLSLVITLYCKRGRKKLLYIFKQPFM |
| | | RPVQTTQEEDGCSCRFPEEEEGGCELRVKFSRSADAPAYKQGQNQLYNEL |
| | | NLGRREEYDVLDKRRGRDPEMGGKPRRKNPQEGLYNELQKDKMAEAYS |
| | | EIGMKGERRRGKGHDGLYQGLSTATKDTYDALHMQALPPR |
| Humanized | | |
| CAR9 | | |
| SEQ ID NO: | CAR9 scFv | EIVMTQSPATLSLSPGERATLSCRASQDISKYLNWYQQKPGQAPRLLIYHT |
| 1086 | domain | SRLHSGIPARFSGSGSGTDYTLTISSLQPEDFAVYFCQQGNTLPYTFGQGTK |
| | | LEIKGGGGSGGGGSGGGGSGGGGSQVQLQESGPGLVKPSETLSLTCTVSG |
| | | VSLPDYGVSWIRQPPGKGLEWIGVIWGSETTYYNSSLKSRVTISKDNSKNQ |
| | | VSLKLSSVTAADTAVYYCAKHYYYGGSYAMDYWGQGTLVTVSS |
| SEQ ID NO: | CAR 9- | MALPVTALLLPLALLLHAARPEIVMTQSPATLSLSPGERATLSCRASQDIS |
| 1087 | Full-aa | KYLNWYQQKPGQAPRLLIYHTSRLHSGIPARFSGSGSGTDYTLTISSLQPE |
| | | DFAVYFCQQGNTLPYTFGQGTKLEIKGGGGSGGGGSGGGGSGGGGSQVQ |
| | | LQESGPGLVKPSETLSLTCTVSGVSLPDYGVSWIRQPPGKGLEWIGVIWGS |
| | | ETTYYNSSLKSRVTISKDNSKNQVSLKLSSVTAADTAVYYCAKHYYYGGS |
| | | YAMDYWGQGTLVTVSSTTTPAPRPPTPAPTIASQPLSLRPEACRPAAGGA |
| | | VHTRGLDFACDIYIWAPLAGTCGVLLLSLVITLYCKRGRKKLLYIFKQPFM |
| | | RPVQTTQEEDGCSCRFPEEEEGGCELRVKFSRSADAPAYKQGQNQLYNEL |
| | | NLGRREEYDVLDKRRGRDPEMGGKPRRKNPQEGLYNELQKDKMAEAYS |
| | | EIGMKGERRRGKGHDGLYQGLSTATKDTYDALHMQALPPR |
| Humanized | | |
| CAR10 | | |
| SEQ ID NO: | CAR10 scFv | QVQLQESGPGLVKPSETLSLTCTVSGVSLPDYGVSWIRQPPGKGLEWIGVI |
| 1088 | domain | WGSETTYYNSSLKSRVTISKDNSKNQVSLKLSSVTAADTAVYYCAKHYY |
| | | YGGSYAMDYWGQGTLVTVSSGGGGSGGGGSGGGGSGGGGSEIVMTQSP |
| | | ATLSLSPGERATLSCRASQDISKYLNWYQQKPGQAPRLLIYHTSRLHSGIP |
| | | ARFSGSGSGTDYTLTISSLQPEDFAVYFCQQGNTLPYTFGQGTKLEIK |
| SEQ ID NO: | CAR 10 | MALPVTALLLPLALLLHAARPEIVMTQSPATLSLSPGERATLSCRASQDIS |
| 1089 | Full-aa | KYLNWYQQKPGQAPRLLIYHTSRLHSGIPARFSGSGSGTDYTLTISSLQPE |
| | | DFAVYFCQQGNTLPYTFGQGTKLEIKGGGGSGGGGSGGGGSGGGGSQVQ |
| | | LQESGPGLVKPSETLSLTCTVSGVSLPDYGVSWIRQPPGKGLEWIGVIWGS |
| | | ETTYYNSSLKSRVTISKDNSKNQVSLKLSSVTAADTAVYYCAKHYYYGGS |
| | | YAMDYWGQGTLVTVSSTTTPAPRPPTPAPTIASQPLSLRPEACRPAAGGA |
| | | VHTRGLDFACDIYIWAPLAGTCGVLLLSLVITLYCKRGRKKLLYIFKQPFM |
| | | RPVQTTQEEDGCSCRFPEEEEGGCELRVKFSRSADAPAYKQGQNQLYNEL |
| | | NLGRREEYDVLDKRRGRDPEMGGKPRRKNPQEGLYNELQKDKMAEAYS |
| | | EIGMKGERRRGKGHDGLYQGLSTATKDTYDALHMQALPPR |
| Humanized | | |
| CAR11 | | |
| SEQ ID NO: | CAR11 | EIVMTQSPATLSLSPGERATLSCRASQDISKYLNWYQQKPGQAPRLLIYHT |
| 1090 | scFv domain | SRLHSGIPARFSGSGSGTDYTLTISSLQPEDFAVYFCQQGNTLPYTFGQGTK |
| | | LEIKGGGGSGGGGSGGGGSQVQLQESGPGLVKPSETLSLTCTVSGVSLPD |
| | | YGVSWIRQPPGKGLEWIGVIWGSETTYYNSSLKSRVTISKDNSKNQVSLK |
| | | LSSVTAADTAVYYCAKHYYYGGSYAMDYWGQGTLVTVSS |
| SEQ ID NO: | CAR 11 | MALPVTALLLPLALLLHAARPQVQLQESGPGLVKPSETLSLTCTVSGVSLP |
| 1091 | Full-aa | DYGVSWIRQPPGKGLEWIGVIWGSETTYYNSSLKSRVTISKDNSKNQVSL |
| | | KLSSVTAADTAVYYCAKHYYYGGSYAMDYWGQGTLVTVSSGGGGSGG |
| | | GGSGGGGSGGGGSEIVMTQSPATLSLSPGERATLSCRASQDISKYLNWYQ |
| | | QKPGQAPRLLIYHTSRLHSGIPARFSGSGSGTDYTLTISSLQPEDFAVYFCQ |
| | | QGNTLPYTFGQGTKLEIKTTTPAPRPPTPAPTIASQPLSLRPEACRPAAGGA |
| | | VHTRGLDFACDIYIWAPLAGTCGVLLLSLVITLYCKRGRKKLLYIFKQPFM |
| | | RPVQTTQEEDGCSCRFPEEEEGGCELRVKFSRSADAPAYKQGQNQLYNEL |
| | | NLGRREEYDVLDKRRGRDPEMGGKPRRKNPQEGLYNELQKDKMAEAYS |
| | | EIGMKGERRRGKGHDGLYQGLSTATKDTYDALHMQALPPR |
| Humanized | | |
| CAR12 | | |
| SEQ ID NO: | CAR12 | QVQLQESGPGLVKPSETLSLTCTVSGVSLPDYGVSWIRQPPGKGLEWIGVI |
| 1092 | scFv domain | WGSETTYYNSSLKSRVTISKDNSKNQVSLKLSSVTAADTAVYYCAKHYY |
| | | YGGSYAMDYWGQGTLVTVSSGGGGSGGGGSGGGGSEIVMTQSPATLSLS |
| | | PGERATLSCRASQDISKYLNWYQQKPGQAPRLLIYHTSRLHSGIPARFSGS |
| | | GSGTDYTLTISSLQPEDFAVYFCQQGNTLPYTFGQGTKLEIK |
| SEQ ID NO: | CAR 12- | MALPVTALLLPLALLLHAARPEIVMTQSPATLSLSPGERATLSCRASQDIS |
| 1093 | Full-aa | KYLNWYQQKPGQAPRLLIYHTSRLHSGIPARFSGSGSGTDYTLTISSLQPE |
| | | DFAVYFCQQGNTLPYTFGQGTKLEIKGGGGSGGGGSGGGGSQVQLQESG |
| | | PGLVKPSETLSLTCTVSGVSLPDYGVSWIRQPPGKGLEWIGVIWGSETTYY |
| | | NSSLKSRVTISKDNSKNQVSLKLSSVTAADTAVYYCAKHYYYGGSYAMD |
| | | YWGQGTLVTVSSTTTPAPRPPTPAPTIASQPLSLRPEACRPAAGGAVHTRG |
| | | LDFACDIYIWAPLAGTCGVLLLSLVITLYCKRGRKKLLYIFKQPFMRPVQT |
| | | TQEEDGCSCRFPEEEEGGCELRVKFSRSADAPAYKQGQNQLYNELNLGRR |
| | | EEYDVLDKRRGRDPEMGGKPRRKNPQEGLYNELQKDKMAEAYSEIGMK |
| | | GERRRGKGHDGLYQGLSTATKDTYDALHMQALPPR |
| Murine | | |
| CART19 | | |
| SEQ ID NO: | HCDR1 | DYGVS |
| 1094 | (Kabat) | |
| SEQ ID NO: | HCDR2 | VIWGSETTYYNSALKS |
| 1095 | (Kabat) | |
| SEQ ID NO: | HCDR3 | HYYYGGSYAMDY |
| 1096 | (Kabat) | |
| SEQ ID NO: | LCDR1 | RASQDISKYLN |
| 1097 | (Kabat) | |
| SEQ ID NO: | LCDR2 | HTSRLHS |
| 1098 | (Kabat) | |
| SEQ ID NO: | LCDR3 | QQGNTLPYT |
| 1099 | (Kabat) | |
| Humanized | | |
| CART19 a | | |
| SEQ ID NO: | HCDR1 | DYGVS |
| 1100 | (Kabat) | |
| SEQ ID NO: | HCDR2 | VIWGSETTYYSSSLKS |
| 1101 | (Kabat) | |
| SEQ ID NO: | HCDR3 | HYYYGGSYAMDY |
| 1102 | (Kabat) | |
| SEQ ID NO: | LCDR1 | RASQDISKYLN |
| 1103 | (Kabat) | |
| SEQ ID NO: | LCDR2 | HTSRLHS |
| 1104 | (Kabat) | |
| SEQ ID NO: | LCDR3 | QQGNTLPYT |
| 1105 | (Kabat) | |
| Humanized | | |
| CART19 b | | |
| SEQ ID NO: | HCDR1 | DYGVS |
| 1106 | (Kabat) | |
| SEQ ID NO: | HCDR2 | VIWGSETTYYQSSLKS |
| 1107 | (Kabat) | |
| SEQ ID NO: | HCDR3 | HYYYGGSYAMDY |
| 1108 | (Kabat) | |
| SEQ ID NO: | LCDR1 | RASQDISKYLN |
| 1109 | (Kabat) | |
| SEQ ID NO: | LCDR2 | HTSRLHS |
| 1110 | (Kabat) | |
| SEQ ID NO: | LCDR3 | QQGNTLPYT |
| 1111 | (Kabat) | |
| Humanized | | |
| CART19 c | | |
| SEQ ID NO: | HCDR1 | DYGVS |
| 1112 | (Kabat) | |
| SEQ ID NO: | HCDR2 | VIWGSETTYYNSSLKS |
| 1113 | (Kabat) | |
| SEQ ID NO: | HCDR3 | HYYYGGSYAMDY |
| 1114 | (Kabat) | |
| SEQ ID NO: | LCDR1 | RASQDISKYLN |
| 1115 | (Kabat) | |
| SEQ ID NO: | LCDR2 | HTSRLHS |
| 1116 | (Kabat) | |
| SEQ ID NO: | LCDR3 | QQGNTLPYT |
| 1117 | (Kabat) | |

CD19 CAR constructs containing humanized anti-CD19 scFv domains are described in PCT publication WO 2014/153270, incorporated herein by reference.

The sequences of murine and humanized CDR sequences of the anti-CD19 scFv domains are shown in Table 4 for the heavy chain variable domains and in Table 5 for the light chain variable domains. The SEQ ID NOs refer to those found in Table 3.

TABLE 4
Heavy Chain Variable Domain CDR (Kabat)
SEQ ID NO's of CD19 Antibodies

| Candidate | HCDR1 | HCDR2 | HCDR3 |
|---|---|---|---|
| murine_CART19 | SEQ ID NO: | SEQ ID NO: | SEQ ID NO: |
| | 1094 | 1095 | 1096 |
| humanized_CART19 a | SEQ ID NO: | SEQ ID NO: | SEQ ID NO: |
| | 1100 | 1101 | 1102 |
| humanized_CART19 b | SEQ ID NO: | SEQ ID NO: | SEQ ID NO: |
| | 1106 | 1107 | 1108 |
| humanized_CART19 c | SEQ ID NO: | SEQ ID NO: | SEQ ID NO: |
| | 1112 | 1113 | 1114 |

TABLE 5
Light Chain Variable Domain CDR (Kabat)
SEQ ID NO's of CD19 Antibodies

| Candidate | LCDR1 | LCDR2 | LCDR3 |
|---|---|---|---|
| murine_CART19 | SEQ ID NO: | SEQ ID NO: | SEQ ID NO: |
| | 1097 | 1098 | 1099 |
| humanized_CART19 a | SEQ ID NO: | SEQ ID NO: | SEQ ID NO: |
| | 1103 | 1104 | 1105 |
| humanized_CART19 b | SEQ ID NO: | SEQ ID NO: | SEQ ID NO: |
| | 1109 | 1110 | 1111 |
| humanized_CART19 c | SEQ ID NO: | SEQ ID NO: | SEQ ID NO: |
| | 1115 | 1116 | 1117 |

Any known CD19 CAR, e.g., the CD19 antigen binding domain of any known CD19 CAR, in the art can be used in accordance with the present disclosure. For example, LG-740; CD19 CAR described in the U.S. Pat. Nos. 8,399,645; 7,446,190; Xu et al., Leuk Lymphoma. 2013 54(2):255-260 (2012); Cruz et al., Blood 122(17):2965-2973 (2013); Brentjens et al., Blood, 118(18):4817-4828 (2011); Kochenderfer et al., Blood 116(20):4099-102 (2010); Kochenderfer et al., Blood 122(25):4129-39 (2013); and 16th Annu Meet Am Soc Gen Cell Ther (ASGCT) (May 15-18, Salt Lake City) 2013, Abst 10.

Exemplary CD19 CARs include CD19 CARs described herein, e.g., in one or more tables described herein, or an anti-CD19 CAR described in Xu et al. Blood 123.24(2014):3750-9; Kochenderfer et al. Blood 122.25(2013):4129-39, Cruz et al. Blood 122.17(2013):2965-73, NCT00586391, NCT01087294, NCT02456350, NCT00840853, NCT02659943, NCT02650999, NCT02640209, NCT01747486, NCT02546739, NCT02656147, NCT02772198, NCT00709033, NCT02081937, NCT00924326, NCT02735083, NCT02794246, NCT02746952, NCT01593696, NCT02134262, NCT01853631, NCT02443831, NCT02277522, NCT02348216, NCT02614066, NCT02030834, NCT02624258, NCT02625480, NCT02030847, NCT02644655, NCT02349698, NCT02813837, NCT02050347, NCT01683279, NCT02529813, NCT02537977, NCT02799550, NCT02672501, NCT02819583, NCT02028455, NCT01840566, NCT01318317, NCT01864889, NCT02706405, NCT01475058, NCT01430390, NCT02146924, NCT02051257, NCT02431988, NCT01815749, NCT02153580, NCT01865617, NCT02208362, NCT02685670, NCT02535364, NCT02631044, NCT02728882, NCT02735291, NCT01860937, NCT02822326, NCT02737085, NCT02465983, NCT02132624, NCT02782351, NCT01493453, NCT02652910, NCT02247609, NCT01029366, NCT01626495, NCT02721407, NCT01044069, NCT00422383, NCT01680991, NCT02794961, or NCT02456207, each of which is incorporated herein by reference in its entirety.

### BCMA CAR and BCMA-Binding Sequences

In some embodiments, the CAR IL-15R/IL-15-expressing cell described herein is a BCMA CAR-expressing cell (e.g., a cell expressing a CAR that binds to human BCMA). Exemplary BCMA CARs can include sequences disclosed in Table 1 or 16 of WO2016/014565, incorporated herein by reference. The BCMA CAR construct can include an optional leader sequence; an optional hinge domain, e.g., a CD8 hinge domain; a transmembrane domain, e.g., a CD8 transmembrane domain; an intracellular domain, e.g., a 4-1BB intracellular domain; and a functional signaling domain, e.g., a CD3 zeta domain. In certain embodiments, the domains are contiguous and in the same reading frame to form a single fusion protein. In other embodiments, the domain are in separate polypeptides, e.g., as in an RCAR molecule as described herein.

The sequences of exemplary BCMA CAR molecules or fragments thereof are disclosed in Tables 6, 7, 8, and 11. In certain embodiments, the full length BCMA CAR molecule includes one or more CDRs, VH, VL, scFv, or full-length sequences of, BCMA-1, BCMA-2, BCMA-3, BCMA-4, BCMA-5, BCMA-6, BCMA-7, BCMA-8, BCMA-9, BCMA-10, BCMA-11, BCMA-12, BCMA-13, BCMA-14, BCMA-15, 149362, 149363, 149364, 149365, 149366, 149367, 149368, 149369, BCMA_EBB-C1978-A4, BCMA_EBB-C1978-G1, BCMA_EBB-C1979-C1, BCMA_EBB-C1978-C7, BCMA_EBB-C1978-D10, BCMA_EBB-C1979-C12, BCMA_EBB-C1980-G4, BCMA_EBB-C1980-D2, BCMA_EBB-C1978-A10, BCMA_EBB-C1978-D4, BCMA_EBB-C1980-A2, BCMA_EBB-C1981-C3, BCMA_EBB-C1978-G4, A7D12.2, C11D5.3, C12A3.2, or C13F12.1, as disclosed in, Tables 6, 7, and 8 or a sequence substantially (e.g., 95-99%) identical thereto.

Additional exemplary BCMA-targeting sequences that can be used in the anti-BCMA CAR constructs are disclosed in WO 2017/021450, WO 2017/011804, WO 2017/025038, WO 2016/090327, WO 2016/130598, WO 2016/210293, WO 2016/090320, WO 2016/014789, WO 2016/094304, WO 2016/154055, WO 2015/166073, WO 2015/188119, WO 2015/158671, U.S. Pat. Nos. 9,243,058, 8,920,776, 9,273,141, 7,083,785, 9,034,324, US 2007/0049735, US 2015/0284467, US 2015/0051266, US 2015/0344844, US 2016/0131655, US 2016/0297884, US 2016/0297885, US 2017/0051308, US 2017/0051252, US 2017/0051252, WO 2016/020332, WO 2016/087531, WO 2016/079177, WO 2015/172800, WO 2017/008169, U.S. Pat. No. 9,340,621, US 2013/0273055, US 2016/0176973, US 2015/0368351, US 2017/0051068, US 2016/0368988, and US 2015/0232557, herein incorporated by reference in their entirety. In some embodiments, additional exemplary BCMA CAR constructs are generated using the VH and VL sequences from PCT Publication WO2012/0163805 (the contents of which are hereby incorporated by reference in its entirety).

TABLE 6
Amino Acid and Nucleic Acid Sequences of exemplary anti-BCMA scFv domains and
BCMA CAR molecules. The amino acid sequences variable heavy chain and variable
light chain sequences for each scFv is also provided.

| | SEQ | |
|---|---|---|
| Name/ | ID | |
| Description | NO: | Sequence |
| 139109 | | |
| 139109-aa | 49 | EVQLVESGGGLVQPGGSLRLSCAVSGFALSNHGMSWVRRAPGKGLEWVSGIVYSGSTY |
| ScFv domain | | YAASVKGRFTISRDNSRNTLYLQMNSLRPEDTAIYYCSAHGGESDVWGQGTTVTVSSA |
| | | SGGGGSGGRASGGGGSDIQLTQSPSSLSASVGDRVTITCRASQSISSYLNWYQQKPGK |
| | | APKLLIYAASSLQSGVPSRFSGSGSGTDFTLTISSLQPEDFATYYCQQSYSTPYTFGQ |
| | | GTKVEIK |
| 139109-nt | 64 | GAAGTGCAATTGGTGGAATCAGGGGGAGGACTTGTGCAGCCTGGAGGATCGCTGAGAC |
| ScFv domain | | TGTCATGTGCCGTGTCCGGCTTTGCCCTGTCCAACCACGGGATGTCCTGGGTCCGCCG |
| | | CGCGCCTGGAAAGGGCCTCGAATGGGTGTCGGGTATTGTGTACAGCGGTAGCACCTAC |
| | | TATGCCGCATCCGTGAAGGGGAGATTCACCATCAGCCGGGACAACTCCAGGAACACTC |
| | | TGTACCTCCAAATGAATTCGCTGAGGCCAGAGGACACTGCCATCTACTACTGCTCCGC |
| | | GCATGGCGGAGAGTCCGACGTCTGGGGACAGGGGACCACCGTGACCGTGTCTAGCGCG |
| | | TCCGGCGGAGGCGGCAGCGGGGGTCGGGCATCAGGGGGCGGCGGATCGGACATCCAGC |
| | | TCACCCAGTCCCCGAGCTCGCTGTCCGCCTCCGTGGGAGATCGGGTCACCATCACGTG |
| | | CCGCGCCAGCCAGTCGATTTCCTCCTACCTGAACTGGTACCAACAGAAGCCCGGAAAA |
| | | GCCCCGAAGCTTCTCATCTACGCCGCCTCGAGCCTGCAGTCAGGAGTGCCCTCACGGT |
| | | TCTCCGGCTCCGGTTCCGGTACTGATTTCACCCTGACCATTTCCTCCCTGCAACCGGA |
| | | GGACTTCGCTACTTACTACTGCCAGCAGTCGTACTCCACCCCCTACACTTTCGGACAA |
| | | GGCACCAAGGTCGAAATCAAG |
| 139109-aa | 79 | EVQLVESGGGLVQPGGSLRLSCAVSGFALSNHGMSWVRRAPGKGLEWVSGIVYSGSTY |
| VH | | YAASVKGRFTISRDNSRNTLYLQMNSLRPEDTAIYYCSAHGGESDVWGQGTTVTVSS |
| 139109-aa | 94 | DIQLTQSPSSLSASVGDRVTITCRASQSISSYLNWYQQKPGKAPKLLIYAASSLQSGV |
| VL | | PSRFSGSGSGTDFTLTISSLQPEDFATYYCQQSYSTPYTFGQGTKVEIK |
| 139109-aa | 109 | MALPVTALLLPLALLLHAARPEVQLVESGGGLVQPGGSLRLSCAVSGFALSNHGMSWV |
| Full CAR | | RRAPGKGLEWVSGIVYSGSTYYAASVKGRFTISRDNSRNTLYLQMNSLRPEDTAIYYC |
| | | SAHGGESDVWGQGTTVTVSSASGGGGSGGRASGGGGSDIQLTQSPSSLSASVGDRVTI |
| | | TCRASQSISSYLNWYQQKPGKAPKLLIYAASSLQSGVPSRFSGSGSGTDFTLTISSLQ |
| | | PEDFATYYCQQSYSTPYTFGQGTKVEIKTTTPAPRPPTPAPTIASQPLSLRPEACRPA |
| | | AGGAVHTRGLDFACDIYIWAPLAGTCGVLLLSLVITLYCKRGRKKLLYIFKQPFMRPV |
| | | QTTQEEDGCSCRFPEEEEGGCELRVKFSRSADAPAYKQGQNQLYNELNLGRREEYDVL |
| | | DKRRGRDPEMGGKPRRKNPQEGLYNELQKDKMAEAYSEIGMKGERRRGKGHDGLYQGL |
| | | STATKDTYDALHMQALPPR |
| 139109-nt | 124 | ATGGCCCTCCCTGTCACCGCCCTGCTGCTTCCGCTGGCTCTTCTGCTCCACGCCGCTC |
| Full CAR | | GGCCCGAAGTGCAATTGGTGGAATCAGGGGGAGGACTTGTGCAGCCTGGAGGATCGCT |
| | | GAGACTGTCATGTGCCGTGTCCGGCTTTGCCCTGTCCAACCACGGGATGTCCTGGGTC |
| | | CGCCGCGCGCCTGGAAAGGGCCTCGAATGGGTGTCGGGTATTGTGTACAGCGGTAGCA |
| | | CCTACTATGCCGCATCCGTGAAGGGGAGATTCACCATCAGCCGGGACAACTCCAGGAA |
| | | CACTCTGTACCTCCAAATGAATTCGCTGAGGCCAGAGGACACTGCCATCTACTACTGC |
| | | TCCGCGCATGGCGGAGAGTCCGACGTCTGGGGACAGGGGACCACCGTGACCGTGTCTA |
| | | GCGCGTCCGGCGGAGGCGGCAGCGGGGGTCGGGCATCAGGGGGCGGCGGATCGGACAT |
| | | CCAGCTCACCCAGTCCCCGAGCTCGCTGTCCGCCTCCGTGGGAGATCGGGTCACCATC |
| | | ACGTGCCGCGCCAGCCAGTCGATTTCCTCCTACCTGAACTGGTACCAACAGAAGCCCG |
| | | GAAAAGCCCCGAAGCTTCTCATCTACGCCGCCTCGAGCCTGCAGTCAGGAGTGCCCTC |
| | | ACGGTTCTCCGGCTCCGGTTCCGGTACTGATTTCACCCTGACCATTTCCTCCCTGCAA |
| | | CCGGAGGACTTCGCTACTTACTACTGCCAGCAGTCGTACTCCACCCCCTACACTTTCG |
| | | GACAAGGCACCAAGGTCGAAATCAAGACCACTACCCCAGCACCGAGGCCACCCACCCC |
| | | GGCTCCTACCATCGCCTCCCAGCCTCTGTCCCTGCGTCCGGAGGCATGTAGACCCGCA |
| | | GCTGGTGGGGCCGTGCATACCCGGGGTCTTGACTTCGCCTGCGATATCTACATTTGGG |
| | | CCCCTCTGGCTGGTACTTGCGGGGTCCTGCTGCTTTCACTCGTGATCACTCTTTACTG |
| | | TAAGCGCGGTCGGAAGAAGCTGCTGTACATCTTTAAGCAACCCTTCATGAGGCCTGTG |
| | | CAGACTACTCAAGAGGAGGACGGCTGTTCATGCCGGTTCCCAGAGGAGGAGGAAGGCG |
| | | GCTGCGAACTGCGCGTGAAATTCAGCCGCAGCGCAGATGCTCCAGCCTACAAGCAGGG |
| | | GCAGAACCAGCTCTACAACGAACTCAATCTTGGTCGGAGAGAGGAGTACGACGTGCTG |
| | | GACAAGCGGAGAGGACGGGACCCAGAAATGGGCGGGAAGCCGCGCAGAAAGAATCCCC |
| | | AAGAGGGCCTGTACAACGAGCTCCAAAAGGATAAGATGGCAGAAGCCTATAGCGAGAT |
| | | TGGTATGAAAGGGGAACGCAGAAGAGGCAAAGGCCACGACGGACTGTACCAGGGACTC |
| | | AGCACCGCCACCAAGGACACCTATGACGCTCTTCACATGCAGGCCCTGCCGCCTCGG |
| Full CAR | 392 | EVQLVESGGGLVQPGGSLRLSCAVSGFALSNHGMSWVRRAPGKGLEWVSGIVYSGSTY |
| without leader | | YAASVKGRFTISRDNSRNTLYLQMNSLRPEDTAIYYCSAHGGESDVWGQGTTVTVSSA |
| sequence | | SGGGGSGGRASGGGGSDIQLTQSPSSLSASVGDRVTITCRASQSISSYLNWYQQKPGK |
| | | APKLLIYAASSLQSGVPSRFSGSGSGTDFTLTISSLQPEDFATYYCQQSYSTPYTFGQ |
| | | GTKVEIKTTTPAPRPPTPAPTIASQPLSLRPEACRPAAGGAVHTRGLDFACDIYIWAP |
| | | LAGTCGVLLLSLVITLYCKRGRKKLLYIFKQPFMRPVQTTQEEDGCSCRFPEEEEGGC |
| | | ELRVKFSRSADAPAYKQGQNQLYNELNLGRREEYDVLDKRRGRDPEMGGKPRRKNPQE |
| | | GLYNELQKDKMAEAYSEIGMKGERRRGKGHDGLYQGLSTATKDTYDALHMQALPPR |
| Full CAR | 393 | EVQLVESGGGLVQPGGSLRLSCAVSGFALSNHGMSWVRRAPGKGLEWVSGIVYSGSTY |
| without linker, | | YAASVKGRFTISRDNSRNTLYLQMNSLRPEDTAIYYCSAHGGESDVWGQGTTVTVSSD |
| without leader | | IQLTQSPSSLSASVGDRVTITCRASQSISSYLNWYQQKPGKAPKLLIYAASSLQSGVP |
| sequence | | SRFSGSGSGTDFTLTISSLQPEDFATYYCQQSYSTPYTFGQGTKVEIKTTTPAPRPPT |
| | | PAPTIASQPLSLRPEACRPAAGGAVHTRGLDFACDIYIWAPLAGTCGVLLLSLVITLY |
| | | CKRGRKKLLYIFKQPFMRPVQTTQEEDGCSCRFPEEEEGGCELRVKFSRSADAPAYKQ |
| | | GQNQLYNELNLGRREEYDVLDKRRGRDPEMGGKPRRKNPQEGLYNELQKDKMAEAYSE |
| | | IGMKGERRRGKGHDGLYQGLSTATKDTYDALHMQALPPR |
| 139103 | | |
| 139103-aa | 39 | QVQLVESGGGLVQPGRSLRLSCAASGFTFSNYAMSWVRQAPGKGLGWVSGISRSGENT |
| ScFv domain | | YYADSVKGRFTISRDNSKNTLYLQMNSLRDEDTAVYYCARSPAHYYGGMDVWGQGTTV |
| | | TVSSASGGGGSGGRASGGGGSDIVLTQSPGTLSLSPGERATLSCRASQSISSSFLAWY |
| | | QQKPGQAPRLLIYGASRRATGIPDRFSGSGSGTDFTLTISRLEPEDSAVYYCQQYHSS |
| | | PSWTFGQGTKLEIK |
| 139103-nt | 54 | CAAGTGCAACTCGTGGAATCTGGTGGAGGACTCGTGCAACCCGGAAGATCGCTTAGAC |
| ScFv domain | | TGTCGTGTGCCGCCAGCGGGTTCACTTTCTCGAACTACGCGATGTCCTGGGTCCGCCA |
| | | GGCACCCGGAAAGGGACTCGGTTGGGTGTCCGGCATTTCCCGGTCCGGCGAAAATACC |
| | | TACTACGCCGACTCCGTGAAGGGCCGCTTCACCATCTCAAGGGACAACAGCAAAAACA |
| | | CCCTGTACTTGCAAATGAACTCCCTGCGGGATGAAGATACAGCCGTGTACTATTGCGC |
| | | CCGGTCGCCTGCCCATTACTACGGCGGAATGGACGTCTGGGGACAGGGAACCACTGTG |
| | | ACTGTCAGCAGCGCGTCGGGTGGCGGCGGCTCAGGGGGTCGGGCCTCCGGGGGGGGAG |
| | | GGTCCGACATCGTGCTGACCCAGTCCCCGGGAACCCTGAGCCTGAGCCCGGGAGAGCG |
| | | CGCGACCCTGTCATGCCGGGCATCCCAGAGCATTAGCTCCTCCTTTCTCGCCTGGTAT |
| | | CAGCAGAAGCCCGGACAGGCCCCGAGGCTGCTGATCTACGGCGCTAGCAGAAGGGCTA |
| | | CCGGAATCCCAGACCGGTTCTCCGGCTCCGGTTCCGGGACCGATTTCACCCTTACTAT |
| | | CTCGCGCCTGGAACCTGAGGACTCCGCCGTCTACTACTGCCAGCAGTACCACTCATCC |
| | | CCGTCGTGGACGTTCGGACAGGGCACCAAGCTGGAGATTAAG |
| 139103-aa | 69 | QVQLVESGGGLVQPGRSLRLSCAASGFTFSNYAMSWVRQAPGKGLGWVSGISRSGENT |
| VH | | YYADSVKGRFTISRDNSKNTLYLQMNSLRDEDTAVYYCARSPAHYYGGMDVWGQGTTV |
| | | TVSS |
| 139103-aa | 84 | DIVLTQSPGTLSLSPGERATLSCRASQSISSSFLAWYQQKPGQAPRLLIYGASRRATG |
| VL | | IPDRFSGSGSGTDFTLTISRLEPEDSAVYYCQQYHSSPSWTFGQGTKLEIK |
| 139103-aa | 99 | MALPVTALLLPLALLLHAARPQVQLVESGGGLVQPGRSLRLSCAASGFTFSNYAMSWV |
| Full CAR | | RQAPGKGLGWVSGISRSGENTYYADSVKGRFTISRDNSKNTLYLQMNSLRDEDTAVYY |
| | | CARSPAHYYGGMDVWGQGTTVTVSSASGGGGSGGRASGGGGSDIVLTQSPGTLSLSPG |
| | | ERATLSCRASQSISSSFLAWYQQKPGQAPRLLIYGASRRATGIPDRFSGSGSGTDFTL |
| | | TISRLEPEDSAVYYCQQYHSSPSWTFGQGTKLEIKTTTPAPRPPTPAPTIASQPLSLR |
| | | PEACRPAAGGAVHTRGLDFACDIYIWAPLAGTCGVLLLSLVITLYCKRGRKKLLYIFK |
| | | QPFMRPVQTTQEEDGCSCRFPEEEEGGCELRVKFSRSADAPAYKQGQNQLYNELNLGR |
| | | REEYDVLDKRRGRDPEMGGKPRRKNPQEGLYNELQKDKMAEAYSEIGMKGERRRGKGH |
| | | DGLYQGLSTATKDTYDALHMQALPPR |
| 139103-nt | 114 | ATGGCCCTCCCTGTCACCGCCCTGCTGCTTCCGCTGGCTCTTCTGCTCCACGCCGCTC |
| Full CAR | | GGCCCCAAGTGCAACTCGTGGAATCTGGTGGAGGACTCGTGCAACCCGGAAGATCGCT |
| | | TAGACTGTCGTGTGCCGCCAGCGGGTTCACTTTCTCGAACTACGCGATGTCCTGGGTC |
| | | CGCCAGGCACCCGGAAAGGGACTCGGTTGGGTGTCCGGCATTTCCCGGTCCGGCGAAA |
| | | ATACCTACTACGCCGACTCCGTGAAGGGCCGCTTCACCATCTCAAGGGACAACAGCAA |
| | | AAACACCCTGTACTTGCAAATGAACTCCCTGCGGGATGAAGATACAGCCGTGTACTAT |
| | | TGCGCCCGGTCGCCTGCCCATTACTACGGCGGAATGGACGTCTGGGGACAGGGAACCA |
| | | CTGTGACTGTCAGCAGCGCGTCGGGTGGCGGCGGCTCAGGGGGTCGGGCCTCCGGGGG |
| | | GGGAGGGTCCGACATCGTGCTGACCCAGTCCCCGGGAACCCTGAGCCTGAGCCCGGGA |
| | | GAGCGCGCGACCCTGTCATGCCGGGCATCCCAGAGCATTAGCTCCTCCTTTCTCGCCT |
| | | GGTATCAGCAGAAGCCCGGACAGGCCCCGAGGCTGCTGATCTACGGCGCTAGCAGAAG |
| | | GGCTACCGGAATCCCAGACCGGTTCTCCGGCTCCGGTTCCGGGACCGATTTCACCCTT |
| | | ACTATCTCGCGCCTGGAACCTGAGGACTCCGCCGTCTACTACTGCCAGCAGTACCACT |
| | | CATCCCCGTCGTGGACGTTCGGACAGGGCACCAAGCTGGAGATTAAGACCACTACCCC |
| | | AGCACCGAGGCCACCCACCCCGGCTCCTACCATCGCCTCCCAGCCTCTGTCCCTGCGT |
| | | CCGGAGGCATGTAGACCCGCAGCTGGTGGGGCCGTGCATACCCGGGGTCTTGACTTCG |
| | | CCTGCGATATCTACATTTGGGCCCCTCTGGCTGGTACTTGCGGGGTCCTGCTGCTTTC |
| | | ACTCGTGATCACTCTTTACTGTAAGCGCGGTCGGAAGAAGCTGCTGTACATCTTTAAG |
| | | CAACCCTTCATGAGGCCTGTGCAGACTACTCAAGAGGAGGACGGCTGTTCATGCCGGT |
| | | TCCCAGAGGAGGAGGAAGGCGGCTGCGAACTGCGCGTGAAATTCAGCCGCAGCGCAGA |
| | | TGCTCCAGCCTACAAGCAGGGGCAGAACCAGCTCTACAACGAACTCAATCTTGGTCGG |
| | | AGAGAGGAGTACGACGTGCTGGACAAGCGGAGAGGACGGGACCCAGAAATGGGCGGGA |
| | | AGCCGCGCAGAAAGAATCCCCAAGAGGGCCTGTACAACGAGCTCCAAAAGGATAAGAT |
| | | GGCAGAAGCCTATAGCGAGATTGGTATGAAAGGGGAACGCAGAAGAGGCAAAGGCCAC |
| | | GACGGACTGTACCAGGGACTCAGCACCGCCACCAAGGACACCTATGACGCTCTTCACA |
| | | TGCAGGCCCTGCCGCCTCGG |
| 139105 | | |
| 139105-aa | 40 | QVQLVESGGGLVQPGRSLRLSCAASGFTFDDYAMHWVRQAPGKGLEWVSGISWNSGSI |
| ScFv domain | | GYADSVKGRFTISRDNAKNSLYLQMNSLRAEDTALYYCSVHSFLAYWGQGTLVTVSSA |
| | | SGGGGSGGRASGGGGSDIVMTQTPLSLPVTPGEPASISCRSSQSLLHSNGYNYLDWYL |
| | | QKPGQSPQLLIYLGSNRASGVPDRFSGSGSGTDFTLKISRVEAEDVGVYYCMQALQTP |
| | | YTFGQGTKVEIK |
| 139105-nt | 55 | CAAGTGCAACTCGTCGAATCCGGTGGAGGTCTGGTCCAACCTGGTAGAAGCCTGAGAC |
| ScFv domain | | TGTCGTGTGCGGCCAGCGGATTCACCTTTGATGACTATGCTATGCACTGGGTGCGGCA |
| | | GGCCCCAGGAAAGGGCCTGGAATGGGTGTCGGGAATTAGCTGGAACTCCGGGTCCATT |
| | | GGCTACGCCGACTCCGTGAAGGGCCGCTTCACCATCTCCCGCGACAACGCAAAGAACT |
| | | CCCTGTACTTGCAAATGAACTCGCTCAGGGCTGAGGATACCGCGCTGTACTACTGCTC |
| | | CGTGCATTCCTTCCTGGCCTACTGGGGACAGGGAACTCTGGTCACCGTGTCGAGCGCC |
| | | TCCGGCGGCGGGGGCTCGGGTGGACGGGCCTCGGGCGGAGGGGGGTCCGACATCGTGA |
| | | TGACCCAGACCCCGCTGAGCTTGCCCGTGACTCCCGGAGAGCCTGCATCCATCTCCTG |
| | | CCGGTCATCCCAGTCCCTTCTCCACTCCAACGGATACAACTACCTCGACTGGTACCTC |
| | | CAGAAGCCGGGACAGAGCCCTCAGCTTCTGATCTACCTGGGGTCAAATAGAGCCTCAG |
| | | GAGTGCCGGATCGGTTCAGCGGATCTGGTTCGGGAACTGATTTCACTCTGAAGATTTC |
| | | CCGCGTGGAAGCCGAGGACGTGGGCGTCTACTACTGTATGCAGGCGCTGCAGACCCCC |
| | | TATACCTTCGGCCAAGGGACGAAAGTGGAGATCAAG |
| 139105-aa | 70 | QVQLVESGGGLVQPGRSLRLSCAASGFTFDDYAMHWVRQAPGKGLEWVSGISWNSGSI |
| VH | | GYADSVKGRFTISRDNAKNSLYLQMNSLRAEDTALYYCSVHSFLAYWGQGTLVTVSS |
| 139105-aa | 85 | DIVMTQTPLSLPVTPGEPASISCRSSQSLLHSNGYNYLDWYLQKPGQSPQLLIYLGSN |
| VL | | RASGVPDRFSGSGSGTDFTLKISRVEAEDVGVYYCMQALQTPYTFGQGTKVEIK |
| 139105-aa | 100 | MALPVTALLLPLALLLHAARPQVQLVESGGGLVQPGRSLRLSCAASGFTFDDYAMHWV |
| Full CAR | | RQAPGKGLEWVSGISWNSGSIGYADSVKGRFTISRDNAKNSLYLQMNSLRAEDTALYY |
| | | CSVHSFLAYWGQGTLVTVSSASGGGGSGGRASGGGGSDIVMTQTPLSLPVTPGEPASI |
| | | SCRSSQSLLHSNGYNYLDWYLQKPGQSPQLLIYLGSNRASGVPDRFSGSGSGTDFTLK |
| | | ISRVEAEDVGVYYCMQALQTPYTFGQGTKVEIKTTTPAPRPPTPAPTIASQPLSLRPE |
| | | ACRPAAGGAVHTRGLDFACDIYIWAPLAGTCGVLLLSLVITLYCKRGRKKLLYIFKQP |
| | | FMRPVQTTQEEDGCSCRFPEEEEGGCELRVKFSRSADAPAYKQGQNQLYNELNLGRRE |
| | | EYDVLDKRRGRDPEMGGKPRRKNPQEGLYNELQKDKMAEAYSEIGMKGERRRGKGHDG |
| | | LYQGLSTATKDTYDALHMQALPPR |
| 139105-nt | 115 | ATGGCCCTCCCTGTCACCGCCCTGCTGCTTCCGCTGGCTCTTCTGCTCCACGCCGCTC |
| Full CAR | | GGCCCCAAGTGCAACTCGTCGAATCCGGTGGAGGTCTGGTCCAACCTGGTAGAAGCCT |
| | | GAGACTGTCGTGTGCGGCCAGCGGATTCACCTTTGATGACTATGCTATGCACTGGGTG |
| | | CGGCAGGCCCCAGGAAAGGGCCTGGAATGGGTGTCGGGAATTAGCTGGAACTCCGGGT |
| | | CCATTGGCTACGCCGACTCCGTGAAGGGCCGCTTCACCATCTCCCGCGACAACGCAAA |
| | | GAACTCCCTGTACTTGCAAATGAACTCGCTCAGGGCTGAGGATACCGCGCTGTACTAC |
| | | TGCTCCGTGCATTCCTTCCTGGCCTACTGGGGACAGGGAACTCTGGTCACCGTGTCGA |
| | | GCGCCTCCGGCGGCGGGGGCTCGGGTGGACGGGCCTCGGGCGGAGGGGGGTCCGACAT |
| | | CGTGATGACCCAGACCCCGCTGAGCTTGCCCGTGACTCCCGGAGAGCCTGCATCCATC |
| | | TCCTGCCGGTCATCCCAGTCCCTTCTCCACTCCAACGGATACAACTACCTCGACTGGT |
| | | ACCTCCAGAAGCCGGGACAGAGCCCTCAGCTTCTGATCTACCTGGGGTCAAATAGAGC |
| | | CTCAGGAGTGCCGGATCGGTTCAGCGGATCTGGTTCGGGAACTGATTTCACTCTGAAG |
| | | ATTTCCCGCGTGGAAGCCGAGGACGTGGGCGTCTACTACTGTATGCAGGCGCTGCAGA |
| | | CCCCCTATACCTTCGGCCAAGGGACGAAAGTGGAGATCAAGACCACTACCCCAGCACC |
| | | GAGGCCACCCACCCCGGCTCCTACCATCGCCTCCCAGCCTCTGTCCCTGCGTCCGGAG |
| | | GCATGTAGACCCGCAGCTGGTGGGGCCGTGCATACCCGGGGTCTTGACTTCGCCTGCG |
| | | ATATCTACATTTGGGCCCCTCTGGCTGGTACTTGCGGGGTCCTGCTGCTTTCACTCGT |
| | | GATCACTCTTTACTGTAAGCGCGGTCGGAAGAAGCTGCTGTACATCTTTAAGCAACCC |
| | | TTCATGAGGCCTGTGCAGACTACTCAAGAGGAGGACGGCTGTTCATGCCGGTTCCCAG |
| | | AGGAGGAGGAAGGCGGCTGCGAACTGCGCGTGAAATTCAGCCGCAGCGCAGATGCTCC |
| | | AGCCTACAAGCAGGGGCAGAACCAGCTCTACAACGAACTCAATCTTGGTCGGAGAGAG |
| | | GAGTACGACGTGCTGGACAAGCGGAGAGGACGGGACCCAGAAATGGGCGGGAAGCCGC |
| | | GCAGAAAGAATCCCCAAGAGGGCCTGTACAACGAGCTCCAAAAGGATAAGATGGCAGA |
| | | AGCCTATAGCGAGATTGGTATGAAAGGGGAACGCAGAAGAGGCAAAGGCCACGACGGA |
| | | CTGTACCAGGGACTCAGCACCGCCACCAAGGACACCTATGACGCTCTTCACATGCAGG |
| | | CCCTGCCGCCTCGG |
| 139111 | | |
| 139111-aa | 41 | EVQLLESGGGLVQPGGSLRLSCAVSGFALSNHGMSWVRRAPGKGLEWVSGIVYSGSTY |
| ScFv domain | | YAASVKGRFTISRDNSRNTLYLQMNSLRPEDTAIYYCSAHGGESDVWGQGTTVTVSSA |
| | | SGGGGSGGRASGGGGSDIVMTQTPLSLSVTPGQPASISCKSSQSLLRNDGKTPLYWYL |
| | | QKAGQPPQLLIYEVSNRFSGVPDRFSGSGSGTDFTLKISRVEAEDVGAYYCMQNIQFP |
| | | SFGGGTKLEIK |
| 139111-nt | 56 | GAAGTGCAATTGTTGGAATCTGGAGGAGGACTTGTGCAGCCTGGAGGATCACTGAGAC |
| ScFv domain | | TTTCGTGTGCGGTGTCAGGCTTCGCCCTGAGCAACCACGGCATGAGCTGGGTGCGGAG |
| | | AGCCCCGGGGAAGGGTCTGGAATGGGTGTCCGGGATCGTCTACTCCGGTTCAACTTAC |
| | | TACGCCGCAAGCGTGAAGGGTCGCTTCACCATTTCCCGCGATAACTCCCGGAACACCC |
| | | TGTACCTCCAAATGAACTCCCTGCGGCCCGAGGACACCGCCATCTACTACTGTTCCGC |
| | | GCATGGAGGAGAGTCCGATGTCTGGGGACAGGGCACTACCGTGACCGTGTCGAGCGCC |
| | | TCGGGGGGAGGAGGCTCCGGCGGTCGCGCCTCCGGGGGGGGTGGCAGCGACATTGTGA |
| | | TGACGCAGACTCCACTCTCGCTGTCCGTGACCCCGGGACAGCCCGCGTCCATCTCGTG |
| | | CAAGAGCTCCCAGAGCCTGCTGAGGAACGACGGAAAGACTCCTCTGTATTGGTACCTC |
| | | CAGAAGGCTGGACAGCCCCCGCAACTGCTCATCTACGAAGTGTCAAATCGCTTCTCCG |
| | | GGGTGCCGGATCGGTTTTCCGGCTCGGGATCGGGCACCGACTTCACCCTGAAAATCTC |
| | | CAGGGTCGAGGCCGAGGACGTGGGAGCCTACTACTGCATGCAAAACATCCAGTTCCCT |
| | | TCCTTCGGCGGCGGCACAAAGCTGGAGATTAAG |
| 139111-aa | 71 | EVQLLESGGGLVQPGGSLRLSCAVSGFALSNHGMSWVRRAPGKGLEWVSGIVYSGSTY |
| VH | | YAASVKGRFTISRDNSRNTLYLQMNSLRPEDTAIYYCSAHGGESDVWGQGTTVTVSS |
| 139111-aa | 86 | DIVMTQTPLSLSVTPGQPASISCKSSQSLLRNDGKTPLYWYLQKAGQPPQLLIYEVSN |
| VL | | RFSGVPDRFSGSGSGTDFTLKISRVEAEDVGAYYCMQNIQFPSFGGGTKLEIK |
| 139111-aa | 101 | MALPVTALLLPLALLLHAARPEVQLLESGGGLVQPGGSLRLSCAVSGFALSNHGMSWV |
| Full CAR | | RRAPGKGLEWVSGIVYSGSTYYAASVKGRFTISRDNSRNTLYLQMNSLRPEDTAIYYC |
| | | SAHGGESDVWGQGTTVTVSSASGGGGSGGRASGGGGSDIVMTQTPLSLSVTPGQPASI |
| | | SCKSSQSLLRNDGKTPLYWYLQKAGQPPQLLIYEVSNRFSGVPDRFSGSGSGTDFTLK |
| | | ISRVEAEDVGAYYCMQNIQFPSFGGGTKLEIKTTTPAPRPPTPAPTIASQPLSLRPEA |
| | | CRPAAGGAVHTRGLDFACDIYIWAPLAGTCGVLLLSLVITLYCKRGRKKLLYIFKQPF |
| | | MRPVQTTQEEDGCSCRFPEEEEGGCELRVKFSRSADAPAYKQGQNQLYNELNLGRREE |
| | | YDVLDKRRGRDPEMGGKPRRKNPQEGLYNELQKDKMAEAYSEIGMKGERRRGKGHDGL |
| | | YQGLSTATKDTYDALHMQALPPR |
| 139111-nt | 116 | ATGGCCCTCCCTGTCACCGCCCTGCTGCTTCCGCTGGCTCTTCTGCTCCACGCCGCTC |
| Full CAR | | GGCCCGAAGTGCAATTGTTGGAATCTGGAGGAGGACTTGTGCAGCCTGGAGGATCACT |
| | | GAGACTTTCGTGTGCGGTGTCAGGCTTCGCCCTGAGCAACCACGGCATGAGCTGGGTG |
| | | CGGAGAGCCCCGGGGAAGGGTCTGGAATGGGTGTCCGGGATCGTCTACTCCGGTTCAA |
| | | CTTACTACGCCGCAAGCGTGAAGGGTCGCTTCACCATTTCCCGCGATAACTCCCGGAA |
| | | CACCCTGTACCTCCAAATGAACTCCCTGCGGCCCGAGGACACCGCCATCTACTACTGT |
| | | TCCGCGCATGGAGGAGAGTCCGATGTCTGGGGACAGGGCACTACCGTGACCGTGTCGA |
| | | GCGCCTCGGGGGGAGGAGGCTCCGGCGGTCGCGCCTCCGGGGGGGGTGGCAGCGACAT |
| | | TGTGATGACGCAGACTCCACTCTCGCTGTCCGTGACCCCGGGACAGCCCGCGTCCATC |
| | | TCGTGCAAGAGCTCCCAGAGCCTGCTGAGGAACGACGGAAAGACTCCTCTGTATTGGT |
| | | ACCTCCAGAAGGCTGGACAGCCCCCGCAACTGCTCATCTACGAAGTGTCAAATCGCTT |
| | | CTCCGGGGTGCCGGATCGGTTTTCCGGCTCGGGATCGGGCACCGACTTCACCCTGAAA |
| | | ATCTCCAGGGTCGAGGCCGAGGACGTGGGAGCCTACTACTGCATGCAAAACATCCAGT |
| | | TCCCTTCCTTCGGCGGCGGCACAAAGCTGGAGATTAAGACCACTACCCCAGCACCGAG |
| | | GCCACCCACCCCGGCTCCTACCATCGCCTCCCAGCCTCTGTCCCTGCGTCCGGAGGCA |
| | | TGTAGACCCGCAGCTGGTGGGGCCGTGCATACCCGGGGTCTTGACTTCGCCTGCGATA |
| | | TCTACATTTGGGCCCCTCTGGCTGGTACTTGCGGGGTCCTGCTGCTTTCACTCGTGAT |
| | | CACTCTTTACTGTAAGCGCGGTCGGAAGAAGCTGCTGTACATCTTTAAGCAACCCTTC |
| | | ATGAGGCCTGTGCAGACTACTCAAGAGGAGGACGGCTGTTCATGCCGGTTCCCAGAGG |
| | | AGGAGGAAGGCGGCTGCGAACTGCGCGTGAAATTCAGCCGCAGCGCAGATGCTCCAGC |
| | | CTACAAGCAGGGGCAGAACCAGCTCTACAACGAACTCAATCTTGGTCGGAGAGAGGAG |
| | | TACGACGTGCTGGACAAGCGGAGAGGACGGGACCCAGAAATGGGCGGGAAGCCGCGCA |
| | | GAAAGAATCCCCAAGAGGGCCTGTACAACGAGCTCCAAAAGGATAAGATGGCAGAAGC |
| | | CTATAGCGAGATTGGTATGAAAGGGGAACGCAGAAGAGGCAAAGGCCACGACGGACTG |
| | | TACCAGGGACTCAGCACCGCCACCAAGGACACCTATGACGCTCTTCACATGCAGGCCC |
| | | TGCCGCCTCGG |
| 139100 | | |
| 139100-aa | 42 | QVQLVQSGAEVRKTGASVKVSCKASGYIFDNFGINWVRQAPGQGLEWMGWINPKNNNT |
| ScFv domain | | NYAQKFQGRVTITADESTNTAYMEVSSLRSEDTAVYYCARGPYYYQSYMDVWGQGTMV |
| | | TVSSASGGGGSGGRASGGGGSDIVMTQTPLSLPVTPGEPASISCRSSQSLLHSNGYNY |
| | | LNWYLQKPGQSPQLLIYLGSKRASGVPDRFSGSGSGTDFTLHITRVGAEDVGVYYCMQ |
| | | ALQTPYTFGQGTKLEIK |
| 139100-nt | 57 | CAAGTCCAACTCGTCCAGTCCGGCGCAGAAGTCAGAAAAACCGGTGCTAGCGTGAAAG |
| ScFv domain | | TGTCCTGCAAGGCCTCCGGCTACATTTTCGATAACTTCGGAATCAACTGGGTCAGACA |
| | | GGCCCCGGGCCAGGGGCTGGAATGGATGGGATGGATCAACCCCAAGAACAACAACACC |
| | | AACTACGCACAGAAGTTCCAGGGCCGCGTGACTATCACCGCCGATGAATCGACCAATA |
| | | CCGCCTACATGGAGGTGTCCTCCCTGCGGTCGGAGGACACTGCCGTGTATTACTGCGC |
| | | GAGGGGCCCATACTACTACCAAAGCTACATGGACGTCTGGGGACAGGGAACCATGGTG |
| | | ACCGTGTCATCCGCCTCCGGTGGTGGAGGCTCCGGGGGGCGGGCTTCAGGAGGCGGAG |
| | | GAAGCGATATTGTGATGACCCAGACTCCGCTTAGCCTGCCCGTGACTCCTGGAGAACC |
| | | GGCCTCCATTTCCTGCCGGTCCTCGCAATCACTCCTGCATTCCAACGGTTACAACTAC |
| | | CTGAATTGGTACCTCCAGAAGCCTGGCCAGTCGCCCCAGTTGCTGATCTATCTGGGCT |
| | | CGAAGCGCGCCTCCGGGGTGCCTGACCGGTTTAGCGGATCTGGGAGCGGCACGGACTT |
| | | CACTCTCCACATCACCCGCGTGGGAGCGGAGGACGTGGGAGTGTACTACTGTATGCAG |
| | | GCGCTGCAGACTCCGTACACATTCGGACAGGGCACCAAGCTGGAGATCAAG |
| 139100-aa | 72 | QVQLVQSGAEVRKTGASVKVSCKASGYIFDNFGINWVRQAPGQGLEWMGWINPKNNNT |
| VH | | NYAQKFQGRVTITADESTNTAYMEVSSLRSEDTAVYYCARGPYYYQSYMDVWGQGTMV |
| | | TVSS |
| 139100-aa | 87 | DIVMTQTPLSLPVTPGEPASISCRSSQSLLHSNGYNYLNWYLQKPGQSPQLLIYLGSK |
| VL | | RASGVPDRFSGSGSGTDFTLHITRVGAEDVGVYYCMQALQTPYTFGQGTKLEIK |
| 139100-aa | 102 | MALPVTALLLPLALLLHAARPQVQLVQSGAEVRKTGASVKVSCKASGYIFDNFGINWV |
| Full CAR | | RQAPGQGLEWMGWINPKNNNTNYAQKFQGRVTITADESTNTAYMEVSSLRSEDTAVYY |
| | | CARGPYYYQSYMDVWGQGTMVTVSSASGGGGSGGRASGGGGSDIVMTQTPLSLPVTPG |
| | | EPASISCRSSQSLLHSNGYNYLNWYLQKPGQSPQLLIYLGSKRASGVPDRFSGSGSGT |
| | | DFTLHITRVGAEDVGVYYCMQALQTPYTFGQGTKLEIKTTTPAPRPPTPAPTIASQPL |
| | | SLRPEACRPAAGGAVHTRGLDFACDIYIWAPLAGTCGVLLLSLVITLYCKRGRKKLLY |
| | | IFKQPFMRPVQTTQEEDGCSCRFPEEEEGGCELRVKFSRSADAPAYKQGQNQLYNELN |
| | | LGRREEYDVLDKRRGRDPEMGGKPRRKNPQEGLYNELQKDKMAEAYSEIGMKGERRRG |
| | | KGHDGLYQGLSTATKDTYDALHMQALPPR |
| 139100-nt | 117 | ATGGCCCTCCCTGTCACCGCCCTGCTGCTTCCGCTGGCTCTTCTGCTCCACGCCGCTC |
| Full CAR | | GGCCCCAAGTCCAACTCGTCCAGTCCGGCGCAGAAGTCAGAAAAACCGGTGCTAGCGT |
| | | GAAAGTGTCCTGCAAGGCCTCCGGCTACATTTTCGATAACTTCGGAATCAACTGGGTC |
| | | AGACAGGCCCCGGGCCAGGGGCTGGAATGGATGGGATGGATCAACCCCAAGAACAACA |
| | | ACACCAACTACGCACAGAAGTTCCAGGGCCGCGTGACTATCACCGCCGATGAATCGAC |
| | | CAATACCGCCTACATGGAGGTGTCCTCCCTGCGGTCGGAGGACACTGCCGTGTATTAC |
| | | TGCGCGAGGGGCCCATACTACTACCAAAGCTACATGGACGTCTGGGGACAGGGAACCA |
| | | TGGTGACCGTGTCATCCGCCTCCGGTGGTGGAGGCTCCGGGGGGCGGGCTTCAGGAGG |
| | | CGGAGGAAGCGATATTGTGATGACCCAGACTCCGCTTAGCCTGCCCGTGACTCCTGGA |
| | | GAACCGGCCTCCATTTCCTGCCGGTCCTCGCAATCACTCCTGCATTCCAACGGTTACA |
| | | ACTACCTGAATTGGTACCTCCAGAAGCCTGGCCAGTCGCCCCAGTTGCTGATCTATCT |
| | | GGGCTCGAAGCGCGCCTCCGGGGTGCCTGACCGGTTTAGCGGATCTGGGAGCGGCACG |
| | | GACTTCACTCTCCACATCACCCGCGTGGGAGCGGAGGACGTGGGAGTGTACTACTGTA |
| | | TGCAGGCGCTGCAGACTCCGTACACATTCGGACAGGGCACCAAGCTGGAGATCAAGAC |
| | | CACTACCCCAGCACCGAGGCCACCCACCCCGGCTCCTACCATCGCCTCCCAGCCTCTG |
| | | TCCCTGCGTCCGGAGGCATGTAGACCCGCAGCTGGTGGGGCCGTGCATACCCGGGGTC |
| | | TTGACTTCGCCTGCGATATCTACATTTGGGCCCCTCTGGCTGGTACTTGCGGGGTCCT |
| | | GCTGCTTTCACTCGTGATCACTCTTTACTGTAAGCGCGGTCGGAAGAAGCTGCTGTAC |
| | | ATCTTTAAGCAACCCTTCATGAGGCCTGTGCAGACTACTCAAGAGGAGGACGGCTGTT |
| | | CATGCCGGTTCCCAGAGGAGGAGGAAGGCGGCTGCGAACTGCGCGTGAAATTCAGCCG |
| | | CAGCGCAGATGCTCCAGCCTACAAGCAGGGGCAGAACCAGCTCTACAACGAACTCAAT |
| | | CTTGGTCGGAGAGAGGAGTACGACGTGCTGGACAAGCGGAGAGGACGGGACCCAGAAA |
| | | TGGGCGGGAAGCCGCGCAGAAAGAATCCCCAAGAGGGCCTGTACAACGAGCTCCAAAA |
| | | GGATAAGATGGCAGAAGCCTATAGCGAGATTGGTATGAAAGGGGAACGCAGAAGAGGC |
| | | AAAGGCCACGACGGACTGTACCAGGGACTCAGCACCGCCACCAAGGACACCTATGACG |
| | | CTCTTCACATGCAGGCCCTGCCGCCTCGG |
| 139101 | | |
| 139101-aa | 43 | QVQLQESGGGLVQPGGSLRLSCAASGFTFSSDAMTWVRQAPGKGLEWVSVISGSGGTT |
| ScFv domain | | YYADSVKGRFTISRDNSKNTLYLQMNSLRAEDTAVYYCAKLDSSGYYYARGPRYWGQG |
| | | TLVTVSSASGGGGSGGRASGGGGSDIQLTQSPSSLSASVGDRVTITCRASQSISSYLN |
| | | WYQQKPGKAPKLLIYGASTLASGVPARFSGSGSGTHFTLTINSLQSEDSATYYCQQSY |
| | | KRASFGQGTKVEIK |
| 139101-nt | 58 | CAAGTGCAACTTCAAGAATCAGGCGGAGGACTCGTGCAGCCCGGAGGATCATTGCGGC |
| ScFv domain | | TCTCGTGCGCCGCCTCGGGCTTCACCTTCTCGAGCGACGCCATGACCTGGGTCCGCCA |
| | | GGCCCCGGGGAAGGGGCTGGAATGGGTGTCTGTGATTTCCGGCTCCGGGGGAACTACG |
| | | TACTACGCCGATTCCGTGAAAGGTCGCTTCACTATCTCCCGGGACAACAGCAAGAACA |
| | | CCCTTTATCTGCAAATGAATTCCCTCCGCGCCGAGGACACCGCCGTGTACTACTGCGC |
| | | CAAGCTGGACTCCTCGGGCTACTACTATGCCCGGGGTCCGAGATACTGGGGACAGGGA |
| | | ACCCTCGTGACCGTGTCCTCCGCGTCCGGCGGAGGAGGGTCGGGAGGGCGGGCCTCCG |
| | | GCGGCGGCGGTTCGGACATCCAGCTGACCCAGTCCCCATCCTCACTGAGCGCAAGCGT |
| | | GGGCGACAGAGTCACCATTACATGCAGGGCGTCCCAGAGCATCAGCTCCTACCTGAAC |
| | | TGGTACCAACAGAAGCCTGGAAAGGCTCCTAAGCTGTTGATCTACGGGGCTTCGACCC |
| | | TGGCATCCGGGGTGCCCGCGAGGTTTAGCGGAAGCGGTAGCGGCACTCACTTCACTCT |
| | | GACCATTAACAGCCTCCAGTCCGAGGATTCAGCCACTTACTACTGTCAGCAGTCCTAC |
| | | AAGCGGGCCAGCTTCGGACAGGGCACTAAGGTCGAGATCAAG |
| 139101-aa | 73 | QVQLQESGGGLVQPGGSLRLSCAASGFTFSSDAMTWVRQAPGKGLEWVSVISGSGGTT |
| VH | | YYADSVKGRFTISRDNSKNTLYLQMNSLRAEDTAVYYCAKLDSSGYYYARGPRYWGQG |
| | | TLVTVSS |
| 139101-aa | 88 | DIQLTQSPSSLSASVGDRVTITCRASQSISSYLNWYQQKPGKAPKLLIYGASTLASGV |
| VL | | PARFSGSGSGTHFTLTINSLQSEDSATYYCQQSYKRASFGQGTKVEIK |
| 139101-aa | 103 | MALPVTALLLPLALLLHAARPQVQLQESGGGLVQPGGSLRLSCAASGFTFSSDAMTWV |
| Full CAR | | RQAPGKGLEWVSVISGSGGTTYYADSVKGRFTISRDNSKNTLYLQMNSLRAEDTAVYY |
| | | CAKLDSSGYYYARGPRYWGQGTLVTVSSASGGGGSGGRASGGGGSDIQLTQSPSSLSA |
| | | SVGDRVTITCRASQSISSYLNWYQQKPGKAPKLLIYGASTLASGVPARFSGSGSGTHF |
| | | TLTINSLQSEDSATYYCQQSYKRASFGQGTKVEIKTTTPAPRPPTPAPTIASQPLSLR |
| | | PEACRPAAGGAVHTRGLDFACDIYIWAPLAGTCGVLLLSLVITLYCKRGRKKLLYIFK |
| | | QPFMRPVQTTQEEDGCSCRFPEEEEGGCELRVKFSRSADAPAYKQGQNQLYNELNLGR |
| | | REEYDVLDKRRGRDPEMGGKPRRKNPQEGLYNELQKDKMAEAYSEIGMKGERRRGKGH |
| | | DGLYQGLSTATKDTYDALHMQALPPR |
| 139101-nt | 118 | ATGGCCCTCCCTGTCACCGCCCTGCTGCTTCCGCTGGCTCTTCTGCTCCACGCCGCTC |
| Full CAR | | GGCCCCAAGTGCAACTTCAAGAATCAGGCGGAGGACTCGTGCAGCCCGGAGGATCATT |
| | | GCGGCTCTCGTGCGCCGCCTCGGGCTTCACCTTCTCGAGCGACGCCATGACCTGGGTC |
| | | CGCCAGGCCCCGGGGAAGGGGCTGGAATGGGTGTCTGTGATTTCCGGCTCCGGGGGAA |
| | | CTACGTACTACGCCGATTCCGTGAAAGGTCGCTTCACTATCTCCCGGGACAACAGCAA |
| | | GAACACCCTTTATCTGCAAATGAATTCCCTCCGCGCCGAGGACACCGCCGTGTACTAC |
| | | TGCGCCAAGCTGGACTCCTCGGGCTACTACTATGCCCGGGGTCCGAGATACTGGGGAC |
| | | AGGGAACCCTCGTGACCGTGTCCTCCGCGTCCGGCGGAGGAGGGTCGGGAGGGCGGGC |
| | | CTCCGGCGGCGGCGGTTCGGACATCCAGCTGACCCAGTCCCCATCCTCACTGAGCGCA |
| | | AGCGTGGGCGACAGAGTCACCATTACATGCAGGGCGTCCCAGAGCATCAGCTCCTACC |
| | | TGAACTGGTACCAACAGAAGCCTGGAAAGGCTCCTAAGCTGTTGATCTACGGGGCTTC |
| | | GACCCTGGCATCCGGGGTGCCCGCGAGGTTTAGCGGAAGCGGTAGCGGCACTCACTTC |
| | | ACTCTGACCATTAACAGCCTCCAGTCCGAGGATTCAGCCACTTACTACTGTCAGCAGT |
| | | CCTACAAGCGGGCCAGCTTCGGACAGGGCACTAAGGTCGAGATCAAGACCACTACCCC |
| | | AGCACCGAGGCCACCCACCCCGGCTCCTACCATCGCCTCCCAGCCTCTGTCCCTGCGT |
| | | CCGGAGGCATGTAGACCCGCAGCTGGTGGGGCCGTGCATACCCGGGGTCTTGACTTCG |
| | | CCTGCGATATCTACATTTGGGCCCCTCTGGCTGGTACTTGCGGGGTCCTGCTGCTTTC |
| | | ACTCGTGATCACTCTTTACTGTAAGCGCGGTCGGAAGAAGCTGCTGTACATCTTTAAG |
| | | CAACCCTTCATGAGGCCTGTGCAGACTACTCAAGAGGAGGACGGCTGTTCATGCCGGT |
| | | TCCCAGAGGAGGAGGAAGGCGGCTGCGAACTGCGCGTGAAATTCAGCCGCAGCGCAGA |
| | | TGCTCCAGCCTACAAGCAGGGGCAGAACCAGCTCTACAACGAACTCAATCTTGGTCGG |
| | | AGAGAGGAGTACGACGTGCTGGACAAGCGGAGAGGACGGGACCCAGAAATGGGCGGGA |
| | | AGCCGCGCAGAAAGAATCCCCAAGAGGGCCTGTACAACGAGCTCCAAAAGGATAAGAT |
| | | GGCAGAAGCCTATAGCGAGATTGGTATGAAAGGGGAACGCAGAAGAGGCAAAGGCCAC |
| | | GACGGACTGTACCAGGGACTCAGCACCGCCACCAAGGACACCTATGACGCTCTTCACA |
| | | TGCAGGCCCTGCCGCCTCGG |
| 139102 | | |
| 139102-aa | 44 | QVQLVQSGAEVKKPGASVKVSCKASGYTFSNYGITWVRQAPGQGLEWMGWISAYNGNT |
| ScFv domain | | NYAQKFQGRVTMTRNTSISTAYMELSSLRSEDTAVYYCARGPYYYYMDVWGKGTMVTV |
| | | SSASGGGGSGGRASGGGGSEIVMTQSPLSLPVTPGEPASISCRSSQSLLYSNGYNYVD |
| | | WYLQKPGQSPQLLIYLGSNRASGVPDRFSGSGSGTDFKLQISRVEAEDVGIYYCMQGR |
| | | QFPYSFGQGTKVEIK |
| 139102-nt | 59 | CAAGTCCAACTGGTCCAGAGCGGTGCAGAAGTGAAGAAGCCCGGAGCGAGCGTGAAAG |
| ScFv domain | | TGTCCTGCAAGGCTTCCGGGTACACCTTCTCCAACTACGGCATCACTTGGGTGCGCCA |
| | | GGCCCCGGGACAGGGCCTGGAATGGATGGGGTGGATTTCCGCGTACAACGGCAATACG |
| | | AACTACGCTCAGAAGTTCCAGGGTAGAGTGACCATGACTAGGAACACCTCCATTTCCA |
| | | CCGCCTACATGGAACTGTCCTCCCTGCGGAGCGAGGACACCGCCGTGTACTATTGCGC |
| | | CCGGGGACCATACTACTACTACATGGATGTCTGGGGGAAGGGGACTATGGTCACCGTG |
| | | TCATCCGCCTCGGGAGGCGGCGGATCAGGAGGACGCGCCTCTGGTGGTGGAGGATCGG |
| | | AGATCGTGATGACCCAGAGCCCTCTCTCCTTGCCCGTGACTCCTGGGGAGCCCGCATC |
| | | CATTTCATGCCGGAGCTCCCAGTCACTTCTCTACTCCAACGGCTATAACTACGTGGAT |
| | | TGGTACCTCCAAAAGCCGGGCCAGAGCCCGCAGCTGCTGATCTACCTGGGCTCGAACA |
| | | GGGCCAGCGGAGTGCCTGACCGGTTCTCCGGGTCGGGAAGCGGGACCGACTTCAAGCT |
| | | GCAAATCTCGAGAGTGGAGGCCGAGGACGTGGGAATCTACTACTGTATGCAGGGCCGC |
| | | CAGTTTCCGTACTCGTTCGGACAGGGCACCAAAGTGGAAATCAAG |
| 139102-aa | 74 | QVQLVQSGAEVKKPGASVKVSCKASGYTFSNYGITWVRQAPGQGLEWMGWISAYNGNT |
| VH | | NYAQKFQGRVTMTRNTSISTAYMELSSLRSEDTAVYYCARGPYYYYMDVWGKGTMVTV |
| | | SS |
| 139102-aa | 89 | EIVMTQSPLSLPVTPGEPASISCRSSQSLLYSNGYNYVDWYLQKPGQSPQLLIYLGSN |
| VL | | RASGVPDRFSGSGSGTDFKLQISRVEAEDVGIYYCMQGRQFPYSFGQGTKVEIK |
| 139102-aa | 104 | MALPVTALLLPLALLLHAARPQVQLVQSGAEVKKPGASVKVSCKASGYTFSNYGITWV |
| Full CAR | | RQAPGQGLEWMGWISAYNGNTNYAQKFQGRVTMTRNTSISTAYMELSSLRSEDTAVYY |
| | | CARGPYYYYMDVWGKGTMVTVSSASGGGGSGGRASGGGGSEIVMTQSPLSLPVTPGEP |
| | | ASISCRSSQSLLYSNGYNYVDWYLQKPGQSPQLLIYLGSNRASGVPDRFSGSGSGTDF |
| | | KLQISRVEAEDVGIYYCMQGRQFPYSFGQGTKVEIKTTTPAPRPPTPAPTIASQPLSL |
| | | RPEACRPAAGGAVHTRGLDFACDIYIWAPLAGTCGVLLLSLVITLYCKRGRKKLLYIF |
| | | KQPFMRPVQTTQEEDGCSCRFPEEEEGGCELRVKFSRSADAPAYKQGQNQLYNELNLG |
| | | RREEYDVLDKRRGRDPEMGGKPRRKNPQEGLYNELQKDKMAEAYSEIGMKGERRRGKG |
| | | HDGLYQGLSTATKDTYDALHMQALPPR |
| 139102-nt | 119 | ATGGCCCTCCCTGTCACCGCCCTGCTGCTTCCGCTGGCTCTTCTGCTCCACGCCGCTC |
| Full CAR | | GGCCCCAAGTCCAACTGGTCCAGAGCGGTGCAGAAGTGAAGAAGCCCGGAGCGAGCGT |
| | | GAAAGTGTCCTGCAAGGCTTCCGGGTACACCTTCTCCAACTACGGCATCACTTGGGTG |
| | | CGCCAGGCCCCGGGACAGGGCCTGGAATGGATGGGGTGGATTTCCGCGTACAACGGCA |
| | | ATACGAACTACGCTCAGAAGTTCCAGGGTAGAGTGACCATGACTAGGAACACCTCCAT |
| | | TTCCACCGCCTACATGGAACTGTCCTCCCTGCGGAGCGAGGACACCGCCGTGTACTAT |
| | | TGCGCCCGGGGACCATACTACTACTACATGGATGTCTGGGGGAAGGGGACTATGGTCA |
| | | CCGTGTCATCCGCCTCGGGAGGCGGCGGATCAGGAGGACGCGCCTCTGGTGGTGGAGG |
| | | ATCGGAGATCGTGATGACCCAGAGCCCTCTCTCCTTGCCCGTGACTCCTGGGGAGCCC |
| | | GCATCCATTTCATGCCGGAGCTCCCAGTCACTTCTCTACTCCAACGGCTATAACTACG |
| | | TGGATTGGTACCTCCAAAAGCCGGGCCAGAGCCCGCAGCTGCTGATCTACCTGGGCTC |
| | | GAACAGGGCCAGCGGAGTGCCTGACCGGTTCTCCGGGTCGGGAAGCGGGACCGACTTC |
| | | AAGCTGCAAATCTCGAGAGTGGAGGCCGAGGACGTGGGAATCTACTACTGTATGCAGG |
| | | GCCGCCAGTTTCCGTACTCGTTCGGACAGGGCACCAAAGTGGAAATCAAGACCACTAC |
| | | CCCAGCACCGAGGCCACCCACCCCGGCTCCTACCATCGCCTCCCAGCCTCTGTCCCTG |
| | | CGTCCGGAGGCATGTAGACCCGCAGCTGGTGGGGCCGTGCATACCCGGGGTCTTGACT |
| | | TCGCCTGCGATATCTACATTTGGGCCCCTCTGGCTGGTACTTGCGGGGTCCTGCTGCT |
| | | TTCACTCGTGATCACTCTTTACTGTAAGCGCGGTCGGAAGAAGCTGCTGTACATCTTT |
| | | AAGCAACCCTTCATGAGGCCTGTGCAGACTACTCAAGAGGAGGACGGCTGTTCATGCC |
| | | GGTTCCCAGAGGAGGAGGAAGGCGGCTGCGAACTGCGCGTGAAATTCAGCCGCAGCGC |
| | | AGATGCTCCAGCCTACAAGCAGGGGCAGAACCAGCTCTACAACGAACTCAATCTTGGT |
| | | CGGAGAGAGGAGTACGACGTGCTGGACAAGCGGAGAGGACGGGACCCAGAAATGGGCG |
| | | GGAAGCCGCGCAGAAAGAATCCCCAAGAGGGCCTGTACAACGAGCTCCAAAAGGATAA |
| | | GATGGCAGAAGCCTATAGCGAGATTGGTATGAAAGGGGAACGCAGAAGAGGCAAAGGC |
| | | CACGACGGACTGTACCAGGGACTCAGCACCGCCACCAAGGACACCTATGACGCTCTTC |
| | | ACATGCAGGCCCTGCCGCCTCGG |
| 139104 | | |
| 139104-aa | 45 | EVQLLETGGGLVQPGGSLRLSCAVSGFALSNHGMSWVRRAPGKGLEWVSGIVYSGSTY |
| ScFv domain | | YAASVKGRFTISRDNSRNTLYLQMNSLRPEDTAIYYCSAHGGESDVWGQGTTVTVSSA |
| | | SGGGGSGGRASGGGGSEIVLTQSPATLSVSPGESATLSCRASQSVSSNLAWYQQKPGQ |
| | | APRLLIYGASTRASGIPDRFSGSGSGTDFTLTISSLQAEDVAVYYCQQYGSSLTFGGG |
| | | TKVEIK |
| 139104-nt | 60 | GAAGTGCAATTGCTCGAAACTGGAGGAGGTCTGGTGCAACCTGGAGGATCACTTCGCC |
| ScFv domain | | TGTCCTGCGCCGTGTCGGGCTTTGCCCTGTCCAACCATGGAATGAGCTGGGTCCGCCG |
| | | CGCGCCGGGGAAGGGCCTCGAATGGGTGTCCGGCATCGTCTACTCCGGCTCCACCTAC |
| | | TACGCCGCGTCCGTGAAGGGCCGGTTCACGATTTCACGGGACAACTCGCGGAACACCC |
| | | TGTACCTCCAAATGAATTCCCTTCGGCCGGAGGATACTGCCATCTACTACTGCTCCGC |
| | | CCACGGTGGCGAATCCGACGTCTGGGGCCAGGGAACCACCGTGACCGTGTCCAGCGCG |
| | | TCCGGGGGAGGAGGAAGCGGGGGTAGAGCATCGGGTGGAGGCGGATCAGAGATCGTGC |
| | | TGACCCAGTCCCCCGCCACCTTGAGCGTGTCACCAGGAGAGTCCGCCACCCTGTCATG |
| | | CCGCGCCAGCCAGTCCGTGTCCTCCAACCTGGCTTGGTACCAGCAGAAGCCGGGGCAG |
| | | GCCCCTAGACTCCTGATCTATGGGGCGTCGACCCGGGCATCTGGAATTCCCGATAGGT |
| | | TCAGCGGATCGGGCTCGGGCACTGACTTCACTCTGACCATCTCCTCGCTGCAAGCCGA |
| | | GGACGTGGCTGTGTACTACTGTCAGCAGTACGGAAGCTCCCTGACTTTCGGTGGCGGG |
| | | ACCAAAGTCGAGATTAAG |
| 139104-aa | 75 | EVQLLETGGGLVQPGGSLRLSCAVSGFALSNHGMSWVRRAPGKGLEWVSGIVYSGSTY |
| VH | | YAASVKGRFTISRDNSRNTLYLQMNSLRPEDTAIYYCSAHGGESDVWGQGTTVTVSS |
| 139104-aa | 90 | EIVLTQSPATLSVSPGESATLSCRASQSVSSNLAWYQQKPGQAPRLLIYGASTRASGI |
| VL | | PDRFSGSGSGTDFTLTISSLQAEDVAVYYCQQYGSSLTFGGGTKVEIK |
| 139104-aa | 105 | MALPVTALLLPLALLLHAARPEVQLLETGGGLVQPGGSLRLSCAVSGFALSNHGMSWV |
| Full CAR | | RRAPGKGLEWVSGIVYSGSTYYAASVKGRFTISRDNSRNTLYLQMNSLRPEDTAIYYC |
| | | SAHGGESDVWGQGTTVTVSSASGGGGSGGRASGGGGSEIVLTQSPATLSVSPGESATL |
| | | SCRASQSVSSNLAWYQQKPGQAPRLLIYGASTRASGIPDRFSGSGSGTDFTLTISSLQ |
| | | AEDVAVYYCQQYGSSLTFGGGTKVEIKTTTPAPRPPTPAPTIASQPLSLRPEACRPAA |
| | | GGAVHTRGLDFACDIYIWAPLAGTCGVLLLSLVITLYCKRGRKKLLYIFKQPFMRPVQ |
| | | TTQEEDGCSCRFPEEEEGGCELRVKFSRSADAPAYKQGQNQLYNELNLGRREEYDVLD |
| | | KRRGRDPEMGGKPRRKNPQEGLYNELQKDKMAEAYSEIGMKGERRRGKGHDGLYQGLS |
| | | TATKDTYDALHMQALPPR |
| 139104-nt | 120 | ATGGCCCTCCCTGTCACCGCCCTGCTGCTTCCGCTGGCTCTTCTGCTCCACGCCGCTC |
| Full CAR | | GGCCCGAAGTGCAATTGCTCGAAACTGGAGGAGGTCTGGTGCAACCTGGAGGATCACT |
| | | TCGCCTGTCCTGCGCCGTGTCGGGCTTTGCCCTGTCCAACCATGGAATGAGCTGGGTC |
| | | CGCCGCGCGCCGGGGAAGGGCCTCGAATGGGTGTCCGGCATCGTCTACTCCGGCTCCA |
| | | CCTACTACGCCGCGTCCGTGAAGGGCCGGTTCACGATTTCACGGGACAACTCGCGGAA |
| | | CACCCTGTACCTCCAAATGAATTCCCTTCGGCCGGAGGATACTGCCATCTACTACTGC |
| | | TCCGCCCACGGTGGCGAATCCGACGTCTGGGGCCAGGGAACCACCGTGACCGTGTCCA |
| | | GCGCGTCCGGGGGAGGAGGAAGCGGGGGTAGAGCATCGGGTGGAGGCGGATCAGAGAT |
| | | CGTGCTGACCCAGTCCCCCGCCACCTTGAGCGTGTCACCAGGAGAGTCCGCCACCCTG |
| | | TCATGCCGCGCCAGCCAGTCCGTGTCCTCCAACCTGGCTTGGTACCAGCAGAAGCCGG |
| | | GGCAGGCCCCTAGACTCCTGATCTATGGGGCGTCGACCCGGGCATCTGGAATTCCCGA |
| | | TAGGTTCAGCGGATCGGGCTCGGGCACTGACTTCACTCTGACCATCTCCTCGCTGCAA |
| | | GCCGAGGACGTGGCTGTGTACTACTGTCAGCAGTACGGAAGCTCCCTGACTTTCGGTG |
| | | GCGGGACCAAAGTCGAGATTAAGACCACTACCCCAGCACCGAGGCCACCCACCCCGGC |
| | | TCCTACCATCGCCTCCCAGCCTCTGTCCCTGCGTCCGGAGGCATGTAGACCCGCAGCT |
| | | GGTGGGGCCGTGCATACCCGGGGTCTTGACTTCGCCTGCGATATCTACATTTGGGCCC |
| | | CTCTGGCTGGTACTTGCGGGGTCCTGCTGCTTTCACTCGTGATCACTCTTTACTGTAA |
| | | GCGCGGTCGGAAGAAGCTGCTGTACATCTTTAAGCAACCCTTCATGAGGCCTGTGCAG |
| | | ACTACTCAAGAGGAGGACGGCTGTTCATGCCGGTTCCCAGAGGAGGAGGAAGGCGGCT |
| | | GCGAACTGCGCGTGAAATTCAGCCGCAGCGCAGATGCTCCAGCCTACAAGCAGGGGCA |
| | | GAACCAGCTCTACAACGAACTCAATCTTGGTCGGAGAGAGGAGTACGACGTGCTGGAC |
| | | AAGCGGAGAGGACGGGACCCAGAAATGGGCGGGAAGCCGCGCAGAAAGAATCCCCAAG |
| | | AGGGCCTGTACAACGAGCTCCAAAAGGATAAGATGGCAGAAGCCTATAGCGAGATTGG |
| | | TATGAAAGGGGAACGCAGAAGAGGCAAAGGCCACGACGGACTGTACCAGGGACTCAGC |
| | | ACCGCCACCAAGGACACCTATGACGCTCTTCACATGCAGGCCCTGCCGCCTCGG |
| 139106 | | |
| 139106-aa | 46 | EVQLVETGGGLVQPGGSLRLSCAVSGFALSNHGMSWVRRAPGKGLEWVSGIVYSGSTY |
| ScFv domain | | YAASVKGRFTISRDNSRNTLYLQMNSLRPEDTAIYYCSAHGGESDVWGQGTTVTVSSA |
| | | SGGGGSGGRASGGGGSEIVMTQSPATLSVSPGERATLSCRASQSVSSKLAWYQQKPGQ |
| | | APRLLMYGASIRATGIPDRFSGSGSGTEFTLTISSLEPEDFAVYYCQQYGSSSWTFGQ |
| | | GTKVEIK |
| 139106-nt | 61 | GAAGTGCAATTGGTGGAAACTGGAGGAGGACTTGTGCAACCTGGAGGATCATTGAGAC |
| ScFv domain | | TGAGCTGCGCAGTGTCGGGATTCGCCCTGAGCAACCATGGAATGTCCTGGGTCAGAAG |
| | | GGCCCCTGGAAAAGGCCTCGAATGGGTGTCAGGGATCGTGTACTCCGGTTCCACTTAC |
| | | TACGCCGCCTCCGTGAAGGGGCGCTTCACTATCTCACGGGATAACTCCCGCAATACCC |
| | | TGTACCTCCAAATGAACAGCCTGCGGCCGGAGGATACCGCCATCTACTACTGTTCCGC |
| | | CCACGGTGGAGAGTCTGACGTCTGGGGCCAGGGAACTACCGTGACCGTGTCCTCCGCG |
| | | TCCGGCGGTGGAGGGAGCGGCGGCCGCGCCAGCGGCGGCGGAGGCTCCGAGATCGTGA |
| | | TGACCCAGAGCCCCGCTACTCTGTCGGTGTCGCCCGGAGAAAGGGCGACCCTGTCCTG |
| | | CCGGGCGTCGCAGTCCGTGAGCAGCAAGCTGGCTTGGTACCAGCAGAAGCCGGGCCAG |
| | | GCACCACGCCTGCTTATGTACGGTGCCTCCATTCGGGCCACCGGAATCCCGGACCGGT |
| | | TCTCGGGGTCGGGGTCCGGTACCGAGTTCACACTGACCATTTCCTCGCTCGAGCCCGA |
| | | GGACTTTGCCGTCTATTACTGCCAGCAGTACGGCTCCTCCTCATGGACGTTCGGCCAG |
| | | GGGACCAAGGTCGAAATCAAG |
| 139106-aa | 76 | EVQLVETGGGLVQPGGSLRLSCAVSGFALSNHGMSWVRRAPGKGLEWVSGIVYSGSTY |
| VH | | YAASVKGRFTISRDNSRNTLYLQMNSLRPEDTAIYYCSAHGGESDVWGQGTTVTVSS |
| 139106-aa | 91 | EIVMTQSPATLSVSPGERATLSCRASQSVSSKLAWYQQKPGQAPRLLMYGASIRATGI |
| VL | | PDRFSGSGSGTEFTLTISSLEPEDFAVYYCQQYGSSSWTFGQGTKVEIK |
| 139106-aa | 106 | MALPVTALLLPLALLLHAARPEVQLVETGGGLVQPGGSLRLSCAVSGFALSNHGMSWV |
| Full CAR | | RRAPGKGLEWVSGIVYSGSTYYAASVKGRFTISRDNSRNTLYLQMNSLRPEDTAIYYC |
| | | SAHGGESDVWGQGTTVTVSSASGGGGSGGRASGGGGSEIVMTQSPATLSVSPGERATL |
| | | SCRASQSVSSKLAWYQQKPGQAPRLLMYGASIRATGIPDRFSGSGSGTEFTLTISSLE |
| | | PEDFAVYYCQQYGSSSWTFGQGTKVEIKTTTPAPRPPTPAPTIASQPLSLRPEACRPA |
| | | AGGAVHTRGLDFACDIYIWAPLAGTCGVLLLSLVITLYCKRGRKKLLYIFKQPFMRPV |
| | | QTTQEEDGCSCRFPEEEEGGCELRVKFSRSADAPAYKQGQNQLYNELNLGRREEYDVL |
| | | DKRRGRDPEMGGKPRRKNPQEGLYNELQKDKMAEAYSEIGMKGERRRGKGHDGLYQGL |
| | | STATKDTYDALHMQALPPR |
| 139106-nt | 121 | ATGGCCCTCCCTGTCACCGCCCTGCTGCTTCCGCTGGCTCTTCTGCTCCACGCCGCTC |
| Full CAR | | GGCCCGAAGTGCAATTGGTGGAAACTGGAGGAGGACTTGTGCAACCTGGAGGATCATT |
| | | GAGACTGAGCTGCGCAGTGTCGGGATTCGCCCTGAGCAACCATGGAATGTCCTGGGTC |
| | | AGAAGGGCCCCTGGAAAAGGCCTCGAATGGGTGTCAGGGATCGTGTACTCCGGTTCCA |
| | | CTTACTACGCCGCCTCCGTGAAGGGGCGCTTCACTATCTCACGGGATAACTCCCGCAA |
| | | TACCCTGTACCTCCAAATGAACAGCCTGCGGCCGGAGGATACCGCCATCTACTACTGT |
| | | TCCGCCCACGGTGGAGAGTCTGACGTCTGGGGCCAGGGAACTACCGTGACCGTGTCCT |
| | | CCGCGTCCGGCGGTGGAGGGAGCGGCGGCCGCGCCAGCGGCGGCGGAGGCTCCGAGAT |
| | | CGTGATGACCCAGAGCCCCGCTACTCTGTCGGTGTCGCCCGGAGAAAGGGCGACCCTG |
| | | TCCTGCCGGGCGTCGCAGTCCGTGAGCAGCAAGCTGGCTTGGTACCAGCAGAAGCCGG |
| | | GCCAGGCACCACGCCTGCTTATGTACGGTGCCTCCATTCGGGCCACCGGAATCCCGGA |
| | | CCGGTTCTCGGGGTCGGGGTCCGGTACCGAGTTCACACTGACCATTTCCTCGCTCGAG |
| | | CCCGAGGACTTTGCCGTCTATTACTGCCAGCAGTACGGCTCCTCCTCATGGACGTTCG |
| | | GCCAGGGGACCAAGGTCGAAATCAAGACCACTACCCCAGCACCGAGGCCACCCACCCC |
| | | GGCTCCTACCATCGCCTCCCAGCCTCTGTCCCTGCGTCCGGAGGCATGTAGACCCGCA |
| | | GCTGGTGGGGCCGTGCATACCCGGGGTCTTGACTTCGCCTGCGATATCTACATTTGGG |
| | | CCCCTCTGGCTGGTACTTGCGGGGTCCTGCTGCTTTCACTCGTGATCACTCTTTACTG |
| | | TAAGCGCGGTCGGAAGAAGCTGCTGTACATCTTTAAGCAACCCTTCATGAGGCCTGTG |
| | | CAGACTACTCAAGAGGAGGACGGCTGTTCATGCCGGTTCCCAGAGGAGGAGGAAGGCG |
| | | GCTGCGAACTGCGCGTGAAATTCAGCCGCAGCGCAGATGCTCCAGCCTACAAGCAGGG |
| | | GCAGAACCAGCTCTACAACGAACTCAATCTTGGTCGGAGAGAGGAGTACGACGTGCTG |
| | | GACAAGCGGAGAGGACGGGACCCAGAAATGGGCGGGAAGCCGCGCAGAAAGAATCCCC |
| | | AAGAGGGCCTGTACAACGAGCTCCAAAAGGATAAGATGGCAGAAGCCTATAGCGAGAT |
| | | TGGTATGAAAGGGGAACGCAGAAGAGGCAAAGGCCACGACGGACTGTACCAGGGACTC |
| | | AGCACCGCCACCAAGGACACCTATGACGCTCTTCACATGCAGGCCCTGCCGCCTCGG |
| 139107 | | |
| 139107-aa | 47 | EVQLVETGGGVVQPGGSLRLSCAVSGFALSNHGMSWVRRAPGKGLEWVSGIVYSGSTY |
| ScFv domain | | YAASVKGRFTISRDNSRNTLYLQMNSLRPEDTAIYYCSAHGGESDVWGQGTTVTVSSA |
| | | SGGGGSGGRASGGGGSEIVLTQSPGTLSLSPGERATLSCRASQSVGSTNLAWYQQKPG |
| | | QAPRLLIYDASNRATGIPDRFSGGGSGTDFTLTISRLEPEDFAVYYCQQYGSSPPWTF |
| | | GQGTKVEIK |
| 139107-nt | 62 | GAAGTGCAATTGGTGGAGACTGGAGGAGGAGTGGTGCAACCTGGAGGAAGCCTGAGAC |
| ScFv domain | | TGTCATGCGCGGTGTCGGGCTTCGCCCTCTCCAACCACGGAATGTCCTGGGTCCGCCG |
| | | GGCCCCTGGGAAAGGACTTGAATGGGTGTCCGGCATCGTGTACTCGGGTTCCACCTAC |
| | | TACGCGGCCTCAGTGAAGGGCCGGTTTACTATTAGCCGCGACAACTCCAGAAACACAC |
| | | TGTACCTCCAAATGAACTCGCTGCGGCCGGAAGATACCGCTATCTACTACTGCTCCGC |
| | | CCATGGGGGAGAGTCGGACGTCTGGGGACAGGGCACCACTGTCACTGTGTCCAGCGCT |
| | | TCCGGCGGTGGTGGAAGCGGGGGACGGGCCTCAGGAGGCGGTGGCAGCGAGATTGTGC |
| | | TGACCCAGTCCCCCGGGACCCTGAGCCTGTCCCCGGGAGAAAGGGCCACCCTCTCCTG |
| | | TCGGGCATCCCAGTCCGTGGGGTCTACTAACCTTGCATGGTACCAGCAGAAGCCCGGC |
| | | CAGGCCCCTCGCCTGCTGATCTACGACGCGTCCAATAGAGCCACCGGCATCCCGGATC |
| | | GCTTCAGCGGAGGCGGATCGGGCACCGACTTCACCCTCACCATTTCAAGGCTGGAACC |
| | | GGAGGACTTCGCCGTGTACTACTGCCAGCAGTATGGTTCGTCCCCACCCTGGACGTTC |
| | | GGCCAGGGGACTAAGGTCGAGATCAAG |
| 139107-aa | 77 | EVQLVETGGGVVQPGGSLRLSCAVSGFALSNHGMSWVRRAPGKGLEWVSGIVYSGSTY |
| VH | | YAASVKGRFTISRDNSRNTLYLQMNSLRPEDTAIYYCSAHGGESDVWGQGTTVTVSS |
| 139107-aa | 92 | EIVLTQSPGTLSLSPGERATLSCRASQSVGSTNLAWYQQKPGQAPRLLIYDASNRATG |
| VL | | IPDRFSGGGSGTDFTLTISRLEPEDFAVYYCQQYGSSPPWTFGQGTKVEIK |
| 139107-aa | 107 | MALPVTALLLPLALLLHAARPEVQLVETGGGVVQPGGSLRLSCAVSGFALSNHGMSWV |
| Full CAR | | RRAPGKGLEWVSGIVYSGSTYYAASVKGRFTISRDNSRNTLYLQMNSLRPEDTAIYYC |
| | | SAHGGESDVWGQGTTVTVSSASGGGGSGGRASGGGGSEIVLTQSPGTLSLSPGERATL |
| | | SCRASQSVGSTNLAWYQQKPGQAPRLLIYDASNRATGIPDRFSGGGSGTDFTLTISRL |
| | | EPEDFAVYYCQQYGSSPPWTFGQGTKVEIKTTTPAPRPPTPAPTIASQPLSLRPEACR |
| | | PAAGGAVHTRGLDFACDIYIWAPLAGTCGVLLLSLVITLYCKRGRKKLLYIFKQPFMR |
| | | PVQTTQEEDGCSCRFPEEEEGGCELRVKFSRSADAPAYKQGQNQLYNELNLGRREEYD |
| | | VLDKRRGRDPEMGGKPRRKNPQEGLYNELQKDKMAEAYSEIGMKGERRRGKGHDGLYQ |
| | | GLSTATKDTYDALHMQALPPR |
| 139107-nt | 122 | ATGGCCCTCCCTGTCACCGCCCTGCTGCTTCCGCTGGCTCTTCTGCTCCACGCCGCTC |
| Full CAR | | GGCCCGAAGTGCAATTGGTGGAGACTGGAGGAGGAGTGGTGCAACCTGGAGGAAGCCT |
| | | GAGACTGTCATGCGCGGTGTCGGGCTTCGCCCTCTCCAACCACGGAATGTCCTGGGTC |
| | | CGCCGGGCCCCTGGGAAAGGACTTGAATGGGTGTCCGGCATCGTGTACTCGGGTTCCA |
| | | CCTACTACGCGGCCTCAGTGAAGGGCCGGTTTACTATTAGCCGCGACAACTCCAGAAA |
| | | CACACTGTACCTCCAAATGAACTCGCTGCGGCCGGAAGATACCGCTATCTACTACTGC |
| | | TCCGCCCATGGGGGAGAGTCGGACGTCTGGGGACAGGGCACCACTGTCACTGTGTCCA |
| | | GCGCTTCCGGCGGTGGTGGAAGCGGGGGACGGGCCTCAGGAGGCGGTGGCAGCGAGAT |
| | | TGTGCTGACCCAGTCCCCCGGGACCCTGAGCCTGTCCCCGGGAGAAAGGGCCACCCTC |
| | | TCCTGTCGGGCATCCCAGTCCGTGGGGTCTACTAACCTTGCATGGTACCAGCAGAAGC |
| | | CCGGCCAGGCCCCTCGCCTGCTGATCTACGACGCGTCCAATAGAGCCACCGGCATCCC |
| | | GGATCGCTTCAGCGGAGGCGGATCGGGCACCGACTTCACCCTCACCATTTCAAGGCTG |
| | | GAACCGGAGGACTTCGCCGTGTACTACTGCCAGCAGTATGGTTCGTCCCCACCCTGGA |
| | | CGTTCGGCCAGGGGACTAAGGTCGAGATCAAGACCACTACCCCAGCACCGAGGCCACC |
| | | CACCCCGGCTCCTACCATCGCCTCCCAGCCTCTGTCCCTGCGTCCGGAGGCATGTAGA |
| | | CCCGCAGCTGGTGGGGCCGTGCATACCCGGGGTCTTGACTTCGCCTGCGATATCTACA |
| | | TTTGGGCCCCTCTGGCTGGTACTTGCGGGGTCCTGCTGCTTTCACTCGTGATCACTCT |
| | | TTACTGTAAGCGCGGTCGGAAGAAGCTGCTGTACATCTTTAAGCAACCCTTCATGAGG |
| | | CCTGTGCAGACTACTCAAGAGGAGGACGGCTGTTCATGCCGGTTCCCAGAGGAGGAGG |
| | | AAGGCGGCTGCGAACTGCGCGTGAAATTCAGCCGCAGCGCAGATGCTCCAGCCTACAA |
| | | GCAGGGGCAGAACCAGCTCTACAACGAACTCAATCTTGGTCGGAGAGAGGAGTACGAC |
| | | GTGCTGGACAAGCGGAGAGGACGGGACCCAGAAATGGGCGGGAAGCCGCGCAGAAAGA |
| | | ATCCCCAAGAGGGCCTGTACAACGAGCTCCAAAAGGATAAGATGGCAGAAGCCTATAG |
| | | CGAGATTGGTATGAAAGGGGAACGCAGAAGAGGCAAAGGCCACGACGGACTGTACCAG |
| | | GGACTCAGCACCGCCACCAAGGACACCTATGACGCTCTTCACATGCAGGCCCTGCCGC |
| | | CTCGG |
| 139108 | | |
| 139108-aa | 48 | QVQLVESGGGLVKPGGSLRLSCAASGFTFSDYYMSWIRQAPGKGLEWVSYISSSGSTI |
| ScFv domain | | YYADSVKGRFTISRDNAKNSLYLQMNSLRAEDTAVYYCARESGDGMDVWGQGTTVTVS |
| | | SASGGGGSGGRASGGGGSDIQMTQSPSSLSASVGDRVTITCRASQSISSYLNWYQQKP |
| | | GKAPKLLIYAASSLQSGVPSRFSGSGSGTDFTLTISSLQPEDFATYYCQQSYTLAFGQ |
| | | GTKVDIK |
| 139108-nt | 63 | CAAGTGCAACTCGTGGAATCTGGTGGAGGACTCGTGAAACCTGGAGGATCATTGAGAC |
| ScFv domain | | TGTCATGCGCGGCCTCGGGATTCACGTTCTCCGATTACTACATGAGCTGGATTCGCCA |
| | | GGCTCCGGGGAAGGGACTGGAATGGGTGTCCTACATTTCCTCATCCGGCTCCACCATC |
| | | TACTACGCGGACTCCGTGAAGGGGAGATTCACCATTAGCCGCGATAACGCCAAGAACA |
| | | GCCTGTACCTTCAGATGAACTCCCTGCGGGCTGAAGATACTGCCGTCTACTACTGCGC |
| | | AAGGGAGAGCGGAGATGGGATGGACGTCTGGGGACAGGGTACCACTGTGACCGTGTCG |
| | | TCGGCCTCCGGCGGAGGGGGTTCGGGTGGAAGGGCCAGCGGCGGCGGAGGCAGCGACA |
| | | TCCAGATGACCCAGTCCCCCTCATCGCTGTCCGCCTCCGTGGGCGACCGCGTCACCAT |
| | | CACATGCCGGGCCTCACAGTCGATCTCCTCCTACCTCAATTGGTATCAGCAGAAGCCC |
| | | GGAAAGGCCCCTAAGCTTCTGATCTACGCAGCGTCCTCCCTGCAATCCGGGGTCCCAT |
| | | CTCGGTTCTCCGGCTCGGGCAGCGGTACCGACTTCACTCTGACCATCTCGAGCCTGCA |
| | | GCCGGAGGACTTCGCCACTTACTACTGTCAGCAAAGCTACACCCTCGCGTTTGGCCAG |
| | | GGCACCAAAGTGGACATCAAG |
| 139108-aa | 78 | QVQLVESGGGLVKPGGSLRLSCAASGFTFSDYYMSWIRQAPGKGLEWVSYISSSGSTI |
| VH | | YYADSVKGRFTISRDNAKNSLYLQMNSLRAEDTAVYYCARESGDGMDVWGQGTTVTVS |
| | | S |
| 139108-aa | 93 | DIQMTQSPSSLSASVGDRVTITCRASQSISSYLNWYQQKPGKAPKLLIYAASSLQSGV |
| VL | | PSRFSGSGSGTDFTLTISSLQPEDFATYYCQQSYTLAFGQGTKVDIK |
| 139108-aa | 108 | MALPVTALLLPLALLLHAARPQVQLVESGGGLVKPGGSLRLSCAASGFTFSDYYMSWI |
| Full CAR | | RQAPGKGLEWVSYISSSGSTIYYADSVKGRFTISRDNAKNSLYLQMNSLRAEDTAVYY |
| | | CARESGDGMDVWGQGTTVTVSSASGGGGSGGRASGGGGSDIQMTQSPSSLSASVGDRV |
| | | TITCRASQSISSYLNWYQQKPGKAPKLLIYAASSLQSGVPSRFSGSGSGTDFTLTISS |
| | | LQPEDFATYYCQQSYTLAFGQGTKVDIKTTTPAPRPPTPAPTIASQPLSLRPEACRPA |
| | | AGGAVHTRGLDFACDIYIWAPLAGTCGVLLLSLVITLYCKRGRKKLLYIFKQPFMRPV |
| | | QTTQEEDGCSCRFPEEEEGGCELRVKFSRSADAPAYKQGQNQLYNELNLGRREEYDVL |
| | | DKRRGRDPEMGGKPRRKNPQEGLYNELQKDKMAEAYSEIGMKGERRRGKGHDGLYQGL |
| | | STATKDTYDALHMQALPPR |
| 139108-nt | 123 | ATGGCCCTCCCTGTCACCGCCCTGCTGCTTCCGCTGGCTCTTCTGCTCCACGCCGCTC |
| Full CAR | | GGCCCCAAGTGCAACTCGTGGAATCTGGTGGAGGACTCGTGAAACCTGGAGGATCATT |
| | | GAGACTGTCATGCGCGGCCTCGGGATTCACGTTCTCCGATTACTACATGAGCTGGATT |
| | | CGCCAGGCTCCGGGGAAGGGACTGGAATGGGTGTCCTACATTTCCTCATCCGGCTCCA |
| | | CCATCTACTACGCGGACTCCGTGAAGGGGAGATTCACCATTAGCCGCGATAACGCCAA |
| | | GAACAGCCTGTACCTTCAGATGAACTCCCTGCGGGCTGAAGATACTGCCGTCTACTAC |
| | | TGCGCAAGGGAGAGCGGAGATGGGATGGACGTCTGGGGACAGGGTACCACTGTGACCG |
| | | TGTCGTCGGCCTCCGGCGGAGGGGGTTCGGGTGGAAGGGCCAGCGGCGGCGGAGGCAG |
| | | CGACATCCAGATGACCCAGTCCCCCTCATCGCTGTCCGCCTCCGTGGGCGACCGCGTC |
| | | ACCATCACATGCCGGGCCTCACAGTCGATCTCCTCCTACCTCAATTGGTATCAGCAGA |
| | | AGCCCGGAAAGGCCCCTAAGCTTCTGATCTACGCAGCGTCCTCCCTGCAATCCGGGGT |
| | | CCCATCTCGGTTCTCCGGCTCGGGCAGCGGTACCGACTTCACTCTGACCATCTCGAGC |
| | | CTGCAGCCGGAGGACTTCGCCACTTACTACTGTCAGCAAAGCTACACCCTCGCGTTTG |
| | | GCCAGGGCACCAAAGTGGACATCAAGACCACTACCCCAGCACCGAGGCCACCCACCCC |
| | | GGCTCCTACCATCGCCTCCCAGCCTCTGTCCCTGCGTCCGGAGGCATGTAGACCCGCA |
| | | GCTGGTGGGGCCGTGCATACCCGGGGTCTTGACTTCGCCTGCGATATCTACATTTGGG |
| | | CCCCTCTGGCTGGTACTTGCGGGGTCCTGCTGCTTTCACTCGTGATCACTCTTTACTG |
| | | TAAGCGCGGTCGGAAGAAGCTGCTGTACATCTTTAAGCAACCCTTCATGAGGCCTGTG |
| | | CAGACTACTCAAGAGGAGGACGGCTGTTCATGCCGGTTCCCAGAGGAGGAGGAAGGCG |
| | | GCTGCGAACTGCGCGTGAAATTCAGCCGCAGCGCAGATGCTCCAGCCTACAAGCAGGG |
| | | GCAGAACCAGCTCTACAACGAACTCAATCTTGGTCGGAGAGAGGAGTACGACGTGCTG |
| | | GACAAGCGGAGAGGACGGGACCCAGAAATGGGCGGGAAGCCGCGCAGAAAGAATCCCC |
| | | AAGAGGGCCTGTACAACGAGCTCCAAAAGGATAAGATGGCAGAAGCCTATAGCGAGAT |
| | | TGGTATGAAAGGGGAACGCAGAAGAGGCAAAGGCCACGACGGACTGTACCAGGGACTC |
| | | AGCACCGCCACCAAGGACACCTATGACGCTCTTCACATGCAGGCCCTGCCGCCTCGG |
| 139110 | | |
| 139110-aa | 50 | QVQLVQSGGGLVKPGGSLRLSCAASGFTFSDYYMSWIRQAPGKGLEWVSYISSSGNTI |
| ScFv domain | | YYADSVKGRFTISRDNAKNSLYLQMNSLRAEDTAVYYCARSTMVREDYWGQGTLVTVS |
| | | SASGGGGSGGRASGGGGSDIVLTQSPLSLPVTLGQPASISCKSSESLVHNSGKTYLNW |
| | | FHQRPGQSPRRLIYEVSNRDSGVPDRFTGSGSGTDFTLKISRVEAEDVGVYYCMQGTH |
| | | WPGTFGQGTKLEIK |
| 139110-nt | 65 | CAAGTGCAACTGGTGCAAAGCGGAGGAGGATTGGTCAAACCCGGAGGAAGCCTGAGAC |
| ScFv domain | | TGTCATGCGCGGCCTCTGGATTCACCTTCTCCGATTACTACATGTCATGGATCAGACA |
| | | GGCCCCGGGGAAGGGCCTCGAATGGGTGTCCTACATCTCGTCCTCCGGGAACACCATC |
| | | TACTACGCCGACAGCGTGAAGGGCCGCTTTACCATTTCCCGCGACAACGCAAAGAACT |
| | | CGCTGTACCTTCAGATGAATTCCCTGCGGGCTGAAGATACCGCGGTGTACTATTGCGC |
| | | CCGGTCCACTATGGTCCGGGAGGACTACTGGGGACAGGGCACACTCGTGACCGTGTCC |
| | | AGCGCGAGCGGGGGTGGAGGCAGCGGTGGACGCGCCTCCGGCGGCGGCGGTTCAGACA |
| | | TCGTGCTGACTCAGTCGCCCCTGTCGCTGCCGGTCACCCTGGGCCAACCGGCCTCAAT |
| | | TAGCTGCAAGTCCTCGGAGAGCCTGGTGCACAACTCAGGAAAGACTTACCTGAACTGG |
| | | TTCCATCAGCGGCCTGGACAGTCCCCACGGAGGCTCATCTATGAAGTGTCCAACAGGG |
| | | ATTCGGGGGTGCCCGACCGCTTCACTGGCTCCGGGTCCGGCACCGACTTCACCTTGAA |
| | | AATCTCCAGAGTGGAAGCCGAGGACGTGGGCGTGTACTACTGTATGCAGGGTACCCAC |
| | | TGGCCTGGAACCTTTGGACAAGGAACTAAGCTCGAGATTAAG |
| 139110-aa | 80 | QVQLVQSGGGLVKPGGSLRLSCAASGFTFSDYYMSWIRQAPGKGLEWVSYISSSGNTI |
| VH | | YYADSVKGRFTISRDNAKNSLYLQMNSLRAEDTAVYYCARSTMVREDYWGQGTLVTVS |
| | | S |
| 139110-aa | 95 | DIVLTQSPLSLPVTLGQPASISCKSSESLVHNSGKTYLNWFHQRPGQSPRRLIYEVSN |
| VL | | RDSGVPDRFTGSGSGTDFTLKISRVEAEDVGVYYCMQGTHWPGTFGQGTKLEIK |
| 139110-aa | 110 | MALPVTALLLPLALLLHAARPQVQLVQSGGGLVKPGGSLRLSCAASGFTFSDYYMSWI |
| Full CAR | | RQAPGKGLEWVSYISSSGNTIYYADSVKGRFTISRDNAKNSLYLQMNSLRAEDTAVYY |
| | | CARSTMVREDYWGQGTLVTVSSASGGGGSGGRASGGGGSDIVLTQSPLSLPVTLGQPA |
| | | SISCKSSESLVHNSGKTYLNWFHQRPGQSPRRLIYEVSNRDSGVPDRFTGSGSGTDFT |
| | | LKISRVEAEDVGVYYCMQGTHWPGTFGQGTKLEIKTTTPAPRPPTPAPTIASQPLSLR |
| | | PEACRPAAGGAVHTRGLDFACDIYIWAPLAGTCGVLLLSLVITLYCKRGRKKLLYIFK |
| | | QPFMRPVQTTQEEDGCSCRFPEEEEGGCELRVKFSRSADAPAYKQGQNQLYNELNLCR |
| | | REEYDVLDKRRGRDPEMGGKPRRKNPQEGLYNELQKDKMAEAYSEIGMKGERRRGKGH |
| | | DGLYQGLSTATKDTYDALHMQALPPR |
| 139110-nt | 125 | ATGGCCCTCCCTGTCACCGCCCTGCTGCTTCCGCTGGCTCTTCTGCTCCACGCCGCTC |
| Full CAR | | GGCCCCAAGTGCAACTGGTGCAAAGCGGAGGAGGATTGGTCAAACCCGGAGGAAGCCT |
| | | GAGACTGTCATGCGCGGCCTCTGGATTCACCTTCTCCGATTACTACATGTCATGGATC |
| | | AGACAGGCCCCGGGGAAGGGCCTCGAATGGGTGTCCTACATCTCGTCCTCCGGGAACA |
| | | CCATCTACTACGCCGACAGCGTGAAGGGCCGCTTTACCATTTCCCGCGACAACGCAAA |
| | | GAACTCGCTGTACCTTCAGATGAATTCCCTGCGGGCTGAAGATACCGCGGTGTACTAT |
| | | TGCGCCCGGTCCACTATGGTCCGGGAGGACTACTGGGGACAGGGCACACTCGTGACCG |
| | | TGTCCAGCGCGAGCGGGGGTGGAGGCAGCGGTGGACGCGCCTCCGGCGGCGGCGGTTC |
| | | AGACATCGTGCTGACTCAGTCGCCCCTGTCGCTGCCGGTCACCCTGGGCCAACCGGCC |
| | | TCAATTAGCTGCAAGTCCTCGGAGAGCCTGGTGCACAACTCAGGAAAGACTTACCTGA |
| | | ACTGGTTCCATCAGCGGCCTGGACAGTCCCCACGGAGGCTCATCTATGAAGTGTCCAA |
| | | CAGGGATTCGGGGGTGCCCGACCGCTTCACTGGCTCCGGGTCCGGCACCGACTTCACC |
| | | TTGAAAATCTCCAGAGTGGAAGCCGAGGACGTGGGCGTGTACTACTGTATGCAGGGTA |
| | | CCCACTGGCCTGGAACCTTTGGACAAGGAACTAAGCTCGAGATTAAGACCACTACCCC |
| | | AGCACCGAGGCCACCCACCCCGGCTCCTACCATCGCCTCCCAGCCTCTGTCCCTGCGT |
| | | CCGGAGGCATGTAGACCCGCAGCTGGTGGGGCCGTGCATACCCGGGGTCTTGACTTCG |
| | | CCTGCGATATCTACATTTGGGCCCCTCTGGCTGGTACTTGCGGGGTCCTGCTGCTTTC |
| | | ACTCGTGATCACTCTTTACTGTAAGCGCGGTCGGAAGAAGCTGCTGTACATCTTTAAG |
| | | CAACCCTTCATGAGGCCTGTGCAGACTACTCAAGAGGAGGACGGCTGTTCATGCCGGT |
| | | TCCCAGAGGAGGAGGAAGGCGGCTGCGAACTGCGCGTGAAATTCAGCCGCAGCGCAGA |
| | | TGCTCCAGCCTACAAGCAGGGGCAGAACCAGCTCTACAACGAACTCAATCTTGGTCGG |
| | | AGAGAGGAGTACGACGTGCTGGACAAGCGGAGAGGACGGGACCCAGAAATGGGCGGGA |
| | | AGCCGCGCAGAAAGAATCCCCAAGAGGGCCTGTACAACGAGCTCCAAAAGGATAAGAT |
| | | GGCAGAAGCCTATAGCGAGATTGGTATGAAAGGGGAACGCAGAAGAGGCAAAGGCCAC |
| | | GACGGACTGTACCAGGGACTCAGCACCGCCACCAAGGACACCTATGACGCTCTTCACA |
| | | TGCAGGCCCTGCCGCCTCGG |
| 139112 | | |
| 139112-aa | 51 | QVQLVESGGGLVQPGGSLRLSCAVSGFALSNHGMSWVRRAPGKGLEWVSGIVYSGSTY |
| ScFv domain | | YAASVKGRFTISRDNSRNTLYLQMNSLRPEDTAIYYCSAHGGESDVWGQGTTVTVSSA |
| | | SGGGGSGGRASGGGGSDIRLTQSPSPLSASVGDRVTITCQASEDINKFLNWYHQTPGK |
| | | APKLLIYDASTLQTGVPSRFSGSGSGTDFTLTINSLQPEDIGTYYCQQYESLPLTFGG |
| | | GTKVEIK |
| 139112-nt | 66 | CAAGTGCAACTCGTGGAATCTGGTGGAGGACTCGTGCAACCCGGTGGAAGCCTTAGGC |
| ScFv domain | | TGTCGTGCGCCGTCAGCGGGTTTGCTCTGAGCAACCATGGAATGTCCTGGGTCCGCCG |
| | | GGCACCGGGAAAAGGGCTGGAATGGGTGTCCGGCATCGTGTACAGCGGGTCAACCTAT |
| | | TACGCCGCGTCCGTGAAGGGCAGATTCACTATCTCAAGAGACAACAGCCGGAACACCC |
| | | TGTACTTGCAAATGAATTCCCTGCGCCCCGAGGACACCGCCATCTACTACTGCTCCGC |
| | | CCACGGAGGAGAGTCGGACGTGTGGGGCCAGGGAACGACTGTGACTGTGTCCAGCGCA |
| | | TCAGGAGGGGGTGGTTCGGGCGGCCGGGCCTCGGGGGGAGGAGGTTCCGACATTCGGC |
| | | TGACCCAGTCCCCGTCCCCACTGTCGGCCTCCGTCGGCGACCGCGTGACCATCACTTG |
| | | TCAGGCGTCCGAGGACATTAACAAGTTCCTGAACTGGTACCACCAGACCCCTGGAAAG |
| | | GCCCCCAAGCTGCTGATCTACGATGCCTCGACCCTTCAAACTGGAGTGCCTAGCCGGT |
| | | TCTCCGGGTCCGGCTCCGGCACTGATTTCACTCTGACCATCAACTCATTGCAGCCGGA |
| | | AGATATCGGGACCTACTATTGCCAGCAGTACGAATCCCTCCCGCTCACATTCGGCGGG |
| | | GGAACCAAGGTCGAGATTAAG |
| 139112-aa | 81 | QVQLVESGGGLVQPGGSLRLSCAVSGFALSNHGMSWVRRAPGKGLEWVSGIVYSGSTY |
| VH | | YAASVKGRFTISRDNSRNTLYLQMNSLRPEDTAIYYCSAHGGESDVWGQGTTVTVSS |
| 139112-aa | 96 | DIRLTQSPSPLSASVGDRVTITCQASEDINKFLNWYHQTPGKAPKLLIYDASTLQTGV |
| VL | | PSRFSGSGSGTDFTLTINSLQPEDIGTYYCQQYESLPLTFGGGTKVEIK |
| 139112-aa | 111 | MALPVTALLLPLALLLHAARPQVQLVESGGGLVQPGGSLRLSCAVSGFALSNHGMSWV |
| Full CAR | | RRAPGKGLEWVSGIVYSGSTYYAASVKGRFTISRDNSRNTLYLQMNSLRPEDTAIYYC |
| | | SAHGGESDVWGQGTTVTVSSASGGGGSGGRASGGGGSDIRLTQSPSPLSASVGDRVTI |
| | | TCQASEDINKFLNWYHQTPGKAPKLLIYDASTLQTGVPSRFSGSGSGTDFTLTINSLQ |
| | | PEDIGTYYCQQYESLPLTFGGGTKVEIKTTTPAPRPPTPAPTIASQPLSLRPEACRPA |
| | | AGGAVHTRGLDFACDIYIWAPLAGTCGVLLLSLVITLYCKRGRKKLLYIFKQPFMRPV |
| | | QTTQEEDGCSCRFPEEEEGGCELRVKFSRSADAPAYKQGQNQLYNELNLGRREEYDVL |
| | | DKRRGRDPEMGGKPRRKNPQEGLYNELQKDKMAEAYSEIGMKGERRRGKGHDGLYQGL |
| | | STATKDTYDALHMQALPPR |
| 139112-nt | 126 | ATGGCCCTCCCTGTCACCGCCCTGCTGCTTCCGCTGGCTCTTCTGCTCCACGCCGCTC |
| Full CAR | | GGCCCCAAGTGCAACTCGTGGAATCTGGTGGAGGACTCGTGCAACCCGGTGGAAGCCT |
| | | TAGGCTGTCGTGCGCCGTCAGCGGGTTTGCTCTGAGCAACCATGGAATGTCCTGGGTC |
| | | CGCCGGGCACCGGGAAAAGGGCTGGAATGGGTGTCCGGCATCGTGTACAGCGGGTCAA |
| | | CCTATTACGCCGCGTCCGTGAAGGGCAGATTCACTATCTCAAGAGACAACAGCCGGAA |
| | | CACCCTGTACTTGCAAATGAATTCCCTGCGCCCCGAGGACACCGCCATCTACTACTGC |
| | | TCCGCCCACGGAGGAGAGTCGGACGTGTGGGGCCAGGGAACGACTGTGACTGTGTCCA |
| | | GCGCATCAGGAGGGGGTGGTTCGGGCGGCCGGGCCTCGGGGGGAGGAGGTTCCGACAT |
| | | TCGGCTGACCCAGTCCCCGTCCCCACTGTCGGCCTCCGTCGGCGACCGCGTGACCATC |
| | | ACTTGTCAGGCGTCCGAGGACATTAACAAGTTCCTGAACTGGTACCACCAGACCCCTG |
| | | GAAAGGCCCCCAAGCTGCTGATCTACGATGCCTCGACCCTTCAAACTGGAGTGCCTAG |
| | | CCGGTTCTCCGGGTCCGGCTCCGGCACTGATTTCACTCTGACCATCAACTCATTGCAG |
| | | CCGGAAGATATCGGGACCTACTATTGCCAGCAGTACGAATCCCTCCCGCTCACATTCG |
| | | GCGGGGGAACCAAGGTCGAGATTAAGACCACTACCCCAGCACCGAGGCCACCCACCCC |
| | | GGCTCCTACCATCGCCTCCCAGCCTCTGTCCCTGCGTCCGGAGGCATGTAGACCCGCA |
| | | GCTGGTGGGGCCGTGCATACCCGGGGTCTTGACTTCGCCTGCGATATCTACATTTGGG |
| | | CCCCTCTGGCTGGTACTTGCGGGGTCCTGCTGCTTTCACTCGTGATCACTCTTTACTG |
| | | TAAGCGCGGTCGGAAGAAGCTGCTGTACATCTTTAAGCAACCCTTCATGAGGCCTGTG |
| | | CAGACTACTCAAGAGGAGGACGGCTGTTCATGCCGGTTCCCAGAGGAGGAGGAAGGCG |
| | | GCTGCGAACTGCGCGTGAAATTCAGCCGCAGCGCAGATGCTCCAGCCTACAAGCAGGG |
| | | GCAGAACCAGCTCTACAACGAACTCAATCTTGGTCGGAGAGAGGAGTACGACGTGCTG |
| | | GACAAGCGGAGAGGACGGGACCCAGAAATGGGCGGGAAGCCGCGCAGAAAGAATCCCC |
| | | AAGAGGGCCTGTACAACGAGCTCCAAAAGGATAAGATGGCAGAAGCCTATAGCGAGAT |
| | | TGGTATGAAAGGGGAACGCAGAAGAGGCAAAGGCCACGACGGACTGTACCAGGGACTC |
| | | AGCACCGCCACCAAGGACACCTATGACGCTCTTCACATGCAGGCCCTGCCGCCTCGG |
| 139113 | | |
| 139113-aa | 52 | EVQLVETGGGLVQPGGSLRLSCAVSGFALSNHGMSWVRRAPGKGLEWVSGIVYSGSTY |
| ScFv domain | | YAASVKGRFTISRDNSRNTLYLQMNSLRPEDTAIYYCSAHGGESDVWGQGTTVTVSSA |
| | | SGGGGSGGRASGGGGSETTLTQSPATLSVSPGERATLSCRASQSVGSNLAWYQQKPGQ |
| | | GPRLLIYGASTRATGIPARFSGSGSGTEFTLTISSLQPEDFAVYYCQQYNDWLPVTFG |
| | | QGTKVEIK |
| 139113-nt | 67 | GAAGTGCAATTGGTGGAAACTGGAGGAGGACTTGTGCAACCTGGAGGATCATTGCGGC |
| ScFv domain | | TCTCATGCGCTGTCTCCGGCTTCGCCCTGTCAAATCACGGGATGTCGTGGGTCAGACG |
| | | GGCCCCGGGAAAGGGTCTGGAATGGGTGTCGGGGATTGTGTACAGCGGCTCCACCTAC |
| | | TACGCCGCTTCGGTCAAGGGCCGCTTCACTATTTCACGGGACAACAGCCGCAACACCC |
| | | TCTATCTGCAAATGAACTCTCTCCGCCCGGAGGATACCGCCATCTACTACTGCTCCGC |
| | | ACACGGCGGCGAATCCGACGTGTGGGGACAGGGAACCACTGTCACCGTGTCGTCCGCA |
| | | TCCGGTGGCGGAGGATCGGGTGGCCGGGCCTCCGGGGGCGGCGGCAGCGAGACTACCC |
| | | TGACCCAGTCCCCTGCCACTCTGTCCGTGAGCCCGGGAGAGAGAGCCACCCTTAGCTG |
| | | CCGGGCCAGCCAGAGCGTGGGCTCCAACCTGGCCTGGTACCAGCAGAAGCCAGGACAG |
| | | GGTCCCAGGCTGCTGATCTACGGAGCCTCCACTCGCGCGACCGGCATCCCCGCGAGGT |
| | | TCTCCGGGTCGGGTTCCGGGACCGAGTTCACCCTGACCATCTCCTCCCTCCAACCGGA |
| | | GGACTTCGCGGTGTACTACTGTCAGCAGTACAACGATTGGCTGCCCGTGACATTTGGA |
| | | CAGGGGACGAAGGTGGAAATCAAA |
| 139113-aa | 82 | EVQLVETGGGLVQPGGSLRLSCAVSGFALSNHGMSWVRRAPGKGLEWVSGIVYSGSTY |
| VH | | YAASVKGRFTISRDNSRNTLYLQMNSLRPEDTAIYYCSAHGGESDVWGQGTTVTVSS |
| 139113-aa | 97 | ETTLTQSPATLSVSPGERATLSCRASQSVGSNLAWYQQKPGQGPRLLIYGASTRATGI |
| VL | | PARFSGSGSGTEFTLTISSLQPEDFAVYYCQQYNDWLPVTFGQGTKVEIK |
| 139113-aa | 112 | MALPVTALLLPLALLLHAARPEVQLVETGGGLVQPGGSLRLSCAVSGFALSNHGMSWV |
| Full CAR | | RRAPGKGLEWVSGIVYSGSTYYAASVKGRFTISRDNSRNTLYLQMNSLRPEDTAIYYC |
| | | SAHGGESDVWGQGTTVTVSSASGGGGSGGRASGGGGSETTLTQSPATLSVSPGERATL |
| | | SCRASQSVGSNLAWYQQKPGQGPRLLIYGASTRATGIPARFSGSGSGTEFTLTISSLQ |
| | | PEDFAVYYCQQYNDWLPVTFGQGTKVEIKTTTPAPRPPTPAPTIASQPLSLRPEACRP |
| | | AAGGAVHTRGLDFACDIYIWAPLAGTCGVLLLSLVITLYCKRGRKKLLYIFKQPFMRP |
| | | VQTTQEEDGCSCRFPEEEEGGCELRVKFSRSADAPAYKQGQNQLYNELNLGRREEYDV |
| | | LDKRRGRDPEMGGKPRRKNPQEGLYNELQKDKMAEAYSEIGMKGERRRGKGHDGLYQG |
| | | LSTATKDTYDALHMQALPPR |
| 139113-nt | 127 | ATGGCCCTCCCTGTCACCGCCCTGCTGCTTCCGCTGGCTCTTCTGCTCCACGCCGCTC |
| Full CAR | | GGCCCGAAGTGCAATTGGTGGAAACTGGAGGAGGACTTGTGCAACCTGGAGGATCATT |
| | | GCGGCTCTCATGCGCTGTCTCCGGCTTCGCCCTGTCAAATCACGGGATGTCGTGGGTC |
| | | AGACGGGCCCCGGGAAAGGGTCTGGAATGGGTGTCGGGGATTGTGTACAGCGGCTCCA |
| | | CCTACTACGCCGCTTCGGTCAAGGGCCGCTTCACTATTTCACGGGACAACAGCCGCAA |
| | | CACCCTCTATCTGCAAATGAACTCTCTCCGCCCGGAGGATACCGCCATCTACTACTGC |
| | | TCCGCACACGGCGGCGAATCCGACGTGTGGGGACAGGGAACCACTGTCACCGTGTCGT |
| | | CCGCATCCGGTGGCGGAGGATCGGGTGGCCGGGCCTCCGGGGGCGGCGGCAGCGAGAC |
| | | TACCCTGACCCAGTCCCCTGCCACTCTGTCCGTGAGCCCGGGAGAGAGAGCCACCCTT |
| | | AGCTGCCGGGCCAGCCAGAGCGTGGGCTCCAACCTGGCCTGGTACCAGCAGAAGCCAG |
| | | GACAGGGTCCCAGGCTGCTGATCTACGGAGCCTCCACTCGCGCGACCGGCATCCCCGC |
| | | GAGGTTCTCCGGGTCGGGTTCCGGGACCGAGTTCACCCTGACCATCTCCTCCCTCCAA |
| | | CCGGAGGACTTCGCGGTGTACTACTGTCAGCAGTACAACGATTGGCTGCCCGTGACAT |
| | | TTGGACAGGGGACGAAGGTGGAAATCAAAACCACTACCCCAGCACCGAGGCCACCCAC |
| | | CCCGGCTCCTACCATCGCCTCCCAGCCTCTGTCCCTGCGTCCGGAGGCATGTAGACCC |
| | | GCAGCTGGTGGGGCCGTGCATACCCGGGGTCTTGACTTCGCCTGCGATATCTACATTT |
| | | GGGCCCCTCTGGCTGGTACTTGCGGGGTCCTGCTGCTTTCACTCGTGATCACTCTTTA |
| | | CTGTAAGCGCGGTCGGAAGAAGCTGCTGTACATCTTTAAGCAACCCTTCATGAGGCCT |
| | | GTGCAGACTACTCAAGAGGAGGACGGCTGTTCATGCCGGTTCCCAGAGGAGGAGGAAG |
| | | GCGGCTGCGAACTGCGCGTGAAATTCAGCCGCAGCGCAGATGCTCCAGCCTACAAGCA |
| | | GGGGCAGAACCAGCTCTACAACGAACTCAATCTTGGTCGGAGAGAGGAGTACGACGTG |
| | | CTGGACAAGCGGAGAGGACGGGACCCAGAAATGGGCGGGAAGCCGCGCAGAAAGAATC |
| | | CCCAAGAGGGCCTGTACAACGAGCTCCAAAAGGATAAGATGGCAGAAGCCTATAGCGA |
| | | GATTGGTATGAAAGGGGAACGCAGAAGAGGCAAAGGCCACGACGGACTGTACCAGGGA |
| | | CTCAGCACCGCCACCAAGGACACCTATGACGCTCTTCACATGCAGGCCCTGCCGCCTC |
| | | GG |
| 139114 | | |
| 139114-aa | 53 | EVQLVESGGGLVQPGGSLRLSCAVSGFALSNHGMSWVRRAPGKGLEWVSGIVYSGSTY |
| ScFv domain | | YAASVKGRFTISRDNSRNTLYLQMNSLRPEDTAIYYCSAHGGESDVWGQGTTVTVSSA |
| | | SGGGGSGGRASGGGGSEIVLTQSPGTLSLSPGERATLSCRASQSIGSSSLAWYQQKPG |
| | | QAPRLLMYGASSRASGIPDRFSGSGSGTDFTLTISRLEPEDFAVYYCQQYAGSPPFTF |
| | | GQGTKVEIK |
| 139114-nt | 68 | GAAGTGCAATTGGTGGAATCTGGTGGAGGACTTGTGCAACCTGGAGGATCACTGAGAC |
| ScFv domain | | TGTCATGCGCGGTGTCCGGTTTTGCCCTGAGCAATCATGGGATGTCGTGGGTCCGGCG |
| | | CGCCCCCGGAAAGGGTCTGGAATGGGTGTCGGGTATCGTCTACTCCGGGAGCACTTAC |
| | | TACGCCGCGAGCGTGAAGGGCCGCTTCACCATTTCCCGCGATAACTCCCGCAACACCC |
| | | TGTACTTGCAAATGAACTCGCTCCGGCCTGAGGACACTGCCATCTACTACTGCTCCGC |
| | | ACACGGAGGAGAATCCGACGTGTGGGGCCAGGGAACTACCGTGACCGTCAGCAGCGCC |
| | | TCCGGCGGCGGGGGCTCAGGCGGACGGGCTAGCGGCGGCGGTGGCTCCGAGATCGTGC |
| | | TGACCCAGTCGCCTGGCACTCTCTCGCTGAGCCCCGGGGAAAGGGCAACCCTGTCCTG |
| | | TCGGGCCAGCCAGTCCATTGGATCATCCTCCCTCGCCTGGTATCAGCAGAAACCGGGA |
| | | CAGGCTCCGCGGCTGCTTATGTATGGGGCCAGCTCAAGAGCCTCCGGCATTCCCGACC |
| | | GGTTCTCCGGGTCCGGTTCCGGCACCGATTTCACCCTGACTATCTCGAGGCTGGAGCC |
| | | AGAGGACTTCGCCGTGTACTACTGCCAGCAGTACGCGGGGTCCCCGCCGTTCACGTTC |
| | | GGACAGGGAACCAAGGTCGAGATCAAG |
| 139114-aa | 83 | EVQLVESGGGLVQPGGSLRLSCAVSGFALSNHGMSWVRRAPGKGLEWVSGIVYSGSTY |
| VH | | YAASVKGRFTISRDNSRNTLYLQMNSLRPEDTAIYYCSAHGGESDVWGQGTTVTVSS |
| 139114-aa | 98 | EIVLTQSPGTLSLSPGERATLSCRASQSIGSSSLAWYQQKPGQAPRLLMYGASSRASG |
| VL | | IPDRFSGSGSGTDFTLTISRLEPEDFAVYYCQQYAGSPPFTFGQGTKVEIK |
| 139114-aa | 113 | MALPVTALLLPLALLLHAARPEVQLVESGGGLVQPGGSLRLSCAVSGFALSNHGMSWV |
| Full CAR | | RRAPGKGLEWVSGIVYSGSTYYAASVKGRFTISRDNSRNTLYLQMNSLRPEDTAIYYC |
| | | SAHGGESDVWGQGTTVTVSSASGGGGSGGRASGGGGSEIVLTQSPGTLSLSPGERATL |
| | | SCRASQSIGSSSLAWYQQKPGQAPRLLMYGASSRASGIPDRFSGSGSGTDFTLTISRL |
| | | EPEDFAVYYCQQYAGSPPFTFGQGTKVEIKTTTPAPRPPTPAPTIASQPLSLRPEACR |
| | | PAAGGAVHTRGLDFACDIYIWAPLAGTCGVLLLSLVITLYCKRGRKKLLYIFKQPFMR |
| | | PVQTTQEEDGCSCRFPEEEEGGCELRVKFSRSADAPAYKQGQNQLYNELNLGRREEYD |
| | | VLDKRRGRDPEMGGKPRRKNPQEGLYNELQKDKMAEAYSEIGMKGERRRGKGHDGLYQ |
| | | GLSTATKDTYDALHMQALPPR |
| 139114-nt | 128 | ATGGCCCTCCCTGTCACCGCCCTGCTGCTTCCGCTGGCTCTTCTGCTCCACGCCGCTC |
| Full CAR | | GGCCCGAAGTGCAATTGGTGGAATCTGGTGGAGGACTTGTGCAACCTGGAGGATCACT |
| | | GAGACTGTCATGCGCGGTGTCCGGTTTTGCCCTGAGCAATCATGGGATGTCGTGGGTC |
| | | CGGCGCGCCCCCGGAAAGGGTCTGGAATGGGTGTCGGGTATCGTCTACTCCGGGAGCA |
| | | CTTACTACGCCGCGAGCGTGAAGGGCCGCTTCACCATTTCCCGCGATAACTCCCGCAA |
| | | CACCCTGTACTTGCAAATGAACTCGCTCCGGCCTGAGGACACTGCCATCTACTACTGC |
| | | TCCGCACACGGAGGAGAATCCGACGTGTGGGGCCAGGGAACTACCGTGACCGTCAGCA |
| | | GCGCCTCCGGCGGCGGGGGCTCAGGCGGACGGGCTAGCGGCGGCGGTGGCTCCGAGAT |
| | | CGTGCTGACCCAGTCGCCTGGCACTCTCTCGCTGAGCCCCGGGGAAAGGGCAACCCTG |
| | | TCCTGTCGGGCCAGCCAGTCCATTGGATCATCCTCCCTCGCCTGGTATCAGCAGAAAC |
| | | CGGGACAGGCTCCGCGGCTGCTTATGTATGGGGCCAGCTCAAGAGCCTCCGGCATTCC |
| | | CGACCGGTTCTCCGGGTCCGGTTCCGGCACCGATTTCACCCTGACTATCTCGAGGCTG |
| | | GAGCCAGAGGACTTCGCCGTGTACTACTGCCAGCAGTACGCGGGGTCCCCGCCGTTCA |
| | | CGTTCGGACAGGGAACCAAGGTCGAGATCAAGACCACTACCCCAGCACCGAGGCCACC |
| | | CACCCCGGCTCCTACCATCGCCTCCCAGCCTCTGTCCCTGCGTCCGGAGGCATGTAGA |
| | | CCCGCAGCTGGTGGGGCCGTGCATACCCGGGGTCTTGACTTCGCCTGCGATATCTACA |
| | | TTTGGGCCCCTCTGGCTGGTACTTGCGGGGTCCTGCTGCTTTCACTCGTGATCACTCT |
| | | TTACTGTAAGCGCGGTCGGAAGAAGCTGCTGTACATCTTTAAGCAACCCTTCATGAGG |
| | | CCTGTGCAGACTACTCAAGAGGAGGACGGCTGTTCATGCCGGTTCCCAGAGGAGGAGG |
| | | AAGGCGGCTGCGAACTGCGCGTGAAATTCAGCCGCAGCGCAGATGCTCCAGCCTACAA |
| | | GCAGGGGCAGAACCAGCTCTACAACGAACTCAATCTTGGTCGGAGAGAGGAGTACGAC |
| | | GTGCTGGACAAGCGGAGAGGACGGGACCCAGAAATGGGCGGGAAGCCGCGCAGAAAGA |
| | | ATCCCCAAGAGGGCCTGTACAACGAGCTCCAAAAGGATAAGATGGCAGAAGCCTATAG |
| | | CGAGATTGGTATGAAAGGGGAACGCAGAAGAGGCAAAGGCCACGACGGACTGTACCAG |
| | | GGACTCAGCACCGCCACCAAGGACACCTATGACGCTCTTCACATGCAGGCCCTGCCGC |
| | | CTCGG |
| 149362 | | |
| 149362-aa | 129 | QVQLQESGPGLVKPSETLSLTCTVSGGSISSSYYYWGWIRQPPGKGLEWIGSIYYSGS |
| ScFv domain | | AYYNPSLKSRVTISVDTSKNQFSLRLSSVTAADTAVYYCARHWQEWPDAFDIWGQGTM |
| | | VTVSSGGGGSGGGGSGGGGSETTLTQSPAFMSATPGDKVIISCKASQDIDDAMNWYQQ |
| | | KPGEAPLFIIQSATSPVPGIPPRFSGSGFGTDFSLTINNIESEDAAYYFCLQHDNFPL |
| | | TFGQGTKLEIK |
| 149362-nt ScFv | 150 | CAAGTGCAGCTTCAGGAAAGCGGACCGGGCCTGGTCAAGCCATCCGAAACTCTCTCCC |
| domain | | TGACTTGCACTGTGTCTGGCGGTTCCATCTCATCGTCGTACTACTACTGGGGCTGGAT |
| | | TAGGCAGCCGCCCGGAAAGGGACTGGAGTGGATCGGAAGCATCTACTATTCCGGCTCG |
| | | GCGTACTACAACCCTAGCCTCAAGTCGAGAGTGACCATCTCCGTGGATACCTCCAAGA |
| | | ACCAGTTTTCCCTGCGCCTGAGCTCCGTGACCGCCGCTGACACCGCCGTGTACTACTG |
| | | TGCTCGGCATTGGCAGGAATGGCCCGATGCCTTCGACATTTGGGGCCAGGGCACTATG |
| | | GTCACTGTGTCATCCGGGGGTGGAGGCAGCGGGGGAGGAGGGTCCGGGGGGGGAGGTT |
| | | CAGAGACAACCTTGACCCAGTCACCCGCATTCATGTCCGCCACTCCGGGAGACAAGGT |
| | | CATCATCTCGTGCAAAGCGTCCCAGGATATCGACGATGCCATGAATTGGTACCAGCAG |
| | | AAGCCTGGCGAAGCGCCGCTGTTCATTATCCAATCCGCAACCTCGCCCGTGCCTGGAA |
| | | TCCCACCGCGGTTCAGCGGCAGCGGTTTCGGAACCGACTTTTCCCTGACCATTAACAA |
| | | CATTGAGTCCGAGGACGCCGCCTACTACTTCTGCCTGCAACACGACAACTTCCCTCTC |
| | | ACGTTCGGCCAGGGAACCAAGCTGGAAATCAAG |
| 149362-aa VH | 171 | QVQLQESGPGLVKPSETLSLTCTVSGGSISSSYYYWGWIRQPPGKGLEWIGSIYYSGS |
| | | AYYNPSLKSRVTISVDTSKNQFSLRLSSVTAADTAVYYCARHWQEWPDAFDIWGQGTM |
| | | VTVSS |
| 149362-aa VL | 192 | ETTLTQSPAFMSATPGDKVIISCKASQDIDDAMNWYQQKPGEAPLFIIQSATSPVPGI |
| | | PPRFSGSGFGTDFSLTINNIESEDAAYYFCLQHDNFPLTFGQGTKLEIK |
| 149362-aa Full | 213 | MALPVTALLLPLALLLHAARPQVQLQESGPGLVKPSETLSLTCTVSGGSISSSYYYWG |
| CAR | | WIRQPPGKGLEWIGSIYYSGSAYYNPSLKSRVTISVDTSKNQFSLRLSSVTAADTAVY |
| | | YCARHWQEWPDAFDIWGQGTMVTVSSGGGGSGGGGSGGGGSETTLTQSPAFMSATPGD |
| | | KVIISCKASQDIDDAMNWYQQKPGEAPLFIIQSATSPVPGIPPRFSGSGFGTDFSLTI |
| | | NNIESEDAAYYFCLQHDNFPLTFGQGTKLEIKTTTPAPRPPTPAPTIASQPLSLRPEA |
| | | CRPAAGGAVHTRGLDFACDIYIWAPLAGTCGVLLLSLVITLYCKRGRKKLLYIFKQPF |
| | | MRPVQTTQEEDGCSCRFPEEEEGGCELRVKFSRSADAPAYKQGQNQLYNELNLGRREE |
| | | YDVLDKRRGRDPEMGGKPRRKNPQEGLYNELQKDKMAEAYSEIGMKGERRRGKGHDGL |
| | | YQGLSTATKDTYDALHMQALPPR |
| 149362-nt | 234 | ATGGCCCTCCCTGTCACCGCCCTGCTGCTTCCGCTGGCTCTTCTGCTCCACGCCGCTC |
| Full CAR | | GGCCCCAAGTGCAGCTTCAGGAAAGCGGACCGGGCCTGGTCAAGCCATCCGAAACTCT |
| | | CTCCCTGACTTGCACTGTGTCTGGCGGTTCCATCTCATCGTCGTACTACTACTGGGGC |
| | | TGGATTAGGCAGCCGCCCGGAAAGGGACTGGAGTGGATCGGAAGCATCTACTATTCCG |
| | | GCTCGGCGTACTACAACCCTAGCCTCAAGTCGAGAGTGACCATCTCCGTGGATACCTC |
| | | CAAGAACCAGTTTTCCCTGCGCCTGAGCTCCGTGACCGCCGCTGACACCGCCGTGTAC |
| | | TACTGTGCTCGGCATTGGCAGGAATGGCCCGATGCCTTCGACATTTGGGGCCAGGGCA |
| | | CTATGGTCACTGTGTCATCCGGGGGTGGAGGCAGCGGGGGAGGAGGGTCCGGGGGGGG |
| | | AGGTTCAGAGACAACCTTGACCCAGTCACCCGCATTCATGTCCGCCACTCCGGGAGAC |
| | | AAGGTCATCATCTCGTGCAAAGCGTCCCAGGATATCGACGATGCCATGAATTGGTACC |
| | | AGCAGAAGCCTGGCGAAGCGCCGCTGTTCATTATCCAATCCGCAACCTCGCCCGTGCC |
| | | TGGAATCCCACCGCGGTTCAGCGGCAGCGGTTTCGGAACCGACTTTTCCCTGACCATT |
| | | AACAACATTGAGTCCGAGGACGCCGCCTACTACTTCTGCCTGCAACACGACAACTTCC |
| | | CTCTCACGTTCGGCCAGGGAACCAAGCTGGAAATCAAGACCACTACCCCAGCACCGAG |
| | | GCCACCCACCCCGGCTCCTACCATCGCCTCCCAGCCTCTGTCCCTGCGTCCGGAGGCA |
| | | TGTAGACCCGCAGCTGGTGGGGCCGTGCATACCCGGGGTCTTGACTTCGCCTGCGATA |
| | | TCTACATTTGGGCCCCTCTGGCTGGTACTTGCGGGGTCCTGCTGCTTTCACTCGTGAT |
| | | CACTCTTTACTGTAAGCGCGGTCGGAAGAAGCTGCTGTACATCTTTAAGCAACCCTTC |
| | | ATGAGGCCTGTGCAGACTACTCAAGAGGAGGACGGCTGTTCATGCCGGTTCCCAGAGG |
| | | AGGAGGAAGGCGGCTGCGAACTGCGCGTGAAATTCAGCCGCAGCGCAGATGCTCCAGC |
| | | CTACAAGCAGGGGCAGAACCAGCTCTACAACGAACTCAATCTTGGTCGGAGAGAGGAG |
| | | TACGACGTGCTGGACAAGCGGAGAGGACGGGACCCAGAAATGGGCGGGAAGCCGCGCA |
| | | GAAAGAATCCCCAAGAGGGCCTGTACAACGAGCTCCAAAAGGATAAGATGGCAGAAGC |
| | | CTATAGCGAGATTGGTATGAAAGGGGAACGCAGAAGAGGCAAAGGCCACGACGGACTG |
| | | TACCAGGGACTCAGCACCGCCACCAAGGACACCTATGACGCTCTTCACATGCAGGCCC |
| | | TGCCGCCTCGG |
| 149363 | | |
| 149363-aa | 130 | VNLRESGPALVKPTQTLTLTCTFSGFSLRTSGMCVSWIRQPPGKALEWLARIDWDEDK |
| ScFv domain | | FYSTSLKTRLTISKDTSDNQVVLRMTNMDPADTATYYCARSGAGGTSATAFDIWGPGT |
| | | MVTVSSGGGGSGGGGSGGGGSDIQMTQSPSSLSASVGDRVTITCRASQDIYNNLAWFQ |
| | | LKPGSAPRSLMYAANKSQSGVPSRFSGSASGTDFTLTISSLQPEDFATYYCQHYYRFP |
| | | YSFGQGTKLEIK |
| 149363-nt ScFv | 151 | CAAGTCAATCTGCGCGAATCCGGCCCCGCCTTGGTCAAGCCTACCCAGACCCTCACTC |
| domain | | TGACCTGTACTTTCTCCGGCTTCTCCCTGCGGACTTCCGGGATGTGCGTGTCCTGGAT |
| | | CAGACAGCCTCCGGGAAAGGCCCTGGAGTGGCTCGCTCGCATTGACTGGGATGAGGAC |
| | | AAGTTCTACTCCACCTCACTCAAGACCAGGCTGACCATCAGCAAAGATACCTCTGACA |
| | | ACCAAGTGGTGCTCCGCATGACCAACATGGACCCAGCCGACACTGCCACTTACTACTG |
| | | CGCGAGGAGCGGAGCGGGCGGAACCTCCGCCACCGCCTTCGATATTTGGGGCCCGGGT |
| | | ACCATGGTCACCGTGTCAAGCGGAGGAGGGGGGTCCGGGGGCGGCGGTTCCGGGGGAG |
| | | GCGGATCGGACATTCAGATGACTCAGTCACCATCGTCCCTGAGCGCTAGCGTGGGCGA |
| | | CAGAGTGACAATCACTTGCCGGGCATCCCAGGACATCTATAACAACCTTGCGTGGTTC |
| | | CAGCTGAAGCCTGGTTCCGCACCGCGGTCACTTATGTACGCCGCCAACAAGAGCCAGT |
| | | CGGGAGTGCCGTCCCGGTTTTCCGGTTCGGCCTCGGGAACTGACTTCACCCTGACGAT |
| | | CTCCAGCCTGCAACCCGAGGATTTCGCCACCTACTACTGCCAGCACTACTACCGCTTT |
| | | CCCTACTCGTTCGGACAGGGAACCAAGCTGGAAATCAAG |
| 149363-aa VH | 172 | QVNLRESGPALVKPTQTLTLTCTFSGFSLRTSGMCVSWIRQPPGKALEWLARIDWDED |
| | | KFYSTSLKTRLTISKDTSDNQVVLRMTNMDPADTATYYCARSGAGGTSATAFDIWGPG |
| | | TMVTVSS |
| 149363-aa VL | 193 | DIQMTQSPSSLSASVGDRVTITCRASQDIYNNLAWFQLKPGSAPRSLMYAANKSQSGV |
| | | PSRFSGSASGTDFTLTISSLQPEDFATYYCQHYYRFPYSFGQGTKLEIK |
| 149363-aa Full | 214 | MALPVTALLLPLALLLHAARPQVNLRESGPALVKPTQTLTLTCTFSGFSLRTSGMCVS |
| CAR | | WIRQPPGKALEWLARIDWDEDKFYSTSLKTRLTISKDTSDNQVVLRMTNMDPADTATY |
| | | YCARSGAGGTSATAFDIWGPGTMVTVSSGGGGSGGGGSGGGGSDIQMTQSPSSLSASV |
| | | GDRVTITCRASQDIYNNLAWFQLKPGSAPRSLMYAANKSQSGVPSRFSGSASGTDFTL |
| | | TISSLQPEDFATYYCQHYYRFPYSFGQGTKLEIKTTTPAPRPPTPAPTIASQPLSLRP |
| | | EACRPAAGGAVHTRGLDFACDIYIWAPLAGTCGVLLLSLVITLYCKRGRKKLLYIFKQ |
| | | PFMRPVQTTQEEDGCSCRFPEEEEGGCELRVKFSRSADAPAYKQGQNQLYNELNLGRR |
| | | EEYDVLDKRRGRDPEMGGKPRRKNPQEGLYNELQKDKMAEAYSEIGMKGERRRGKGHD |
| | | GLYQGLSTATKDTYDALHMQALPPR |
| 149363-nt | 235 | ATGGCCCTCCCTGTCACCGCCCTGCTGCTTCCGCTGGCTCTTCTGCTCCACGCCGCTC |
| Full CAR | | GGCCCCAAGTCAATCTGCGCGAATCCGGCCCCGCCTTGGTCAAGCCTACCCAGACCCT |
| | | CACTCTGACCTGTACTTTCTCCGGCTTCTCCCTGCGGACTTCCGGGATGTGCGTGTCC |
| | | TGGATCAGACAGCCTCCGGGAAAGGCCCTGGAGTGGCTCGCTCGCATTGACTGGGATG |
| | | AGGACAAGTTCTACTCCACCTCACTCAAGACCAGGCTGACCATCAGCAAAGATACCTC |
| | | TGACAACCAAGTGGTGCTCCGCATGACCAACATGGACCCAGCCGACACTGCCACTTAC |
| | | TACTGCGCGAGGAGCGGAGCGGGCGGAACCTCCGCCACCGCCTTCGATATTTGGGGCC |
| | | CGGGTACCATGGTCACCGTGTCAAGCGGAGGAGGGGGGTCCGGGGGCGGCGGTTCCGG |
| | | GGGAGGCGGATCGGACATTCAGATGACTCAGTCACCATCGTCCCTGAGCGCTAGCGTG |
| | | GGCGACAGAGTGACAATCACTTGCCGGGCATCCCAGGACATCTATAACAACCTTGCGT |
| | | GGTTCCAGCTGAAGCCTGGTTCCGCACCGCGGTCACTTATGTACGCCGCCAACAAGAG |
| | | CCAGTCGGGAGTGCCGTCCCGGTTTTCCGGTTCGGCCTCGGGAACTGACTTCACCCTG |
| | | ACGATCTCCAGCCTGCAACCCGAGGATTTCGCCACCTACTACTGCCAGCACTACTACC |
| | | GCTTTCCCTACTCGTTCGGACAGGGAACCAAGCTGGAAATCAAGACCACTACCCCAGC |
| | | ACCGAGGCCACCCACCCCGGCTCCTACCATCGCCTCCCAGCCTCTGTCCCTGCGTCCG |
| | | GAGGCATGTAGACCCGCAGCTGGTGGGGCCGTGCATACCCGGGGTCTTGACTTCGCCT |
| | | GCGATATCTACATTTGGGCCCCTCTGGCTGGTACTTGCGGGGTCCTGCTGCTTTCACT |
| | | CGTGATCACTCTTTACTGTAAGCGCGGTCGGAAGAAGCTGCTGTACATCTTTAAGCAA |
| | | CCCTTCATGAGGCCTGTGCAGACTACTCAAGAGGAGGACGGCTGTTCATGCCGGTTCC |
| | | CAGAGGAGGAGGAAGGCGGCTGCGAACTGCGCGTGAAATTCAGCCGCAGCGCAGATGC |
| | | TCCAGCCTACAAGCAGGGGCAGAACCAGCTCTACAACGAACTCAATCTTGGTCGGAGA |
| | | GAGGAGTACGACGTGCTGGACAAGCGGAGAGGACGGGACCCAGAAATGGGCGGGAAGC |
| | | CGCGCAGAAAGAATCCCCAAGAGGGCCTGTACAACGAGCTCCAAAAGGATAAGATGGC |
| | | AGAAGCCTATAGCGAGATTGGTATGAAAGGGGAACGCAGAAGAGGCAAAGGCCACGAC |
| | | GGACTGTACCAGGGACTCAGCACCGCCACCAAGGACACCTATGACGCTCTTCACATGC |
| | | AGGCCCTGCCGCCTCGG |
| 149364 | | |
| 149364-aa | 131 | EVQLVESGGGLVKPGGSLRLSCAASGFTFSSYSMNWVRQAPGKGLEWVSSISSSSSYI |
| ScFv domain | | YYADSVKGRFTISRDNAKNSLYLQMNSLRAEDTAVYYCAKTIAAVYAFDIWGQGTTVT |
| | | VSSGGGGSGGGGSGGGGSEIVLTQSPLSLPVTPEEPASISCRSSQSLLHSNGYNYLDW |
| | | YLQKPGQSPQLLIYLGSNRASGVPDRFSGSGSGTDFTLKISRVEAEDVGVYYCMQALQ |
| | | TPYTFGQGTKLEIK |
| 149364-nt ScFv | 152 | GAAGTGCAGCTTGTCGAATCCGGGGGGGGACTGGTCAAGCCGGGCGGATCACTGAGAC |
| domain | | TGTCCTGCGCCGCGAGCGGCTTCACGTTCTCCTCCTACTCCATGAACTGGGTCCGCCA |
| | | AGCCCCCGGGAAGGGACTGGAATGGGTGTCCTCTATCTCCTCGTCGTCGTCCTACATC |
| | | TACTACGCCGACTCCGTGAAGGGAAGATTCACCATTTCCCGCGACAACGCAAAGAACT |
| | | CACTGTACTTGCAAATGAACTCACTCCGGGCCGAAGATACTGCTGTGTACTATTGCGC |
| | | CAAGACTATTGCCGCCGTCTACGCTTTCGACATCTGGGGCCAGGGAACCACCGTGACT |
| | | GTGTCGTCCGGTGGTGGTGGCTCGGGCGGAGGAGGAAGCGGCGGCGGGGGGTCCGAGA |
| | | TTGTGCTGACCCAGTCGCCACTGAGCCTCCCTGTGACCCCCGAGGAACCCGCCAGCAT |
| | | CAGCTGCCGGTCCAGCCAGTCCCTGCTCCACTCCAACGGATACAATTACCTCGATTGG |
| | | TACCTTCAGAAGCCTGGACAAAGCCCGCAGCTGCTCATCTACTTGGGATCAAACCGCG |
| | | CGTCAGGAGTGCCTGACCGGTTCTCCGGCTCGGGCAGCGGTACCGATTTCACCCTGAA |
| | | AATCTCCAGGGTGGAGGCAGAGGACGTGGGAGTGTATTACTGTATGCAGGCGCTGCAG |
| | | ACTCCGTACACATTTGGGCAGGGCACCAAGCTGGAGATCAAG |
| 149364-aa VH | 173 | EVQLVESGGGLVKPGGSLRLSCAASGFTFSSYSMNWVRQAPGKGLEWVSSISSSSSYI |
| | | YYADSVKGRFTISRDNAKNSLYLQMNSLRAEDTAVYYCAKTIAAVYAFDIWGQGTTVT |
| | | VSS |
| 149364-aa VL | 194 | EIVLTQSPLSLPVTPEEPASISCRSSQSLLHSNGYNYLDWYLQKPGQSPQLLIYLGSN |
| | | RASGVPDRFSGSGSGTDFTLKISRVEAEDVGVYYCMQALQTPYTFGQGTKLEIK |
| 149364-aa Full | 215 | MALPVTALLLPLALLLHAARPEVQLVESGGGLVKPGGSLRLSCAASGFTFSSYSMNWV |
| CAR | | RQAPGKGLEWVSSISSSSSYIYYADSVKGRFTISRDNAKNSLYLQMNSLRAEDTAVYY |
| | | CAKTIAAVYAFDIWGQGTTVTVSSGGGGSGGGGSGGGGSEIVLTQSPLSLPVTPEEPA |
| | | SISCRSSQSLLHSNGYNYLDWYLQKPGQSPQLLIYLGSNRASGVPDRFSGSGSGTDFT |
| | | LKISRVEAEDVGVYYCMQALQTPYTFGQGTKLEIKTTTPAPRPPTPAPTIASQPLSLR |
| | | PEACRPAAGGAVHTRGLDFACDIYIWAPLAGTCGVLLLSLVITLYCKRGRKKLLYIFK |
| | | QPFMRPVQTTQEEDGCSCRFPEEEEGGCELRVKFSRSADAPAYKQGQNQLYNELNLGR |
| | | REEYDVLDKRRGRDPEMGGKPRRKNPQEGLYNELQKDKMAEAYSEIGMKGERRRGKGH |
| | | DGLYQGLSTATKDTYDALHMQALPPR |
| 149364-nt | 236 | ATGGCCCTCCCTGTCACCGCCCTGCTGCTTCCGCTGGCTCTTCTGCTCCACGCCGCTC |
| Full CAR | | GGCCCGAAGTGCAGCTTGTCGAATCCGGGGGGGGACTGGTCAAGCCGGGCGGATCACT |
| | | GAGACTGTCCTGCGCCGCGAGCGGCTTCACGTTCTCCTCCTACTCCATGAACTGGGTC |
| | | CGCCAAGCCCCCGGGAAGGGACTGGAATGGGTGTCCTCTATCTCCTCGTCGTCGTCCT |
| | | ACATCTACTACGCCGACTCCGTGAAGGGAAGATTCACCATTTCCCGCGACAACGCAAA |
| | | GAACTCACTGTACTTGCAAATGAACTCACTCCGGGCCGAAGATACTGCTGTGTACTAT |
| | | TGCGCCAAGACTATTGCCGCCGTCTACGCTTTCGACATCTGGGGCCAGGGAACCACCG |
| | | TGACTGTGTCGTCCGGTGGTGGTGGCTCGGGCGGAGGAGGAAGCGGCGGCGGGGGGTC |
| | | CGAGATTGTGCTGACCCAGTCGCCACTGAGCCTCCCTGTGACCCCCGAGGAACCCGCC |
| | | AGCATCAGCTGCCGGTCCAGCCAGTCCCTGCTCCACTCCAACGGATACAATTACCTCG |
| | | ATTGGTACCTTCAGAAGCCTGGACAAAGCCCGCAGCTGCTCATCTACTTGGGATCAAA |
| | | CCGCGCGTCAGGAGTGCCTGACCGGTTCTCCGGCTCGGGCAGCGGTACCGATTTCACC |
| | | CTGAAAATCTCCAGGGTGGAGGCAGAGGACGTGGGAGTGTATTACTGTATGCAGGCGC |
| | | TGCAGACTCCGTACACATTTGGGCAGGGCACCAAGCTGGAGATCAAGACCACTACCCC |
| | | AGCACCGAGGCCACCCACCCCGGCTCCTACCATCGCCTCCCAGCCTCTGTCCCTGCGT |
| | | CCGGAGGCATGTAGACCCGCAGCTGGTGGGGCCGTGCATACCCGGGGTCTTGACTTCG |
| | | CCTGCGATATCTACATTTGGGCCCCTCTGGCTGGTACTTGCGGGGTCCTGCTGCTTTC |
| | | ACTCGTGATCACTCTTTACTGTAAGCGCGGTCGGAAGAAGCTGCTGTACATCTTTAAG |
| | | CAACCCTTCATGAGGCCTGTGCAGACTACTCAAGAGGAGGACGGCTGTTCATGCCGGT |
| | | TCCCAGAGGAGGAGGAAGGCGGCTGCGAACTGCGCGTGAAATTCAGCCGCAGCGCAGA |
| | | TGCTCCAGCCTACAAGCAGGGGCAGAACCAGCTCTACAACGAACTCAATCTTGGTCGG |
| | | AGAGAGGAGTACGACGTGCTGGACAAGCGGAGAGGACGGGACCCAGAAATGGGCGGGA |
| | | AGCCGCGCAGAAAGAATCCCCAAGAGGGCCTGTACAACGAGCTCCAAAAGGATAAGAT |
| | | GGCAGAAGCCTATAGCGAGATTGGTATGAAAGGGGAACGCAGAAGAGGCAAAGGCCAC |
| | | GACGGACTGTACCAGGGACTCAGCACCGCCACCAAGGACACCTATGACGCTCTTCACA |
| | | TGCAGGCCCTGCCGCCTCGG |
| 149365 | | |
| 149365-aa | 132 | EVQLVESGGGLVKPGGSLRLSCAASGFTFSDYYMSWIRQAPGKGLEWVSYISSSGSTI |
| ScFv domain | | YYADSVKGRFTISRDNAKNSLYLQMNSLRAEDTAVYYCARDLRGAFDIWGQGTMVTVS |
| | | SGGGGSGGGGSGGGGSSYVLTQSPSVSAAPGYTATISCGGNNIGTKSVHWYQQKPGQA |
| | | PLLVIRDDSVRPSKIPGRFSGSNSGNMATLTISGVQAGDEADFYCQVWDSDSEHVVFG |
| | | GGTKLTVL |
| 149365-nt ScFv | 153 | GAAGTCCAGCTCGTGGAGTCCGGCGGAGGCCTTGTGAAGCCTGGAGGTTCGCTGAGAC |
| domain | | TGTCCTGCGCCGCCTCCGGCTTCACCTTCTCCGACTACTACATGTCCTGGATCAGACA |
| | | GGCCCCGGGAAAGGGCCTGGAATGGGTGTCCTACATCTCGTCATCGGGCAGCACTATC |
| | | TACTACGCGGACTCAGTGAAGGGGCGGTTCACCATTTCCCGGGATAACGCGAAGAACT |
| | | CGCTGTATCTGCAAATGAACTCACTGAGGGCCGAGGACACCGCCGTGTACTACTGCGC |
| | | CCGCGATCTCCGCGGGGCATTTGACATCTGGGGACAGGGAACCATGGTCACAGTGTCC |
| | | AGCGGAGGGGGAGGATCGGGTGGCGGAGGTTCCGGGGGTGGAGGCTCCTCCTACGTGC |
| | | TGACTCAGAGCCCAAGCGTCAGCGCTGCGCCCGGTTACACGGCAACCATCTCCTGTGG |
| | | CGGAAACAACATTGGGACCAAGTCTGTGCACTGGTATCAGCAGAAGCCGGGCCAAGCT |
| | | CCCCTGTTGGTGATCCGCGATGACTCCGTGCGGCCTAGCAAAATTCCGGGACGGTTCT |
| | | CCGGCTCCAACAGCGGCAATATGGCCACTCTCACCATCTCGGGAGTGCAGGCCGGAGA |
| | | TGAAGCCGACTTCTACTGCCAAGTCTGGGACTCAGACTCCGAGCATGTGGTGTTCGGG |
| | | GGCGGAACCAAGCTGACTGTGCTC |
| 149365-aa VH | 174 | EVQLVESGGGLVKPGGSLRLSCAASGFTFSDYYMSWIRQAPGKGLEWVSYISSSGSTI |
| | | YYADSVKGRFTISRDNAKNSLYLQMNSLRAEDTAVYYCARDLRGAFDIWGQGTMVTVS |
| | | S |
| 149365-aa VL | 195 | SYVLTQSPSVSAAPGYTATISCGGNNIGTKSVHWYQQKPGQAPLLVIRDDSVRPSKIP |
| | | GRFSGSNSGNMATLTISGVQAGDEADFYCQVWDSDSEHVVFGGGTKLTVL |
| 149365-aa Full | 216 | MALPVTALLLPLALLLHAARPEVQLVESGGGLVKPGGSLRLSCAASGFTFSDYYMSWI |
| CAR | | RQAPGKGLEWVSYISSSGSTIYYADSVKGRFTISRDNAKNSLYLQMNSLRAEDTAVYY |
| | | CARDLRGAFDIWGQGTMVTVSSGGGGSGGGGSGGGGSSYVLTQSPSVSAAPGYTATIS |
| | | CGGNNIGTKSVHWYQQKPGQAPLLVIRDDSVRPSKIPGRFSGSNSGNMATLTISGVQA |
| | | GDEADFYCQVWDSDSEHVVFGGGTKLTVLTTTPAPRPPTPAPTIASQPLSLRPEACRP |
| | | AAGGAVHTRGLDFACDIYIWAPLAGTCGVLLLSLVITLYCKRGRKKLLYIFKQPFMRP |
| | | VQTTQEEDGCSCRFPEEEEGGCELRVKFSRSADAPAYKQGQNQLYNELNLGRREEYDV |
| | | LDKRRGRDPEMGGKPRRKNPQEGLYNELQKDKMAEAYSEIGMKGERRRGKGHDGLYQG |
| | | LSTATKDTYDALHMQALPPR |
| 149365-nt | 237 | ATGGCCCTCCCTGTCACCGCCCTGCTGCTTCCGCTGGCTCTTCTGCTCCACGCCGCTC |
| Full CAR | | GGCCCGAAGTCCAGCTCGTGGAGTCCGGCGGAGGCCTTGTGAAGCCTGGAGGTTCGCT |
| | | GAGACTGTCCTGCGCCGCCTCCGGCTTCACCTTCTCCGACTACTACATGTCCTGGATC |
| | | AGACAGGCCCCGGGAAAGGGCCTGGAATGGGTGTCCTACATCTCGTCATCGGGCAGCA |
| | | CTATCTACTACGCGGACTCAGTGAAGGGGCGGTTCACCATTTCCCGGGATAACGCGAA |
| | | GAACTCGCTGTATCTGCAAATGAACTCACTGAGGGCCGAGGACACCGCCGTGTACTAC |
| | | TGCGCCCGCGATCTCCGCGGGGCATTTGACATCTGGGGACAGGGAACCATGGTCACAG |
| | | TGTCCAGCGGAGGGGGAGGATCGGGTGGCGGAGGTTCCGGGGGTGGAGGCTCCTCCTA |
| | | CGTGCTGACTCAGAGCCCAAGCGTCAGCGCTGCGCCCGGTTACACGGCAACCATCTCC |
| | | TGTGGCGGAAACAACATTGGGACCAAGTCTGTGCACTGGTATCAGCAGAAGCCGGGCC |
| | | AAGCTCCCCTGTTGGTGATCCGCGATGACTCCGTGCGGCCTAGCAAAATTCCGGGACG |
| | | GTTCTCCGGCTCCAACAGCGGCAATATGGCCACTCTCACCATCTCGGGAGTGCAGGCC |
| | | GGAGATGAAGCCGACTTCTACTGCCAAGTCTGGGACTCAGACTCCGAGCATGTGGTGT |
| | | TCGGGGGCGGAACCAAGCTGACTGTGCTCACCACTACCCCAGCACCGAGGCCACCCAC |
| | | CCCGGCTCCTACCATCGCCTCCCAGCCTCTGTCCCTGCGTCCGGAGGCATGTAGACCC |
| | | GCAGCTGGTGGGGCCGTGCATACCCGGGGTCTTGACTTCGCCTGCGATATCTACATTT |
| | | GGGCCCCTCTGGCTGGTACTTGCGGGGTCCTGCTGCTTTCACTCGTGATCACTCTTTA |
| | | CTGTAAGCGCGGTCGGAAGAAGCTGCTGTACATCTTTAAGCAACCCTTCATGAGGCCT |
| | | GTGCAGACTACTCAAGAGGAGGACGGCTGTTCATGCCGGTTCCCAGAGGAGGAGGAAG |
| | | GCGGCTGCGAACTGCGCGTGAAATTCAGCCGCAGCGCAGATGCTCCAGCCTACAAGCA |
| | | GGGGCAGAACCAGCTCTACAACGAACTCAATCTTGGTCGGAGAGAGGAGTACGACGTG |
| | | CTGGACAAGCGGAGAGGACGGGACCCAGAAATGGGCGGGAAGCCGCGCAGAAAGAATC |
| | | CCCAAGAGGGCCTGTACAACGAGCTCCAAAAGGATAAGATGGCAGAAGCCTATAGCGA |
| | | GATTGGTATGAAAGGGGAACGCAGAAGAGGCAAAGGCCACGACGGACTGTACCAGGGA |
| | | CTCAGCACCGCCACCAAGGACACCTATGACGCTCTTCACATGCAGGCCCTGCCGCCTC |
| | | GG |
| 149366 | | |
| 149366-aa | 133 | QVQLVQSGAEVKKPGASVKVSCKPSGYTVTSHYIHWVRRAPGQGLEWMGMINPSGGVT |
| ScFv domain | | AYSQTLQGRVTMTSDTSSSTVYMELSSLRSEDTAMYYCAREGSGSGWYFDFWGRGTLV |
| | | TVSSGGGGSGGGGSGGGGSSYVLTQPPSVSVSPGQTASITCSGDGLSKKYVSWYQQKA |
| | | GQSPVVLISRDKERPSGIPDRFSGSNSADTATLTISGTQAMDEADYYCQAWDDTTVVF |
| | | GGGTKLTVL |
| 149366-nt ScFv | 154 | CAAGTGCAGCTGGTGCAGAGCGGGGCCGAAGTCAAGAAGCCGGGAGCCTCCGTGAAAG |
| domain | | TGTCCTGCAAGCCTTCGGGATACACCGTGACCTCCCACTACATTCATTGGGTCCGCCG |
| | | CGCCCCCGGCCAAGGACTCGAGTGGATGGGCATGATCAACCCTAGCGGCGGAGTGACC |
| | | GCGTACAGCCAGACGCTGCAGGGACGCGTGACTATGACCTCGGATACCTCCTCCTCCA |
| | | CCGTCTATATGGAACTGTCCAGCCTGCGGTCCGAGGATACCGCCATGTACTACTGCGC |
| | | CCGGGAAGGATCAGGCTCCGGGTGGTATTTCGACTTCTGGGGAAGAGGCACCCTCGTG |
| | | ACTGTGTCATCTGGGGGAGGGGGTTCCGGTGGTGGCGGATCGGGAGGAGGCGGTTCAT |
| | | CCTACGTGCTGACCCAGCCACCCTCCGTGTCCGTGAGCCCCGGCCAGACTGCATCGAT |
| | | TACATGTAGCGGCGACGGCCTCTCCAAGAAATACGTGTCGTGGTACCAGCAGAAGGCC |
| | | GGACAGAGCCCGGTGGTGCTGATCTCAAGAGATAAGGAGCGGCCTAGCGGAATCCCGG |
| | | ACAGGTTCTCGGGTTCCAACTCCGCGGACACTGCTACTCTGACCATCTCGGGGACCCA |
| | | GGCTATGGACGAAGCCGATTACTACTGCCAAGCCTGGGACGACACTACTGTCGTGTTT |
| | | GGAGGGGGCACCAAGTTGACCGTCCTT |
| 149366-aa VH | 175 | QVQLVQSGAEVKKPGASVKVSCKPSGYTVTSHYIHWVRRAPGQGLEWMGMINPSGGVT |
| | | AYSQTLQGRVTMTSDTSSSTVYMELSSLRSEDTAMYYCAREGSGSGWYFDFWGRGTLV |
| | | TVSS |
| 149366-aa VL | 196 | SYVLTQPPSVSVSPGQTASITCSGDGLSKKYVSWYQQKAGQSPVVLISRDKERPSGIP |
| | | DRFSGSNSADTATLTISGTQAMDEADYYCQAWDDTTVVFGGGTKLTVL |
| 149366-aa Full | 217 | MALPVTALLLPLALLLHAARPQVQLVQSGAEVKKPGASVKVSCKPSGYTVTSHYIHWV |
| CAR | | RRAPGQGLEWMGMINPSGGVTAYSQTLQGRVTMTSDTSSSTVYMELSSLRSEDTAMYY |
| | | CAREGSGSGWYFDFWGRGTLVTVSSGGGGSGGGGSGGGGSSYVLTQPPSVSVSPGQTA |
| | | SITCSGDGLSKKYVSWYQQKAGQSPVVLISRDKERPSGIPDRFSGSNSADTATLTISG |
| | | TQAMDEADYYCQAWDDTTVVFGGGTKLTVLTTTPAPRPPTPAPTIASQPLSLRPEACR |
| | | PAAGGAVHTRGLDFACDIYIWAPLAGTCGVLLLSLVITLYCKRGRKKLLYIFKQPFMR |
| | | PVQTTQEEDGCSCRFPEEEEGGCELRVKFSRSADAPAYKQGQNQLYNELNLGRREEYD |
| | | VLDKRRGRDPEMGGKPRRKNPQEGLYNELQKDKMAEAYSEIGMKGERRRGKGHDGLYQ |
| | | GLSTATKDTYDALHMQALPPR |
| 149366-nt | 238 | ATGGCCCTCCCTGTCACCGCCCTGCTGCTTCCGCTGGCTCTTCTGCTCCACGCCGCTC |
| Full CAR | | GGCCCCAAGTGCAGCTGGTGCAGAGCGGGGCCGAAGTCAAGAAGCCGGGAGCCTCCGT |
| | | GAAAGTGTCCTGCAAGCCTTCGGGATACACCGTGACCTCCCACTACATTCATTGGGTC |
| | | CGCCGCGCCCCCGGCCAAGGACTCGAGTGGATGGGCATGATCAACCCTAGCGGCGGAG |
| | | TGACCGCGTACAGCCAGACGCTGCAGGGACGCGTGACTATGACCTCGGATACCTCCTC |
| | | CTCCACCGTCTATATGGAACTGTCCAGCCTGCGGTCCGAGGATACCGCCATGTACTAC |
| | | TGCGCCCGGGAAGGATCAGGCTCCGGGTGGTATTTCGACTTCTGGGGAAGAGGCACCC |
| | | TCGTGACTGTGTCATCTGGGGGAGGGGGTTCCGGTGGTGGCGGATCGGGAGGAGGCGG |
| | | TTCATCCTACGTGCTGACCCAGCCACCCTCCGTGTCCGTGAGCCCCGGCCAGACTGCA |
| | | TCGATTACATGTAGCGGCGACGGCCTCTCCAAGAAATACGTGTCGTGGTACCAGCAGA |
| | | AGGCCGGACAGAGCCCGGTGGTGCTGATCTCAAGAGATAAGGAGCGGCCTAGCGGAAT |
| | | CCCGGACAGGTTCTCGGGTTCCAACTCCGCGGACACTGCTACTCTGACCATCTCGGGG |
| | | ACCCAGGCTATGGACGAAGCCGATTACTACTGCCAAGCCTGGGACGACACTACTGTCG |
| | | TGTTTGGAGGGGGCACCAAGTTGACCGTCCTTACCACTACCCCAGCACCGAGGCCACC |
| | | CACCCCGGCTCCTACCATCGCCTCCCAGCCTCTGTCCCTGCGTCCGGAGGCATGTAGA |
| | | CCCGCAGCTGGTGGGGCCGTGCATACCCGGGGTCTTGACTTCGCCTGCGATATCTACA |
| | | TTTGGGCCCCTCTGGCTGGTACTTGCGGGGTCCTGCTGCTTTCACTCGTGATCACTCT |
| | | TTACTGTAAGCGCGGTCGGAAGAAGCTGCTGTACATCTTTAAGCAACCCTTCATGAGG |
| | | CCTGTGCAGACTACTCAAGAGGAGGACGGCTGTTCATGCCGGTTCCCAGAGGAGGAGG |
| | | AAGGCGGCTGCGAACTGCGCGTGAAATTCAGCCGCAGCGCAGATGCTCCAGCCTACAA |
| | | GCAGGGGCAGAACCAGCTCTACAACGAACTCAATCTTGGTCGGAGAGAGGAGTACGAC |
| | | GTGCTGGACAAGCGGAGAGGACGGGACCCAGAAATGGGCGGGAAGCCGCGCAGAAAGA |
| | | ATCCCCAAGAGGGCCTGTACAACGAGCTCCAAAAGGATAAGATGGCAGAAGCCTATAG |
| | | CGAGATTGGTATGAAAGGGGAACGCAGAAGAGGCAAAGGCCACGACGGACTGTACCAG |
| | | GGACTCAGCACCGCCACCAAGGACACCTATGACGCTCTTCACATGCAGGCCCTGCCGC |
| | | CTCGG |
| 149367 | | |
| 149367-aa | 134 | QVQLQESGPGLVKPSQTLSLTCTVSGGSISSGGYYWSWIRQHPGKGLEWIGYIYYSGS |
| ScFv domain | | TYYNPSLKSRVTISVDTSKNQFSLKLSSVTAADTAVYYCARAGIAARLRGAFDIWGQG |
| | | TMVTVSSGGGGSGGGGSGGGGSDIVMTQSPSSVSASVGDRVIITCRASQGIRNWLAWY |
| | | QQKPGKAPNLLIYAASNLQSGVPSRFSGSGSGADFTLTISSLQPEDVATYYCQKYNSA |
| | | PFTFGPGTKVDIK |
| 149367-nt ScFv | 155 | CAAGTGCAGCTTCAGGAGAGCGGCCCGGGACTCGTGAAGCCGTCCCAGACCCTGTCCC |
| domain | | TGACTTGCACCGTGTCGGGAGGAAGCATCTCGAGCGGAGGCTACTATTGGTCGTGGAT |
| | | TCGGCAGCACCCTGGAAAGGGCCTGGAATGGATCGGCTACATCTACTACTCCGGCTCG |
| | | ACCTACTACAACCCATCGCTGAAGTCCAGAGTGACAATCTCAGTGGACACGTCCAAGA |
| | | ATCAGTTCAGCCTGAAGCTCTCTTCCGTGACTGCGGCCGACACCGCCGTGTACTACTG |
| | | CGCACGCGCTGGAATTGCCGCCCGGCTGAGGGGTGCCTTCGACATTTGGGGACAGGGC |
| | | ACCATGGTCACCGTGTCCTCCGGCGGCGGAGGTTCCGGGGGTGGAGGCTCAGGAGGAG |
| | | GGGGGTCCGACATCGTCATGACTCAGTCGCCCTCAAGCGTCAGCGCGTCCGTCGGGGA |
| | | CAGAGTGATCATCACCTGTCGGGCGTCCCAGGGAATTCGCAACTGGCTGGCCTGGTAT |
| | | CAGCAGAAGCCCGGAAAGGCCCCCAACCTGTTGATCTACGCCGCCTCAAACCTCCAAT |
| | | CCGGGGTGCCGAGCCGCTTCAGCGGCTCCGGTTCGGGTGCCGATTTCACTCTGACCAT |
| | | CTCCTCCCTGCAACCTGAAGATGTGGCTACCTACTACTGCCAAAAGTACAACTCCGCA |
| | | CCTTTTACTTTCGGACCGGGGACCAAAGTGGACATTAAG |
| 149367-aa VH | 176 | QVQLQESGPGLVKPSQTLSLTCTVSGGSISSGGYYWSWIRQHPGKGLEWIGYIYYSGS |
| | | TYYNPSLKSRVTISVDTSKNQFSLKLSSVTAADTAVYYCARAGIAARLRGAFDIWGQG |
| | | TMVTVSS |
| 149367-aa VL | 197 | DIVMTQSPSSVSASVGDRVIITCRASQGIRNWLAWYQQKPGKAPNLLIYAASNLQSGV |
| | | PSRFSGSGSGADFTLTISSLQPEDVATYYCQKYNSAPFTFGPGTKVDIK |
| 149367-aa Full | 218 | MALPVTALLLPLALLLHAARPQVQLQESGPGLVKPSQTLSLTCTVSGGSISSGGYYWS |
| CAR | | WIRQHPGKGLEWIGYIYYSGSTYYNPSLKSRVTISVDTSKNQFSLKLSSVTAADTAVY |
| | | YCARAGIAARLRGAFDIWGQGTMVTVSSGGGGSGGGGSGGGGSDIVMTQSPSSVSASV |
| | | GDRVIITCRASQGIRNWLAWYQQKPGKAPNLLIYAASNLQSGVPSRFSGSGSGADFTL |
| | | TISSLQPEDVATYYCQKYNSAPFTFGPGTKVDIKTTTPAPRPPTPAPTIASQPLSLRP |
| | | EACRPAAGGAVHTRGLDFACDIYIWAPLAGTCGVLLLSLVITLYCKRGRKKLLYIFKQ |
| | | PFMRPVQTTQEEDGCSCRFPEEEEGGCELRVKFSRSADAPAYKQGQNQLYNELNLGRR |
| | | EEYDVLDKRRGRDPEMGGKPRRKNPQEGLYNELQKDKMAEAYSEIGMKGERRRGKGHD |
| | | GLYQGLSTATKDTYDALHMQALPPR |
| 149367-nt | 239 | ATGGCCCTCCCTGTCACCGCCCTGCTGCTTCCGCTGGCTCTTCTGCTCCACGCCGCTC |
| Full CAR | | GGCCCCAAGTGCAGCTTCAGGAGAGCGGCCCGGGACTCGTGAAGCCGTCCCAGACCCT |
| | | GTCCCTGACTTGCACCGTGTCGGGAGGAAGCATCTCGAGCGGAGGCTACTATTGGTCG |
| | | TGGATTCGGCAGCACCCTGGAAAGGGCCTGGAATGGATCGGCTACATCTACTACTCCG |
| | | GCTCGACCTACTACAACCCATCGCTGAAGTCCAGAGTGACAATCTCAGTGGACACGTC |
| | | CAAGAATCAGTTCAGCCTGAAGCTCTCTTCCGTGACTGCGGCCGACACCGCCGTGTAC |
| | | TACTGCGCACGCGCTGGAATTGCCGCCCGGCTGAGGGGTGCCTTCGACATTTGGGGAC |
| | | AGGGCACCATGGTCACCGTGTCCTCCGGCGGCGGAGGTTCCGGGGGTGGAGGCTCAGG |
| | | AGGAGGGGGGTCCGACATCGTCATGACTCAGTCGCCCTCAAGCGTCAGCGCGTCCGTC |
| | | GGGGACAGAGTGATCATCACCTGTCGGGCGTCCCAGGGAATTCGCAACTGGCTGGCCT |
| | | GGTATCAGCAGAAGCCCGGAAAGGCCCCCAACCTGTTGATCTACGCCGCCTCAAACCT |
| | | CCAATCCGGGGTGCCGAGCCGCTTCAGCGGCTCCGGTTCGGGTGCCGATTTCACTCTG |
| | | ACCATCTCCTCCCTGCAACCTGAAGATGTGGCTACCTACTACTGCCAAAAGTACAACT |
| | | CCGCACCTTTTACTTTCGGACCGGGGACCAAAGTGGACATTAAGACCACTACCCCAGC |
| | | ACCGAGGCCACCCACCCCGGCTCCTACCATCGCCTCCCAGCCTCTGTCCCTGCGTCCG |
| | | GAGGCATGTAGACCCGCAGCTGGTGGGGCCGTGCATACCCGGGGTCTTGACTTCGCCT |
| | | GCGATATCTACATTTGGGCCCCTCTGGCTGGTACTTGCGGGGTCCTGCTGCTTTCACT |
| | | CGTGATCACTCTTTACTGTAAGCGCGGTCGGAAGAAGCTGCTGTACATCTTTAAGCAA |
| | | CCCTTCATGAGGCCTGTGCAGACTACTCAAGAGGAGGACGGCTGTTCATGCCGGTTCC |
| | | CAGAGGAGGAGGAAGGCGGCTGCGAACTGCGCGTGAAATTCAGCCGCAGCGCAGATGC |
| | | TCCAGCCTACAAGCAGGGGCAGAACCAGCTCTACAACGAACTCAATCTTGGTCGGAGA |
| | | GAGGAGTACGACGTGCTGGACAAGCGGAGAGGACGGGACCCAGAAATGGGCGGGAAGC |
| | | CGCGCAGAAAGAATCCCCAAGAGGGCCTGTACAACGAGCTCCAAAAGGATAAGATGGC |
| | | AGAAGCCTATAGCGAGATTGGTATGAAAGGGGAACGCAGAAGAGGCAAAGGCCACGAC |
| | | GGACTGTACCAGGGACTCAGCACCGCCACCAAGGACACCTATGACGCTCTTCACATGC |
| | | AGGCCCTGCCGCCTCGG |
| 149368 | | |
| 149368-aa | 135 | QVQLVQSGAEVKKPGSSVKVSCKASGGTFSSYAISWVRQAPGQGLEWMGGIIPIFGTA |
| ScFv domain | | NYAQKFQGRVTITADESTSTAYMELSSLRSEDTAVYYCARRGGYQLLRWDVGLLRSAF |
| | | DIWGQGTMVTVSSGGGGSGGGGSGGGGSSYVLTQPPSVSVAPGQTARITCGGNNIGSK |
| | | SVHWYQQKPGQAPVLVLYGKNNRPSGVPDRFSGSRSGTTASLTITGAQAEDEADYYCS |
| | | SRDSSGDHLRVFGTGTKVTVL |
| 149368-nt ScFv | 156 | CAAGTGCAGCTGGTCCAGTCGGGCGCCGAGGTCAAGAAGCCCGGGAGCTCTGTGAAAG |
| domain | | TGTCCTGCAAGGCCTCCGGGGGCACCTTTAGCTCCTACGCCATCTCCTGGGTCCGCCA |
| | | AGCACCGGGTCAAGGCCTGGAGTGGATGGGGGGAATTATCCCTATCTTCGGCACTGCC |
| | | AACTACGCCCAGAAGTTCCAGGGACGCGTGACCATTACCGCGGACGAATCCACCTCCA |
| | | CCGCTTATATGGAGCTGTCCAGCTTGCGCTCGGAAGATACCGCCGTGTACTACTGCGC |
| | | CCGGAGGGGTGGATACCAGCTGCTGAGATGGGACGTGGGCCTCCTGCGGTCGGCGTTC |
| | | GACATCTGGGGCCAGGGCACTATGGTCACTGTGTCCAGCGGAGGAGGCGGATCGGGAG |
| | | GCGGCGGATCAGGGGGAGGCGGTTCCAGCTACGTGCTTACTCAACCCCCTTCGGTGTC |
| | | CGTGGCCCCGGGACAGACCGCCAGAATCACTTGCGGAGGAAACAACATTGGGTCCAAG |
| | | AGCGTGCATTGGTACCAGCAGAAGCCAGGACAGGCCCCTGTGCTGGTGCTCTACGGGA |
| | | AGAACAATCGGCCCAGCGGAGTGCCGGACAGGTTCTCGGGTTCACGCTCCGGTACAAC |
| | | CGCTTCACTGACTATCACCGGGGCCCAGGCAGAGGATGAAGCGGACTACTACTGTTCC |
| | | TCCCGGGATTCATCCGGCGACCACCTCCGGGTGTTCGGAACCGGAACGAAGGTCACCG |
| | | TGCTG |
| 149368-aa VH | 177 | QVQLVQSGAEVKKPGSSVKVSCKASGGTFSSYAISWVRQAPGQGLEWMGGIIPIFGTA |
| | | NYAQKFQGRVTITADESTSTAYMELSSLRSEDTAVYYCARRGGYQLLRWDVGLLRSAF |
| | | DIWGQGTMVTVSS |
| 149368-aa VL | 198 | SYVLTQPPSVSVAPGQTARITCGGNNIGSKSVHWYQQKPGQAPVLVLYGKNNRPSGVP |
| | | DRFSGSRSGTTASLTITGAQAEDEADYYCSSRDSSGDHLRVFGTGTKVTVL |
| 149368-aa Full | 219 | MALPVTALLLPLALLLHAARPQVQLVQSGAEVKKPGSSVKVSCKASGGTFSSYAISWV |
| CAR | | RQAPGQGLEWMGGIIPIFGTANYAQKFQGRVTITADESTSTAYMELSSLRSEDTAVYY |
| | | CARRGGYQLLRWDVGLLRSAFDIWGQGTMVTVSSGGGGSGGGGSGGGGSSYVLTQPPS |
| | | VSVAPGQTARITCGGNNIGSKSVHWYQQKPGQAPVLVLYGKNNRPSGVPDRFSGSRSG |
| | | TTASLTITGAQAEDEADYYCSSRDSSGDHLRVFGTGTKVTVLTTTPAPRPPTPAPTIA |
| | | SQPLSLRPEACRPAAGGAVHTRGLDFACDIYIWAPLAGTCGVLLLSLVITLYCKRGRK |
| | | KLLYIFKQPFMRPVQTTQEEDGCSCRFPEEEEGGCELRVKFSRSADAPAYKQGQNQLY |
| | | NELNLGRREEYDVLDKRRGRDPEMGGKPRRKNPQEGLYNELQKDKMAEAYSEIGMKGE |
| | | RRRGKGHDGLYQGLSTATKDTYDALHMQALPPR |
| 149368-nt | 240 | ATGGCCCTCCCTGTCACCGCCCTGCTGCTTCCGCTGGCTCTTCTGCTCCACGCCGCTC |
| Full CAR | | GGCCCCAAGTGCAGCTGGTCCAGTCGGGCGCCGAGGTCAAGAAGCCCGGGAGCTCTGT |
| | | GAAAGTGTCCTGCAAGGCCTCCGGGGGCACCTTTAGCTCCTACGCCATCTCCTGGGTC |
| | | CGCCAAGCACCGGGTCAAGGCCTGGAGTGGATGGGGGGAATTATCCCTATCTTCGGCA |
| | | CTGCCAACTACGCCCAGAAGTTCCAGGGACGCGTGACCATTACCGCGGACGAATCCAC |
| | | CTCCACCGCTTATATGGAGCTGTCCAGCTTGCGCTCGGAAGATACCGCCGTGTACTAC |
| | | TGCGCCCGGAGGGGTGGATACCAGCTGCTGAGATGGGACGTGGGCCTCCTGCGGTCGG |
| | | CGTTCGACATCTGGGGCCAGGGCACTATGGTCACTGTGTCCAGCGGAGGAGGCGGATC |
| | | GGGAGGCGGCGGATCAGGGGGAGGCGGTTCCAGCTACGTGCTTACTCAACCCCCTTCG |
| | | GTGTCCGTGGCCCCGGGACAGACCGCCAGAATCACTTGCGGAGGAAACAACATTGGGT |
| | | CCAAGAGCGTGCATTGGTACCAGCAGAAGCCAGGACAGGCCCCTGTGCTGGTGCTCTA |
| | | CGGGAAGAACAATCGGCCCAGCGGAGTGCCGGACAGGTTCTCGGGTTCACGCTCCGGT |
| | | ACAACCGCTTCACTGACTATCACCGGGGCCCAGGCAGAGGATGAAGCGGACTACTACT |
| | | GTTCCTCCCGGGATTCATCCGGCGACCACCTCCGGGTGTTCGGAACCGGAACGAAGGT |
| | | CACCGTGCTGACCACTACCCCAGCACCGAGGCCACCCACCCCGGCTCCTACCATCGCC |
| | | TCCCAGCCTCTGTCCCTGCGTCCGGAGGCATGTAGACCCGCAGCTGGTGGGGCCGTGC |
| | | ATACCCGGGGTCTTGACTTCGCCTGCGATATCTACATTTGGGCCCCTCTGGCTGGTAC |
| | | TTGCGGGGTCCTGCTGCTTTCACTCGTGATCACTCTTTACTGTAAGCGCGGTCGGAAG |
| | | AAGCTGCTGTACATCTTTAAGCAACCCTTCATGAGGCCTGTGCAGACTACTCAAGAGG |
| | | AGGACGGCTGTTCATGCCGGTTCCCAGAGGAGGAGGAAGGCGGCTGCGAACTGCGCGT |
| | | GAAATTCAGCCGCAGCGCAGATGCTCCAGCCTACAAGCAGGGGCAGAACCAGCTCTAC |
| | | AACGAACTCAATCTTGGTCGGAGAGAGGAGTACGACGTGCTGGACAAGCGGAGAGGAC |
| | | GGGACCCAGAAATGGGCGGGAAGCCGCGCAGAAAGAATCCCCAAGAGGGCCTGTACAA |
| | | CGAGCTCCAAAAGGATAAGATGGCAGAAGCCTATAGCGAGATTGGTATGAAAGGGGAA |
| | | CGCAGAAGAGGCAAAGGCCACGACGGACTGTACCAGGGACTCAGCACCGCCACCAAGG |
| | | ACACCTATGACGCTCTTCACATGCAGGCCCTGCCGCCTCGG |
| 149369 | | |
| 149369-aa | 136 | EVQLQQSGPGLVKPSQTLSLTCAISGDSVSSNSAAWNWIRQSPSRGLEWLGRTYYRSK |
| ScFv domain | | WYSFYAISLKSRIIINPDTSKNQFSLQLKSVTPEDTAVYYCARSSPEGLFLYWFDPWG |
| | | QGTLVTVSSGGDGSGGGGSGGGGSSSELTQDPAVSVALGQTIRITCQGDSLGNYYATW |
| | | YQQKPGQAPVLVIYGTNNRPSGIPDRFSASSSGNTASLTITGAQAEDEADYYCNSRDS |
| | | SGHHLLFGTGTKVTVL |
| 149369-nt ScFv | 157 | GAAGTGCAGCTCCAACAGTCAGGACCGGGGCTCGTGAAGCCATCCCAGACCCTGTCCC |
| domain | | TGACTTGTGCCATCTCGGGAGATAGCGTGTCATCGAACTCCGCCGCCTGGAACTGGAT |
| | | TCGGCAGAGCCCGTCCCGCGGACTGGAGTGGCTTGGAAGGACCTACTACCGGTCCAAG |
| | | TGGTACTCTTTCTACGCGATCTCGCTGAAGTCCCGCATTATCATTAACCCTGATACCT |
| | | CCAAGAATCAGTTCTCCCTCCAACTGAAATCCGTCACCCCCGAGGACACAGCAGTGTA |
| | | TTACTGCGCACGGAGCAGCCCCGAAGGACTGTTCCTGTATTGGTTTGACCCCTGGGGC |
| | | CAGGGGACTCTTGTGACCGTGTCGAGCGGCGGAGATGGGTCCGGTGGCGGTGGTTCGG |
| | | GGGGCGGCGGATCATCATCCGAACTGACCCAGGACCCGGCTGTGTCCGTGGCGCTGGG |
| | | ACAAACCATCCGCATTACGTGCCAGGGAGACTCCCTGGGCAACTACTACGCCACTTGG |
| | | TACCAGCAGAAGCCGGGCCAAGCCCCTGTGTTGGTCATCTACGGGACCAACAACAGAC |
| | | CTTCCGGCATCCCCGACCGGTTCAGCGCTTCGTCCTCCGGCAACACTGCCAGCCTGAC |
| | | CATCACTGGAGCGCAGGCCGAAGATGAGGCCGACTACTACTGCAACAGCAGAGACTCC |
| | | TCGGGTCATCACCTCTTGTTCGGAACTGGAACCAAGGTCACCGTGCTG |
| 149369-aa VH | 178 | EVQLQQSGPGLVKPSQTLSLTCAISGDSVSSNSAAWNWIRQSPSRGLEWLGRTYYRSK |
| | | WYSFYAISLKSRIIINPDTSKNQFSLQLKSVTPEDTAVYYCARSSPEGLFLYWFDPWG |
| | | QGTLVTVSS |
| 149369-aa VL | 199 | SSELTQDPAVSVALGQTIRITCQGDSLGNYYATWYQQKPGQAPVLVIYGTNNRPSGIP |
| | | DRFSASSSGNTASLTITGAQAEDEADYYCNSRDSSGHHLLFGTGTKVTVL |
| 149369-aa Full | 220 | MALPVTALLLPLALLLHAARPEVQLQQSGPGLVKPSQTLSLTCAISGDSVSSNSAAWN |
| CAR | | WIRQSPSRGLEWLGRTYYRSKWYSFYAISLKSRIIINPDTSKNQFSLQLKSVTPEDTA |
| | | VYYCARSSPEGLFLYWFDPWGQGTLVTVSSGGDGSGGGGSGGGGSSSELTQDPAVSVA |
| | | LGQTIRITCQGDSLGNYYATWYQQKPGQAPVLVIYGTNNRPSGIPDRFSASSSGNTAS |
| | | LTITGAQAEDEADYYCNSRDSSGHHLLFGTGTKVTVLTTTPAPRPPTPAPTIASQPLS |
| | | LRPEACRPAAGGAVHTRGLDFACDIYIWAPLAGTCGVLLLSLVITLYCKRGRKKLLYI |
| | | FKQPFMRPVQTTQEEDGCSCRFPEEEEGGCELRVKFSRSADAPAYKQGQNQLYNELNL |
| | | GRREEYDVLDKRRGRDPEMGGKPRRKNPQEGLYNELQKDKMAEAYSEIGMKGERRRGK |
| | | GHDGLYQGLSTATKDTYDALHMQALPPR |
| 149369-nt | 241 | ATGGCCCTCCCTGTCACCGCCCTGCTGCTTCCGCTGGCTCTTCTGCTCCACGCCGCTC |
| Full CAR | | GGCCCGAAGTGCAGCTCCAACAGTCAGGACCGGGGCTCGTGAAGCCATCCCAGACCCT |
| | | GTCCCTGACTTGTGCCATCTCGGGAGATAGCGTGTCATCGAACTCCGCCGCCTGGAAC |
| | | TGGATTCGGCAGAGCCCGTCCCGCGGACTGGAGTGGCTTGGAAGGACCTACTACCGGT |
| | | CCAAGTGGTACTCTTTCTACGCGATCTCGCTGAAGTCCCGCATTATCATTAACCCTGA |
| | | TACCTCCAAGAATCAGTTCTCCCTCCAACTGAAATCCGTCACCCCCGAGGACACAGCA |
| | | GTGTATTACTGCGCACGGAGCAGCCCCGAAGGACTGTTCCTGTATTGGTTTGACCCCT |
| | | GGGGCCAGGGGACTCTTGTGACCGTGTCGAGCGGCGGAGATGGGTCCGGTGGCGGTGG |
| | | TTCGGGGGGCGGCGGATCATCATCCGAACTGACCCAGGACCCGGCTGTGTCCGTGGCG |
| | | CTGGGACAAACCATCCGCATTACGTGCCAGGGAGACTCCCTGGGCAACTACTACGCCA |
| | | CTTGGTACCAGCAGAAGCCGGGCCAAGCCCCTGTGTTGGTCATCTACGGGACCAACAA |
| | | CAGACCTTCCGGCATCCCCGACCGGTTCAGCGCTTCGTCCTCCGGCAACACTGCCAGC |
| | | CTGACCATCACTGGAGCGCAGGCCGAAGATGAGGCCGACTACTACTGCAACAGCAGAG |
| | | ACTCCTCGGGTCATCACCTCTTGTTCGGAACTGGAACCAAGGTCACCGTGCTGACCAC |
| | | TACCCCAGCACCGAGGCCACCCACCCCGGCTCCTACCATCGCCTCCCAGCCTCTGTCC |
| | | CTGCGTCCGGAGGCATGTAGACCCGCAGCTGGTGGGGCCGTGCATACCCGGGGTCTTG |
| | | ACTTCGCCTGCGATATCTACATTTGGGCCCCTCTGGCTGGTACTTGCGGGGTCCTGCT |
| | | GCTTTCACTCGTGATCACTCTTTACTGTAAGCGCGGTCGGAAGAAGCTGCTGTACATC |
| | | TTTAAGCAACCCTTCATGAGGCCTGTGCAGACTACTCAAGAGGAGGACGGCTGTTCAT |
| | | GCCGGTTCCCAGAGGAGGAGGAAGGCGGCTGCGAACTGCGCGTGAAATTCAGCCGCAG |
| | | CGCAGATGCTCCAGCCTACAAGCAGGGGCAGAACCAGCTCTACAACGAACTCAATCTT |
| | | GGTCGGAGAGAGGAGTACGACGTGCTGGACAAGCGGAGAGGACGGGACCCAGAAATGG |
| | | GCGGGAAGCCGCGCAGAAAGAATCCCCAAGAGGGCCTGTACAACGAGCTCCAAAAGGA |
| | | TAAGATGGCAGAAGCCTATAGCGAGATTGGTATGAAAGGGGAACGCAGAAGAGGCAAA |
| | | GGCCACGACGGACTGTACCAGGGACTCAGCACCGCCACCAAGGACACCTATGACGCTC |
| | | TTCACATGCAGGCCCTGCCGCCTCGG |
| BCMA_EBB- | | |
| C1978-A4 | | |
| BCMA_EBB- | 137 | EVQLVESGGGLVQPGGSLRLSCAASGFTFSSYAMSWVRQAPGKGLEWVSAISGSGGST |
| C1978-A4-aa | | YYADSVKGRFTISRDNSKNTLYLQMNSLRAEDTAVYYCAKVEGSGSLDYWGQGTLVTV |
| ScFv domain | | SSGGGGSGGGGSGGGGSEIVMTQSPGTLSLSPGERATLSCRASQSVSSAYLAWYQQKP |
| | | GQPPRLLISGASTRATGIPDRFGGSGSGTDFTLTISRLEPEDFAVYYCQHYGSSFNGS |
| | | SLFTFGQGTRLEIK |
| BCMA_EBB- | 158 | GAAGTGCAGCTCGTGGAGTCAGGAGGCGGCCTGGTCCAGCCGGGAGGGTCCCTTAGAC |
| C1978-A4-nt | | TGTCATGCGCCGCAAGCGGATTCACTTTCTCCTCCTATGCCATGAGCTGGGTCCGCCA |
| ScFv domain | | AGCCCCCGGAAAGGGACTGGAATGGGTGTCCGCCATCTCGGGGTCTGGAGGCTCAACT |
| | | TACTACGCTGACTCCGTGAAGGGACGGTTCACCATTAGCCGCGACAACTCCAAGAACA |
| | | CCCTCTACCTCCAAATGAACTCCCTGCGGGCCGAGGATACCGCCGTCTACTACTGCGC |
| | | CAAAGTGGAAGGTTCAGGATCGCTGGACTACTGGGGACAGGGTACTCTCGTGACCGTG |
| | | TCATCGGGCGGAGGAGGTTCCGGCGGTGGCGGCTCCGGCGGCGGAGGGTCGGAGATCG |
| | | TGATGACCCAGAGCCCTGGTACTCTGAGCCTTTCGCCGGGAGAAAGGGCCACCCTGTC |
| | | CTGCCGCGCTTCCCAATCCGTGTCCTCCGCGTACTTGGCGTGGTACCAGCAGAAGCCG |
| | | GGACAGCCCCCTCGGCTGCTGATCAGCGGGGCCAGCACCCGGGCAACCGGAATCCCAG |
| | | ACAGATTCGGGGGTTCCGGCAGCGGCACAGATTTCACCCTGACTATTTCGAGGTTGGA |
| | | GCCCGAGGACTTTGCGGTGTATTACTGTCAGCACTACGGGTCGTCCTTTAATGGCTCC |
| | | AGCCTGTTCACGTTCGGACAGGGGACCCGCCTGGAAATCAAG |
| BCMA_EBB- | 179 | EVQLVESGGGLVQPGGSLRLSCAASGFTFSSYAMSWVRQAPGKGLEWVSAISGSGGST |
| C1978-A4-aa | | YYADSVKGRFTISRDNSKNTLYLQMNSLRAEDTAVYYCAKVEGSGSLDYWGQGTLVTV |
| VH | | SS |
| BCMA_EBB- | 200 | EIVMTQSPGTLSLSPGERATLSCRASQSVSSAYLAWYQQKPGQPPRLLISGASTRATG |
| C1978-A4-aa | | IPDRFGGSGSGTDFTLTISRLEPEDFAVYYCQHYGSSFNGSSLFTFGQGTRLEIK |
| VL | | |
| BCMA_EBB- | 221 | MALPVTALLLPLALLLHAARPEVQLVESGGGLVQPGGSLRLSCAASGFTFSSYAMSWV |
| C1978-A4-aa | | RQAPGKGLEWVSAISGSGGSTYYADSVKGRFTISRDNSKNTLYLQMNSLRAEDTAVYY |
| Full CART | | CAKVEGSGSLDYWGQGTLVTVSSGGGGSGGGGSGGGGSEIVMTQSPGTLSLSPGERAT |
| | | LSCRASQSVSSAYLAWYQQKPGQPPRLLISGASTRATGIPDRFGGSGSGTDFTLTISR |
| | | LEPEDFAVYYCQHYGSSFNGSSLFTFGQGTRLEIKTTTPAPRPPTPAPTIASQPLSLR |
| | | PEACRPAAGGAVHTRGLDFACDIYIWAPLAGTCGVLLLSLVITLYCKRGRKKLLYIFK |
| | | QPFMRPVQTTQEEDGCSCRFPEEEEGGCELRVKFSRSADAPAYKQGQNQLYNELNLGR |
| | | REEYDVLDKRRGRDPEMGGKPRRKNPQEGLYNELQKDKMAEAYSEIGMKGERRRGKGH |
| | | DGLYQGLSTATKDTYDALHMQALPPR |
| BCMA_EBB- | 242 | ATGGCCCTCCCTGTCACCGCCCTGCTGCTTCCGCTGGCTCTTCTGCTCCACGCCGCTC |
| C1978-A4-nt | | GGCCCGAAGTGCAGCTCGTGGAGTCAGGAGGCGGCCTGGTCCAGCCGGGAGGGTCCCT |
| Full CART | | TAGACTGTCATGCGCCGCAAGCGGATTCACTTTCTCCTCCTATGCCATGAGCTGGGTC |
| | | CGCCAAGCCCCCGGAAAGGGACTGGAATGGGTGTCCGCCATCTCGGGGTCTGGAGGCT |
| | | CAACTTACTACGCTGACTCCGTGAAGGGACGGTTCACCATTAGCCGCGACAACTCCAA |
| | | GAACACCCTCTACCTCCAAATGAACTCCCTGCGGGCCGAGGATACCGCCGTCTACTAC |
| | | TGCGCCAAAGTGGAAGGTTCAGGATCGCTGGACTACTGGGGACAGGGTACTCTCGTGA |
| | | CCGTGTCATCGGGCGGAGGAGGTTCCGGCGGTGGCGGCTCCGGCGGCGGAGGGTCGGA |
| | | GATCGTGATGACCCAGAGCCCTGGTACTCTGAGCCTTTCGCCGGGAGAAAGGGCCACC |
| | | CTGTCCTGCCGCGCTTCCCAATCCGTGTCCTCCGCGTACTTGGCGTGGTACCAGCAGA |
| | | AGCCGGGACAGCCCCCTCGGCTGCTGATCAGCGGGGCCAGCACCCGGGCAACCGGAAT |
| | | CCCAGACAGATTCGGGGGTTCCGGCAGCGGCACAGATTTCACCCTGACTATTTCGAGG |
| | | TTGGAGCCCGAGGACTTTGCGGTGTATTACTGTCAGCACTACGGGTCGTCCTTTAATG |
| | | GCTCCAGCCTGTTCACGTTCGGACAGGGGACCCGCCTGGAAATCAAGACCACTACCCC |
| | | AGCACCGAGGCCACCCACCCCGGCTCCTACCATCGCCTCCCAGCCTCTGTCCCTGCGT |
| | | CCGGAGGCATGTAGACCCGCAGCTGGTGGGGCCGTGCATACCCGGGGTCTTGACTTCG |
| | | CCTGCGATATCTACATTTGGGCCCCTCTGGCTGGTACTTGCGGGGTCCTGCTGCTTTC |
| | | ACTCGTGATCACTCTTTACTGTAAGCGCGGTCGGAAGAAGCTGCTGTACATCTTTAAG |
| | | CAACCCTTCATGAGGCCTGTGCAGACTACTCAAGAGGAGGACGGCTGTTCATGCCGGT |
| | | TCCCAGAGGAGGAGGAAGGCGGCTGCGAACTGCGCGTGAAATTCAGCCGCAGCGCAGA |
| | | TGCTCCAGCCTACAAGCAGGGGCAGAACCAGCTCTACAACGAACTCAATCTTGGTCGG |
| | | AGAGAGGAGTACGACGTGCTGGACAAGCGGAGAGGACGGGACCCAGAAATGGGCGGGA |
| | | AGCCGCGCAGAAAGAATCCCCAAGAGGGCCTGTACAACGAGCTCCAAAAGGATAAGAT |
| | | GGCAGAAGCCTATAGCGAGATTGGTATGAAAGGGGAACGCAGAAGAGGCAAAGGCCAC |
| | | GACGGACTGTACCAGGGACTCAGCACCGCCACCAAGGACACCTATGACGCTCTTCACA |
| | | TGCAGGCCCTGCCGCCTCGG |
| BCMA_EBB- | | |
| C1978-G1 | | |
| BCMA_EBB- | 138 | EVQLVETGGGLVQPGGSLRLSCAASGITFSRYPMSWVRQAPGKGLEWVSGISD |
| C1978-G1-aa | | SGVSTYYADSAKGRFTISRDNSKNTLFLQMSSLRDEDTAVYYCVTRAGSEASD |
| ScFv domain | | IWGQGTMVTVSSGGGGSGGGGSGGGGSEIVLTQSPATLSLSPGERATLSCRAS |
| | | QSVSNSLAWYQQKPGQAPRLLIYDASSRATGIPDRFSGSGSGTDFTLTISRLEPE |
| | | DFAIYYCQQFGTSSGLTFGGGTKLEIK |
| BCMA_EBB- | 159 | GAAGTGCAACTGGTGGAAACCGGTGGCGGCCTGGTGCAGCCTGGAGGATCATTGAGGC |
| C1978-G1-nt | | TGTCATGCGCGGCCAGCGGTATTACCTTCTCCCGGTACCCCATGTCCTGGGTCAGACA |
| ScFv domain | | GGCCCCGGGGAAAGGGCTTGAATGGGTGTCCGGGATCTCGGACTCCGGTGTCAGCACT |
| | | TACTACGCCGACTCCGCCAAGGGACGCTTCACCATTTCCCGGGACAACTCGAAGAACA |
| | | CCCTGTTCCTCCAAATGAGCTCCCTCCGGGACGAGGATACTGCAGTGTACTACTGCGT |
| | | GACCCGCGCCGGGTCCGAGGCGTCTGACATTTGGGGACAGGGCACTATGGTCACCGTG |
| | | TCGTCCGGCGGAGGGGGCTCGGGAGGCGGTGGCAGCGGAGGAGGAGGGTCCGAGATCG |
| | | TGCTGACCCAATCCCCGGCCACCCTCTCGCTGAGCCCTGGAGAAAGGGCAACCTTGTC |
| | | CTGTCGCGCGAGCCAGTCCGTGAGCAACTCCCTGGCCTGGTACCAGCAGAAGCCCGGA |
| | | CAGGCTCCGAGACTTCTGATCTACGACGCTTCGAGCCGGGCCACTGGAATCCCCGACC |
| | | GCTTTTCGGGGTCCGGCTCAGGAACCGATTTCACCCTGACAATCTCACGGCTGGAGCC |
| | | AGAGGATTTCGCCATCTATTACTGCCAGCAGTTCGGTACTTCCTCCGGCCTGACTTTC |
| | | GGAGGCGGCACGAAGCTCGAAATCAAG |
| BCMA_EBB- | 180 | EVQLVETGGGLVQPGGSLRLSCAASGITFSRYPMSWVRQAPGKGLEWVSGISDSGVST |
| C1978-G1-aa | | YYADSAKGRFTISRDNSKNTLFLQMSSLRDEDTAVYYCVTRAGSEASDIWGQGTMVTV |
| VH | | SS |
| BCMA_EBB- | 201 | EIVLTQSPATLSLSPGERATLSCRASQSVSNSLAWYQQKPGQAPRLLIYDASSRATGI |
| C1978-G1-aa | | PDRFSGSGSGTDFTLTISRLEPEDFAIYYCQQFGTSSGLTFGGGTKLEIK |
| VL | | |
| BCMA_EBB- | 222 | MALPVTALLLPLALLLHAARPEVQLVETGGGLVQPGGSLRLSCAASGITFSRYP |
| C1978-G1-aa | | MSWVRQAPGKGLEWVSGISDSGVSTYYADSAKGRFTISRDNSKNTLFLQMSS |
| Full CART | | LRDEDTAVYYCVTRAGSEASDIWGQGTMVTVSSGGGGSGGGGSGGGGSEIVL |
| | | TQSPATLSLSPGERATLSCRASQSVSNSLAWYQQKPGQAPRLLIYDASSRATGI |
| | | PDRFSGSGSGTDFTLTISRLEPEDFAIYYCQQFGTSSGLTFGGGTKLEIKTTTPAP |
| | | RPPTPAPTIASQPLSLRPEACRPAAGGAVHTRGLDFACDIYIWAPLAGTCGVLL |
| | | LSLVITLYCKRGRKKLLYIFKQPFMRPVQTTQLEDGCSCRFPEELEGGCELRVK |
| | | FSRSADAPAYKQGQNQLYNELNLGRREEYDVLDKRRGRDPEMGGKPRRKNP |
| | | QEGLYNELQKDKMAEAYSEIGMKGERRRGKGHDGLYQGLSTATKDTYDALH |
| | | MQALPPR |
| BCMA_EBB- | 243 | ATGGCCCTCCCTGTCACCGCCCTGCTGCTTCCGCTGGCTCTTCTGCTCCACGCCGCTC |
| C1978-G1-nt | | GGCCCGAAGTGCAACTGGTGGAAACCGGTGGCGGCCTGGTGCAGCCTGGAGGATCATT |
| Full CART | | GAGGCTGTCATGCGCGGCCAGCGGTATTACCTTCTCCCGGTACCCCATGTCCTGGGTC |
| | | AGACAGGCCCCGGGGAAAGGGCTTGAATGGGTGTCCGGGATCTCGGACTCCGGTGTCA |
| | | GCACTTACTACGCCGACTCCGCCAAGGGACGCTTCACCATTTCCCGGGACAACTCGAA |
| | | GAACACCCTGTTCCTCCAAATGAGCTCCCTCCGGGACGAGGATACTGCAGTGTACTAC |
| | | TGCGTGACCCGCGCCGGGTCCGAGGCGTCTGACATTTGGGGACAGGGCACTATGGTCA |
| | | CCGTGTCGTCCGGCGGAGGGGGCTCGGGAGGCGGTGGCAGCGGAGGAGGAGGGTCCGA |
| | | GATCGTGCTGACCCAATCCCCGGCCACCCTCTCGCTGAGCCCTGGAGAAAGGGCAACC |
| | | TTGTCCTGTCGCGCGAGCCAGTCCGTGAGCAACTCCCTGGCCTGGTACCAGCAGAAGC |
| | | CCGGACAGGCTCCGAGACTTCTGATCTACGACGCTTCGAGCCGGGCCACTGGAATCCC |
| | | CGACCGCTTTTCGGGGTCCGGCTCAGGAACCGATTTCACCCTGACAATCTCACGGCTG |
| | | GAGCCAGAGGATTTCGCCATCTATTACTGCCAGCAGTTCGGTACTTCCTCCGGCCTGA |
| | | CTTTCGGAGGCGGCACGAAGCTCGAAATCAAGACCACTACCCCAGCACCGAGGCCACC |
| | | CACCCCGGCTCCTACCATCGCCTCCCAGCCTCTGTCCCTGCGTCCGGAGGCATGTAGA |
| | | CCCGCAGCTGGTGGGGCCGTGCATACCCGGGGTCTTGACTTCGCCTGCGATATCTACA |
| | | TTTGGGCCCCTCTGGCTGGTACTTGCGGGGTCCTGCTGCTTTCACTCGTGATCACTCT |
| | | TTACTGTAAGCGCGGTCGGAAGAAGCTGCTGTACATCTTTAAGCAACCCTTCATGAGG |
| | | CCTGTGCAGACTACTCAAGAGGAGGACGGCTGTTCATGCCGGTTCCCAGAGGAGGAGG |
| | | AAGGCGGCTGCGAACTGCGCGTGAAATTCAGCCGCAGCGCAGATGCTCCAGCCTACAA |
| | | GCAGGGGCAGAACCAGCTCTACAACGAACTCAATCTTGGTCGGAGAGAGGAGTACGAC |
| | | GTGCTGGACAAGCGGAGAGGACGGGACCCAGAAATGGGCGGGAAGCCGCGCAGAAAGA |
| | | ATCCCCAAGAGGGCCTGTACAACGAGCTCCAAAAGGATAAGATGGCAGAAGCCTATAG |
| | | CGAGATTGGTATGAAAGGGGAACGCAGAAGAGGCAAAGGCCACGACGGACTGTACCAG |
| | | GGACTCAGCACCGCCACCAAGGACACCTATGACGCTCTTCACATGCAGGCCCTGCCGC |
| | | CTCGG |
| BCMA_EBB- | | |
| C1979-C1 | | |
| BCMA_EBB- | 139 | QVQLVESGGGLVQPGGSLRLSCAASGFTFSSYAMSWVRQAPGKGLEWVSAISGSGGST |
| C1979-C1-aa | | YYADSVKGRFTISRDNAKNSLYLQMNSLRAEDTAIYYCARATYKRELRYYYGMDVWGQ |
| ScFv domain | | GTMVTVSSGGGGSGGGGSGGGGSEIVMTQSPGTVSLSPGERATLSCRASQSVSSSFLA |
| | | WYQQKPGQAPRLLIYGASSRATGIPDRFSGSGSGTDFTLTISRLEPEDSAVYYCQQYH |
| | | SSPSWTFGQGTRLEIK |
| BCMA_EBB- | 160 | CAAGTGCAGCTCGTGGAATCGGGTGGCGGACTGGTGCAGCCGGGGGGCTCACTTAGAC |
| C1979-C1-nt | | TGTCCTGCGCGGCCAGCGGATTCACTTTCTCCTCCTACGCCATGTCCTGGGTCAGACA |
| ScFv domain | | GGCCCCTGGAAAGGGCCTGGAATGGGTGTCCGCAATCAGCGGCAGCGGCGGCTCGACC |
| | | TATTACGCGGATTCAGTGAAGGGCAGATTCACCATTTCCCGGGACAACGCCAAGAACT |
| | | CCTTGTACCTTCAAATGAACTCCCTCCGCGCGGAAGATACCGCAATCTACTACTGCGC |
| | | TCGGGCCACTTACAAGAGGGAACTGCGCTACTACTACGGGATGGACGTCTGGGGCCAG |
| | | GGAACCATGGTCACCGTGTCCAGCGGAGGAGGAGGATCGGGAGGAGGCGGTAGCGGGG |
| | | GTGGAGGGTCGGAGATCGTGATGACCCAGTCCCCCGGCACTGTGTCGCTGTCCCCCGG |
| | | CGAACGGGCCACCCTGTCATGTCGGGCCAGCCAGTCAGTGTCGTCAAGCTTCCTCGCC |
| | | TGGTACCAGCAGAAACCGGGACAAGCTCCCCGCCTGCTGATCTACGGAGCCAGCAGCC |
| | | GGGCCACCGGTATTCCTGACCGGTTCTCCGGTTCGGGGTCCGGGACCGACTTTACTCT |
| | | GACTATCTCTCGCCTCGAGCCAGAGGACTCCGCCGTGTATTACTGCCAGCAGTACCAC |
| | | TCCTCCCCGTCCTGGACGTTCGGACAGGGCACAAGGCTGGAGATTAAG |
| BCMA_EBB- | 181 | QVQLVESGGGLVQPGGSLRLSCAASGFTFSSYAMSWVRQAPGKGLEWVSAISGSGGST |
| C1979-C1-aa | | YYADSVKGRFTISRDNAKNSLYLQMNSLRAEDTAIYYCARATYKRELRYYYGMDVWGQ |
| VH | | GTMVTVSS |
| BCMA_EBB- | 202 | EIVMTQSPGTVSLSPGERATLSCRASQSVSSSFLAWYQQKPGQAPRLLIYGASSRATG |
| C1979-C1-aa | | IPDRFSGSGSGTDFTLTISRLEPEDSAVYYCQQYHSSPSWTFGQGTRLEIK |
| VL | | |
| BCMA_EBB- | 223 | MALPVTALLLPLALLLHAARPQVQLVESGGGLVQPGGSLRLSCAASGFTFSSYAMSWV |
| C1979-C1-aa | | RQAPGKGLEWVSAISGSGGSTYYADSVKGRFTISRDNAKNSLYLQMNSLRAEDTAIYY |
| Full CART | | CARATYKRELRYYYGMDVWGQGTMVTVSSGGGGSGGGGSGGGGSEIVMTQSPGTVSLS |
| | | PGERATLSCRASQSVSSSFLAWYQQKPGQAPRLLIYGASSRATGIPDRFSGSGSGTDF |
| | | TLTISRLEPEDSAVYYCQQYHSSPSWTFGQGTRLEIKTTTPAPRPPTPAPTIASQPLS |
| | | LRPEACRPAAGGAVHTRGLDFACDIYIWAPLAGTCGVLLLSLVITLYCKRGRKKLLYI |
| | | FKQPFMRPVQTTQEEDGCSCRFPEEEEGGCELRVKFSRSADAPAYKQGQNQLYNELNL |
| | | GRREEYDVLDKRRGRDPEMGGKPRRKNPQEGLYNELQKDKMAEAYSEIGMKGERRRGK |
| | | GHDGLYQGLSTATKDTYDALHMQALPPR |
| BCMA_EBB- | 244 | ATGGCCCTCCCTGTCACCGCCCTGCTGCTTCCGCTGGCTCTTCTGCTCCACGCCGCTC |
| C1979-C1-nt | | GGCCCCAAGTGCAGCTCGTGGAATCGGGTGGCGGACTGGTGCAGCCGGGGGGCTCACT |
| Full CART | | TAGACTGTCCTGCGCGGCCAGCGGATTCACTTTCTCCTCCTACGCCATGTCCTGGGTC |
| | | AGACAGGCCCCTGGAAAGGGCCTGGAATGGGTGTCCGCAATCAGCGGCAGCGGCGGCT |
| | | CGACCTATTACGCGGATTCAGTGAAGGGCAGATTCACCATTTCCCGGGACAACGCCAA |
| | | GAACTCCTTGTACCTTCAAATGAACTCCCTCCGCGCGGAAGATACCGCAATCTACTAC |
| | | TGCGCTCGGGCCACTTACAAGAGGGAACTGCGCTACTACTACGGGATGGACGTCTGGG |
| | | GCCAGGGAACCATGGTCACCGTGTCCAGCGGAGGAGGAGGATCGGGAGGAGGCGGTAG |
| | | CGGGGGTGGAGGGTCGGAGATCGTGATGACCCAGTCCCCCGGCACTGTGTCGCTGTCC |
| | | CCCGGCGAACGGGCCACCCTGTCATGTCGGGCCAGCCAGTCAGTGTCGTCAAGCTTCC |
| | | TCGCCTGGTACCAGCAGAAACCGGGACAAGCTCCCCGCCTGCTGATCTACGGAGCCAG |
| | | CAGCCGGGCCACCGGTATTCCTGACCGGTTCTCCGGTTCGGGGTCCGGGACCGACTTT |
| | | ACTCTGACTATCTCTCGCCTCGAGCCAGAGGACTCCGCCGTGTATTACTGCCAGCAGT |
| | | ACCACTCCTCCCCGTCCTGGACGTTCGGACAGGGCACAAGGCTGGAGATTAAGACCAC |
| | | TACCCCAGCACCGAGGCCACCCACCCCGGCTCCTACCATCGCCTCCCAGCCTCTGTCC |
| | | CTGCGTCCGGAGGCATGTAGACCCGCAGCTGGTGGGGCCGTGCATACCCGGGGTCTTG |
| | | ACTTCGCCTGCGATATCTACATTTGGGCCCCTCTGGCTGGTACTTGCGGGGTCCTGCT |
| | | GCTTTCACTCGTGATCACTCTTTACTGTAAGCGCGGTCGGAAGAAGCTGCTGTACATC |
| | | TTTAAGCAACCCTTCATGAGGCCTGTGCAGACTACTCAAGAGGAGGACGGCTGTTCAT |
| | | GCCGGTTCCCAGAGGAGGAGGAAGGCGGCTGCGAACTGCGCGTGAAATTCAGCCGCAG |
| | | CGCAGATGCTCCAGCCTACAAGCAGGGGCAGAACCAGCTCTACAACGAACTCAATCTT |
| | | GGTCGGAGAGAGGAGTACGACGTGCTGGACAAGCGGAGAGGACGGGACCCAGAAATGG |
| | | GCGGGAAGCCGCGCAGAAAGAATCCCCAAGAGGGCCTGTACAACGAGCTCCAAAAGGA |
| | | TAAGATGGCAGAAGCCTATAGCGAGATTGGTATGAAAGGGGAACGCAGAAGAGGCAAA |
| | | GGCCACGACGGACTGTACCAGGGACTCAGCACCGCCACCAAGGACACCTATGACGCTC |
| | | TTCACATGCAGGCCCTGCCGCCTCGG |
| BCMA_EBB- | | |
| C1978-C7 | | |
| BCMA_EBB- | 140 | EVQLVETGGGLVQPGGSLRLSCAASGFTFSSYAMSWVRQAPGKGLEWVSAISGSGGST |
| C1978-C7-aa | | YYADSVKGRFTISRDNSKNTLYLQMNTLKAEDTAVYYCARATYKRELRYYYGMDVWGQ |
| ScFv domain | | GTTVTVSSGGGGSGGGGSGGGGSEIVLTQSPSTLSLSPGESATLSCRASQSVSTTFLA |
| | | WYQQKPGQAPRLLIYGSSNRATGIPDRFSGSGSGTDFTLTIRRLEPEDFAVYYCQQYH |
| | | SSPSWTFGQGTKVEIK |
| BCMA_EBB- | 161 | GAGGTGCAGCTTGTGGAAACCGGTGGCGGACTGGTGCAGCCCGGAGGAAGCCTCAGGC |
| C1978-C7-nt | | TGTCCTGCGCCGCGTCCGGCTTCACCTTCTCCTCGTACGCCATGTCCTGGGTCCGCCA |
| ScFv domain | | GGCCCCCGGAAAGGGCCTGGAATGGGTGTCCGCCATCTCTGGAAGCGGAGGTTCCACG |
| | | TACTACGCGGACAGCGTCAAGGGAAGGTTCACAATCTCCCGCGATAATTCGAAGAACA |
| | | CTCTGTACCTTCAAATGAACACCCTGAAGGCCGAGGACACTGCTGTGTACTACTGCGC |
| | | ACGGGCCACCTACAAGAGAGAGCTCCGGTACTACTACGGAATGGACGTCTGGGGCCAG |
| | | GGAACTACTGTGACCGTGTCCTCGGGAGGGGGTGGCTCCGGGGGGGGCGGCTCCGGCG |
| | | GAGGCGGTTCCGAGATTGTGCTGACCCAGTCACCTTCAACTCTGTCGCTGTCCCCGGG |
| | | AGAGAGCGCTACTCTGAGCTGCCGGGCCAGCCAGTCCGTGTCCACCACCTTCCTCGCC |
| | | TGGTATCAGCAGAAGCCGGGGCAGGCACCACGGCTCTTGATCTACGGGTCAAGCAACA |
| | | GAGCGACCGGAATTCCTGACCGCTTCTCGGGGAGCGGTTCAGGCACCGACTTCACCCT |
| | | GACTATCCGGCGCCTGGAACCCGAAGATTTCGCCGTGTATTACTGTCAACAGTACCAC |
| | | TCCTCGCCGTCCTGGACCTTTGGCCAAGGAACCAAAGTGGAAATCAAG |
| BCMA_EBB- | 182 | EVQLVETGGGLVQPGGSLRLSCAASGFTFSSYAMSWVRQAPGKGLEWVSAISGSGGST |
| C1978-C7-aa | | YYADSVKGRFTISRDNSKNTLYLQMNTLKAEDTAVYYCARATYKRELRYYYGMDVWGQ |
| VH | | GTTVTVSS |
| BCMA_EBB- | 203 | EIVLTQSPSTLSLSPGESATLSCRASQSVSTTFLAWYQQKPGQAPRLLIYGSSNRATG |
| C1978-C7-aa | | IPDRFSGSGSGTDFTLTIRRLEPEDFAVYYCQQYHSSPSWTFGQGTKVEIK |
| VL | | |
| BCMA_EBB- | 224 | MALPVTALLLPLALLLHAARPEVQLVETGGGLVQPGGSLRLSCAASGFTFSSYAMSWV |
| C1978-C7-aa | | RQAPGKGLEWVSAISGSGGSTYYADSVKGRFTISRDNSKNTLYLQMNTLKAEDTAVYY |
| Full CART | | CARATYKRELRYYYGMDVWGQGTTVTVSSGGGGSGGGGSGGGGSEIVLTQSPSTLSLS |
| | | PGESATLSCRASQSVSTTFLAWYQQKPGQAPRLLIYGSSNRATGIPDRFSGSGSGTDF |
| | | TLTIRRLEPEDFAVYYCQQYHSSPSWTFGQGTKVEIKTTTPAPRPPTPAPTIASQPLS |
| | | LRPEACRPAAGGAVHTRGLDFACDIYIWAPLAGTCGVLLLSLVITLYCKRGRKKLLYI |
| | | FKQPFMRPVQTTQEEDGCSCRFPEEEEGGCELRVKFSRSADAPAYKQGQNQLYNELNL |
| | | GRREEYDVLDKRRGRDPEMGGKPRRKNPQEGLYNELQKDKMAEAYSEIGMKGERRRGK |
| | | GHDGLYQGLSTATKDTYDALHMQALPPR |
| BCMA_EBB- | 245 | ATGGCCCTCCCTGTCACCGCCCTGCTGCTTCCGCTGGCTCTTCTGCTCCACGCCGCTC |
| C1978-C7-nt | | GGCCCGAGGTGCAGCTTGTGGAAACCGGTGGCGGACTGGTGCAGCCCGGAGGAAGCCT |
| Full CART | | CAGGCTGTCCTGCGCCGCGTCCGGCTTCACCTTCTCCTCGTACGCCATGTCCTGGGTC |
| | | CGCCAGGCCCCCGGAAAGGGCCTGGAATGGGTGTCCGCCATCTCTGGAAGCGGAGGTT |
| | | CCACGTACTACGCGGACAGCGTCAAGGGAAGGTTCACAATCTCCCGCGATAATTCGAA |
| | | GAACACTCTGTACCTTCAAATGAACACCCTGAAGGCCGAGGACACTGCTGTGTACTAC |
| | | TGCGCACGGGCCACCTACAAGAGAGAGCTCCGGTACTACTACGGAATGGACGTCTGGG |
| | | GCCAGGGAACTACTGTGACCGTGTCCTCGGGAGGGGGTGGCTCCGGGGGGGGCGGCTC |
| | | CGGCGGAGGCGGTTCCGAGATTGTGCTGACCCAGTCACCTTCAACTCTGTCGCTGTCC |
| | | CCGGGAGAGAGCGCTACTCTGAGCTGCCGGGCCAGCCAGTCCGTGTCCACCACCTTCC |
| | | TCGCCTGGTATCAGCAGAAGCCGGGGCAGGCACCACGGCTCTTGATCTACGGGTCAAG |
| | | CAACAGAGCGACCGGAATTCCTGACCGCTTCTCGGGGAGCGGTTCAGGCACCGACTTC |
| | | ACCCTGACTATCCGGCGCCTGGAACCCGAAGATTTCGCCGTGTATTACTGTCAACAGT |
| | | ACCACTCCTCGCCGTCCTGGACCTTTGGCCAAGGAACCAAAGTGGAAATCAAGACCAC |
| | | TACCCCAGCACCGAGGCCACCCACCCCGGCTCCTACCATCGCCTCCCAGCCTCTGTCC |
| | | CTGCGTCCGGAGGCATGTAGACCCGCAGCTGGTGGGGCCGTGCATACCCGGGGTCTTG |
| | | ACTTCGCCTGCGATATCTACATTTGGGCCCCTCTGGCTGGTACTTGCGGGGTCCTGCT |
| | | GCTTTCACTCGTGATCACTCTTTACTGTAAGCGCGGTCGGAAGAAGCTGCTGTACATC |
| | | TTTAAGCAACCCTTCATGAGGCCTGTGCAGACTACTCAAGAGGAGGACGGCTGTTCAT |
| | | GCCGGTTCCCAGAGGAGGAGGAAGGCGGCTGCGAACTGCGCGTGAAATTCAGCCGCAG |
| | | CGCAGATGCTCCAGCCTACAAGCAGGGGCAGAACCAGCTCTACAACGAACTCAATCTT |
| | | GGTCGGAGAGAGGAGTACGACGTGCTGGACAAGCGGAGAGGACGGGACCCAGAAATGG |
| | | GCGGGAAGCCGCGCAGAAAGAATCCCCAAGAGGGCCTGTACAACGAGCTCCAAAAGGA |
| | | TAAGATGGCAGAAGCCTATAGCGAGATTGGTATGAAAGGGGAACGCAGAAGAGGCAAA |
| | | GGCCACGACGGACTGTACCAGGGACTCAGCACCGCCACCAAGGACACCTATGACGCTC |
| | | TTCACATGCAGGCCCTGCCGCCTCGG |
| BCMA_EBB- | | |
| C1978-D10 | | |
| BCMA_EBB- | 141 | EVQLVETGGGLVQPGRSLRLSCAASGFTFDDYAMHWVRQAPGKGLEWVSGISWNSGSI |
| C1978-D10-aa | | GYADSVKGRFTISRDNAKNSLYLQMNSLRDEDTAVYYCARVGKAVPDVWGQGTTVTVS |
| ScFv domain | | SGGGGSGGGGSGGGGSDIVMTQTPSSLSASVGDRVTITCRASQSISSYLNWYQQKPGK |
| | | APKLLIYAASSLQSGVPSRFSGSGSGTDFTLTISSLQPEDFATYYCQQSYSTPYSFGQ |
| | | GTRLEIK |
| BCMA_EBB- | 162 | GAAGTGCAGCTCGTGGAAACTGGAGGTGGACTCGTGCAGCCTGGACGGTCGCTGCGGC |
| C1978-D10-nt | | TGAGCTGCGCTGCATCCGGCTTCACCTTCGACGATTATGCCATGCACTGGGTCAGACA |
| ScFv domain | | GGCGCCAGGGAAGGGACTTGAGTGGGTGTCCGGTATCAGCTGGAATAGCGGCTCAATC |
| | | GGATACGCGGACTCCGTGAAGGGAAGGTTCACCATTTCCCGCGACAACGCCAAGAACT |
| | | CCCTGTACTTGCAAATGAACAGCCTCCGGGATGAGGACACTGCCGTGTACTACTGCGC |
| | | CCGCGTCGGAAAAGCTGTGCCCGACGTCTGGGGCCAGGGAACCACTGTGACCGTGTCC |
| | | AGCGGCGGGGGTGGATCGGGCGGTGGAGGGTCCGGTGGAGGGGGCTCAGATATTGTGA |
| | | TGACCCAGACCCCCTCGTCCCTGTCCGCCTCGGTCGGCGACCGCGTGACTATCACATG |
| | | TAGAGCCTCGCAGAGCATCTCCAGCTACCTGAACTGGTATCAGCAGAAGCCGGGGAAG |
| | | GCCCCGAAGCTCCTGATCTACGCGGCATCATCACTGCAATCGGGAGTGCCGAGCCGGT |
| | | TTTCCGGGTCCGGCTCCGGCACCGACTTCACGCTGACCATTTCTTCCCTGCAACCCGA |
| | | GGACTTCGCCACTTACTACTGCCAGCAGTCCTACTCCACCCCTTACTCCTTCGGCCAA |
| | | GGAACCAGGCTGGAAATCAAG |
| BCMA_EBB- | 183 | EVQLVETGGGLVQPGRSLRLSCAASGFTFDDYAMHWVRQAPGKGLEWVSGISWNSGSI |
| C1978-1D10-aa | | GYADSVKGRFTISRDNAKNSLYLQMNSLRDEDTAVYYCARVGKAVPDVWGQGTTVTVS |
| VH | | S |
| BCMA_EBB- | 204 | DIVMTQTPSSLSASVGDRVTITCRASQSISSYLNWYQQKPGKAPKLLIYAASSLQSGV |
| C1978-1D10-aa | | PSRFSGSGSGTDFTLTISSLQPEDFATYYCQQSYSTPYSFGQGTRLEIK |
| VL | | |
| BCMA_EBB- | 225 | MALPVTALLLPLALLLHAARPEVQLVETGGGLVQPGRSLRLSCAASGFTFDDYAMHWV |
| C1978-1D10-aa | | RQAPGKGLEWVSGISWNSGSIGYADSVKGRFTISRDNAKNSLYLQMNSLRDEDTAVYY |
| Full CART | | CARVGKAVPDVWGQGTTVTVSSGGGGSGGGGSGGGGSDIVMTQTPSSLSASVGDRVTI |
| | | TCRASQSISSYLNWYQQKPGKAPKLLIYAASSLQSGVPSRFSGSGSGTDFTLTISSLQ |
| | | PEDFATYYCQQSYSTPYSFGQGTRLEIKTTTPAPRPPTPAPTIASQPLSLRPEACRPA |
| | | AGGAVHTRGLDFACDIYIWAPLAGTCGVLLLSLVITLYCKRGRKKLLYIFKQPFMRPV |
| | | QTTQEEDGCSCRFPEEEEGGCELRVKFSRSADAPAYKQGQNQLYNELNLGRREEYDVL |
| | | DKRRGRDPEMGGKPRRKNPQEGLYNELQKDKMAEAYSEIGMKGERRRGKGHDGLYQGL |
| | | STATKDTYDALHMQALPPR |
| BCMA_EBB- | 246 | ATGGCCCTCCCTGTCACCGCCCTGCTGCTTCCGCTGGCTCTTCTGCTCCACGCCGCTC |
| C1978-1D10-nt | | GGCCCGAAGTGCAGCTCGTGGAAACTGGAGGTGGACTCGTGCAGCCTGGACGGTCGCT |
| Full CART | | GCGGCTGAGCTGCGCTGCATCCGGCTTCACCTTCGACGATTATGCCATGCACTGGGTC |
| | | AGACAGGCGCCAGGGAAGGGACTTGAGTGGGTGTCCGGTATCAGCTGGAATAGCGGCT |
| | | CAATCGGATACGCGGACTCCGTGAAGGGAAGGTTCACCATTTCCCGCGACAACGCCAA |
| | | GAACTCCCTGTACTTGCAAATGAACAGCCTCCGGGATGAGGACACTGCCGTGTACTAC |
| | | TGCGCCCGCGTCGGAAAAGCTGTGCCCGACGTCTGGGGCCAGGGAACCACTGTGACCG |
| | | TGTCCAGCGGCGGGGGTGGATCGGGCGGTGGAGGGTCCGGTGGAGGGGGCTCAGATAT |
| | | TGTGATGACCCAGACCCCCTCGTCCCTGTCCGCCTCGGTCGGCGACCGCGTGACTATC |
| | | ACATGTAGAGCCTCGCAGAGCATCTCCAGCTACCTGAACTGGTATCAGCAGAAGCCGG |
| | | GGAAGGCCCCGAAGCTCCTGATCTACGCGGCATCATCACTGCAATCGGGAGTGCCGAG |
| | | CCGGTTTTCCGGGTCCGGCTCCGGCACCGACTTCACGCTGACCATTTCTTCCCTGCAA |
| | | CCCGAGGACTTCGCCACTTACTACTGCCAGCAGTCCTACTCCACCCCTTACTCCTTCG |
| | | GCCAAGGAACCAGGCTGGAAATCAAGACCACTACCCCAGCACCGAGGCCACCCACCCC |
| | | GGCTCCTACCATCGCCTCCCAGCCTCTGTCCCTGCGTCCGGAGGCATGTAGACCCGCA |
| | | GCTGGTGGGGCCGTGCATACCCGGGGTCTTGACTTCGCCTGCGATATCTACATTTGGG |
| | | CCCCTCTGGCTGGTACTTGCGGGGTCCTGCTGCTTTCACTCGTGATCACTCTTTACTG |
| | | TAAGCGCGGTCGGAAGAAGCTGCTGTACATCTTTAAGCAACCCTTCATGAGGCCTGTG |
| | | CAGACTACTCAAGAGGAGGACGGCTGTTCATGCCGGTTCCCAGAGGAGGAGGAAGGCG |
| | | GCTGCGAACTGCGCGTGAAATTCAGCCGCAGCGCAGATGCTCCAGCCTACAAGCAGGG |
| | | GCAGAACCAGCTCTACAACGAACTCAATCTTGGTCGGAGAGAGGAGTACGACGTGCTG |
| | | GACAAGCGGAGAGGACGGGACCCAGAAATGGGCGGGAAGCCGCGCAGAAAGAATCCCC |
| | | AAGAGGGCCTGTACAACGAGCTCCAAAAGGATAAGATGGCAGAAGCCTATAGCGAGAT |
| | | TGGTATGAAAGGGGAACGCAGAAGAGGCAAAGGCCACGACGGACTGTACCAGGGACTC |
| | | AGCACCGCCACCAAGGACACCTATGACGCTCTTCACATGCAGGCCCTGCCGCCTCGG |
| BCMA_EBB- | | |
| C1979-C12 | | |
| BCMA_EBB- | 142 | EVQLVESGGGLVQPGRSLRLSCTASGFTFDDYAMHWVRQRPGKGLEWVASINWKGNSL |
| C1979-C12-aa | | AYGDSVKGRFAISRDNAKNTVFLQMNSLRTEDTAVYYCASHQGVAYYNYAMDVWGRGT |
| ScFv domain | | LVTVSSGGGGSGGGGSGGGGSEIVLTQSPGTLSLSPGERATLSCRATQSIGSSFLAWY |
| | | QQRPGQAPRLLIYGASQRATGIPDRFSGRGSGTDFTLTISRVEPEDSAVYYCQHYESS |
| | | PSWTFGQGTKVEIK |
| BCMA_EBB- | 163 | GAAGTGCAGCTCGTGGAGAGCGGGGGAGGATTGGTGCAGCCCGGAAGGTCCCTGCGGC |
| C1979-C12-nt | | TCTCCTGCACTGCGTCTGGCTTCACCTTCGACGACTACGCGATGCACTGGGTCAGACA |
| ScFv domain | | GCGCCCGGGAAAGGGCCTGGAATGGGTCGCCTCAATCAACTGGAAGGGAAACTCCCTG |
| | | GCCTATGGCGACAGCGTGAAGGGCCGCTTCGCCATTTCGCGCGACAACGCCAAGAACA |
| | | CCGTGTTTCTGCAAATGAATTCCCTGCGGACCGAGGATACCGCTGTGTACTACTGCGC |
| | | CAGCCACCAGGGCGTGGCATACTATAACTACGCCATGGACGTGTGGGGAAGAGGGACG |
| | | CTCGTCACCGTGTCCTCCGGGGGCGGTGGATCGGGTGGAGGAGGAAGCGGTGGCGGGG |
| | | GCAGCGAAATCGTGCTGACTCAGAGCCCGGGAACTCTTTCACTGTCCCCGGGAGAACG |
| | | GGCCACTCTCTCGTGCCGGGCCACCCAGTCCATCGGCTCCTCCTTCCTTGCCTGGTAC |
| | | CAGCAGAGGCCAGGACAGGCGCCCCGCCTGCTGATCTACGGTGCTTCCCAACGCGCCA |
| | | CTGGCATTCCTGACCGGTTCAGCGGCAGAGGGTCGGGAACCGATTTCACACTGACCAT |
| | | TTCCCGGGTGGAGCCCGAAGATTCGGCAGTCTACTACTGTCAGCATTACGAGTCCTCC |
| | | CCTTCATGGACCTTCGGTCAAGGGACCAAAGTGGAGATCAAG |
| BCMA_EBB- | 184 | EVQLVESGGGLVQPGRSLRLSCTASGFTFDDYAMHWVRQRPGKGLEWVASINWKGNSL |
| C1979-C12-aa | | AYGDSVKGRFAISRDNAKNTVFLQMNSLRTEDTAVYYCASHQGVAYYNYAMDVWGRGT |
| VH | | LVTVSS |
| BCMA_EBB- | 205 | EIVLTQSPGTLSLSPGERATLSCRATQSIGSSFLAWYQQRPGQAPRLLIYGASQRATG |
| C1979-C12-aa | | IPDRFSGRGSGTDFTLTISRVEPEDSAVYYCQHYESSPSWTFGQGTKVEIK |
| VL | | |
| BCMA_EBB- | 226 | MALPVTALLLPLALLLHAARPEVQLVESGGGLVQPGRSLRLSCTASGFTFDDYAMHWV |
| C1979-C12-aa | | RQRPGKGLEWVASINWKGNSLAYGDSVKGRFAISRDNAKNTVFLQMNSLRTEDTAVYY |
| Full CART | | CASHQGVAYYNYAMDVWGRGTLVTVSSGGGGSGGGGSGGGGSEIVLTQSPGTLSLSPG |
| | | ERATLSCRATQSIGSSFLAWYQQRPGQAPRLLIYGASQRATGIPDRFSGRGSGTDFTL |
| | | TISRVEPEDSAVYYCQHYESSPSWTFGQGTKVEIKTTTPAPRPPTPAPTIASQPLSLR |
| | | PEACRPAAGGAVHTRGLDFACDIYIWAPLAGTCGVLLLSLVITLYCKRGRKKLLYIFK |
| | | QPFMRPVQTTQEEDGCSCRFPEEEEGGCELRVKFSRSADAPAYKQGQNQLYNELNLGR |
| | | REEYDVLDKRRGRDPEMGGKPRRKNPQEGLYNELQKDKMAEAYSEIGMKGERRRGKGH |
| | | DGLYQGLSTATKDTYDALHMQALPPR |
| BCMA_EBB- | 247 | ATGGCCCTCCCTGTCACCGCCCTGCTGCTTCCGCTGGCTCTTCTGCTCCACGCCGCTC |
| C1979-C12-nt | | GGCCCGAAGTGCAGCTCGTGGAGAGCGGGGGAGGATTGGTGCAGCCCGGAAGGTCCCT |
| Full CART | | GCGGCTCTCCTGCACTGCGTCTGGCTTCACCTTCGACGACTACGCGATGCACTGGGTC |
| | | AGACAGCGCCCGGGAAAGGGCCTGGAATGGGTCGCCTCAATCAACTGGAAGGGAAACT |
| | | CCCTGGCCTATGGCGACAGCGTGAAGGGCCGCTTCGCCATTTCGCGCGACAACGCCAA |
| | | GAACACCGTGTTTCTGCAAATGAATTCCCTGCGGACCGAGGATACCGCTGTGTACTAC |
| | | TGCGCCAGCCACCAGGGCGTGGCATACTATAACTACGCCATGGACGTGTGGGGAAGAG |
| | | GGACGCTCGTCACCGTGTCCTCCGGGGGCGGTGGATCGGGTGGAGGAGGAAGCGGTGG |
| | | CGGGGGCAGCGAAATCGTGCTGACTCAGAGCCCGGGAACTCTTTCACTGTCCCCGGGA |
| | | GAACGGGCCACTCTCTCGTGCCGGGCCACCCAGTCCATCGGCTCCTCCTTCCTTGCCT |
| | | GGTACCAGCAGAGGCCAGGACAGGCGCCCCGCCTGCTGATCTACGGTGCTTCCCAACG |
| | | CGCCACTGGCATTCCTGACCGGTTCAGCGGCAGAGGGTCGGGAACCGATTTCACACTG |
| | | ACCATTTCCCGGGTGGAGCCCGAAGATTCGGCAGTCTACTACTGTCAGCATTACGAGT |
| | | CCTCCCCTTCATGGACCTTCGGTCAAGGGACCAAAGTGGAGATCAAGACCACTACCCC |
| | | AGCACCGAGGCCACCCACCCCGGCTCCTACCATCGCCTCCCAGCCTCTGTCCCTGCGT |
| | | CCGGAGGCATGTAGACCCGCAGCTGGTGGGGCCGTGCATACCCGGGGTCTTGACTTCG |
| | | CCTGCGATATCTACATTTGGGCCCCTCTGGCTGGTACTTGCGGGGTCCTGCTGCTTTC |
| | | ACTCGTGATCACTCTTTACTGTAAGCGCGGTCGGAAGAAGCTGCTGTACATCTTTAAG |
| | | CAACCCTTCATGAGGCCTGTGCAGACTACTCAAGAGGAGGACGGCTGTTCATGCCGGT |
| | | TCCCAGAGGAGGAGGAAGGCGGCTGCGAACTGCGCGTGAAATTCAGCCGCAGCGCAGA |
| | | TGCTCCAGCCTACAAGCAGGGGCAGAACCAGCTCTACAACGAACTCAATCTTGGTCGG |
| | | AGAGAGGAGTACGACGTGCTGGACAAGCGGAGAGGACGGGACCCAGAAATGGGCGGGA |
| | | AGCCGCGCAGAAAGAATCCCCAAGAGGGCCTGTACAACGAGCTCCAAAAGGATAAGAT |
| | | GGCAGAAGCCTATAGCGAGATTGGTATGAAAGGGGAACGCAGAAGAGGCAAAGGCCAC |
| | | GACGGACTGTACCAGGGACTCAGCACCGCCACCAAGGACACCTATGACGCTCTTCACA |
| | | TGCAGGCCCTGCCGCCTCGG |
| BCMA_EBB- | | |
| C1980-G4 | | |
| BCMA_EBB- | 143 | EVQLVESGGGLVQPGGSLRLSCAASGFTFSSYAMSWVRQAPGKGLEWVSAIS |
| C1980-G4-aa | | GSGGSTYYADSVKGRFTISRDNSKNTLYLQMNSLRAEDTAVYYCAKVVRDG |
| ScFv domain | | MDVWGQGTTVTVSSGGGGSGGGGSGGGGSEIVLTQSPATLSLSPGERATLSCR |
| | | ASQSVSSSYLAWYQQKPGQAPRLLIYGASSRATGIPDRFSGNGSGTDFTLTISR |
| | | LEPEDFAVYYCQQYGSPPRFTFGPGTKVDIK |
| BCMA_EBB- | 164 | GAGGTGCAGTTGGTCGAAAGCGGGGGCGGGCTTGTGCAGCCTGGCGGATCACTGCGGC |
| C1980-G4-nt | | TGTCCTGCGCGGCATCAGGCTTCACGTTTTCTTCCTACGCCATGTCCTGGGTGCGCCA |
| ScFv domain | | GGCCCCTGGAAAGGGACTGGAATGGGTGTCCGCGATTTCGGGGTCCGGCGGGAGCACC |
| | | TACTACGCCGATTCCGTGAAGGGCCGCTTCACTATCTCGCGGGACAACTCCAAGAACA |
| | | CCCTCTACCTCCAAATGAATAGCCTGCGGGCCGAGGATACCGCCGTCTACTATTGCGC |
| | | TAAGGTCGTGCGCGACGGAATGGACGTGTGGGGACAGGGTACCACCGTGACAGTGTCC |
| | | TCGGGGGGAGGCGGTAGCGGCGGAGGAGGAAGCGGTGGTGGAGGTTCCGAGATTGTGC |
| | | TGACTCAATCACCCGCGACCCTGAGCCTGTCCCCCGGCGAAAGGGCCACTCTGTCCTG |
| | | TCGGGCCAGCCAATCAGTCTCCTCCTCGTACCTGGCCTGGTACCAGCAGAAGCCAGGA |
| | | CAGGCTCCGAGACTCCTTATCTATGGCGCATCCTCCCGCGCCACCGGAATCCCGGATA |
| | | GGTTCTCGGGAAACGGATCGGGGACCGACTTCACTCTCACCATCTCCCGGCTGGAACC |
| | | GGAGGACTTCGCCGTGTACTACTGCCAGCAGTACGGCAGCCCGCCTAGATTCACTTTC |
| | | GGCCCCGGCACCAAAGTGGACATCAAG |
| BCMA_EBB- | 185 | EVQLVESGGGLVQPGGSLRLSCAASGFTFSSYAMSWVRQAPGKGLEWVSAISGSGGST |
| C1980-G4-aa | | YYADSVKGRFTISRDNSKNTLYLQMNSLRAEDTAVYYCAKVVRDGMDVWGQGTTVTVS |
| VH | | S |
| BCMA_EBB- | 206 | EIVLTQSPATLSLSPGERATLSCRASQSVSSSYLAWYQQKPGQAPRLLIYGASSRATG |
| C1980-G4-aa | | IPDRFSGNGSGTDFTLTISRLEPEDFAVYYCQQYGSPPRFTFGPGTKVDIK |
| VL | | |
| BCMA_EBB- | 227 | MALPVTALLLPLALLLHAARPEVQLVESGGGLVQPGGSLRLSCAASGFTFSSY |
| C1980-G4-aa | | AMSWVRQAPGKGLEWVSAISGSGGSTYYADSVKGRFTISRDNSKNTLYLQMN |
| Full CART | | SLRAEDTAVYYCAKVVRDGMDVWGQGTTVTVSSGGGGSGGGGSGGGGSEIV |
| | | LTQSPATLSLSPGERATLSCRASQSVSSSYLAWYQQKPGQAPRLLIYGASSRAT |
| | | GIPDRFSGNGSGTDFTLTISRLEPEDFAVYYCQQYGSPPRFTFGPGTKVDIKTTT |
| | | PAPRPPTPAPTIASQPLSLRPEACRPAAGGAVHTRGLDFACDIYIWAPLAGTCG |
| | | VLLLSLVITLYCKRGRKKLLYIFKQPFMRPVQTTQLEDGCSCRFPEELEGGCEL |
| | | RVKFSRSADAPAYKQGQNQLYNELNLGRREEYDVLDKRRGRDPEMGGKPRR |
| | | KNPQEGLYNELQKDKMALAYSEIGMKGERRRGKGHDGLYQGLSTATKDTYD |
| | | ALHMQALPPR |
| BCMA_EBB- | 248 | ATGGCCCTCCCTGTCACCGCCCTGCTGCTTCCGCTGGCTCTTCTGCTCCACGCCGCTC |
| C1980-G4-nt | | GGCCCGAGGTGCAGTTGGTCGAAAGCGGGGGCGGGCTTGTGCAGCCTGGCGGATCACT |
| Full CART | | GCGGCTGTCCTGCGCGGCATCAGGCTTCACGTTTTCTTCCTACGCCATGTCCTGGGTG |
| | | CGCCAGGCCCCTGGAAAGGGACTGGAATGGGTGTCCGCGATTTCGGGGTCCGGCGGGA |
| | | GCACCTACTACGCCGATTCCGTGAAGGGCCGCTTCACTATCTCGCGGGACAACTCCAA |
| | | GAACACCCTCTACCTCCAAATGAATAGCCTGCGGGCCGAGGATACCGCCGTCTACTAT |
| | | TGCGCTAAGGTCGTGCGCGACGGAATGGACGTGTGGGGACAGGGTACCACCGTGACAG |
| | | TGTCCTCGGGGGGAGGCGGTAGCGGCGGAGGAGGAAGCGGTGGTGGAGGTTCCGAGAT |
| | | TGTGCTGACTCAATCACCCGCGACCCTGAGCCTGTCCCCCGGCGAAAGGGCCACTCTG |
| | | TCCTGTCGGGCCAGCCAATCAGTCTCCTCCTCGTACCTGGCCTGGTACCAGCAGAAGC |
| | | CAGGACAGGCTCCGAGACTCCTTATCTATGGCGCATCCTCCCGCGCCACCGGAATCCC |
| | | GGATAGGTTCTCGGGAAACGGATCGGGGACCGACTTCACTCTCACCATCTCCCGGCTG |
| | | GAACCGGAGGACTTCGCCGTGTACTACTGCCAGCAGTACGGCAGCCCGCCTAGATTCA |
| | | CTTTCGGCCCCGGCACCAAAGTGGACATCAAGACCACTACCCCAGCACCGAGGCCACC |
| | | CACCCCGGCTCCTACCATCGCCTCCCAGCCTCTGTCCCTGCGTCCGGAGGCATGTAGA |
| | | CCCGCAGCTGGTGGGGCCGTGCATACCCGGGGTCTTGACTTCGCCTGCGATATCTACA |
| | | TTTGGGCCCCTCTGGCTGGTACTTGCGGGGTCCTGCTGCTTTCACTCGTGATCACTCT |
| | | TTACTGTAAGCGCGGTCGGAAGAAGCTGCTGTACATCTTTAAGCAACCCTTCATGAGG |
| | | CCTGTGCAGACTACTCAAGAGGAGGACGGCTGTTCATGCCGGTTCCCAGAGGAGGAGG |
| | | AAGGCGGCTGCGAACTGCGCGTGAAATTCAGCCGCAGCGCAGATGCTCCAGCCTACAA |
| | | GCAGGGGCAGAACCAGCTCTACAACGAACTCAATCTTGGTCGGAGAGAGGAGTACGAC |
| | | GTGCTGGACAAGCGGAGAGGACGGGACCCAGAAATGGGCGGGAAGCCGCGCAGAAAGA |
| | | ATCCCCAAGAGGGCCTGTACAACGAGCTCCAAAAGGATAAGATGGCAGAAGCCTATAG |
| | | CGAGATTGGTATGAAAGGGGAACGCAGAAGAGGCAAAGGCCACGACGGACTGTACCAG |
| | | GGACTCAGCACCGCCACCAAGGACACCTATGACGCTCTTCACATGCAGGCCCTGCCGC |
| | | CTCGG |
| BCMA_EBB- | | |
| C1980-D2 | | |
| BCMA_EBB- | 144 | EVQLLESGGGLVQPGGSLRLSCAASGFTFSSYAMSWVRQAPGKGLEWVSAISGSGGST |
| C1980-D2-aa | | YYADSVKGRFTISRDNSKNTLYLQMNSLRAEDTAVYYCAKIPQTGTFDYWGQGTLVTV |
| ScFv domain | | SSGGGGSGGGGSGGGGSEIVLTQSPGTLSLSPGERATLSCRASQSVSSSYLAWYQQRP |
| | | GQAPRLLIYGASSRATGIPDRFSGSGSGTDFTLTISRLEPEDFAVYYCQHYGSSPSWT |
| | | FGQGTRLEIK |
| BCMA_EBB- | 165 | GAAGTGCAGCTGCTGGAGTCCGGCGGTGGATTGGTGCAACCGGGGGGATCGCTCAGAC |
| C1980-D2-nt | | TGTCCTGTGCGGCGTCAGGCTTCACCTTCTCGAGCTACGCCATGTCATGGGTCAGACA |
| ScFv domain | | GGCCCCTGGAAAGGGTCTGGAATGGGTGTCCGCCATTTCCGGGAGCGGGGGATCTACA |
| | | TACTACGCCGATAGCGTGAAGGGCCGCTTCACCATTTCCCGGGACAACTCCAAGAACA |
| | | CTCTCTATCTGCAAATGAACTCCCTCCGCGCTGAGGACACTGCCGTGTACTACTGCGC |
| | | CAAAATCCCTCAGACCGGCACCTTCGACTACTGGGGACAGGGGACTCTGGTCACCGTC |
| | | AGCAGCGGTGGCGGAGGTTCGGGGGGAGGAGGAAGCGGCGGCGGAGGGTCCGAGATTG |
| | | TGCTGACCCAGTCACCCGGCACTTTGTCCCTGTCGCCTGGAGAAAGGGCCACCCTTTC |
| | | CTGCCGGGCATCCCAATCCGTGTCCTCCTCGTACCTGGCCTGGTACCAGCAGAGGCCC |
| | | GGACAGGCCCCACGGCTTCTGATCTACGGAGCAAGCAGCCGCGCGACCGGTATCCCGG |
| | | ACCGGTTTTCGGGCTCGGGCTCAGGAACTGACTTCACCCTCACCATCTCCCGCCTGGA |
| | | ACCCGAAGATTTCGCTGTGTATTACTGCCAGCACTACGGCAGCTCCCCGTCCTGGACG |
| | | TTCGGCCAGGGAACTCGGCTGGAGATCAAG |
| BCMA_EBB- | 186 | EVQLLESGGGLVQPGGSLRLSCAASGFTFSSYAMSWVRQAPGKGLEWVSAISGSGGST |
| C1980-D2-aa | | YYADSVKGRFTISRDNSKNTLYLQMNSLRAEDTAVYYCAKIPQTGTFDYWGQGTLVTV |
| VH | | SS |
| BCMA_EBB- | 207 | EIVLTQSPGTLSLSPGERATLSCRASQSVSSSYLAWYQQRPGQAPRLLIYGASSRATG |
| C1980-D2-aa | | IPDRFSGSGSGTDFTLTISRLEPEDFAVYYCQHYGSSPSWTFGQGTRLEIK |
| VL | | |
| BCMA_EBB- | 228 | MALPVTALLLPLALLLHAARPEVQLLESGGGLVQPGGSLRLSCAASGFTFSSYAMSWV |
| C1980-D2-aa | | RQAPGKGLEWVSAISGSGGSTYYADSVKGRFTISRDNSKNTLYLQMNSLRAEDTAVYY |
| Full CART | | CAKIPQTGTFDYWGQGTLVTVSSGGGGSGGGGSGGGGSEIVLTQSPGTLSLSPGERAT |
| | | LSCRASQSVSSSYLAWYQQRPGQAPRLLIYGASSRATGIPDRFSGSGSGTDFTLTISR |
| | | LEPEDFAVYYCQHYGSSPSWTFGQGTRLEIKTTTPAPRPPTPAPTIASQPLSLRPEAC |
| | | RPAAGGAVHTRGLDFACDIYIWAPLAGTCGVLLLSLVITLYCKRGRKKLLYIFKQPFM |
| | | RPVQTTQEEDGCSCRFPEEEEGGCELRVKFSRSADAPAYKQGQNQLYNELNLGRREEY |
| | | DVLDKRRGRDPEMGGKPRRKNPQEGLYNELQKDKMAEAYSEIGMKGERRRGKGHDGLY |
| | | QGLSTATKDTYDALHMQALPPR |
| BCMA_EBB- | 249 | ATGGCCCTCCCTGTCACCGCCCTGCTGCTTCCGCTGGCTCTTCTGCTCCACGCCGCTC |
| C1980-D2-nt | | GGCCCGAAGTGCAGCTGCTGGAGTCCGGCGGTGGATTGGTGCAACCGGGGGGATCGCT |
| Full CART | | CAGACTGTCCTGTGCGGCGTCAGGCTTCACCTTCTCGAGCTACGCCATGTCATGGGTC |
| | | AGACAGGCCCCTGGAAAGGGTCTGGAATGGGTGTCCGCCATTTCCGGGAGCGGGGGAT |
| | | CTACATACTACGCCGATAGCGTGAAGGGCCGCTTCACCATTTCCCGGGACAACTCCAA |
| | | GAACACTCTCTATCTGCAAATGAACTCCCTCCGCGCTGAGGACACTGCCGTGTACTAC |
| | | TGCGCCAAAATCCCTCAGACCGGCACCTTCGACTACTGGGGACAGGGGACTCTGGTCA |
| | | CCGTCAGCAGCGGTGGCGGAGGTTCGGGGGGAGGAGGAAGCGGCGGCGGAGGGTCCGA |
| | | GATTGTGCTGACCCAGTCACCCGGCACTTTGTCCCTGTCGCCTGGAGAAAGGGCCACC |
| | | CTTTCCTGCCGGGCATCCCAATCCGTGTCCTCCTCGTACCTGGCCTGGTACCAGCAGA |
| | | GGCCCGGACAGGCCCCACGGCTTCTGATCTACGGAGCAAGCAGCCGCGCGACCGGTAT |
| | | CCCGGACCGGTTTTCGGGCTCGGGCTCAGGAACTGACTTCACCCTCACCATCTCCCGC |
| | | CTGGAACCCGAAGATTTCGCTGTGTATTACTGCCAGCACTACGGCAGCTCCCCGTCCT |
| | | GGACGTTCGGCCAGGGAACTCGGCTGGAGATCAAGACCACTACCCCAGCACCGAGGCC |
| | | ACCCACCCCGGCTCCTACCATCGCCTCCCAGCCTCTGTCCCTGCGTCCGGAGGCATGT |
| | | AGACCCGCAGCTGGTGGGGCCGTGCATACCCGGGGTCTTGACTTCGCCTGCGATATCT |
| | | ACATTTGGGCCCCTCTGGCTGGTACTTGCGGGGTCCTGCTGCTTTCACTCGTGATCAC |
| | | TCTTTACTGTAAGCGCGGTCGGAAGAAGCTGCTGTACATCTTTAAGCAACCCTTCATG |
| | | AGGCCTGTGCAGACTACTCAAGAGGAGGACGGCTGTTCATGCCGGTTCCCAGAGGAGG |
| | | AGGAAGGCGGCTGCGAACTGCGCGTGAAATTCAGCCGCAGCGCAGATGCTCCAGCCTA |
| | | CAAGCAGGGGCAGAACCAGCTCTACAACGAACTCAATCTTGGTCGGAGAGAGGAGTAC |
| | | GACGTGCTGGACAAGCGGAGAGGACGGGACCCAGAAATGGGCGGGAAGCCGCGCAGAA |
| | | AGAATCCCCAAGAGGGCCTGTACAACGAGCTCCAAAAGGATAAGATGGCAGAAGCCTA |
| | | TAGCGAGATTGGTATGAAAGGGGAACGCAGAAGAGGCAAAGGCCACGACGGACTGTAC |
| | | CAGGGACTCAGCACCGCCACCAAGGACACCTATGACGCTCTTCACATGCAGGCCCTGC |
| | | CGCCTCGG |
| BCMA_EBB- | | |
| C1978-A10 | | |
| BCMA_EBB- | 145 | EVQLVETGGGLVQPGGSLRLSCAASGFTFSSYAMSWVRQAPGKGLEWVSAIS |
| C1978-A10-aa | | GSGGSTYYADSVKGRFTMSRENDKNSVFLQMNSLRVEDTGVYYCARANYKR |
| ScFv domain | | ELRYYYGMDVWGQGTMVTVSSGGGGSGGGGSGGGGSEIVMTQSPGTLSLSP |
| | | GESATLSCRASQRVASNYLAWYQHKPGQAPSLLISGASSRATGVPDRFSGSGS |
| | | GTDFTLAISRLEPEDSAVYYCQHYDSSPSWTFGQGTKVEIK |
| BCMA_EBB- | 166 | GAAGTGCAACTGGTGGAAACCGGTGGAGGACTCGTGCAGCCTGGCGGCAGCCTCCGGC |
| C1978-A10-nt | | TGAGCTGCGCCGCTTCGGGATTCACCTTTTCCTCCTACGCGATGTCTTGGGTCAGACA |
| ScFv domain | | GGCCCCCGGAAAGGGGCTGGAATGGGTGTCAGCCATCTCCGGCTCCGGCGGATCAACG |
| | | TACTACGCCGACTCCGTGAAAGGCCGGTTCACCATGTCGCGCGAGAATGACAAGAACT |
| | | CCGTGTTCCTGCAAATGAACTCCCTGAGGGTGGAGGACACCGGAGTGTACTATTGTGC |
| | | GCGCGCCAACTACAAGAGAGAGCTGCGGTACTACTACGGAATGGACGTCTGGGGACAG |
| | | GGAACTATGGTGACCGTGTCATCCGGTGGAGGGGGAAGCGGCGGTGGAGGCAGCGGGG |
| | | GCGGGGGTTCAGAAATTGTCATGACCCAGTCCCCGGGAACTCTTTCCCTCTCCCCCGG |
| | | GGAATCCGCGACTTTGTCCTGCCGGGCCAGCCAGCGCGTGGCCTCGAACTACCTCGCA |
| | | TGGTACCAGCATAAGCCAGGCCAAGCCCCTTCCCTGCTGATTTCCGGGGCTAGCAGCC |
| | | GCGCCACTGGCGTGCCGGATAGGTTCTCGGGAAGCGGCTCGGGTACCGATTTCACCCT |
| | | GGCAATCTCGCGGCTGGAACCGGAGGATTCGGCCGTGTACTACTGCCAGCACTATGAC |
| | | TCATCCCCCTCCTGGACATTCGGACAGGGCACCAAGGTCGAGATCAAG |
| BCMA_EBB- | 187 | EVQLVETGGGLVQPGGSLRLSCAASGFTFSSYAMSWVRQAPGKGLEWVSAISGSGGST |
| C1978-A10-aa | | YYADSVKGRFTMSRENDKNSVFLQMNSLRVEDTGVYYCARANYKRELRYYYGMDVWGQ |
| VH | | GTMVTVSS |
| BCMA_EBB- | 208 | EIVMTQSPGTLSLSPGESATLSCRASQRVASNYLAWYQHKPGQAPSLLISGASSRATG |
| C1978-A10-aa | | VPDRFSGSGSGTDFTLAISRLEPEDSAVYYCQHYDSSPSWTFGQGTKVEIK |
| VL | | |
| BCMA_EBB- | 229 | MALPVTALLLPLALLLHAARPEVQLVETGGGLVQPGGSLRLSCAASGFTFSSY |
| C1978-A10-aa | | AMSWVRQAPGKGLEWVSAISGSGGSTYYADSVKGRFTMSRENDKNSVFLQM |
| Full CART | | NSLRVEDTGVYYCARANYKRELRYYYGMDVWGQGTMVTVSSGGGGSGGGG |
| | | SGGGGSEIVMTQSPGTLSLSPGESATLSCRASQRVASNYLAWYQHKPGQAPSL |
| | | LISGASSRATGVPDRFSGSGSGTDFTLAISRLEPEDSAVYYCQHYDSSPSWTFG |
| | | QGTKVEIKTTTPAPRPPTPAPTIASQPLSLRPEACRPAAGGAVHTRGLDFACDIY |
| | | IWAPLAGTCGVLLLSLVITLYCKRGRKKLLYIFKQPFMRPVQTTQEEDGCSCRF |
| | | PEEEEGGCELRVKFSRSADAPAYKQGQNQLYNELNLGRREEYDVLDKRRGRD |
| | | PEMGGKPRRKNPQEGLYNELQKDKMAEAYSEIGMKGERRRGKGHDGLYQGL |
| | | STATKDTYDALHMQALPPR |
| BCMA_EBB- | 250 | ATGGCCCTCCCTGTCACCGCCCTGCTGCTTCCGCTGGCTCTTCTGCTCCACGCCGCTC |
| C1978-A10-nt | | GGCCCGAAGTGCAACTGGTGGAAACCGGTGGAGGACTCGTGCAGCCTGGCGGCAGCCT |
| Full CART | | CCGGCTGAGCTGCGCCGCTTCGGGATTCACCTTTTCCTCCTACGCGATGTCTTGGGTC |
| | | AGACAGGCCCCCGGAAAGGGGCTGGAATGGGTGTCAGCCATCTCCGGCTCCGGCGGAT |
| | | CAACGTACTACGCCGACTCCGTGAAAGGCCGGTTCACCATGTCGCGCGAGAATGACAA |
| | | GAACTCCGTGTTCCTGCAAATGAACTCCCTGAGGGTGGAGGACACCGGAGTGTACTAT |
| | | TGTGCGCGCGCCAACTACAAGAGAGAGCTGCGGTACTACTACGGAATGGACGTCTGGG |
| | | GACAGGGAACTATGGTGACCGTGTCATCCGGTGGAGGGGGAAGCGGCGGTGGAGGCAG |
| | | CGGGGGCGGGGGTTCAGAAATTGTCATGACCCAGTCCCCGGGAACTCTTTCCCTCTCC |
| | | CCCGGGGAATCCGCGACTTTGTCCTGCCGGGCCAGCCAGCGCGTGGCCTCGAACTACC |
| | | TCGCATGGTACCAGCATAAGCCAGGCCAAGCCCCTTCCCTGCTGATTTCCGGGGCTAG |
| | | CAGCCGCGCCACTGGCGTGCCGGATAGGTTCTCGGGAAGCGGCTCGGGTACCGATTTC |
| | | ACCCTGGCAATCTCGCGGCTGGAACCGGAGGATTCGGCCGTGTACTACTGCCAGCACT |
| | | ATGACTCATCCCCCTCCTGGACATTCGGACAGGGCACCAAGGTCGAGATCAAGACCAC |
| | | TACCCCAGCACCGAGGCCACCCACCCCGGCTCCTACCATCGCCTCCCAGCCTCTGTCC |
| | | CTGCGTCCGGAGGCATGTAGACCCGCAGCTGGTGGGGCCGTGCATACCCGGGGTCTTG |
| | | ACTTCGCCTGCGATATCTACATTTGGGCCCCTCTGGCTGGTACTTGCGGGGTCCTGCT |
| | | GCTTTCACTCGTGATCACTCTTTACTGTAAGCGCGGTCGGAAGAAGCTGCTGTACATC |
| | | TTTAAGCAACCCTTCATGAGGCCTGTGCAGACTACTCAAGAGGAGGACGGCTGTTCAT |
| | | GCCGGTTCCCAGAGGAGGAGGAAGGCGGCTGCGAACTGCGCGTGAAATTCAGCCGCAG |
| | | CGCAGATGCTCCAGCCTACAAGCAGGGGCAGAACCAGCTCTACAACGAACTCAATCTT |
| | | GGTCGGAGAGAGGAGTACGACGTGCTGGACAAGCGGAGAGGACGGGACCCAGAAATGG |
| | | GCGGGAAGCCGCGCAGAAAGAATCCCCAAGAGGGCCTGTACAACGAGCTCCAAAAGGA |
| | | TAAGATGGCAGAAGCCTATAGCGAGATTGGTATGAAAGGGGAACGCAGAAGAGGCAAA |
| | | GGCCACGACGGACTGTACCAGGGACTCAGCACCGCCACCAAGGACACCTATGACGCTC |
| | | TTCACATGCAGGCCCTGCCGCCTCGG |
| BCMA_EBB- | | |
| C1978-D4 | | |
| BCMA_EBB- | 146 | EVQLLETGGGLVQPGGSLRLSCAASGFSFSSYAMSWVRQAPGKGLEWVSAISGSGGST |
| C1978-D4-aa | | YYADSVKGRFTISRDNSKNTLYLQMNSLRAEDTAVYYCAKALVGATGAFDIWGQGTLV |
| ScFv domain | | TVSSGGGGSGGGGSGGGGSEIVLTQSPGTLSLSPGERATLSCRASQSLSSNFLAWYQQ |
| | | KPGQAPGLLIYGASNWATGTPDRFSGSGSGTDFTLTITRLEPEDFAVYYCQYYGTSPM |
| | | YTFGQGTKVEIK |
| BCMA_EBB- | 167 | GAAGTGCAGCTGCTCGAAACCGGTGGAGGGCTGGTGCAGCCAGGGGGCTCCCTGAGGC |
| C1978-D4-nt | | TTTCATGCGCCGCTAGCGGATTCTCCTTCTCCTCTTACGCCATGTCGTGGGTCCGCCA |
| ScFv domain | | AGCCCCTGGAAAAGGCCTGGAATGGGTGTCCGCGATTTCCGGGAGCGGAGGTTCGACC |
| | | TATTACGCCGACTCCGTGAAGGGCCGCTTTACCATCTCCCGGGATAACTCCAAGAACA |
| | | CTCTGTACCTCCAAATGAACTCGCTGAGAGCCGAGGACACCGCCGTGTATTACTGCGC |
| | | GAAGGCGCTGGTCGGCGCGACTGGGGCATTCGACATCTGGGGACAGGGAACTCTTGTG |
| | | ACCGTGTCGAGCGGAGGCGGCGGCTCCGGCGGAGGAGGGAGCGGGGGCGGTGGTTCCG |
| | | AAATCGTGTTGACTCAGTCCCCGGGAACCCTGAGCTTGTCACCCGGGGAGCGGGCCAC |
| | | TCTCTCCTGTCGCGCCTCCCAATCGCTCTCATCCAATTTCCTGGCCTGGTACCAGCAG |
| | | AAGCCCGGACAGGCCCCGGGCCTGCTCATCTACGGCGCTTCAAACTGGGCAACGGGAA |
| | | CCCCTGATCGGTTCAGCGGAAGCGGATCGGGTACTGACTTTACCCTGACCATCACCAG |
| | | ACTGGAACCGGAGGACTTCGCCGTGTACTACTGCCAGTACTACGGCACCTCCCCCATG |
| | | TACACATTCGGACAGGGTACCAAGGTCGAGATTAAG |
| BCMA_EBB- | 188 | EVQLLETGGGLVQPGGSLRLSCAASGFSFSSYAMSWVRQAPGKGLEWVSAISGSGGST |
| C1978-D4-aa | | YYADSVKGRFTISRDNSKNTLYLQMNSLRAEDTAVYYCAKALVGATGAFDIWGQGTLV |
| VH | | TVSS |
| BCMA_EBB- | 209 | EIVLTQSPGTLSLSPGERATLSCRASQSLSSNFLAWYQQKPGQAPGLLIYGASNWATG |
| C1978-D4-aa | | TPDRFSGSGSGTDFTLTITRLEPEDFAVYYCQYYGTSPMYTFGQGTKVEIK |
| VL | | |
| BCMA_EBB- | 230 | MALPVTALLLPLALLLHAARPEVQLLETGGGLVQPGGSLRLSCAASGFSFSSYAMSWV |
| C1978-D4-aa | | RQAPGKGLEWVSAISGSGGSTYYADSVKGRFTISRDNSKNTLYLQMNSLRAEDTAVYY |
| Full CART | | CAKALVGATGAFDIWGQGTLVTVSSGGGGSGGGGSGGGGSEIVLTQSPGTLSLSPGER |
| | | ATLSCRASQSLSSNFLAWYQQKPGQAPGLLIYGASNWATGTPDRFSGSGSGTDFTLTI |
| | | TRLEPEDFAVYYCQYYGTSPMYTFGQGTKVEIKTTTPAPRPPTPAPTIASQPLSLRPE |
| | | ACRPAAGGAVHTRGLDFACDIYIWAPLAGTCGVLLLSLVITLYCKRGRKKLLYIFKQP |
| | | FMRPVQTTQEEDGCSCRFPEEEEGGCELRVKFSRSADAPAYKQGQNQLYNELNLGRRE |
| | | EYDVLDKRRGRDPEMGGKPRRKNPQEGLYNELQKDKMAEAYSEIGMKGERRRGKGHDG |
| | | LYQGLSTATKDTYDALHMQALPPR |
| BCMA_EBB- | 251 | ATGGCCCTCCCTGTCACCGCCCTGCTGCTTCCGCTGGCTCTTCTGCTCCACGCCGCTC |
| C1978-D4-nt | | GGCCCGAAGTGCAGCTGCTCGAAACCGGTGGAGGGCTGGTGCAGCCAGGGGGCTCCCT |
| Full CART | | GAGGCTTTCATGCGCCGCTAGCGGATTCTCCTTCTCCTCTTACGCCATGTCGTGGGTC |
| | | CGCCAAGCCCCTGGAAAAGGCCTGGAATGGGTGTCCGCGATTTCCGGGAGCGGAGGTT |
| | | CGACCTATTACGCCGACTCCGTGAAGGGCCGCTTTACCATCTCCCGGGATAACTCCAA |
| | | GAACACTCTGTACCTCCAAATGAACTCGCTGAGAGCCGAGGACACCGCCGTGTATTAC |
| | | TGCGCGAAGGCGCTGGTCGGCGCGACTGGGGCATTCGACATCTGGGGACAGGGAACTC |
| | | TTGTGACCGTGTCGAGCGGAGGCGGCGGCTCCGGCGGAGGAGGGAGCGGGGGCGGTGG |
| | | TTCCGAAATCGTGTTGACTCAGTCCCCGGGAACCCTGAGCTTGTCACCCGGGGAGCGG |
| | | GCCACTCTCTCCTGTCGCGCCTCCCAATCGCTCTCATCCAATTTCCTGGCCTGGTACC |
| | | AGCAGAAGCCCGGACAGGCCCCGGGCCTGCTCATCTACGGCGCTTCAAACTGGGCAAC |
| | | GGGAACCCCTGATCGGTTCAGCGGAAGCGGATCGGGTACTGACTTTACCCTGACCATC |
| | | ACCAGACTGGAACCGGAGGACTTCGCCGTGTACTACTGCCAGTACTACGGCACCTCCC |
| | | CCATGTACACATTCGGACAGGGTACCAAGGTCGAGATTAAGACCACTACCCCAGCACC |
| | | GAGGCCACCCACCCCGGCTCCTACCATCGCCTCCCAGCCTCTGTCCCTGCGTCCGGAG |
| | | GCATGTAGACCCGCAGCTGGTGGGGCCGTGCATACCCGGGGTCTTGACTTCGCCTGCG |
| | | ATATCTACATTTGGGCCCCTCTGGCTGGTACTTGCGGGGTCCTGCTGCTTTCACTCGT |
| | | GATCACTCTTTACTGTAAGCGCGGTCGGAAGAAGCTGCTGTACATCTTTAAGCAACCC |
| | | TTCATGAGGCCTGTGCAGACTACTCAAGAGGAGGACGGCTGTTCATGCCGGTTCCCAG |
| | | AGGAGGAGGAAGGCGGCTGCGAACTGCGCGTGAAATTCAGCCGCAGCGCAGATGCTCC |
| | | AGCCTACAAGCAGGGGCAGAACCAGCTCTACAACGAACTCAATCTTGGTCGGAGAGAG |
| | | GAGTACGACGTGCTGGACAAGCGGAGAGGACGGGACCCAGAAATGGGCGGGAAGCCGC |
| | | GCAGAAAGAATCCCCAAGAGGGCCTGTACAACGAGCTCCAAAAGGATAAGATGGCAGA |
| | | AGCCTATAGCGAGATTGGTATGAAAGGGGAACGCAGAAGAGGCAAAGGCCACGACGGA |
| | | CTGTACCAGGGACTCAGCACCGCCACCAAGGACACCTATGACGCTCTTCACATGCAGG |
| | | CCCTGCCGCCTCGG |
| BCMA_EBB- | | |
| C1980-A2 | | |
| BCMA_EBB- | 147 | EVQLLESGGGLVQPGGSLRLSCAASGFTFSSYAMSWVRQAPGKGLEWVSAISGSGGST |
| C1980-A2-aa | | YYADSVKGRFTISRDNSKNTLYLQMNSLRAEDTAVYYCVLWFGEGFDPWGQGTLVTVS |
| ScFv domain | | SGGGGSGGGGSGGGGSDIVLTQSPLSLPVTPGEPASISCRSSQSLLHSNGYNYLDWYL |
| | | QKPGQSPQLLIYLGSNRASGVPDRFSGSGSGTDFTLKISRVEAEDVGVYYCMQALQTP |
| | | LTFGGGTKVDIK |
| BCMA_EBB- | 168 | GAAGTGCAGCTGCTTGAGAGCGGTGGAGGTCTGGTGCAGCCCGGGGGATCACTGCGCC |
| C1980-A2-nt | | TGTCCTGTGCCGCGTCCGGTTTCACTTTCTCCTCGTACGCCATGTCGTGGGTCAGACA |
| ScFv domain | | GGCACCGGGAAAGGGACTGGAATGGGTGTCAGCCATTTCGGGTTCGGGGGGCAGCACC |
| | | TACTACGCTGACTCCGTGAAGGGCCGGTTCACCATTTCCCGCGACAACTCCAAGAACA |
| | | CCTTGTACCTCCAAATGAACTCCCTGCGGGCCGAAGATACCGCCGTGTATTACTGCGT |
| | | GCTGTGGTTCGGAGAGGGATTCGACCCGTGGGGACAAGGAACACTCGTGACTGTGTCA |
| | | TCCGGCGGAGGCGGCAGCGGTGGCGGCGGTTCCGGCGGCGGCGGATCTGACATCGTGT |
| | | TGACCCAGTCCCCTCTGAGCCTGCCGGTCACTCCTGGCGAACCAGCCAGCATCTCCTG |
| | | CCGGTCGAGCCAGTCCCTCCTGCACTCCAATGGGTACAACTACCTCGATTGGTATCTG |
| | | CAAAAGCCGGGCCAGAGCCCCCAGCTGCTGATCTACCTTGGGTCAAACCGCGCTTCCG |
| | | GGGTGCCTGATAGATTCTCCGGGTCCGGGAGCGGAACCGACTTTACCCTGAAAATCTC |
| | | GAGGGTGGAGGCCGAGGACGTCGGAGTGTACTACTGCATGCAGGCGCTCCAGACTCCC |
| | | CTGACCTTCGGAGGAGGAACGAAGGTCGACATCAAGA |
| BCMA_EBB- | 189 | EVQLLESGGGLVQPGGSLRLSCAASGFTFSSYAMSWVRQAPGKGLEWVSAISGSGGST |
| C1980-A2-aa | | YYADSVKGRFTISRDNSKNTLYLQMNSLRAEDTAVYYCVLWFGEGFDPWGQGTLVTVS |
| VH | | S |
| BCMA_EBB- | 210 | DIVLTQSPLSLPVTPGEPASISCRSSQSLLHSNGYNYLDWYLQKPGQSPQLLIYLGSN |
| C1980-A2-aa | | RASGVPDRFSGSGSGTDFTLKISRVEAEDVGVYYCMQALQTPLTFGGGTKVDIK |
| VL | | |
| BCMA_EBB- | 231 | MALPVTALLLPLALLLHAARPEVQLLESGGGLVQPGGSLRLSCAASGFTFSSYAMSWV |
| C1980-A2-aa | | RQAPGKGLEWVSAISGSGGSTYYADSVKGRFTISRDNSKNTLYLQMNSLRAEDTAVYY |
| Full CART | | CVLWFGEGFDPWGQGTLVTVSSGGGGSGGGGSGGGGSDIVLTQSPLSLPVTPGEPASI |
| | | SCRSSQSLLHSNGYNYLDWYLQKPGQSPQLLIYLGSNRASGVPDRFSGSGSGTDFTLK |
| | | ISRVEAEDVGVYYCMQALQTPLTFGGGTKVDIKTTTPAPRPPTPAPTIASQPLSLRPE |
| | | ACRPAAGGAVHTRGLDFACDIYIWAPLAGTCGVLLLSLVITLYCKRGRKKLLYIFKQP |
| | | FMRPVQTTQEEDGCSCRFPEEEEGGCELRVKFSRSADAPAYKQGQNQLYNELNLGRRE |
| | | EYDVLDKRRGRDPEMGGKPRRKNPQEGLYNELQKDKMAEAYSEIGMKGERRRGKGHDG |
| | | LYQGLSTATKDTYDALHMQALPPR |
| BCMA_EBB- | 252 | ATGGCCCTCCCTGTCACCGCCCTGCTGCTTCCGCTGGCTCTTCTGCTCCACGCCGCTC |
| C1980-A2-nt | | GGCCCGAAGTGCAGCTGCTTGAGAGCGGTGGAGGTCTGGTGCAGCCCGGGGGATCACT |
| Full CART | | GCGCCTGTCCTGTGCCGCGTCCGGTTTCACTTTCTCCTCGTACGCCATGTCGTGGGTC |
| | | AGACAGGCACCGGGAAAGGGACTGGAATGGGTGTCAGCCATTTCGGGTTCGGGGGGCA |
| | | GCACCTACTACGCTGACTCCGTGAAGGGCCGGTTCACCATTTCCCGCGACAACTCCAA |
| | | GAACACCTTGTACCTCCAAATGAACTCCCTGCGGGCCGAAGATACCGCCGTGTATTAC |
| | | TGCGTGCTGTGGTTCGGAGAGGGATTCGACCCGTGGGGACAAGGAACACTCGTGACTG |
| | | TGTCATCCGGCGGAGGCGGCAGCGGTGGCGGCGGTTCCGGCGGCGGCGGATCTGACAT |
| | | CGTGTTGACCCAGTCCCCTCTGAGCCTGCCGGTCACTCCTGGCGAACCAGCCAGCATC |
| | | TCCTGCCGGTCGAGCCAGTCCCTCCTGCACTCCAATGGGTACAACTACCTCGATTGGT |
| | | ATCTGCAAAAGCCGGGCCAGAGCCCCCAGCTGCTGATCTACCTTGGGTCAAACCGCGC |
| | | TTCCGGGGTGCCTGATAGATTCTCCGGGTCCGGGAGCGGAACCGACTTTACCCTGAAA |
| | | ATCTCGAGGGTGGAGGCCGAGGACGTCGGAGTGTACTACTGCATGCAGGCGCTCCAGA |
| | | CTCCCCTGACCTTCGGAGGAGGAACGAAGGTCGACATCAAGACCACTACCCCAGCACC |
| | | GAGGCCACCCACCCCGGCTCCTACCATCGCCTCCCAGCCTCTGTCCCTGCGTCCGGAG |
| | | GCATGTAGACCCGCAGCTGGTGGGGCCGTGCATACCCGGGGTCTTGACTTCGCCTGCG |
| | | ATATCTACATTTGGGCCCCTCTGGCTGGTACTTGCGGGGTCCTGCTGCTTTCACTCGT |
| | | GATCACTCTTTACTGTAAGCGCGGTCGGAAGAAGCTGCTGTACATCTTTAAGCAACCC |
| | | TTCATGAGGCCTGTGCAGACTACTCAAGAGGAGGACGGCTGTTCATGCCGGTTCCCAG |
| | | AGGAGGAGGAAGGCGGCTGCGAACTGCGCGTGAAATTCAGCCGCAGCGCAGATGCTCC |
| | | AGCCTACAAGCAGGGGCAGAACCAGCTCTACAACGAACTCAATCTTGGTCGGAGAGAG |
| | | GAGTACGACGTGCTGGACAAGCGGAGAGGACGGGACCCAGAAATGGGCGGGAAGCCGC |
| | | GCAGAAAGAATCCCCAAGAGGGCCTGTACAACGAGCTCCAAAAGGATAAGATGGCAGA |
| | | AGCCTATAGCGAGATTGGTATGAAAGGGGAACGCAGAAGAGGCAAAGGCCACGACGGA |
| | | CTGTACCAGGGACTCAGCACCGCCACCAAGGACACCTATGACGCTCTTCACATGCAGG |
| | | CCCTGCCGCCTCGG |
| BCMA_EBB- | | |
| C1981-C3 | | |
| BCMA_EBB- | 148 | QVQLVESGGGLVQPGGSLRLSCAASGFTFSSYAMSWVRQAPGKGLEWVSAISGSGGST |
| C1981-C3-aa | | YYADSVKGRFTISRDNSKNTLYLQMNSLRAEDTAVYYCAKVGYDSSGYYRDYYGMDVW |
| ScFv domain | | GQGTTVTVSSGGGGSGGGGSGGGGSEIVLTQSPGTLSLSPGERATLSCRASQSVSSSY |
| | | LAWYQQKPGQAPRLLIYGTSSRATGISDRFSGSGSGTDFTLTISRLEPEDFAVYYCQH |
| | | YGNSPPKFTFGPGTKLEIK |
| BCMA_EBB- | 169 | CAAGTGCAGCTCGTGGAGTCAGGCGGAGGACTGGTGCAGCCCGGGGGCTCCCTGAGAC |
| C1981-C3-nt | | TTTCCTGCGCGGCATCGGGTTTTACCTTCTCCTCCTATGCTATGTCCTGGGTGCGCCA |
| ScFv domain | | GGCCCCGGGAAAGGGACTGGAATGGGTGTCCGCAATCAGCGGTAGCGGGGGCTCAACA |
| | | TACTACGCCGACTCCGTCAAGGGTCGCTTCACTATTTCCCGGGACAACTCCAAGAATA |
| | | CCCTGTACCTCCAAATGAACAGCCTCAGGGCCGAGGATACTGCCGTGTACTACTGCGC |
| | | CAAAGTCGGATACGATAGCTCCGGTTACTACCGGGACTACTACGGAATGGACGTGTGG |
| | | GGACAGGGCACCACCGTGACCGTGTCAAGCGGCGGAGGCGGTTCAGGAGGGGGAGGCT |
| | | CCGGCGGTGGAGGGTCCGAAATCGTCCTGACTCAGTCGCCTGGCACTCTGTCGTTGTC |
| | | CCCGGGGGAGCGCGCTACCCTGTCGTGTCGGGCGTCGCAGTCCGTGTCGAGCTCCTAC |
| | | CTCGCGTGGTACCAGCAGAAGCCCGGACAGGCCCCTAGACTTCTGATCTACGGCACTT |
| | | CTTCACGCGCCACCGGGATCAGCGACAGGTTCAGCGGCTCCGGCTCCGGGACCGACTT |
| | | CACCCTGACCATTAGCCGGCTGGAGCCTGAAGATTTCGCCGTGTATTACTGCCAACAC |
| | | TACGGAAACTCGCCGCCAAAGTTCACGTTCGGACCCGGAACCAAGCTGGAAATCAAG |
| BCMA_EBB- | 190 | QVQLVESGGGLVQPGGSLRLSCAASGFTFSSYAMSWVRQAPGKGLEWVSAISGSGGST |
| C1981-C3-aa | | YYADSVKGRFTISRDNSKNTLYLQMNSLRAEDTAVYYCAKVGYDSSGYYRDYYGMDVW |
| VH | | GQGTTVTVSS |
| BCMA_EBB- | 211 | EIVLTQSPGTLSLSPGERATLSCRASQSVSSSYLAWYQQKPGQAPRLLIYGTSSRATG |
| C1981-C3-aa | | ISDRFSGSGSGTDFTLTISRLEPEDFAVYYCQHYGNSPPKFTFGPGTKLEIK |
| VL | | |
| BCMA_EBB- | 232 | MALPVTALLLPLALLLHAARPQVQLVESGGGLVQPGGSLRLSCAASGFTFSSYAMSWV |
| C1981-C3-aa | | RQAPGKGLEWVSAISGSGGSTYYADSVKGRFTISRDNSKNTLYLQMNSLRAEDTAVYY |
| Full CART | | CAKVGYDSSGYYRDYYGMDVWGQGTTVTVSSGGGGSGGGGSGGGGSEIVLTQSPGTLS |
| | | LSPGERATLSCRASQSVSSSYLAWYQQKPGQAPRLLIYGTSSRATGISDRFSGSGSGT |
| | | DFTLTISRLEPEDFAVYYCQHYGNSPPKFTFGPGTKLEIKTTTPAPRPPTPAPTIASQ |
| | | PLSLRPEACRPAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAYKQGQNQLYNE |
| | | LNLGRREEYDVLDKRRGRDPEMGGKPRRKNPQEGLYNELQKDKMAEAYSEIGMKGERR |
| | | RGKGHDGLYQGLSTATKDTYDALHMQALPPR |
| BCMA_EBB- | 253 | ATGGCCCTCCCTGTCACCGCCCTGCTGCTTCCGCTGGCTCTTCTGCTCCACGCCGCTC |
| C1981-C3-nt | | GGCCCCAAGTGCAGCTCGTGGAGTCAGGCGGAGGACTGGTGCAGCCCGGGGGCTCCCT |
| Full CART | | GAGACTTTCCTGCGCGGCATCGGGTTTTACCTTCTCCTCCTATGCTATGTCCTGGGTG |
| | | CGCCAGGCCCCGGGAAAGGGACTGGAATGGGTGTCCGCAATCAGCGGTAGCGGGGGCT |
| | | CAACATACTACGCCGACTCCGTCAAGGGTCGCTTCACTATTTCCCGGGACAACTCCAA |
| | | GAATACCCTGTACCTCCAAATGAACAGCCTCAGGGCCGAGGATACTGCCGTGTACTAC |
| | | TGCGCCAAAGTCGGATACGATAGCTCCGGTTACTACCGGGACTACTACGGAATGGACG |
| | | TGTGGGGACAGGGCACCACCGTGACCGTGTCAAGCGGCGGAGGCGGTTCAGGAGGGGG |
| | | AGGCTCCGGCGGTGGAGGGTCCGAAATCGTCCTGACTCAGTCGCCTGGCACTCTGTCG |
| | | TTGTCCCCGGGGGAGCGCGCTACCCTGTCGTGTCGGGCGTCGCAGTCCGTGTCGAGCT |
| | | CCTACCTCGCGTGGTACCAGCAGAAGCCCGGACAGGCCCCTAGACTTCTGATCTACGG |
| | | CACTTCTTCACGCGCCACCGGGATCAGCGACAGGTTCAGCGGCTCCGGCTCCGGGACC |
| | | GACTTCACCCTGACCATTAGCCGGCTGGAGCCTGAAGATTTCGCCGTGTATTACTGCC |
| | | AACACTACGGAAACTCGCCGCCAAAGTTCACGTTCGGACCCGGAACCAAGCTGGAAAT |
| | | CAAGACCACTACCCCAGCACCGAGGCCACCCACCCCGGCTCCTACCATCGCCTCCCAG |
| | | CCTCTGTCCCTGCGTCCGGAGGCATGTAGACCCGCAGCTGGTGGGGCCGTGCATACCC |
| | | GGGGTCTTGACTTCGCCTGCGATATCTACATTTGGGCCCCTCTGGCTGGTACTTGCGG |
| | | GGTCCTGCTGCTTTCACTCGTGATCACTCTTTACTGTAAGCGCGGTCGGAAGAAGCTG |
| | | CTGTACATCTTTAAGCAACCCTTCATGAGGCCTGTGCAGACTACTCAAGAGGAGGACG |
| | | GCTGTTCATGCCGGTTCCCAGAGGAGGAGGAAGGCGGCTGCGAACTGCGCGTGAAATT |
| | | CAGCCGCAGCGCAGATGCTCCAGCCTACAAGCAGGGGCAGAACCAGCTCTACAACGAA |
| | | CTCAATCTTGGTCGGAGAGAGGAGTACGACGTGCTGGACAAGCGGAGAGGACGGGACC |
| | | CAGAAATGGGCGGGAAGCCGCGCAGAAAGAATCCCCAAGAGGGCCTGTACAACGAGCT |
| | | CCAAAAGGATAAGATGGCAGAAGCCTATAGCGAGATTGGTATGAAAGGGGAACGCAGA |
| | | AGAGGCAAAGGCCACGACGGACTGTACCAGGGACTCAGCACCGCCACCAAGGACACCT |
| | | ATGACGCTCTTCACATGCAGGCCCTGCCGCCTCGG |
| BCMA_EBB- | | |
| C1978-G4 | | |
| BCMA_EBB- | 149 | EVQLVESGGGLVQPGGSLRLSCAASGFTFSSYAMSWVRQAPGKGLEWVSAISGSGGST |
| C1978-G4-aa | | YYADSVKGRFTISRDNSKNTLYLQMNSLRAEDTAVYYCAKMGWSSGYLGAFDIWGQGT |
| ScFv domain | | TVTVSSGGGGSGGGGSGGGGSEIVLTQSPGTLSLSPGERATLSCRASQSVASSFLAWY |
| | | QQKPGQAPRLLIYGASGRATGIPDRFSGSGSGTDFTLTISRLEPEDFAVYYCQHYGGS |
| | | PRLTFGGGTKVDIK |
| BCMA_EBB- | 170 | GAAGTCCAACTGGTGGAGTCCGGGGGAGGGCTCGTGCAGCCCGGAGGCAGCCTTCGGC |
| C1978-G4-nt | | TGTCGTGCGCCGCCTCCGGGTTCACGTTCTCATCCTACGCGATGTCGTGGGTCAGACA |
| ScFv domain | | GGCACCAGGAAAGGGACTGGAATGGGTGTCCGCCATTAGCGGCTCCGGCGGTAGCACC |
| | | TACTATGCCGACTCAGTGAAGGGAAGGTTCACTATCTCCCGCGACAACAGCAAGAACA |
| | | CCCTGTACCTCCAAATGAACTCTCTGCGGGCCGAGGATACCGCGGTGTACTATTGCGC |
| | | CAAGATGGGTTGGTCCAGCGGATACTTGGGAGCCTTCGACATTTGGGGACAGGGCACT |
| | | ACTGTGACCGTGTCCTCCGGGGGTGGCGGATCGGGAGGCGGCGGCTCGGGTGGAGGGG |
| | | GTTCCGAAATCGTGTTGACCCAGTCACCGGGAACCCTCTCGCTGTCCCCGGGAGAACG |
| | | GGCTACACTGTCATGTAGAGCGTCCCAGTCCGTGGCTTCCTCGTTCCTGGCCTGGTAC |
| | | CAGCAGAAGCCGGGACAGGCACCCCGCCTGCTCATCTACGGAGCCAGCGGCCGGGCGA |
| | | CCGGCATCCCTGACCGCTTCTCCGGTTCCGGCTCGGGCACCGACTTTACTCTGACCAT |
| | | TAGCAGGCTTGAGCCCGAGGATTTTGCCGTGTACTACTGCCAACACTACGGGGGGAGC |
| | | CCTCGCCTGACCTTCGGAGGCGGAACTAAGGTCGATATCAAAA |
| BCMA_EBB- | 191 | EVQLVESGGGLVQPGGSLRLSCAASGFTFSSYAMSWVRQAPGKGLEWVSAISGSGGST |
| C1978-G4-aa | | YYADSVKGRFTISRDNSKNTLYLQMNSLRAEDTAVYYCAKMGWSSGYLGAFDIWGQGT |
| VH | | TVTVSS |
| BCMA_EBB- | 212 | EIVLTQSPGTLSLSPGERATLSCRASQSVASSFLAWYQQKPGQAPRLLIYGASGRATG |
| C1978-G4-aa | | IPDRFSGSGSGTDFTLTISRLEPEDFAVYYCQHYGGSPRLTFGGGTKVDIK |
| VL | | |
| BCMA_EBB- | 233 | MALPVTALLLPLALLLHAARPEVQLVESGGGLVQPGGSLRLSCAASGFTFSSYAMSWV |
| C1978-G4-aa | | RQAPGKGLEWVSAISGSGGSTYYADSVKGRFTISRDNSKNTLYLQMNSLRAEDTAVYY |
| Full CART | | CAKMGWSSGYLGAFDIWGQGTTVTVSSGGGGSGGGGSGGGGSEIVLTQSPGTLSLSPG |
| | | ERATLSCRASQSVASSFLAWYQQKPGQAPRLLIYGASGRATGIPDRFSGSGSGTDFTL |
| | | TISRLEPEDFAVYYCQHYGGSPRLTFGGGTKVDIKTTTPAPRPPTPAPTIASQPLSLR |
| | | PEACRPAAGGAVHTRGLDFACDIYIWAPLAGTCGVLLLSLVITLYCKRGRKKLLYIFK |
| | | QPFMRPVQTTQEEDGCSCRFPEEEEGGCELRVKFSRSADAPAYKQGQNQLYNELNLGR |
| | | REEYDVLDKRRGRDPEMGGKPRRKNPQEGLYNELQKDKMAEAYSEIGMKGERRRGKGH |
| | | DGLYQGLSTATKDTYDALHMQALPPR |
| BCMA_EBB- | 254 | ATGGCCCTCCCTGTCACCGCCCTGCTGCTTCCGCTGGCTCTTCTGCTCCACGCCGCTC |
| C1978-G4-nt | | GGCCCGAAGTCCAACTGGTGGAGTCCGGGGGAGGGCTCGTGCAGCCCGGAGGCAGCCT |
| Full CART | | TCGGCTGTCGTGCGCCGCCTCCGGGTTCACGTTCTCATCCTACGCGATGTCGTGGGTC |
| | | AGACAGGCACCAGGAAAGGGACTGGAATGGGTGTCCGCCATTAGCGGCTCCGGCGGTA |
| | | GCACCTACTATGCCGACTCAGTGAAGGGAAGGTTCACTATCTCCCGCGACAACAGCAA |
| | | GAACACCCTGTACCTCCAAATGAACTCTCTGCGGGCCGAGGATACCGCGGTGTACTAT |
| | | TGCGCCAAGATGGGTTGGTCCAGCGGATACTTGGGAGCCTTCGACATTTGGGGACAGG |
| | | GCACTACTGTGACCGTGTCCTCCGGGGGTGGCGGATCGGGAGGCGGCGGCTCGGGTGG |
| | | AGGGGGTTCCGAAATCGTGTTGACCCAGTCACCGGGAACCCTCTCGCTGTCCCCGGGA |
| | | GAACGGGCTACACTGTCATGTAGAGCGTCCCAGTCCGTGGCTTCCTCGTTCCTGGCCT |
| | | GGTACCAGCAGAAGCCGGGACAGGCACCCCGCCTGCTCATCTACGGAGCCAGCGGCCG |
| | | GGCGACCGGCATCCCTGACCGCTTCTCCGGTTCCGGCTCGGGCACCGACTTTACTCTG |
| | | ACCATTAGCAGGCTTGAGCCCGAGGATTTTGCCGTGTACTACTGCCAACACTACGGGG |
| | | GGAGCCCTCGCCTGACCTTCGGAGGCGGAACTAAGGTCGATATCAAAACCACTACCCC |
| | | AGCACCGAGGCCACCCACCCCGGCTCCTACCATCGCCTCCCAGCCTCTGTCCCTGCGT |
| | | CCGGAGGCATGTAGACCCGCAGCTGGTGGGGCCGTGCATACCCGGGGTCTTGACTTCG |
| | | CCTGCGATATCTACATTTGGGCCCCTCTGGCTGGTACTTGCGGGGTCCTGCTGCTTTC |
| | | ACTCGTGATCACTCTTTACTGTAAGCGCGGTCGGAAGAAGCTGCTGTACATCTTTAAG |
| | | CAACCCTTCATGAGGCCTGTGCAGACTACTCAAGAGGAGGACGGCTGTTCATGCCGGT |
| | | TCCCAGAGGAGGAGGAAGGCGGCTGCGAACTGCGCGTGAAATTCAGCCGCAGCGCAGA |
| | | TGCTCCAGCCTACAAGCAGGGGCAGAACCAGCTCTACAACGAACTCAATCTTGGTCGG |
| | | AGAGAGGAGTACGACGTGCTGGACAAGCGGAGAGGACGGGACCCAGAAATGGGCGGGA |
| | | AGCCGCGCAGAAAGAATCCCCAAGAGGGCCTGTACAACGAGCTCCAAAAGGATAAGAT |
| | | GGCAGAAGCCTATAGCGAGATTGGTATGAAAGGGGAACGCAGAAGAGGCAAAGGCCAC |
| | | GACGGACTGTACCAGGGACTCAGCACCGCCACCAAGGACACCTATGACGCTCTTCACA |
| | | TGCAGGCCCTGCCGCCTCGG |

TABLE 7
Heavy Chain Variable Domain CDRs according to the Kabat numbering scheme
(Kabat et al. (1991), “Sequences of Proteins of Immunological Interest,”
5th Ed. Public Health Service, National Institutes of Health, Bethesda, MD)

| | | SEQ | | SEQ | | SEQ |
|---|---|---|---|---|---|---|
| Candidate | HCDR1 | ID NO | HCDR2 | ID NO | HCDR3 | ID NO |
| 139109 | NHGMS | 1118 | GIVYSGSTYYAASVKG | 1158 | HGGESDV | 1198 |
| 139103 | NYAMS | 1119 | GISRSGENTYYADSVKG | 1159 | SPAHYYGGMDV | 1199 |
| 139105 | DYAMH | 1120 | GISWNSGSIGYADSVKG | 1160 | HSFLAY | 1200 |
| 139111 | NHGMS | 1121 | GIVYSGSTYYAASVKG | 1161 | HGGESDV | 1201 |
| 139100 | NFGIN | 1122 | WINPKNNNTNYAQKFQG | 1162 | GPYYYQSYMDV | 1202 |
| 139101 | SDAMT | 1123 | VISGSGGTTYYADSVKG | 1163 | LDSSGYYYARGPRY | 1203 |
| 139102 | NYGIT | 1124 | WISAYNGNTNYAQKFQG | 1164 | GPYYYYMDV | 1204 |
| 139104 | NHGMS | 1125 | GIVYSGSTYYAASVKG | 1165 | HGGESDV | 1205 |
| 139106 | NHGMS | 1126 | GIVYSGSTYYAASVKG | 1166 | HGGESDV | 1206 |
| 139107 | NHGMS | 1127 | GIVYSGSTYYAASVKG | 1167 | HGGESDV | 1207 |
| 139108 | DYYMS | 1128 | YISSSGSTIYYADSVKG | 1168 | ESGDGMDV | 1208 |
| 139110 | DYYMS | 1129 | YISSSGNTIYYADSVKG | 1169 | STMVREDY | 1209 |
| 139112 | NHGMS | 1130 | GIVYSGSTYYAASVKG | 1170 | HGGESDV | 1210 |
| 139113 | NHGMS | 1131 | GIVYSGSTYYAASVKG | 1171 | HGGESDV | 1211 |
| 139114 | NHGMS | 1132 | GIVYSGSTYYAASVKG | 1172 | HGGESDV | 1212 |
| 149362 | SSYYYWG | 1133 | SIYYSGSAYYNPSLKS | 1173 | HWQEWPDAFDI | 1213 |
| 149363 | TSGMCVS | 1134 | RIDWDEDKFYSTSLKT | 1174 | SGAGGTSATAFDI | 1214 |
| 149364 | SYSMN | 1135 | SISSSSSYIYYADSVKG | 1175 | TIAAVYAFDI | 1215 |
| 149365 | DYYMS | 1136 | YISSSGSTIYYADSVKG | 1176 | DLRGAFDI | 1216 |
| 149366 | SHYIH | 1137 | MINPSGGVTAYSQTLQG | 1177 | EGSGSGWYFDF | 1217 |
| 149367 | SGGYYWS | 1138 | YIYYSGSTYYNPSLKS | 1178 | AGIAARLRGAFDI | 1218 |
| 149368 | SYAIS | 1139 | GIIPIFGTANYAQKFQG | 1179 | RGGYQLLRWDVGLLRSAFDI | 1219 |
| 149369 | SNSAAWN | 1140 | RTYYRSKWYSFYAISLKS | 1180 | SSPEGLFLYWFDP | 1220 |
| BCMA_EB | SYAMS | 1141 | AISGSGGSTYYADSVKG | 1181 | VEGSGSLDY | 1221 |
| B-C1978-A4 | | | | | | |
| BCMA_EB | RYPMS | 1142 | GISDSGVSTYYADSAKG | 1182 | RAGSEASDI | 1222 |
| B-C1978-G1 | | | | | | |
| BCMA_EB | SYAMS | 1143 | AISGSGGSTYYADSVKG | 1183 | ATYKRELRYYYGMDV | 1223 |
| B-C1979-C1 | | | | | | |
| BCMA_EB | SYAMS | 1144 | AISGSGGSTYYADSVKG | 1184 | ATYKRELRYYYGMDV | 1224 |
| B-C1978-C7 | | | | | | |
| BCMA_EB | DYAMH | 1145 | GISWNSGSIGYADSVKG | 1185 | VGKAVPDV | 1225 |
| B-C1978-D10 | | | | | | |
| BCMA_EB | DYAMH | 1146 | SINWKGNSLAYGDSVKG | 1186 | HQGVAYYNYAMDV | 1226 |
| B-C1979-C12 | | | | | | |
| BCMA_EB | SYAMS | 1147 | AISGSGGSTYYADSVKG | 1187 | VVRDGMDV | 1227 |
| B-C1980-G4 | | | | | | |
| BCMA_EB | SYAMS | 1148 | AISGSGGSTYYADSVKG | 1188 | IPQTGTFDY | 1228 |
| B-C1980-D2 | | | | | | |
| BCMA_EB | SYAMS | 1149 | AISGSGGSTYYADSVKG | 1189 | ANYKRELRYYYGMDV | 1229 |
| B-C1978-A10 | | | | | | |
| BCMA_EB | SYAMS | 1150 | AISGSGGSTYYADSVKG | 1190 | ALVGATGAFDI | 1230 |
| B-C1978-D4 | | | | | | |
| BCMA_EB | SYAMS | 1151 | AISGSGGSTYYADSVKG | 1191 | WFGEGFDP | 1231 |
| B-C1980-A2 | | | | | | |
| BCMA_EB | SYAMS | 1152 | AISGSGGSTYYADSVKG | 1192 | VGYDSSGYYRDYYGMDV | 1232 |
| B-C1981-C3 | | | | | | |
| BCMA_EB | SYAMS | 1153 | AISGSGGSTYYADSVKG | 1193 | MGWSSGYLGAFDI | 1233 |
| B-C1978-G4 | | | | | | |
| A7D12.2 | NFGMN | 1154 | WINTYTGESYFADDFKG | 1194 | GEIYYGYDGGFAY | 1234 |
| C11D5.3 | DYSIN | 1155 | WINTETREPAYAYDFRG | 1195 | DYSYAMDY | 1235 |
| C12A3.2 | HYSMN | 1156 | RINTESGVPIYADDFKG | 1196 | DYLYSLDF | 1236 |
| C13F12.1 | HYSMN | 1157 | RINTETGEPLYADDFKG | 1197 | DYLYSCDY | 1237 |

TABLE 8
Light Chain Variable Domain CDRs according to the Kabat numbering scheme
(Kabat et al. (1991), “Sequences of Proteins of Immunological Interest,”
5th Ed. Public Health Service, National Institutes of Health, Bethesda, MD)

| | | SEQ | | SEQ | | SEQ ID |
|---|---|---|---|---|---|---|
| Candidate | LCDR1 | ID NO | LCDR2 | ID NO | LCDR3 | NO |
| 139109 | RASQSISSYLN | 1238 | AASSLQS | 1278 | QQSYSTPYT | 1318 |
| 139103 | RASQSISSSFLA | 1239 | GASRRAT | 1279 | QQYHSSPSWT | 1319 |
| 139105 | RSSQSLLHSNGYNYLD | 1240 | LGSNRAS | 1280 | MQALQTPYT | 1320 |
| 139111 | KSSQSLLRNDGKTPLY | 1241 | EVSNRFS | 1281 | MQNIQFPS | 1321 |
| 139100 | RSSQSLLHSNGYNYLN | 1242 | LGSKRAS | 1282 | MQALQTPYT | 1322 |
| 139101 | RASQSISSYLN | 1243 | GASTLAS | 1283 | QQSYKRAS | 1323 |
| 139102 | RSSQSLLYSNGYNYVD | 1244 | LGSNRAS | 1284 | MQGRQFPYS | 1324 |
| 139104 | RASQSVSSNLA | 1245 | GASTRAS | 1285 | QQYGSSLT | 1325 |
| 139106 | RASQSVSSKLA | 1246 | GASIRAT | 1286 | QQYGSSSWT | 1326 |
| 139107 | RASQSVGSTNLA | 1247 | DASNRAT | 1287 | QQYGSSPPWT | 1327 |
| 139108 | RASQSISSYLN | 1248 | AASSLQS | 1288 | QQSYTLA | 1328 |
| 139110 | KSSESLVHNSGKTYLN | 1249 | EVSNRDS | 1289 | MQGTHWPGT | 1329 |
| 139112 | QASEDINKFLN | 1250 | DASTLQT | 1290 | QQYESLPLT | 1330 |
| 139113 | RASQSVGSNLA | 1251 | GASTRAT | 1291 | QQYNDWLPVT | 1331 |
| 139114 | RASQSIGSSSLA | 1252 | GASSRAS | 1292 | QQYAGSPPFT | 1332 |
| 149362 | KASQDIDDAMN | 1253 | SATSPVP | 1293 | LQHDNFPLT | 1333 |
| 149363 | RASQDIYNNLA | 1254 | AANKSQS | 1294 | QHYYRFPYS | 1334 |
| 149364 | RSSQSLLHSNGYNYLD | 1255 | LGSNRAS | 1295 | MQALQTPYT | 1335 |
| 149365 | GGNNIGTKSVH | 1256 | DDSVRPS | 1296 | QVWDSDSEHVV | 1336 |
| 149366 | SGDGLSKKYVS | 1257 | RDKERPS | 1297 | QAWDDTTVv | 1337 |
| 149367 | RASQGIRNWLA | 1258 | AASNLQS | 1298 | QKYNSAPFT | 1338 |
| 149368 | GGNNIGSKSVH | 1259 | GKNNRPS | 1299 | SSRDSSGDHLRV | 1339 |
| 149369 | QGDSLGNYYAT | 1260 | GTNNRPS | 1300 | NSRDSSGHHLL | 1340 |
| BCMA_EBB- | RASQSVSSAYLA | 1261 | GASTRAT | 1301 | QHYGSSFNGSSLFT | 1341 |
| C1978-A4 | | | | | | |
| BCMA_EBB- | RASQSVSNSLA | 1262 | DASSRAT | 1302 | QQFGTSSGLT | 1342 |
| C1978-G1 | | | | | | |
| BCMA_EBB- | RASQSVSSSFLA | 1263 | GASSRAT | 1303 | QQYHSSPSWT | 1343 |
| C1979-C1 | | | | | | |
| BCMA_EBB- | RASQSVSTTFLA | 1264 | GSSNRAT | 1304 | QQYHSSPSWT | 1344 |
| C1978-C7 | | | | | | |
| BCMA_EBB- | RASQSISSYLN | 1265 | AASSLQS | 1305 | QQSYSTPYS | 1345 |
| C1978-D10 | | | | | | |
| BCMA_EBB- | RATQSIGSSFLA | 1266 | GASQRAT | 1306 | QHYESSPSWT | 1346 |
| C1979-C12 | | | | | | |
| BCMA_EBB- | RASQSVSSSYLA | 1267 | GASSRAT | 1307 | QQYGSPPRFT | 1347 |
| C1980-G4 | | | | | | |
| BCMA_EBB- | RASQSVSSSYLA | 1268 | GASSRAT | 1308 | QHYGSSPSWT | 1348 |
| C1980-D2 | | | | | | |
| BCMA_EBB- | RASQRVASNYLA | 1269 | GASSRAT | 1309 | QHYDSSPSWT | 1349 |
| C1978-A10 | | | | | | |
| BCMA_EBB- | RASQSLSSNFLA | 1270 | GASNWAT | 1310 | QYYGTSPMYT | 1350 |
| C1978-D4 | | | | | | |
| BCMA_EBB- | RSSQSLLHSNGYNYLD | 1271 | LGSNRAS | 1311 | MQALQTPLT | 1351 |
| C1980-A2 | | | | | | |
| BCMA_EBB- | RASQSVSSSYLA | 1272 | GTSSRAT | 1312 | QHYGNSPPKFT | 1352 |
| C1981-C3 | | | | | | |
| BCMA_EBB- | RASQSVASSFLA | 1273 | GASGRAT | 1313 | QHYGGSPRLT | 1353 |
| C1978-G4 | | | | | | |
| A7D12.2 | RASQDVNTAVS | 1274 | SASYRYT | 1314 | QQHYSTPWT | 1354 |
| C11D5.3 | RASESVSVIGAHLIH | 1275 | LASNLET | 1315 | LQSRIFPRT | 1355 |
| C12A3.2 | RASESVTILGSHLIY | 1276 | LASNVQT | 1316 | LQSRTIPRT | 1356 |
| C13F12.1 | RASESVTILGSHLIY | 1277 | LASNVQT | 1317 | LQSRTIPRT | 1357 |

### CD20 CAR and CD20-Binding Sequences

In some embodiments, the CAR IL-15R/IL-15-expressing cell described herein is a CD20 CAR-expressing cell (e.g., a cell expressing a CAR that binds to human CD20). In some embodiments, the CD20 CAR-expressing cell includes an antigen binding domain according to WO2016/164731 and PCT/US2017/055627, incorporated herein by reference. Exemplary CD20-binding sequences or CD20 CAR sequences are disclosed in, e.g., Tables 1-5 of PCT/US2017/055627. In some embodiments, the CD20-binding sequences or CD20 CAR comprises a CDR, variable region, scFv, or full-length sequence of a CD20 CAR disclosed in PCT/US2017/055627 or WO2016/164731.

### CD22 CAR and CD22-Binding Sequences

In some embodiments, the CAR IL-15R/IL-15-expressing cell described herein is a CD22 CAR-expressing cell (e.g., a cell expressing a CAR that binds to human CD22). In some embodiments, the CD22 CAR-expressing cell includes an antigen binding domain according to WO2016/164731 and PCT/US2017/055627, incorporated herein by reference. Exemplary CD22-binding sequences or CD22 CAR sequences are disclosed in, e.g., Tables 6A, 6B, 7A, 7B, 7C, 8A, 8B, 9A, 9B, 10A, and 10B of WO2016/164731 and Tables 6-10 of PCT/US2017/055627. In some embodiments, the CD22-binding sequences or CD22 CAR sequences comprise a CDR, variable region, scFv or full-length sequence of a CD22 CAR disclosed in PCT/US2017/055627 or WO2016/164731.

### EGFR CAR and EGFR-Binding Sequences

In some embodiments, the CAR IL-15R/IL-15-expressing cell described herein is an EGFR CAR-expressing cell (e.g., a cell expressing a CAR that binds to human EGFR). In some embodiments, the CAR-expressing cell described herein is an EGFRVIII CAR-expressing cell (e.g., a cell expressing a CAR that binds to human EGFRvIII). Exemplary EGFRvIII CARs can include sequences disclosed in WO2014/130657, e.g., Table 2 of WO2014/130657, incorporated herein by reference.

Exemplary EGFRvIII-binding sequences or EGFR CAR sequences may comprise a CDR, a variable region, an scFv, or a full-length CAR sequence of a sequence disclosed in Table 9 (or a sequence at least about 85%, 90%, 95%, 99% or more identical thereto, and/or having one, two, three or more substitutions, insertions, deletions, or modifications).

TABLE 9
Humanized EGFRvIII CAR Constructs

| | | |
|---|---|---|
| | SEQ ID | |
| Name | NO: | Sequence |

CAR 1

| | | |
|---|---|---|
| CAR1 scFv | SEQ ID | eiqlvqsgaevkkpgatvkisckgsgfniedyyihwvqqapgkglewmgridpendetkygpifqgrvtita |
| domain | NO: 1358 | dtstntvymelsslrsedtavyycafrggvywgqgttvtvssggggsggggsggggsggggsdvvmtqspd |
| | | slayslgeratinckssqslldsdgktylnwlqqkpgqpplalislvskldsgvpdrfsgsgsgtdftltisslqaed |
| | | vavyycwqgthfpgtfgggtkveik |
| CAR1 | SEQ ID | gaaatccagctggtccaatcgggagctgaggtcaagaagccgggagccaccgtcaagatctcatgcaaggggt |
| scFv domain | NO: 1359 | cgggattcaacatcgaggactactacattcactgggtgcagcaagctccgggaaaaggcctggaatggatgggc |
| nt | | agaatcgacccagaaaacgacgaaactaagtacggaccgattttccaaggaagagtgactatcaccgccgatact |
| | | tcaaccaataccgtctacatggaactgagctcgctccggtccgaagatactgcagtgtattactgtgcctttcgcgga |
| | | ggggtgtactggggccaaggaactactgtcactgtctcgtcaggaggcggagggtcgggaggaggcgggagc |
| | | ggaggcggtggctcgggtggcggaggaagcgacgtggtgatgacccagtccccggactccctcgccgtgagc |
| | | ctcggagagagggcgactatcaattgcaagtcgtcccagtcacttctggattccgatggtaaaacgtacctcaactg |
| | | gctgcagcaaaagccagggcagccacccaaacggttgatctcccttgtgtccaaactggatagcggagtgcctga |
| | | ccgcttctcgggttccggtagcgggaccgacttcaccctgacgatcagctcactgcaggcggaggacgtggcag |
| | | tgtactactgctggcagggaacccacttccctggcacctttggaggtggcaccaaggtggagatcaag |
| CAR1 | SEQ ID | atggccctccctgtcaccgccctgctgcttccgctggctcttctgctccacgccgctcggcccgaaatccagctggt |
| Soluble scFv - | NO: 1360 | ccaatcgggagctgaggtcaagaagccgggagccaccgtcaagatctcatgcaaggggtcgggattcaacatc |
| nt | | gaggactactacattcactgggtgcagcaagctccgggaaaaggcctggaatggatgggcagaatcgacccag |
| | | aaaacgacgaaactaagtacggaccgattttccaaggaagagtgactatcaccgccgatacttcaaccaataccgt |
| | | ctacatggaactgagctcgctccggtccgaagatactgcagtgtattactgtgcctttcgcggaggggtgtactggg |
| | | gccaaggaactactgtcactgtctcgtcaggaggcggagggtcgggaggaggcgggagcggaggcggtggct |
| | | cgggtggcggaggaagcgacgtggtgatgacccagtccccggactccctcgccgtgagcctcggagagaggg |
| | | cgactatcaattgcaagtcgtcccagtcacttctggattccgatggtaaaacgtacctcaactggctgcagcaaaag |
| | | ccagggcagccacccaaacggttgatctcccttgtgtccaaactggatagcggagtgcctgaccgcttctcgggtt |
| | | ccggtagcgggaccgacttcaccctgacgatcagctcactgcaggcggaggacgtggcagtgtactactgctgg |
| | | cagggaacccacttccctggcacctttggaggtggcaccaaggtggagatcaagggatcgcaccaccatcacca |
| | | tcatcatcac |
| CAR1 | SEQ ID | Malpvtalllplalllhaarpeiqlvqsgaevkkpgatvkisckgsgfniedyyihwvqqapgkglewmgrid |
| Soluble scFv - | NO: 1361 | pendetkygpifqgrvtitadtstntvymelsslrsedtavyycafrggvywgqgttvtvssggggsggggsgg |
| aa | | ggsggggsdvvmtqspdslayslgeratinckssqslldsdgktylnwlqqkpgqppkrlislvskldsgvpdr |
| | | fsgsgsgtdftltisslqaedvavyycwqgthfpgtfgggtkveikgshhhhhhhh |
| CAR1 - Full - | SEQ ID | atggccctccctgtcaccgccctgctgcttccgctggctcttctgctccacgccgctcggcccgagatccagctggt |
| nt | NO: 1362 | gcagtcgggagctgaagtcaaaaagcctggcgcaaccgtcaagatctcgtgcaaaggatcagggttcaacatcg |
| lentivirus | | aggactactacatccattgggtgcaacaggcacccggaaaaggcctggagtggatggggaggattgacccaga |
| | | aaatgacgaaaccaagtacggaccgatcttccaaggacgggtgaccatcacggctgacacttccactaacaccgt |
| | | ctacatggaactctcgagccttcgctcggaagataccgcggtgtactactgcgcctttagaggtggagtctactggg |
| | | gacaagggactaccgtcaccgtgtcgtcaggtggcggaggatcaggcggaggcggctccggtggaggaggaa |
| | | gcggaggaggtggctccgacgtggtgatgacgcagtcaccggactccttggcggtgagcctgggtgaacgcgc |
| | | cactatcaactgcaagagctcccagagcttgctggactccgatggaaagacttatctcaattggctgcaacagaag |
| | | cctggccagccgccaaagagactcatctcactggtgagcaagctggatagcggagtgccagatcggttttcggga |
| | | tcgggctcaggcaccgacttcaccctgactatttcctccctccaagccgaggatgtggccgtctactactgttggca |
| | | ggggactcacttcccggggaccttcggtggaggcactaaggtggagatcaaaaccactaccccagcaccgagg |
| | | ccacccaccccggctcctaccatcgcctcccagcctctgtccctgcgtccggaggcatgtagacccgcagctggt |
| | | ggggccgtgcatacccggggtcttgacttcgcctgcgatatctacatttgggcccctctggctggtacttgcggggt |
| | | cctgctgctttcactcgtgatcactctttactgtaagcgcggtcggaagaagctgctgtacatctttaagcaaccc |
| | | ttcatgaggcctgtgcagactactcaagaggaggacggctgttcatgccggttcccagaggaggaggaaggcggct |
| | | gcgaactgcgcgtgaaattcagccgcagcgcagatgctccagcctacaagcaggggcagaaccagctctacaa |
| | | cgaactcaatcttggtcggagagaggagtacgacgtgctggacaagcggagaggacgggacccagaaatggg |
| | | cgggaagccgcgcagaaagaatccccaagagggcctgtacaacgagctccaaaaggataagatggcagaagc |
| | | ctatagcgagattggtatgaaaggggaacgcagaagaggcaaaggccacgacggactgtaccagggactcagc |
| | | accgccaccaaggacacctatgacgctcttcacatgcaggccctgccgcctcgg |
| CAR1 - Full - | SEQ ID | malpvtalllplalllhaarpeiqlvqsgaevkkpgatvkisckgsgfniedyyihwvqqapgkglewmgri |
| aa | NO: 1363 | dpendetkvgpifqgrvtitadtstntvymelsslisedtavyycafrggvywgqgttvtvssggggsggggs |
| | | ggggsggggsdvvmtqspdslayslgeratinckssqslldsdgktylnwlqqkpgqppkrlislvskldsg |
| | | vpdrfsgsgsgtdftltisslqaedvavyycwqgthfpgtfgggtkveiktttpaprpptpaptiasqplslrpea |
| | | crpaaggavhfigldfacdiyiwaplagtcgvlllslvitlyckrgrkkllyifkqpfmrpvqttqeedgcscrfpe |
| | | eeeggcelrvkfsrsadapaykqgqnqlynelnlgrreeydvldlargrdpemggkprrknpqeglynelqk |
| | | dkmaeayseigmkgerrrgkghdglyqglstatkdtydalhmqalppr |

CAR 2

| | | |
|---|---|---|
| CAR2 scFv | SEQ ID | dvvmtqspdslayslgeratinckssqslldsdgktylnwlqqkpgqpplalislvskldsgvpdrfsgsgsgtd |
| domain | NO: 1364 | ftltisslqaedvavyycwqgthfpgtfgggtkveikggggsggggsggggsggggseiqlvqsgaevkkpg |
| | | atvkisckgsgfniedyyihwvqqapgkglewmgridpendetkygpifqgrvtitadtstntvymelsslrs |
| | | edtavyycafrggvywgqgttvtvss |
| CAR2 scFv | SEQ ID | gatgtcgtgatgacccagtccccagactccctcgcagtgtccttgggagaacgggccaccatcaactgcaaatcg |
| domain - nt | NO: 1365 | agccagtcactgctggactcagacggaaagacctacctcaactggctgcagcagaagcctggccagccaccga |
| | | agcgcctgatctccctggtgtccaagctggactcgggcgtcccggacaggtttagcggtagcggctcgggaacc |
| | | gacttcactctgaccattagctcgctccaagctgaagatgtggcggtctactactgctggcaggggacccacttccc |
| | | cgggacctttggcggaggaactaaagtcgaaatcaaaggaggaggcggatcaggtggaggaggcagcggag |
| | | gaggagggagcggcggtggcggctccgaaattcaacttgtgcaatccggtgccgaggtgaagaaacctggtgc |
| | | cactgtcaagatctcgtgtaagggatcgggattcaatatcgaggactactacatccactgggtgcaacaggcgcca |
| | | ggaaagggattggagtggatgggtcgcatcgacccggaaaacgatgagactaagtacggaccgatcttccaagg |
| | | ccgggtcacgatcactgcggatacctccactaataccgtgtatatggagctctcgtcactgagaagcgaagatacg |
| | | gccgtgtactactgcgcattcagaggaggtgtgtactggggccagggaactactgtgaccgtgtcgtcg |
| CAR2 - | SEQ ID | atggccctccctgtcaccgccctgctgcttccgctggctcttctgctccacgccgctcggcccgatgtcgtgatgac |
| Soluble scFv - | NO: 1366 | ccagtccccagactccctcgcagtgtccttgggagaacgggccaccatcaactgcaaatcgagccagtcactgct |
| nt | | ggactcagacggaaagacctacctcaactggctgcagcagaagcctggccagccaccgaagcgcctgatctcc |
| | | ctggtgtccaagctggactcgggcgtcccggacaggtttagcggtagcggctcgggaaccgacttcactctgacc |
| | | attagctcgctccaagctgaagatgtggcggtctactactgctggcaggggacccacttccccgggacctttggcg |
| | | gaggaactaaagtcgaaatcaaaggaggaggcggatcaggtggaggaggcagcggaggaggagggagcgg |
| | | cggtggcggctccgaaattcaacttgtgcaatccggtgccgaggtgaagaaacctggtgccactgtcaagatctc |
| | | gtgtaagggatcgggattcaatatcgaggactactacatccactgggtgcaacaggcgccaggaaagggattgg |
| | | agtggatgggtcgcatcgacccggaaaacgatgagactaagtacggaccgatcttccaaggccgggtcacgatc |
| | | actgcggatacctccactaataccgtgtatatggagctctcgtcactgagaagcgaagatacggccgtgtactactg |
| | | cgcattcagaggaggtgtgtactggggccagggaactactgtgaccgtgtcgtcggggtcacatcaccaccatca |
| | | tcatcaccac |
| CAR2 - | SEQ ID | malpvtalllplalllhaarpdvvmtqspdslayslgeratinckssqslldsdgktylnwlqqkpgqpplalisl |
| Soluble scFv - | NO: 1367 | vskldsgvpdrfsgsgsgtdftltisslqaedvavyycwqgthfpgtfgggtkveikggggsggggsggggsg |
| aa | | gggseiqlvqsgaevkkpgatvkisckgsgfniedyyihwvqqapgkglewmgridpendetkygpifqgr |
| | | vtitadtstntvymelsslrsedtavyycafrggvywgqgttvtvssgshhhhhhhh |
| CAR2 - Full - | SEQ ID | atggccctccctgtcaccgccctgctgcttccgctggctcttctgctccacgccgctcggcccgacgtggtcatgac |
| nt | NO: 1368 | tcaaagcccagattccttggctgtctcccttggagaaagagcaacgatcaattgcaaaagctcgcagtccctgttgg |
| | | actccgatggaaaaacctacctcaactggctgcagcagaagccgggacaaccaccaaagcggctgatttccctc |
| | | gtgtccaagctggacagcggcgtgccggatcgcttctcgggcagcggctcgggaaccgattttactctcactatttc |
| | | gtcactgcaagcggaggacgtggcggtgtattactgctggcagggcactcacttcccgggtacttttggtggaggt |
| | | accaaagtcgaaatcaagggtggaggcgggagcggaggaggcgggtcgggaggaggaggatcgggtggcg |
| | | gaggctcagaaatccagctggtgcagtcaggtgccgaagtgaagaagcctggggccacggtgaagatctcgtg |
| | | caaggggagcggattcaacatcgaggattactacatccattgggtgcaacaggcccctggcaaagggctggaat |
| | | ggatgggaaggatcgaccccgagaatgacgagactaagtacggcccgatcttccaaggacgggtgaccatcact |
| | | gcagacacttcaaccaacaccgtctacatggaactctcctcgctgcgctccgaggacaccgccgtgtactactgtg |
| | | ctttcagaggaggagtctactggggacagggaacgaccgtgaccgtcagctcaaccactaccccagcaccgag |
| | | gccacccaccccggctcctaccatcgcctcccagcctctgtccctgcgtccggaggcatgtagacccgcagctgg |
| | | tggggccgtgcatacccggggtcttgacttcgcctgcgatatctacatttgggcccctctggctggtacttgcgggg |
| | | tcctgctgctttcactcgtgatcactctttactgtaagcgcggtcggaagaagctgctgtacatctttaagcaaccc |
| | | ttcatgaggcctgtgcagactactcaagaggaggacggctgttcatgccggttcccagaggaggaggaaggcggct |
| | | gcgaactgcgcgtgaaattcagccgcagcgcagatgctccagcctacaagcaggggcagaaccagctctacaa |
| | | cgaactcaatcttggtcggagagaggagtacgacgtgctggacaagcggagaggacgggacccagaaatggg |
| | | cgggaagccgcgcagaaagaatccccaagagggcctgtacaacgagctccaaaaggataagatggcagaagc |
| | | ctatagcgagattggtatgaaaggggaacgcagaagaggcaaaggccacgacggactgtaccagggactcagc |
| | | accgccaccaaggacacctatgacgctcttcacatgcaggccctgccgcctcgg |
| CAR2 - Full - | SEQ ID | malpvtalllplalllhaarpdvvmtqspdslavslgeratinckssqslldsdgktylnwlqqkpgqppkrlis |
| aa | NO: 1369 | lvskldsgvpdrfsgsgsgtdftltisslqaedvavyycwqgthfpgtfgggtkveikggggsggggsggggs |
| | | ggggseiqlvqsgaevkkpgatvkisckgsgfniedyyihwvqqapgkglewmgridpendetkygpif |
| | | qgrvtitadtstntvymelsslrsedtavyycafrggvywgqgttvtvsstttpaprpptpaptiasqplslrpeac |
| | | rpaaggavhtrgldfacdiyiwaplagtcgvlllslvitlyckrgrkkllyifkqpfmrpvqttqeedgcscrfpee |
| | | eeggcelrvkfsrsadapaykqgqnqlynelnlgrreeydvldlargrdpemggkprrknpqeglynelqkd |
| | | kmaeayseigmkgerrrgkghdglyqglstatkdtydalhmqalppr |

CAR 3

| | | |
|---|---|---|
| CAR3 scFv | SEQ ID | eiqlvqsgaevkkpgeslrisckgsgfniedyyihwvrqmpgkglewmgridpendetkygpifqghvtis |
| domain | NO: 1370 | adtsintvylqwsslkasdtamyycafrggvywgqgttvtvssggggsggggsggggsggggsdvvmtqs |
| | | plslpvtlgqpasisckssqslldsdgktylnwlqqrpgqsprrlislvskldsgvpdrfsgsgsgtdftlkisrvea |
| | | edvgvyycwqgthfpgtfgggtkveik |
| CAR3 scFv | SEQ ID | gagattcagctggtccaaagcggcgcagaagtgaaaaagccaggggaatcgttgcgcatcagctgtaaaggttc |
| domain nt | NO: 1371 | cggcttcaacatcgaggactattacatccattgggtgcggcagatgccaggaaaggggctggaatggatgggac |
| | | ggattgacccggagaacgacgaaaccaagtacggaccgatctttcaaggacacgtgactatctccgccgacacc |
| | | agcatcaatacggtgtacctccaatggtcctcactcaaggcctcggataccgcgatgtactactgcgcgttcagag |
| | | gaggcgtctactggggacaagggactactgtgactgtctcatcaggaggtggaggaagcggaggaggtggctc |
| | | gggcggaggtggatcgggaggaggagggtccgatgtggtgatgacccagtccccactgtcgctcccggtgacc |
| | | ctcggacagcctgctagcatctcgtgcaaatcctcgcaatccctgctggactcggacggaaaaacgtacctcaatt |
| | | ggctgcagcagcgccctggccagagcccgagaaggcttatctcgctggtgtcaaagctggatagcggtgtgccc |
| | | gaccggttcagcggctcagggtcaggaaccgatttcaccttgaagatctcccgcgtggaagccgaagatgtcgga |
| | | gtctactactgctggcagggtactcacttcccggggacctttggtggcggcactaaggtcgagattaag |
| CAR3 - | SEQ ID | atggccctccctgtcaccgccctgctgcttccgctggctcttctgctccacgccgctcggcccgagattcagctggt |
| Soluble scFv - | NO: 1372 | ccaaagcggcgcagaagtgaaaaagccaggggaatcgttgcgcatcagctgtaaaggttccggcttcaacatcg |
| nt | | aggactattacatccattgggtgcggcagatgccaggaaaggggctggaatggatgggacggattgacccggag |
| | | aacgacgaaaccaagtacggaccgatctttcaaggacacgtgactatctccgccgacaccagcatcaatacggtg |
| | | tacctccaatggtcctcactcaaggcctcggataccgcgatgtactactgcgcgttcagaggaggcgtctactggg |
| | | gacaagggactactgtgactgtctcatcaggaggtggaggaagcggaggaggtggctcgggcggaggtggatc |
| | | gggaggaggagggtccgatgtggtgatgacccagtccccactgtcgctcccggtgaccctcggacagcctgcta |
| | | gcatctcgtgcaaatcctcgcaatccctgctggactcggacggaaaaacgtacctcaattggctgcagcagcgcc |
| | | ctggccagagcccgagaaggcttatctcgctggtgtcaaagctggatagcggtgtgcccgaccggttcagcggct |
| | | cagggtcaggaaccgatttcaccttgaagatctcccgcgtggaagccgaagatgtcggagtctactactgctggca |
| | | gggtactcacttcccggggacctttggtggcggcactaaggtcgagattaagggctcacaccatcatcaccatcac |
| | | caccac |
| CAR3 - | SEQ ID | malpvtalllplalllhaarpeiqlvqsgaevkkpgeslrisckgsgfniedyyihwvrqmpgkglewmgrid |
| Soluble scFv - | NO: 1373 | pendetkygpifqghvtisadtsintvylqwsslkasdtamyycafrggvywgqgttvtvssggggsggggs |
| aa | | ggggsggggsdvvmtqsplslpvtlgqpasisckssqslldsdgktylnwlqqrpgqsprrlislvskldsgvp |
| | | drfsgsgsgtdftlkisrveaedvgvyycwqgthfpgtfgggtkveikgshhhhhhhh |
| CAR3 - Full - | SEQ ID | atggccctccctgtcaccgccctgctgcttccgctggctcttctgctccacgccgctcggcccgaaatccagctggt |
| nt | NO: 1374 | gcaaagcggagccgaggtgaagaagcccggagaatccctgcgcatctcgtgtaagggttccggctttaacatcg |
| | | aggattactacatccactgggtgagacagatgccgggcaaaggtctggaatggatgggccgcatcgacccggag |
| | | aacgacgaaaccaaatacggaccaatcttccaaggacatgtgactatttccgcggatacctccatcaacactgtcta |
| | | cttgcagtggagctcgctcaaggcgtcggataccgccatgtactactgcgcattcagaggaggtgtgtactgggg |
| | | ccagggcactacggtcaccgtgtcctcgggaggtggagggtcaggaggcggaggctcgggcggtggaggatc |
| | | aggcggaggaggaagcgatgtggtcatgactcaatccccactgtcactgcctgtcactctggggcaaccggcttc |
| | | catctcatgcaagtcaagccaatcgctgctcgactccgacggaaaaacctacctcaattggcttcagcagcgccca |
| | | ggccagtcgcctcggaggctgatctcactcgtgtcgaagcttgactccggggtgccggatcggtttagcggaagc |
| | | ggatcggggaccgacttcacgttgaagattagccgggtggaagccgaggacgtgggagtctattactgctggca |
| | | ggggacccacttcccggggactttcggaggaggcaccaaagtcgagattaagaccactaccccagcaccgagg |
| | | ccacccaccccggctcctaccatcgcctcccagcctctgtccctgcgtccggaggcatgtagacccgcagctggt |
| | | ggggccgtgcatacccggggtcttgacttcgcctgcgatatctacatttgggcccctctggctggtacttgcggggt |
| | | cctgctgctttcactcgtgatcactctttactgtaagcgcggtcggaagaagctgctgtacatctttaagcaaccc |
| | | ttcatgaggcctgtgcagactactcaagaggaggacggctgttcatgccggttcccagaggaggaggaaggcggct |
| | | gcgaactgcgcgtgaaattcagccgcagcgcagatgctccagcctacaagcaggggcagaaccagctctacaa |
| | | cgaactcaatcttggtcggagagaggagtacgacgtgctggacaagcggagaggacgggacccagaaatggg |
| | | cgggaagccgcgcagaaagaatccccaagagggcctgtacaacgagctccaaaaggataagatggcagaagc |
| | | ctatagcgagattggtatgaaaggggaacgcagaagaggcaaaggccacgacggactgtaccagggactcagc |
| | | accgccaccaaggacacctatgacgctcttcacatgcaggccctgccgcctcgg |
| CAR3 Full - | SEQ ID | malpvtalllplalllhaarpeiqlvqsgaevkkpgeslrisckgsgfniedyyihwvrqmpgkglewmgrid |
| aa | NO: 1375 | pendetkygpifqghvtisadtsintvylqwsslkasdtamyycafrggvywgqgttvtvssggggsgggg |
| | | sggggsggggsdvvmtqsplslpvtlgqpasisckssqslldsdgktylnwlqqrpgqsprrlislvskldsg |
| | | vpdrfsgsgsgtdftlkisrveaedvgvyycwqgthfpgtfgggtkveiktttpaprpptpaptiasqplslrpe |
| | | acrpaaggavhtrgldfacdiyiwaplagtcgvlllslvitlyckrgrkkllyifkqpfmrpvqttqeedgcscrfp |
| | | eeeeggcelrvkfsrsadapaykqgqnqlynelnlgrreeydvldlargrdpemggkprrknpqeglynelq |
| | | kdkmaeayseigmkgerrrgkghdglyqglstatkdtydalhmqalppr |

CAR 4

| | | |
|---|---|---|
| CAR4 scFv | SEQ ID | dvvmtqsplslpvtlgqpasisckssqslldsdgktylnwlqqrpgqsprrlislvskldsgvpdrfsgsgsgtdf |
| domain | NO: 1376 | tlkisrveaedvgvyycwqgthfpgtfgggtkveikggggsggggsggggsggggseiqlvqsgaevkkpg |
| | | eslrisckgsgfniedyyihwvrqmpgkglewmgridpendetkygpifqghvtisadtsintvylqwsslka |
| | | sdtamyycafrggvywgqgttvtvss |
| CAR4 scFv | SEQ ID | gacgtcgtcatgacccagagcccgctgtcactgcctgtgaccctgggccagccggcgtccattagctgcaaatcc |
| domain nt | NO: 1377 | tcgcaatccctgctcgactcagacggaaaaacgtacttgaactggctccaacagcgccctgggcaatccccaagg |
| | | cggcttatctcactcgtcagcaagctcgatagcggtgtcccagacagattttcgggctcgggatcgggcactgattt |
| | | cactctgaagatctcgcgggtggaagccgaggatgtgggagtgtactattgctggcagggcactcacttccccgg |
| | | gacgtttggcggaggaactaaggtcgagatcaaaggaggaggtggatcaggcggaggtgggagcggaggag |
| | | gaggaagcggtggtggaggttccgaaatccagctggtgcaatcaggagccgaggtgaagaagccgggagaat |
| | | ccctgcgcatctcgtgcaagggctcgggcttcaacatcgaggattactacatccactgggtgcggcagatgccgg |
| | | gaaaggggttggaatggatgggacgcattgacccggaaaatgatgaaaccaaatacgggccaatcttccaaggc |
| | | cacgtgaccattagcgctgacacttccatcaacaccgtgtaccttcagtggtcctcactgaaggcgtcggacactgc |
| | | catgtactactgtgcattcagaggaggggtctactggggacagggcaccaccgtgaccgtgagctcc |
| CAR4 - | SEQ ID | atggccctccctgtcaccgccctgctgcttccgctggctcttctgctccacgccgctcggcccgacgtcgtcatgac |
| Soluble scFv - | NO: 1378 | ccagagcccgctgtcactgcctgtgaccctgggccagccggcgtccattagctgcaaatcctcgcaatccctgctc |
| nt | | gactcagacggaaaaacgtacttgaactggctccaacagcgccctgggcaatccccaaggcggcttatctcactc |
| | | gtcagcaagctcgatagcggtgtcccagacagattttcgggctcgggatcgggcactgatttcactctgaagatctc |
| | | gcgggtggaagccgaggatgtgggagtgtactattgctggcagggcactcacttccccgggacgtttggcggag |
| | | gaactaaggtcgagatcaaaggaggaggtggatcaggcggaggtgggagcggaggaggaggaagcggtggt |
| | | ggaggttccgaaatccagctggtgcaatcaggagccgaggtgaagaagccgggagaatccctgcgcatctcgtg |
| | | caagggctcgggcttcaacatcgaggattactacatccactgggtgcggcagatgccgggaaaggggttggaat |
| | | ggatgggacgcattgacccggaaaatgatgaaaccaaatacgggccaatcttccaaggccacgtgaccattagc |
| | | gctgacacttccatcaacaccgtgtaccttcagtggtcctcactgaaggcgtcggacactgccatgtactactgtgc |
| | | attcagaggaggggtctactggggacagggcaccaccgtgaccgtgagctccggctcgcatcaccatcatcacc |
| | | accatcac |
| CAR4 - | SEQ ID | malpvtalllplalllhaarpdvvmtqsplslpvtlgqpasisckssqslldsdgktylnwlqqrpgqsprrlislv |
| Soluble scFv - | NO: 1379 | skldsgvpdrfsgsgsgtdftlkisrveaedvgvyycwqgthfpgtfgggtkveikggggsggggsggggsg |
| aa | | gggseiqlvqsgaevkkpgeslrisckgsgfniedyyihwvrqmpgkglewmgridpendetkygpifqg |
| | | hvtisadtsintvylqwsslkasdtamyycafrggvywgqgttvtvssgshhhhhhhh |
| CAR4 - Full - | SEQ ID | atggccctccctgtcaccgccctgctgcttccgctggctcttctgctccacgccgctcggcccgacgtcgtcatgac |
| nt | NO: 1380 | ccaatcccctctctccctgccggtcaccctgggtcagccggcgtcgatctcatgcaaaagctcacagtccctgctg |
| | | gattcggacggaaaaacctacttgaactggctccaacagaggccgggtcagtcccctcgcagactgatctcgctg |
| | | gtgagcaagctcgactcgggtgtgccggatcggttctccgggtcaggatcgggcaccgactttacgctcaagattt |
| | | cgagagtggaggccgaggatgtgggagtgtactattgctggcagggcacgcatttccccgggacctttggaggc |
| | | gggactaaggtggaaatcaagggaggtggcggatcaggcggaggaggcagcggcggaggtggatcaggag |
| | | gcggagggtcagagatccagctggtccaaagcggagcagaggtgaagaagccaggcgagtcccttcgcatttc |
| | | gtgcaaagggagcggcttcaacattgaagattactacatccactgggtgcggcaaatgccaggaaagggtctgga |
| | | atggatgggacggatcgacccagaaaatgatgaaactaagtacggaccgatcttccaaggacacgtcactatctc |
| | | cgcggacacttcgatcaacaccgtgtacctccagtggagcagcttgaaagcctccgacaccgctatgtactactgt |
| | | gccttccgcggaggagtctactggggacaggggactactgtgaccgtgtcgtccaccactaccccagcaccgag |
| | | gccacccaccccggctcctaccatcgcctcccagcctctgtccctgcgtccggaggcatgtagacccgcagctgg |
| | | tggggccgtgcatacccggggtcttgacttcgcctgcgatatctacatttgggcccctctggctggtacttgcgggg |
| | | tcctgctgctttcactcgtgatcactctttactgtaagcgcggtcggaagaagctgctgtacatctttaagcaaccc |
| | | ttcatgaggcctgtgcagactactcaagaggaggacggctgttcatgccggttcccagaggaggaggaaggcggct |
| | | gcgaactgcgcgtgaaattcagccgcagcgcagatgctccagcctacaagcaggggcagaaccagctctacaa |
| | | cgaactcaatcttggtcggagagaggagtacgacgtgctggacaagcggagaggacgggacccagaaatggg |
| | | cgggaagccgcgcagaaagaatccccaagagggcctgtacaacgagctccaaaaggataagatggcagaagc |
| | | ctatagcgagattggtatgaaaggggaacgcagaagaggcaaaggccacgacggactgtaccagggactcagc |
| | | accgccaccaaggacacctatgacgctcttcacatgcaggccctgccgcctcgg |
| CAR4 - Full - | SEQ ID | malpvtalllplalllhaarpdvvmtqsplslpvtlgqpasisckssqslldsdgktylnwlqqrpgqsprrlisl |
| aa | NO: 1381 | vskldsgvpdrfsgsgsgtdftlkisrveaedvgvyycwqgthfpgtfgggtkveikggggsggggsggggs |
| | | ggggseiqlvqsgaevkkpgeslrisckgsgfniedyyihwvrqmpgkglewmgridpendetkygpif |
| | | qghvtisadtsintvylqwsslkasdtamyycafrggvywgqgttvtvsstapaprpptpaptiasqplslrpe |
| | | acrpaaggavhtrgldfacdiyiwaplagtcgvlllslvitlyckrgrkkllyifkqpfmrpvqttqeedgcscrfp |
| | | eeeeggcelrvkfsrsadapaykqgqnqlynelnlgrreeydvldlargrdpemggkprrknpqeglynelq |
| | | kdkmaeayseigmkgerrrgkghdglyqglstatkdtydalhmqalppr |

CAR 5

| | | |
|---|---|---|
| CAR5 scFv | SEQ ID | eiqlvqsgaevkkpgatvkisckgsgfniedyyihwvqqapgkglewmgridpendetkygpifqgrvtita |
| domain | NO: 1382 | dtstntvymelsslrsedtavyycafrggvywgqgttvtvssggggsggggsggggsggggsdvvmtqspls |
| | | lpvtlgqpasisckssqslldsdgktylnwlqqrpgqsprrlislvskldsgvpdrfsgsgsgtdftlkisrveaed |
| | | vgvyycwqgthfpgtfgggtkveik |
| CAR5 scFv | SEQ ID | gaaatccagctcgtgcagagcggagccgaggtcaagaaaccgggtgctaccgtgaagatttcatgcaagggatc |
| domain nt | NO: 1383 | gggcttcaacatcgaggattactacatccactgggtgcagcaggcaccaggaaaaggacttgaatggatgggcc |
| | | ggatcgacccggaaaatgacgagactaagtacggccctatcttccaaggacgggtgacgatcaccgcagacact |
| | | agcaccaacaccgtctatatggaactctcgtccctgaggtccgaagatactgccgtgtactactgtgcgtttcgcgg |
| | | aggtgtgtactggggacagggtaccaccgtcaccgtgtcatcgggcggtggaggctccggtggaggagggtca |
| | | ggaggcggtggaagcggaggaggcggcagcgacgtggtcatgactcaatcgccgctgtcgctgcccgtcactc |
| | | tgggacaacccgcgtccatcagctgcaaatcctcgcagtcactgcttgactccgatggaaagacctacctcaactg |
| | | gctgcagcaacgcccaggccaatccccaagacgcctgatctcgttggtgtcaaagctggactcaggggtgccgg |
| | | accggttctccgggagcgggtcgggcacggatttcactctcaagatctccagagtggaagccgaggatgtggga |
| | | gtctactactgctggcagggaacccatttccctggaacttttggcggaggaactaaggtcgagattaaa |
| CAR5 - | SEQ ID | atggccctccctgtcaccgccctgctgcttccgctggctcttctgctccacgccgctcggcccgaaatccagctcgt |
| Soluble scFv - | NO: 1384 | gcagagcggagccgaggtcaagaaaccgggtgctaccgtgaagatttcatgcaagggatcgggcttcaacatcg |
| nt | | aggattactacatccactgggtgcagcaggcaccaggaaaaggacttgaatggatgggccggatcgacccgga |
| | | aaatgacgagactaagtacggccctatcttccaaggacgggtgacgatcaccgcagacactagcaccaacaccg |
| | | tctatatggaactctcgtccctgaggtccgaagatactgccgtgtactactgtgcgtttcgcggaggtgtgtactggg |
| | | gacagggtaccaccgtcaccgtgtcatcgggcggtggaggctccggtggaggagggtcaggaggcggtggaa |
| | | gcggaggaggcggcagcgacgtggtcatgactcaatcgccgctgtcgctgcccgtcactctgggacaacccgc |
| | | gtccatcagctgcaaatcctcgcagtcactgcttgactccgatggaaagacctacctcaactggctgcagcaacgc |
| | | ccaggccaatccccaagacgcctgatctcgttggtgtcaaagctggactcaggggtgccggaccggttctccggg |
| | | agcgggtcgggcacggatttcactctcaagatctccagagtggaagccgaggatgtgggagtctactactgctgg |
| | | cagggaacccatttccctggaacttttggcggaggaactaaggtcgagattaaagggagccaccatcatcatcacc |
| | | accaccac |
| CAR5 - | SEQ ID | malpvtalllplalllhaarpeiqlvqsgaevkkpgatvkisckgsgfniedyyihwvqqapgkglewmgrid |
| Soluble scFv - | NO: 1385 | pendetkygpifqgrvtitadtstntvymelsslrsedtavyycafrggvywgqgttvtvssggggsggggsgg |
| aa | | ggsggggsdvvmtqsplslpvtlgqpasisckssqslldsdgktylnwlqqrpgqsprrlislvskldsgvpdrf |
| | | sgsgsgtdftlkisrveaedvgvyycwqgthfpgtfgggtkveikgshhhhhhhh |
| CAR5 - Full - | SEQ ID | atggccctccctgtcaccgccctgctgcttccgctggctcttctgctccacgccgctcggcccgaaatccagctcgt |
| nt | NO: 1386 | gcagagcggagccgaggtcaagaaaccgggtgctaccgtgaagatttcatgcaagggatcgggcttcaacatcg |
| | | aggattactacatccactgggtgcagcaggcaccaggaaaaggacttgaatggatgggccggatcgacccgga |
| | | aaatgacgagactaagtacggccctatcttccaaggacgggtgacgatcaccgcagacactagcaccaacaccg |
| | | tctatatggaactctcgtccctgaggtccgaagatactgccgtgtactactgtgcgtttcgcggaggtgtgtactggg |
| | | gacagggtaccaccgtcaccgtgtcatcgggcggtggaggctccggtggaggagggtcaggaggcggtggaa |
| | | gcggaggaggcggcagcgacgtggtcatgactcaatcgccgctgtcgctgcccgtcactctgggacaacccgc |
| | | gtccatcagctgcaaatcctcgcagtcactgcttgactccgatggaaagacctacctcaactggctgcagcaacgc |
| | | ccaggccaatccccaagacgcctgatctcgttggtgtcaaagctggactcaggggtgccggaccggttctccggg |
| | | agcgggtcgggcacggatttcactctcaagatctccagagtggaagccgaggatgtgggagtctactactgctgg |
| | | cagggaacccatttccctggaacttttggcggaggaactaaggtcgagattaaaaccactaccccagcaccgagg |
| | | ccacccaccccggctcctaccatcgcctcccagcctctgtccctgcgtccggaggcatgtagacccgcagctggt |
| | | ggggccgtgcatacccggggtcttgacttcgcctgcgatatctacatttgggcccctctggctggtacttgcggggt |
| | | cctgctgctttcactcgtgatcactctttactgtaagcgcggtcggaagaagctgctgtacatctttaagcaaccc |
| | | ttcatgaggcctgtgcagactactcaagaggaggacggctgttcatgccggttcccagaggaggaggaaggcggct |
| | | gcgaactgcgcgtgaaattcagccgcagcgcagatgctccagcctacaagcaggggcagaaccagctctacaa |
| | | cgaactcaatcttggtcggagagaggagtacgacgtgctggacaagcggagaggacgggacccagaaatggg |
| | | cgggaagccgcgcagaaagaatccccaagagggcctgtacaacgagctccaaaaggataagatggcagaagc |
| | | ctatagcgagattggtatgaaaggggaacgcagaagaggcaaaggccacgacggactgtaccagggactcagc |
| | | accgccaccaaggacacctatgacgctcttcacatgcaggccctgccgcctcgg |
| CAR5 - Full - | SEQ ID | malpvtalllplalllhaarpeiqlvqsgaevkkpgatvkisckgsgfniedyyihwvqqapgkglewmgri |
| aa | NO: 1387 | dpendetkygpifqgrvtitadtstntvymelsslrsedtavyycafrggvywgqgttvtvssggggsggggs |
| | | ggggsggggsdvvmtqsplslpvtlgqpasisckssqslldsdgtylnwlqqrpgqsprrlislvskldsgv |
| | | pdrfsgsgsgtdftlkisrveaedvgvyycwqgthfpgtfgggtkveiktttpaprpptpaptiasqplslrpeac |
| | | rpaaggavhtrgldfacdiyiwaplagtcgvlllslvitlyckrgrkkllyifkqpfmrpvqttqeedgcscrfpee |
| | | eeggcelrvkfsrsadapaykqgqnqlynelnlgrreeydvldlargrdpemggkprrknpqeglynelqkd |
| | | kmaeayseigmkgerrrgkghdglyqglstatkdtydalhmqalppr |

CAR 6

| | | |
|---|---|---|
| CAR6 | SEQ ID | eiqlvqsgaevkkpgeslrisckgsgfniedyyihwvrqmpgkglewmgridpendetkygpifqghvtis |
| scFv domain | NO: 1388 | adtsintvylqwsslkasdtamyycafrggvywgqgttvtvssggggsggggsggggsggggsdvvmtqs |
| | | pdslayslgeratinckssqslldsdgktylnwlqqkpgqppkrlislvskldsgvpdrfsgsgsgtdftltisslqa |
| | | edvavyycwqgthfpgtfgggtkveik |
| CAR6 scFv | SEQ ID | gaaatccagctggtgcagtcaggcgccgaggtcaagaagccgggagagtcgctgagaatctcgtgcaagggct |
| domain nt | NO: 1389 | cggggttcaacatcgaggactactacattcactgggtcaggcagatgccgggaaagggactggaatggatgggc |
| | | cggatcgacccagaaaatgacgaaaccaaatacgggccgatttttcaaggccacgtgactatcagcgcagacac |
| | | gagcatcaacactgtctacctccagtggtcctcgcttaaggccagcgataccgctatgtactactgcgcattcagag |
| | | gcggggtgtactggggacaaggaaccactgtgaccgtgagcagcggaggtggcggctcgggaggaggtggg |
| | | agcggaggaggaggttccggcggtggaggatcagatgtcgtgatgacccagtccccggactccctcgctgtctc |
| | | actgggcgagcgcgcgaccatcaactgcaaatcgagccagtcgctgttggactccgatggaaagacttatctgaa |
| | | ttggctgcaacagaaaccaggacaacctcccaagcggctcatctcgcttgtgtcaaaactcgattcgggagtgcca |
| | | gaccgcttctcggggtccgggagcggaactgactttactttgaccatttcctcactgcaagcggaggatgtggccg |
| | | tgtattactgttggcagggcacgcatttccctggaaccttcggtggcggaactaaggtggaaatcaag |
| CAR6 - | SEQ ID | atggccctccctgtcaccgccctgctgcttccgctggctcttctgctccacgccgctcggcccgaaatccagctggt |
| Soluble scFv - | NO: 1390 | gcagtcaggcgccgaggtcaagaagccgggagagtcgctgagaatctcgtgcaagggctcggggttcaacatc |
| nt | | gaggactactacattcactgggtcaggcagatgccgggaaagggactggaatggatgggccggatcgacccag |
| | | aaaatgacgaaaccaaatacgggccgatttttcaaggccacgtgactatcagcgcagacacgagcatcaacactg |
| | | tctacctccagtggtcctcgcttaaggccagcgataccgctatgtactactgcgcattcagaggcggggtgtactgg |
| | | ggacaaggaaccactgtgaccgtgagcagcggaggtggcggctcgggaggaggtgggagcggaggaggag |
| | | gttccggcggtggaggatcagatgtcgtgatgacccagtccccggactccctcgctgtctcactgggcgagcgcg |
| | | cgaccatcaactgcaaatcgagccagtcgctgttggactccgatggaaagacttatctgaattggctgcaacagaa |
| | | accaggacaacctcccaagcggctcatctcgcttgtgtcaaaactcgattcgggagtgccagaccgcttctcggg |
| | | gtccgggagcggaactgactttactttgaccatttcctcactgcaagcggaggatgtggccgtgtattactgttggca |
| | | gggcacgcatttccctggaaccttcggtggcggaactaaggtggaaatcaagggatcacaccaccatcatcacca |
| | | tcaccaccat |
| CAR6 - | SEQ ID | malpvtalllplalllhaarpeiqlvqsgaevkkpgeslrisckgsgfniedyyihwvrqmpgkglewmgrid |
| Soluble scFv - | NO: 1391 | pendetkygpifqghvtisadtsintvylqwsslkasdtamyycafrggvywgqgttvtvssggggsggggs |
| aa | | ggggsggggsdvvmtqspdslayslgeratinckssqslldsdgktylnwlqqkpgqpplalislvskldsgv |
| | | pdrfsgsgsgtdftltisslqaedvavyycwqgthfpgtfgggtkveikgshhhhhhhhh |
| CAR6 - | SEQ ID | atggccctccctgtcaccgccctgctgcttccgctggctcttctgctccacgccgctcggcccgagattcagctcgt |
| Full - nt | NO: 1392 | gcaatcgggagcggaagtcaagaagccaggagagtccttgcggatctcatgcaagggtagcggctttaacatcg |
| | | aggattactacatccactgggtgaggcagatgccggggaagggactcgaatggatgggacggatcgacccaga |
| | | aaacgacgaaactaagtacggtccgatcttccaaggccatgtgactattagcgccgatacttcaatcaataccgtgt |
| | | atctgcaatggtcctcattgaaagcctcagataccgcgatgtactactgtgctttcagaggaggggtctactgggga |
| | | cagggaactaccgtgactgtctcgtccggcggaggcgggtcaggaggtggcggcagcggaggaggagggtc |
| | | cggcggaggtgggtccgacgtcgtgatgacccagagccctgacagcctggcagtgagcctgggcgaaagagc |
| | | taccattaactgcaaatcgtcgcagagcctgctggactcggacggaaaaacgtacctcaattggctgcagcaaaa |
| | | gcctggccagccaccgaagcgccttatctcactggtgtcgaagctggattcgggagtgcccgatcgcttctccgg |
| | | ctcgggatcgggtactgacttcaccctcactatctcctcgcttcaagcagaggacgtggccgtctactactgctggc |
| | | agggaacccactttccgggaaccttcggcggagggacgaaagtggagatcaagaccactaccccagcaccgag |
| | | gccacccaccccggctcctaccatcgcctcccagcctctgtccctgcgtccggaggcatgtagacccgcagctgg |
| | | tggggccgtgcatacccggggtcttgacttcgcctgcgatatctacatttgggcccctctggctggtacttgcgggg |
| | | tcctgctgctttcactcgtgatcactctttactgtaagcgcggtcggaagaagctgctgtacatctttaagcaaccc |
| | | ttcatgaggcctgtgcagactactcaagaggaggacggctgttcatgccggttcccagaggaggaggaaggcggct |
| | | gcgaactgcgcgtgaaattcagccgcagcgcagatgctccagcctacaagcaggggcagaaccagctctacaa |
| | | cgaactcaatcttggtcggagagaggagtacgacgtgctggacaagcggagaggacgggacccagaaatggg |
| | | cgggaagccgcgcagaaagaatccccaagagggcctgtacaacgagctccaaaaggataagatggcagaagc |
| | | ctatagcgagattggtatgaaaggggaacgcagaagaggcaaaggccacgacggactgtaccagggactcagc |
| | | accgccaccaaggacacctatgacgctcttcacatgcaggccctgccgcctcgg |
| CAR6 - | SEQ ID | malpvtalllplalllhaarpeiqlvqsgaevkkpgeslrisckgsgfniedyyihwvrqmpgkglewmgrid |
| Full - aa | NO: 1393 | pendetkygpifqghvtisadtsintvylqwsslkasdtamyycafrqqvywgqgttvtvssggggsgggg |
| | | sggggsggggsdvvmtqspdslayslgeratinckssqslldsdgktylnwlqqkpgqpplalislvsklds |
| | | gvpdrfsgsgsgtdftltisslqaedvavyycwqgthfpgtfgggtkveiktttpaprpptpaptiasqplslrpe |
| | | acrpaaggavhtrgldfacdiyiwaplagtcgvlllslvitlyckrgrkkllyifkqpfmrpvqttqeedgcscrfp |
| | | eeeeggcelrvkfsrsadapaykqgqnqlynelnlgrreeydvldlargrdpemggkprrknpqeglynelq |
| | | kdkmaeayseigmkgerrrgkghdglyqglstatkdtydalhmqalppr |

CAR 7

| | | |
|---|---|---|
| CAR7 scFv | SEQ ID | dvvmtqspdslayslgeratinckssqslldsdgktylnwlqqkpgqpplalislvskldsgvpdrfsgsgsgtd |
| domain | NO: 1394 | ftltisslqaedvavyycwqgthfpgtfgggtkveikggggsggggsggggsggggseiqlvqsgaevkkpg |
| | | eslrisckgsgfniedyyihwvrqmpgkglewmgridpendetkygpifqghvtisadtsintvylqwsslka |
| | | sdtamyycafrggvywgqgttvtvss |
| CAR7 scFv | SEQ ID | gacgtggtgatgacccaatcgccagattccctggcagtgtccctgggcgaacgcgccactattaactgcaaatcgt |
| domain nt | NO: 1395 | cacagtccttgcttgattccgacggaaagacctacctcaattggctccagcagaagccaggacaaccgccaaaga |
| | | gactgatctccctggtgtcaaagctggactcgggagtgcctgatcggttctcgggtagcgggagcggcaccgact |
| | | tcactctgaccatctcgtcactccaggctgaggacgtggccgtgtattactgttggcagggtactcactttccgggca |
| | | ctttcggaggcggcaccaaggtggagattaaaggaggaggcggaagcggaggtggaggatcgggaggtggtg |
| | | ggagcggcggaggagggagcgagatccagctcgtccaatcgggagcggaagtgaagaagcccggagagtca |
| | | cttagaatctcatgcaaggggtcgggcttcaacatcgaggattactacatccattgggtccgccagatgcctggtaa |
| | | aggactggaatggatggggaggattgacccggaaaacgacgaaactaagtacggaccgatctttcaagggcac |
| | | gtgactatctccgctgatacctcaatcaatactgtctacctccagtggtcctcgctgaaagcaagcgacaccgcgat |
| | | gtactactgcgccttccggggaggagtgtactggggccaaggcaccacggtcacggtcagctcc |
| CAR7 - | SEQ ID | atggccctccctgtcaccgccctgctgcttccgctggctcttctgctccacgccgctcggcccgacgtggtgatgac |
| Soluble scFv - | NO: 1396 | ccaatcgccagattccctggcagtgtccctgggcgaacgcgccactattaactgcaaatcgtcacagtccttgcttg |
| nt | | attccgacggaaagacctacctcaattggctccagcagaagccaggacaaccgccaaagagactgatctccctg |
| | | gtgtcaaagctggactcgggagtgcctgatcggttctcgggtagcgggagcggcaccgacttcactctgaccatct |
| | | cgtcactccaggctgaggacgtggccgtgtattactgttggcagggtactcactttccgggcactttcggaggcgg |
| | | caccaaggtggagattaaaggaggaggcggaagcggaggtggaggatcgggaggtggtgggagcggcgga |
| | | ggagggagcgagatccagctcgtccaatcgggagcggaagtgaagaagcccggagagtcacttagaatctcat |
| | | gcaaggggtcgggcttcaacatcgaggattactacatccattgggtccgccagatgcctggtaaaggactggaat |
| | | ggatggggaggattgacccggaaaacgacgaaactaagtacggaccgatctttcaagggcacgtgactatctcc |
| | | gctgatacctcaatcaatactgtctacctccagtggtcctcgctgaaagcaagcgacaccgcgatgtactactgcgc |
| | | cttccggggaggagtgtactggggccaaggcaccacggtcacggtcagctccggctcccatcaccaccaccatc |
| | | accatcatcac |
| CAR7 - | SEQ ID | malpvtalllplalllhaarpdvvmtqspdslayslgeratinckssqslldsdgktylnwlqqkpgqppkrlisl |
| Soluble scFv - | NO: 1397 | vskldsgvpdrfsgsgsgtdftltisslqaedvavyycwqgthfpgtfgggtkveikggggsggggsggggsg |
| aa | | gggseiqlvqsgaevkkpgeslrisckgsgfniedyyihwvrqmpgkglewmgridpendetkygpifqg |
| | | hvtisadtsintvylqwsslkasdtamyycafrggvywgqgttvtvssgshhhhhhhhh |
| CAR7 | SEQ ID | atggccctccctgtcaccgccctgctgcttccgctggctcttctgctccacgccgctcggcccgacgtggtgatgac |
| Full - nt | NO: 1398 | tcagtcgcctgactcgctggctgtgtcccttggagagcgggccactatcaattgcaagtcatcccagtcgctgctgg |
| | | attccgacgggaaaacctacctcaattggctgcagcaaaaaccgggacagcctccaaagcggctcatcagcctg |
| | | gtgtccaagttggacagcggcgtgccagaccgcttctccggttcgggaagcggtactgatttcacgctgaccatct |
| | | catccctccaagcggaggatgtggcagtctactactgttggcagggcacgcattttccgggcacttttggaggagg |
| | | gaccaaggtcgaaatcaagggaggaggtggctcgggcggaggaggctcgggaggaggaggatcaggaggc |
| | | ggtggaagcgagattcaactggtccagagcggcgcagaagtcaagaagccgggtgaatcgctcagaatctcgtg |
| | | caaaggatcgggattcaacatcgaggactactacattcactgggtcagacaaatgccgggcaaagggctggaat |
| | | ggatggggaggatcgaccccgaaaacgatgaaaccaagtacggaccaatcttccaagggcacgtgaccatttcg |
| | | gcggacacctcaatcaacactgtgtacctccagtggagctcacttaaggccagcgataccgccatgtactattgcg |
| | | ctttccgcggaggggtgtactggggacagggcactactgtgaccgtgtcatccaccactaccccagcaccgagg |
| | | ccacccaccccggctcctaccatcgcctcccagcctctgtccctgcgtccggaggcatgtagacccgcagctggt |
| | | ggggccgtgcatacccggggtcttgacttcgcctgcgatatctacatttgggcccctctggctggtacttgcggggt |
| | | cctgctgctttcactcgtgatcactctttactgtaagcgcggtcggaagaagctgctgtacatctttaagcaaccc |
| | | ttcatgaggcctgtgcagactactcaagaggaggacggctgttcatgccggttcccagaggaggaggaaggcggct |
| | | gcgaactgcgcgtgaaattcagccgcagcgcagatgctccagcctacaagcaggggcagaaccagctctacaa |
| | | cgaactcaatcttggtcggagagaggagtacgacgtgctggacaagcggagaggacgggacccagaaatggg |
| | | cgggaagccgcgcagaaagaatccccaagagggcctgtacaacgagctccaaaaggataagatggcagaagc |
| | | ctatagcgagattggtatgaaaggggaacgcagaagaggcaaaggccacgacggactgtaccagggactcagc |
| | | accgccaccaaggacacctatgacgctcttcacatgcaggccctgccgcctcgg |
| CAR7 | SEQ ID | malpvtalllplalllhaarpdvvmtqspdslavslgeratinckssqslldsdgktylnwlqqkpgqppkrlis |
| Full - aa | NO: 1399 | lvskldsgvpdrfsgsgsgtdftltisslqaedvavyycwqgthfpgtfgggtkveikggggsggggsggggs |
| | | ggggseiqlvqsgaevkkpgeslrisckgsgfniedyyihwvrqmpgkglewmgridpendetkygpif |
| | | qghvtisadtsintvylqwsslkasdtamyycafrggvywgqgttvtvsstttpaprpptpaptiasqplslrpe |
| | | acrpaaggavhtrgldfacdiyiwaplagtcgvlllslvitlyckrgrkkllyifkqpfmrpvqttqeedgcscrfp |
| | | eeeeggcelrvkfsrsadapaykqgqnqlynelnlgrreeydvldkrrgrdpemggkprrknpqeglynelq |
| | | kdkmaeayseigmkgerrrgkghdglyqglstatkdtydalhmqalppr |

CAR 8

| | | |
|---|---|---|
| CAR8 scFv | SEQ ID | dvvmtqsplslpvtlgqpasisckssqslldsdgktylnwlqqrpgqsprrlislvskldsgvpdrfsgsgsgtdf |
| domain | NO: 1400 | tlkisrveaedvgvyycwqgthfpgtfgggtkveikggggsggggsggggsggggseiqlvqsgaevkkpg |
| | | atvkisckgsgfniedyyihwvqqapgkglewmgridpendetkygpifqgrvtitadtstntvymelsslrs |
| | | edtavyycafrggvywgqgttvtvss |
| CAR8 scFv | SEQ ID | gatgtggtcatgacgcagtcaccactgtccctccccgtgacccttggacagccagcgtcgattagctgcaagtcat |
| domain nt | NO: 1401 | cccaatccctgctcgattcggatggaaagacctatctcaactggctgcagcaaagacccggtcagagccctagga |
| | | gactcatctcgttggtgtcaaagctggacagcggagtgccggaccggttttccggttcgggatcggggacggactt |
| | | cactctgaagatttcacgggtggaagctgaggatgtgggagtgtactactgctggcagggaacccatttccctggc |
| | | acttttggcggaggaactaaggtcgaaatcaagggaggaggtggctcgggaggaggcggatcgggcggaggc |
| | | gggagcggcggaggagggtccgaaatccaacttgtccagtcaggagccgaagtgaagaaaccgggagccacc |
| | | gtcaaaatcagctgtaagggatcgggattcaatatcgaggactactacatccactgggtgcagcaagctccgggc |
| | | aaaggactggagtggatggggcgcatcgacccagagaacgacgaaaccaaatacggcccgatcttccaaggg |
| | | cgggtgaccatcaccgcggacacctcaactaacactgtgtacatggagctgagctccctgcgctccgaagatact |
| | | gcagtctactactgcgccttccgcggtggtgtgtactggggacagggcaccactgtgactgtcagctcg |
| CAR8 - | SEQ ID | atggccctccctgtcaccgccctgctgcttccgctggctcttctgctccacgccgctcggcccgatgtggtcatgac |
| Soluble scFv - | NO: 1402 | gcagtcaccactgtccctccccgtgacccttggacagccagcgtcgattagctgcaagtcatcccaatccctgctc |
| nt | | gattcggatggaaagacctatctcaactggctgcagcaaagacccggtcagagccctaggagactcatctcgttg |
| | | gtgtcaaagctggacagcggagtgccggaccggttttccggttcgggatcggggacggacttcactctgaagattt |
| | | cacgggtggaagctgaggatgtgggagtgtactactgctggcagggaacccatttccctggcacttttggcggag |
| | | gaactaaggtcgaaatcaagggaggaggtggctcgggaggaggcggatcgggcggaggcgggagcggcgg |
| | | aggagggtccgaaatccaacttgtccagtcaggagccgaagtgaagaaaccgggagccaccgtcaaaatcagc |
| | | tgtaagggatcgggattcaatatcgaggactactacatccactgggtgcagcaagctccgggcaaaggactggag |
| | | tggatggggcgcatcgacccagagaacgacgaaaccaaatacggcccgatcttccaagggcgggtgaccatca |
| | | ccgcggacacctcaactaacactgtgtacatggagctgagctccctgcgctccgaagatactgcagtctactactg |
| | | cgccttccgcggtggtgtgtactggggacagggcaccactgtgactgtcagctcggggtcccaccatcatcacca |
| | | ccaccatcac |
| CAR8 - | SEQ ID | malpvtalllplalllhaarpdvvmtqsplslpvtlgqpasisckssqslldsdgktylnwlqqrpgqsprrlislv |
| Soluble scFv - | NO: 1403 | skldsgvpdrfsgsgsgtdftlkisrveaedvgvyycwqgthfpgtfgggtkveikggggsggggsggggsg |
| aa | | gggseiqlvqsgaevkkpgatvkisckgsgfniedyyihwvqqapgkglewmgridpendetkygpifqgr |
| | | vtitadtstntvymelsslrsedtavyycafrggvywgqgttvtvssgshhhhhhhh |
| CAR8 - Full - | SEQ ID | atggccctccctgtcaccgccctgctgcttccgctggctcttctgctccacgccgctcggcccgatgtggtcatgac |
| nt | NO: 1404 | gcagtcaccactgtccctccccgtgacccttggacagccagcgtcgattagctgcaagtcatcccaatccctgctc |
| | | gattcggatggaaagacctatctcaactggctgcagcaaagacccggtcagagccctaggagactcatctcgttg |
| | | gtgtcaaagctggacagcggagtgccggaccggttttccggttcgggatcggggacggacttcactctgaagattt |
| | | cacgggtggaagctgaggatgtgggagtgtactactgctggcagggaacccatttccctggcacttttggcggag |
| | | gaactaaggtcgaaatcaagggaggaggtggctcgggaggaggcggatcgggcggaggcgggagcggcgg |
| | | aggagggtccgaaatccaacttgtccagtcaggagccgaagtgaagaaaccgggagccaccgtcaaaatcagc |
| | | tgtaagggatcgggattcaatatcgaggactactacatccactgggtgcagcaagctccgggcaaaggactggag |
| | | tggatggggcgcatcgacccagagaacgacgaaaccaaatacggcccgatcttccaagggcgggtgaccatca |
| | | ccgcggacacctcaactaacactgtgtacatggagctgagctccctgcgctccgaagatactgcagtctactactg |
| | | cgccttccgcggtggtgtgtactggggacagggcaccactgtgactgtcagctcgaccactaccccagcaccga |
| | | ggccacccaccccggctcctaccatcgcctcccagcctctgtccctgcgtccggaggcatgtagacccgcagctg |
| | | gtggggccgtgcatacccggggtcttgacttcgcctgcgatatctacatttgggcccctctggctggtacttgcggg |
| | | gtcctgctgctttcactcgtgatcactctttactgtaagcgcggtcggaagaagctgctgtacatctttaagcaacc |
| | | cttcatgaggcctgtgcagactactcaagaggaggacggctgttcatgccggttcccagaggaggaggaaggcggc |
| | | tgcgaactgcgcgtgaaattcagccgcagcgcagatgctccagcctacaagcaggggcagaaccagctctacaa |
| | | cgaactcaatcttggtcggagagaggagtacgacgtgctggacaagcggagaggacgggacccagaaatggg |
| | | cgggaagccgcgcagaaagaatccccaagagggcctgtacaacgagctccaaaaggataagatggcagaagc |
| | | ctatagcgagattggtatgaaaggggaacgcagaagaggcaaaggccacgacggactgtaccagggactcagc |
| | | accgccaccaaggacacctatgacgctcttcacatgcaggccctgccgcctcgg |
| CAR8 - Full - | SEQ ID | malpvtalllplalllhaarpdvvmtqsplslpvtlgqpasisckssqslldsdgktylnwlqqrpgqsprrlisl |
| aa | NO: 1405 | vskldsgvpdrfsgsgsgtdftlkisrveaedvgvyycwqgthfpgtfgggtkveikggggsggggsggggs |
| | | ggggseiqlvqsgaevkkpgatvkisckgsgfniedyyihwvqqapgkglewmgridpendetkygpif |
| | | qgrvtitadtstntvymelsslrsedtavyycafrggvywgqgttvtvsstapaprpptpaptiasqplslrpeac |
| | | rpaaggavhtrgldfacdiyiwaplagtcgvlllslvitlyckrgrkkllyifkqpfmrpvqttqeedgcscrfpee |
| | | eeggcelrvkfsrsadapaykqgqnqlynelnlgrreeydvldlargrdpemggkprrknpqeglynelqkd |
| | | kmaeayseigmkgerrrgkghdglyqglstatkdtydalhmqalppr |

CAR 9 Mouse anti-EGFRvIII clone 3C10

| | | |
|---|---|---|
| CAR9 scFv | SEQ ID | eiqlqqsgaelvkpgasvklsctgsgfniedyyihwvkqrteqglewigridpendetkygpifqgratitadts |
| domain | NO: 1406 | sntvylqlssltsedtavyycafrggvywgpgaltvssggggsggggsggggshmdvvmtqspltlsvaigq |
| | | sasisckssqslldsdgktylnwllqrpgqspkrlislvskldsgvpdrftgsgsgtdftlrisrveaedlgiyycw |
| | | qgthfpgtfgggtkleik |
| CAR9 scFv | SEQ ID | gagatccagctccaacagagcggagccgaactggtcaaaccgggagcgtcggtgaagttgtcatgcactggatc |
| domain nt | NO: 1407 | gggcttcaacatcgaggattactacatccactgggtcaagcaacgcaccgagcaggggctggaatggatcggac |
| | | ggatcgaccccgaaaacgatgaaaccaagtacgggcctatcttccaaggacgggccaccattacggctgacacg |
| | | tcaagcaataccgtctacctccagctttccagcctgacctccgaggacactgccgtgtactactgcgccttcagagg |
| | | aggcgtgtactggggaccaggaaccactttgaccgtgtccagcggaggcggtggatcaggaggaggaggctca |
| | | ggcggtggcggctcgcacatggacgtggtcatgactcagtccccgctgaccctgtcggtggcaattggacagag |
| | | cgcatccatctcgtgcaagagctcacagtcgctgctggattccgacggaaagacttatctgaactggctgctccaa |
| | | agaccagggcaatcaccgaaacgccttatctccctggtgtcgaaactcgactcgggtgtgccggatcggtttaccg |
| | | gtagcgggtccggcacggacttcactctccgcatttcgagggtggaagcggaggatctcgggatctactactgttg |
| | | gcagggaacccacttccctgggacttttggaggcggaactaagctggaaatcaag |
| CAR9 - | SEQ ID | atggccctccctgtcaccgccctgctgcttccgctggctcttctgctccacgccgctcggcccgagatccagctcca |
| Soluble scFv - | NO: 1408 | acagagcggagccgaactggtcaaaccgggagcgtcggtgaagttgtcatgcactggatcgggcttcaacatcg |
| nt | | aggattactacatccactgggtcaagcaacgcaccgagcaggggctggaatggatcggacggatcgaccccga |
| | | aaacgatgaaaccaagtacgggcctatcttccaaggacgggccaccattacggctgacacgtcaagcaataccgt |
| | | ctacctccagctttccagcctgacctccgaggacactgccgtgtactactgcgccttcagaggaggcgtgtactgg |
| | | ggaccaggaaccactttgaccgtgtccagcggaggcggtggatcaggaggaggaggctcaggcggtggcggc |
| | | tcgcacatggacgtggtcatgactcagtccccgctgaccctgtcggtggcaattggacagagcgcatccatctcgt |
| | | gcaagagctcacagtcgctgctggattccgacggaaagacttatctgaactggctgctccaaagaccagggcaat |
| | | caccgaaacgccttatctccctggtgtcgaaactcgactcgggtgtgccggatcggtttaccggtagcgggtccgg |
| | | cacggacttcactctccgcatttcgagggtggaagcggaggatctcgggatctactactgttggcagggaaccca |
| | | cttccctgggacttttggaggcggaactaagctggaaatcaagggtagccatcaccatcaccaccaccatcat |
| CAR9 - | SEQ ID | malpvtalllplalllhaarpeiqlqqsgaelvkpgasvklsctgsgfniedyyihwvkqrteqglewigridpe |
| Soluble scFv - | NO: 1409 | ndetkygpifqgratitadtssntvylqlssltsedtavyycafrggvywgpgttltvssggggsggggsggggs |
| aa | | hmdvvmtqspltlsvaigqsasisckssqslldsdgktylnwllqrpgqspkrlislvskldsgvpdrftgsgsgt |
| | | dftlrisrveaedlgiyycwqgthfpgtfgggtkleikgshhhhhhhh |
| CAR9 - Full - | SEQ ID | atggccctccctgtcaccgccctgctgcttccgctggctcttctgctccacgccgctcggcccgagatccagctcca |
| nt | NO: 1410 | acagagcggagccgaactggtcaaaccgggagcgtcggtgaagttgtcatgcactggatcgggcttcaacatcg |
| | | aggattactacatccactgggtcaagcaacgcaccgagcaggggctggaatggatcggacggatcgaccccga |
| | | aaacgatgaaaccaagtacgggcctatcttccaaggacgggccaccattacggctgacacgtcaagcaataccgt |
| | | ctacctccagctttccagcctgacctccgaggacactgccgtgtactactgcgccttcagaggaggcgtgtactgg |
| | | ggaccaggaaccactttgaccgtgtccagcggaggcggtggatcaggaggaggaggctcaggcggtggcggc |
| | | tcgcacatggacgtggtcatgactcagtccccgctgaccctgtcggtggcaattggacagagcgcatccatctcgt |
| | | gcaagagctcacagtcgctgctggattccgacggaaagacttatctgaactggctgctccaaagaccagggcaat |
| | | caccgaaacgccttatctccctggtgtcgaaactcgactcgggtgtgccggatcggtttaccggtagcgggtccgg |
| | | cacggacttcactctccgcatttcgagggtggaagcggaggatctcgggatctactactgttggcagggaaccca |
| | | cttccctgggacttttggaggcggaactaagctggaaatcaagaccactaccccagcaccgaggccacccaccc |
| | | cggctcctaccatcgcctcccagcctctgtccctgcgtccggaggcatgtagacccgcagctggtggggccgtgc |
| | | atacccggggtcttgacttcgcctgcgatatctacatttgggcccctctggctggtacttgcggggtcctgctgc |
| | | tttcactcgtgatcactctttactgtaagcgcggtcggaagaagctgctgtacatctttaagcaacccttcatga |
| | | ggcctgtgcagactactcaagaggaggacggctgttcatgccggttcccagaggaggaggaaggcggctgcgaac |
| | | tgcgcgtgaaattcagccgcagcgcagatgctccagcctacaagcaggggcagaaccagctctacaacgaactca |
| | | atcttggtcggagagaggagtacgacgtgctggacaagcggagaggacgggacccagaaatgggcgggaagccgc |
| | | gcagaaagaatccccaagagggcctgtacaacgagctccaaaaggataagatggcagaagcctatagcgagatt |
| | | ggtatgaaaggggaacgcagaagaggcaaaggccacgacggactgtaccagggactcagcaccgccaccaa |
| | | ggacacctatgacgctcttcacatgcaggccctgccgcctcgg |
| CAR9 - Full - | SEQ ID | malpvtalllplalllhaarpeiqlqqsgaelvkpgasvklsctgsgfniedyyihwvkqrteqglewigridpe |
| aa | NO: 1411 | ndetkyrgpifqgratitadtssntvylqlssltsedtavyycafrggvywgpgttltvssggggsggggsgggg |
| | | shmdvvmtqspltlsvaigqsasisckssqslldsdgktylnwllqrpgqspkrlislvskldsgvpdrftgsg |
| | | sgtdftlrisrveaedlgiyycwqgthfpgtfgggtkleiktttpaprpptpaptiasqplslrpeacrpaaggavh |
| | | trgldfacdiyiwaplagtcgvlllslvitlyckrgrkkllyifkqpfmrpvqttqeedgcscrfpeeeeggcelrv |
| | | kfsrsadapaykqgqnqlynelnlgrreeydvldkrrgrdpemggkprrknpqeglynelqkdkmaeayse |
| | | igmkgerrrgkghdglyqglstatkdtydalhmqalppr |

CAR10 Anti-EGFRvIII clone 139

| | | |
|---|---|---|
| CAR10 scFv | SEQ ID | diqmtqspsslsasvgdrvtitcrasqgirnnlawyqqkpgkapkrliyaasnlqsgvpsrftgsgsgteftlivs |
| domain | NO: 1412 | slqpedfatyyclqhhsypltsgggtkveikrtgstsgsgkpgsgegsevqvlesggglvqpggslrlscaasgft |
| | | fssyamswvrqapgkglewvsaisgsggstnyadsvkgrftisrdnskntlylqmnslraedtavyycagssg |
| | | wseywgqgtivtvss |
| CAR9 scFv | SEQ ID | gatatccaaatgactcagagcccttcatccctgagcgccagcgtcggagacagggtgaccatcacgtgccgggc |
| domain nt | NO: 1413 | atcccaaggcattagaaataacttggcgtggtatcagcaaaaaccaggaaaggccccgaagcgcctgatctacgc |
| | | ggcctccaaccttcagtcaggagtgccctcgcgcttcaccgggagcggtagcggaactgagtttacccttatcgtg |
| | | tcgtccctgcagccagaggacttcgcgacctactactgcctccagcatcactcgtacccgttgacttcgggaggcg |
| | | gaaccaaggtcgaaatcaaacgcactggctcgacgtcagggtccggtaaaccgggatcgggagaaggatcgga |
| | | agtccaagtgctggagagcggaggcggactcgtgcaacctggcgggtcgctgcggctcagctgtgccgcgtcg |
| | | ggttttactttcagctcgtacgctatgtcatgggtgcggcaggctccgggaaaggggctggaatgggtgtccgctat |
| | | ttccggctcgggtggaagcaccaattacgccgactccgtgaagggacgcttcaccatctcacgggataactccaa |
| | | gaatactctgtacctccagatgaactcgctgagagccgaggacaccgcagtgtactactgcgcagggtcaagcg |
| | | gctggtccgaatactggggacagggcaccctcgtcactgtcagctcc |
| CAR10 - | SEQ ID | atggccctccctgtcaccgccctgctgcttccgctggctcttctgctccacgccgctcggcccgatatccaaatgac |
| Soluble scFv - | NO: 1414 | tcagagcccttcatccctgagcgccagcgtcggagacagggtgaccatcacgtgccgggcatcccaaggcatta |
| nt | | gaaataacttggcgtggtatcagcaaaaaccaggaaaggccccgaagcgcctgatctacgcggcctccaaccttc |
| | | agtcaggagtgccctcgcgcttcaccgggagcggtagcggaactgagtttacccttatcgtgtcgtccctgcagcc |
| | | agaggacttcgcgacctactactgcctccagcatcactcgtacccgttgacttcgggaggcggaaccaaggtcga |
| | | aatcaaacgcactggctcgacgtcagggtccggtaaaccgggatcgggagaaggatcggaagtccaagtgctg |
| | | gagagcggaggcggactcgtgcaacctggcgggtcgctgcggctcagctgtgccgcgtcgggttttactttcagc |
| | | tcgtacgctatgtcatgggtgcggcaggctccgggaaaggggctggaatgggtgtccgctatttccggctcgggt |
| | | ggaagcaccaattacgccgactccgtgaagggacgcttcaccatctcacgggataactccaagaatactctgtacc |
| | | tccagatgaactcgctgagagccgaggacaccgcagtgtactactgcgcagggtcaagcggctggtccgaatac |
| | | tggggacagggcaccctcgtcactgtcagctcccatcaccatcaccaccaccatcac |
| CAR10 - | SEQ ID | malpvtalllplalllhaarpdiqmtqspsslsasvgdrvtitcrasqgirnnlawyqqkpgkapkrliyaasnlq |
| Soluble scFv - | NO: 1415 | sgvpsrftgsgsgteftlivsslqpedfatyyclqhhsypltsgggtkveikrtgstsgsgkpgsgegsevqvlesg |
| aa | | gglvqpggslrlscaasgftfssyamswvrqapgkglewvsaisgsggstnyadsvkgrftisrdnskntlylq |
| | | mnslraedtavyycagssgwseywgqgtlvtvsshhhhhhhh |
| CAR10 Full - | SEQ ID | atggccctccctgtcaccgccctgctgcttccgctggctcttctgctccacgccgctcggcccgatatccaaatgac |
| nt | NO: 1416 | tcagagcccttcatccctgagcgccagcgtcggagacagggtgaccatcacgtgccgggcatcccaaggcatta |
| | | gaaataacttggcgtggtatcagcaaaaaccaggaaaggccccgaagcgcctgatctacgcggcctccaaccttc |
| | | agtcaggagtgccctcgcgcttcaccgggagcggtagcggaactgagtttacccttatcgtgtcgtccctgcagcc |
| | | agaggacttcgcgacctactactgcctccagcatcactcgtacccgttgacttcgggaggcggaaccaaggtcga |
| | | aatcaaacgcactggctcgacgtcagggtccggtaaaccgggatcgggagaaggatcggaagtccaagtgctg |
| | | gagagcggaggcggactcgtgcaacctggcgggtcgctgcggctcagctgtgccgcgtcgggttttactttcagc |
| | | tcgtacgctatgtcatgggtgcggcaggctccgggaaaggggctggaatgggtgtccgctatttccggctcgggt |
| | | ggaagcaccaattacgccgactccgtgaagggacgcttcaccatctcacgggataactccaagaatactctgtacc |
| | | tccagatgaactcgctgagagccgaggacaccgcagtgtactactgcgcagggtcaagcggctggtccgaatac |
| | | tggggacagggcaccctcgtcactgtcagctccaccactaccccagcaccgaggccacccaccccggctcctac |
| | | catcgcctcccagcctctgtccctgcgtccggaggcatgtagacccgcagctggtggggccgtgcatacccggg |
| | | gtcttgacttcgcctgcgatatctacatttgggcccctctggctggtacttgcggggtcctgctgctttcactc |
| | | gtgatcactctttactgtaagcgcggtcggaagaagctgctgtacatctttaagcaacccttcatgaggcctgt |
| | | gcagactactcaagaggaggacggctgttcatgccggttcccagaggaggaggaaggcggctgcgaactgcgcg |
| | | tgaaattcagccgcagcgcagatgctccagcctacaagcaggggcagaaccagctctacaacgaactcaatctt |
| | | ggtcggagagaggagtacgacgtgctggacaagcggagaggacgggacccagaaatgggcgggaagccgcgcag |
| | | aaagaatccccaagagggcctgtacaacgagctccaaaaggataagatggcagaagcctatagcgagattggta |
| | | tgaaaggggaacgcagaagaggcaaaggccacgacggactgtaccagggactcagcaccgccaccaaggacaccta |
| | | tgacgctcttcacatgcaggccctgccgcctcgg |
| CAR10 Full - | SEQ ID | malpvtalllplalllhaarpdiqmtqspsslsasvgdrvtitcrasqgirnnlawyqqkpgkapkrliyaasnlq |
| aa | NO: 1417 | sgvpsrftgsgsgteftlivsslqpedfatyyclqhhsypltsgggtkveikrtgstsgsgkpgsgegsevqvlesg |
| | | gglvqpggslrlscaasgftfssyamswvrqapgkglewvsaisgsggstnyadsvkgrftisrdnskntlylq |
| | | mnslraedtavyycagssgwseywgqgtivtvsstapaprpptpaptiasqplslrpeacrpaaggavhtrgld |
| | | facdiyiwaplagtcgvlllslvitlyckrgrkkllyiflawfmrpvqttqeedgcscrfpeeeeggcelrvkfsrs |
| | | adapaykqgqnqlynelnlgrreeydvldlargrdpemggkprrknpqeglynelqkdkmaeayseigmk |
| | | gerrrgkghdglyqglstatkdtydalhmqalppr |

### Mesothelin CAR and Mesothelin-Binding Sequences

In some embodiments, the CAR IL-15R/IL-15-expressing cell described herein is a mesothelin CAR-expressing cell (e.g., a cell expressing a CAR that binds to human mesothelin). Exemplary mesothelin CARs can include sequences disclosed in WO2015090230 and WO2017112741, e.g., Tables 2, 3, 4, and 5 of WO2017112741, incorporated herein by reference.

Exemplary mesothelin-binding sequences or mesothelin CAR sequences may comprise a CDR, a variable region, an scFv, or a full-length CAR sequence of a sequence disclosed in Table 10 (or a sequence at least about 85%, 90%, 95%, 99% or more identical thereto, and/or having one, two, three or more substitutions, insertions, deletions, or modifications).

TABLE 10
Amino Acid Sequences of Human scFvs and CARs that bind to mesothelin
(bold underline is the leader sequence and grey box is a linker sequence).
In the case of the scFvs, the remaining amino acids are the heavy chain
variable region and light chain variable regions, with each of the HC CDRs
(HC CDR1, HC CDR2, HC CDR3) and LC CDRs (LC CDR1, LC CDR2, LCCDR3) underlined.
In the case of the CARs, the further remaining amino acids
are the remaining amino acids of the CARs.

| SEQ ID | | |
|---|---|---|
| NO: | Description | Amino Acid Sequence |
| SEQ ID | M1 (ScFv | QVQLQQSGAEVKKPGASVKVSCKASGYTFTGYYMHWVRQ |
| NO: | domain) | APGQGLEWMGRINPNSGGTNYAQKFQGRVTMTRDTSISTAYMELSRLRSEDTAVYYCARG |
| 1418 | | RYYGMDVWGQGTMVTVSSGGGGSGGGGSGGGGSGGGGSEIVLTQSPATLSLSPGERATIS |
| | | CRASQSVSSNFAWYQQRPGQAPRLLIYDASNRATGIPPRFSGSGSGTDFTLTISSLEPED |
| | | FAAYYCHQRSNWLYTFGQGTKVDIK |
| SEQ ID | M1 (full) | MALPVTALLLPLALLLHAARPQVQLQQSGAEVKKPGASVKVSCKASGYTFTGYYMHWVRQ |
| NO: | >ZA53- | APGQGLEWMGRINPNSGGTNYAQKFQGRVTMTRDTSISTAYMELSRLRSEDTAVYYCARG |
| 1419 | 27BC | RYYGMDVWGQGTMVTVSSGGGGSGGGGSGGGGSGGGGSEIVLTQSPATLSLSPGERATIS |
| | (M1 | CRASQSVSSNFAWYQQRPGQAPRLLIYDASNRATGIPPRFSGSGSGTDFTLTISSLEPED |
| | ZA53- | FAAYYCHQRSNWLYTFGQGTKVDIKTTTPAPRPPTPAPTIASQPLSLRPEACRPAAGGAV |
| | 27BC | HTRGLDFACDIYIWAPLAGTCGVLLLSLVITLYCKRGRKKLLYIFKQPFMRPVQTTQEED |
| | R001- | GCSCRFPEEEEGGCELRVKFSRSADAPAYKQGQNQLYNELNLGRREEYDVLDKRRGRDPE |
| | A11 | MGGKPRRKNPQEGLYNELQKDKMAEAYSEIGMKGERRRGKGHDGLYQGLSTATKDTYDAL |
| | 126161) | HMQALPPR |
| SEQ ID | M2 (ScFv | QVQLVQSGAEVKKPGASVKVSCKASGYTFTGYYMHWVRQ |
| NO: | domain) | APGQGLEWMGWINPNSGGTNYAQKFQGRVTMTRDTSISTAYMELSRLRSDDTAVYYCARD |
| 1420 | | LRRTVVTPRAYYGMDVWGQGTTVTVSSGGGGSGGGGSGGGGSGGGGSDIQLTQSPSTLSA |
| | | SVGDRVTITCQASQDISNSLNWYQQKAGKAPKLLIYDASTLETGVPSRFSGSGSGTDFSF |
| | | TISSLQPEDIATYYCQQHDNLPLTFGQGTKVEIK |
| SEQ ID | M2 (full) | MALPVTALLLPLALLLHAARPQVQLVQSGAEVKKPGASVKVSCKASGYTFTGYYMHWVRQ |
| NO: | >FA56- | APGQGLEWMGWINPNSGGTNYAQKFQGRVTMTRDTSISTAYMELSRLRSDDTAVYYCARD |
| 1421 | 26RC | LRRTVVTPRAYYGMDVWGQGTTVTVSSGGGGSGGGGSGGGGSGGGGSDIQLTQSPSTLSA |
| | (M2 | SVGDRVTITCQASQDISNSLNWYQQKAGKAPKLLIYDASTLETGVPSRFSGSGSGTDFSF |
| | FA56- | TISSLQPEDIATYYCQQHDNLPLTFGQGTKVEIKTTTPAPRPPTPAPTIASQPLSLRPEA |
| | 26RC | CRPAAGGAVHTRGLDFACDIYIWAPLAGTCGVLLLSLVITLYCKRGRKKLLYIFKQPFMR |
| | R001- | PVQTTQEEDGCSCRFPEEEEGGCELRVKFSRSADAPAYKQGQNQLYNELNLGRREEYDVL |
| | A10 | DKRRGRDPEMGGKPRRKNPQEGLYNELQKDKMAEAYSEIGMKGERRRGKGHDGLYQGLST |
| | 126162) | ATKDTYDALHMQALPPR |
| SEQ ID | M3 (ScFv | QVQLVQSGAEVKKPGAPVKVSCKASGYTFTGYYMHWVRQ |
| NO: | domain) | APGQGLEWMGWINPNSGGTNYAQKFQGRVTMTRDTSISTAYMELSRLRSDDTAVYYCARG |
| 1422 | | EWDGSYYYDYWGQGTLVTVSSGGGGSGGGGSGGGGSGGGGSDIVLTQTPSSLSASVGDRV |
| | | TITCRASQSINTYLNWYQHKPGKAPKLLIYAASSLQSGVPSRFSGSGSGTDFTLTISSLQ |
| | | PEDFATYYCQQSFSPLTFGGGTKLEIK |
| SEQ ID | M3 | MALPVTALLLPLALLLHAARPQVQLVQSGAEVKKPGAPVKVSCKASGYTFTGYYMHWVRQ |
| NO: | >VA58- | APGQGLEWMGWINPNSGGTNYAQKFQGRVTMTRDTSISTAYMELSRLRSDDTAVYYCARG |
| 1423 | 21LC | EWDGSYYYDYWGQGTLVTVSSGGGGSGGGGSGGGGSGGGGSDIVLTQTPSSLSASVGDRV |
| | (M3 | TITCRASQSINTYLNWYQHKPGKAPKLLIYAASSLQSGVPSRFSGSGSGTDFTLTISSLQ |
| | VA58- | PEDFATYYCQQSFSPLTFGGGTKLEIKTTTPAPRPPTPAPTIASQPLSLRPEACRPAAGG |
| | 21LC | AVHTRGLDFACDIYIWAPLAGTCGVLLLSLVITLYCKRGRKKLLYIFKQPFMRPVQTTQE |
| | R001-A1 | EDGCSCRFPEEEEGGCELRVKFSRSADAPAYKQGQNQLYNELNLGRREEYDVLDKRRGRD |
| | 126163) | PEMGGKPRRKNPQEGLYNELQKDKMAEAYSEIGMKGERRRGKGHDGLYQGLSTATKDTYD |
| | | ALHMQALPPR |
| SEQ ID | M4 (ScFv | QVQLVESGGGLVQPGGSLRLSCAASGFTFSSYWMHWVRQ |
| NO: | domain) | VPGKGLVWVSRINTDGSTTTYADSVEGRFTISRDNAKNTLYLQMNSLRDDDTAVYYCVGG |
| 1424 | | HWAVWGQGTTVTVSSGGGGSGGGGSGGGGSGGGGSDIQMTQSPSTLSASVGDRVTITCRA |
| | | SQSISDRLAWYQQKPGKAPKLLIYKASSLESGVPSRFSGSGSGTEFTLTISSLQPDDFAV |
| | | YYCQQYGHLPMYTFGQGTKVEIK |
| SEQ ID | M4 | MALPVTALLLPLALLLHAARPQVQLVESGGGLVQPGGSLRLSCAASGFTFSSYWMHWVRQ |
| NO: | >DP37- | VPGKGLVWVSRINTDGSTTTYADSVEGRFTISRDNAKNTLYLQMNSLRDDDTAVYYCVGG |
| 1425 | 07IC | HWAVWGQGTTVTVSSGGGGSGGGGSGGGGSGGGGSDIQMTQSPSTLSASVGDRVTITCRA |
| | (M4 | SQSISDRLAWYQQKPGKAPKLLIYKASSLESGVPSRFSGSGSGTEFTLTISSLQPDDFAV |
| | DP37- | YYCQQYGHLPMYTFGQGTKVEIKTTTPAPRPPTPAPTIASQPLSLRPEACRPAAGGAVHT |
| | 07IC | RGLDFACDIYIWAPLAGTCGVLLLSLVITLYCKRGRKKLLYIFKQPFMRPVQTTQEEDGC |
| | R001-C6 | SCRFPEEEEGGCELRVKFSRSADAPAYKQGQNQLYNELNLGRREEYDVLDKRRGRDPEMG |
| | 126164) | GKPRRKNPQEGLYNELQKDKMAEAYSEIGMKGERRRGKGHDGLYQGLSTATKDTYDALHM |
| | | QALPPR |
| SEQ ID | M5 (ScFv | QVQLVQSGAEVEKPGASVKVSCKASGYTFTDYYMHWVRQ |
| NO: | domain) | APGQGLEWMGWINPNSGGTNYAQKFQGRVTMTRDTSISTAYMELSRLRSDDTAVYYCASG |
| 1426 | | WDFDYWGQGTLVTVSSGGGGSGGGGSGGGGSGGGGSDIVMTQSPSSLSASVGDRVTITCR |
| | | ASQSIRYYLSWYQQKPGKAPKLLIYTASILQNGVPSRFSGSGSGTDFTLTISSLQPEDFA |
| | | TYYCLQTYTTPDFGPGTKVEIK |
| SEQ ID | M5 | MALPVTALLLPLALLLHAARPQVQLVQSGAEVEKPGASVKVSCKASGYTFTDYYMHWVRQ |
| NO: | >XP31- | APGQGLEWMGWINPNSGGTNYAQKFQGRVTMTRDTSISTAYMELSRLRSDDTAVYYCASG |
| 1427 | 20LC | WDFDYWGQGTLVTVSSGGGGSGGGGSGGGGSGGGGSDIVMTQSPSSLSASVGDRVTITCR |
| | (M5 | ASQSIRYYLSWYQQKPGKAPKLLIYTASILQNGVPSRFSGSGSGTDFTLTISSLQPEDFA |
| | XP31- | TYYCLQTYTTPDFGPGTKVEIKTTTPAPRPPTPAPTIASQPLSLRPEACRPAAGGAVHTR |
| | 20LC | GLDFACDIYIWAPLAGTCGVLLLSLVITLYCKRGRKKLLYIFKQPFMRPVQTTQEEDGCS |
| | R001-B4 | CRFPEEEEGGCELRVKFSRSADAPAYKQGQNQLYNELNLGRREEYDVLDKRRGRDPEMGG |
| | 126165) | KPRRKNPQEGLYNELQKDKMAEAYSEIGMKGERRRGKGHDGLYQGLSTATKDTYDALHMQ |
| | | ALPPR |
| SEQ ID | M6 (ScFv | QVQLVQSGAEVKKPGASVKVSCKASGYTFTSYYMHWVRQ |
| NO: | domain) | APGQGLEWMGIINPSGGSTSYAQKFQGRVTMTRDTSTSTVYMELSSLRSEDTAVYYCARY |
| 1428 | | RLIAVAGDYYYYGMDVWGQGTMVTVSSGGGGSGGGGSGGGGSGGGGSDIQMTQSPSSVSA |
| | | SVGDRVTITCRASQGVGRWLAWYQQKPGTAPKLLIYAASTLQSGVPSRFSGSGSGTDFTL |
| | | TINNLQPEDFATYYCQQANSFPLTFGGGTRLEIK |
| SEQ ID | M6 | MALPVTALLLPLALLLHAARPQVQLVQSGAEVKKPGASVKVSCKASGYTFTSYYMHWVRQ |
| NO: | >FE10- | APGQGLEWMGIINPSGGSTSYAQKFQGRVTMTRDTSTSTVYMELSSLRSEDTAVYYCARY |
| 1429 | 06ID | RLIAVAGDYYYYGMDVWGQGTMVTVSSGGGGSGGGGSGGGGSGGGGSDIQMTQSPSSVSA |
| | (M6 | SVGDRVTITCRASQGVGRWLAWYQQKPGTAPKLLIYAASTLQSGVPSRFSGSGSGTDFTL |
| | 46FE10- | TINNLQPEDFATYYCQQANSFPLTFGGGTRLEIKTTTPAPRPPTPAPTIASQPLSLRPEA |
| | 06ID | CRPAAGGAVHTRGLDFACDIYIWAPLAGTCGVLLLSLVITLYCKRGRKKLLYIFKQPFMR |
| | R001-A4 | PVQTTQEEDGCSCRFPEEEEGGCELRVKFSRSADAPAYKQGQNQLYNELNLGRREEYDVL |
| | 126166) | DKRRGRDPEMGGKPRRKNPQEGLYNELQKDKMAEAYSEIGMKGERRRGKGHDGLYQGLST |
| | | ATKDTYDALHMQALPPR |
| SEQ ID | M7 (ScFv | QVQLVQSGGGVVQPGRSLRLSCAASGFTFSSYAMHWVRQ |
| NO: | domain) | APGKGLEWVAVISYDGSNKYYADSVKGRFTISRDNSKNTLYLQMNSLRAEDTAVYYCARW |
| 1430 | | KVSSSSPAFDYWGQGTLVTVSSGGGGSGGGGSGGGGSGGGGSEIVLTQSPATLSLSPGER |
| | | AILSCRASQSVYTKYLGWYQQKPGQAPRLLIYDASTRATGIPDRFSGSGSGTDFTLTINR |
| | | LEPEDFAVYYCQHYGGSPLITFGQGTRLEIK |
| SEQ ID | M7 | MALPVTALLLPLALLLHAARPQVQLVQSGGGVVQPGRSLRLSCAASGFTFSSYAMHWVRQ |
| NO: | >VE12- | APGKGLEWVAVISYDGSNKYYADSVKGRFTISRDNSKNTLYLQMNSLRAEDTAVYYCARW |
| 1431 | 01CD | KVSSSSPAFDYWGQGTLVTVSSGGGGSGGGGSGGGGSGGGGSEIVLTQSPATLSLSPGER |
| | (M7 | AILSCRASQSVYTKYLGWYQQKPGQAPRLLIYDASTRATGIPDRFSGSGSGTDFTLTINR |
| | VE12- | LEPEDFAVYYCQHYGGSPLITFGQGTRLEIKTTTPAPRPPTPAPTIASQPLSLRPEACRP |
| | 01CD | AAGGAVHTRGLDFACDIYIWAPLAGTCGVLLLSLVITLYCKRGRKKLLYIFKQPFMRPVQ |
| | R001-A5 | TTQEEDGCSCRFPEEEEGGCELRVKFSRSADAPAYKQGQNQLYNELNLGRREEYDVLDKR |
| | 126167) | RGRDPEMGGKPRRKNPQEGLYNELQKDKMAEAYSEIGMKGERRRGKGHDGLYQGLSTATK |
| | | DTYDALHMQALPPR |
| SEQ ID | M8 (ScFv | QVQLQQSGAEVKKPGASVKVSCKTSGYPFTGYSLHWVRQ |
| NO: | domain) | APGQGLEWMGWINPNSGGTNYAQKFQGRVTMTRDTSISTAYMELSRLRSDDTAVYYCARD |
| 1432 | | HYGGNSLFYWGQGTLVTVSSGGGGSGGGGSGGGGSGGGGSDIQLTQSPSSISASVGDTVS |
| | | ITCRASQDSGTWLAWYQQKPGKAPNLLMYDASTLEDGVPSRFSGSASGTEFTLTVNRLQP |
| | | EDSATYYCQQYNSYPLTFGGGTKVDIK |
| SEQ ID | M8 | MALPVTALLLPLALLLHAARPQVQLQQSGAEVKKPGASVKVSCKTSGYPFTGYSLHWVRQ |
| NO: | >LE13- | APGQGLEWMGWINPNSGGTNYAQKFQGRVTMTRDTSISTAYMELSRLRSDDTAVYYCARD |
| 1433 | 05XD | HYGGNSLFYWGQGTLVTVSSGGGGSGGGGSGGGGSGGGGSDIQLTQSPSSISASVGDTVS |
| | (M8 | ITCRASQDSGTWLAWYQQKPGKAPNLLMYDASTLEDGVPSRFSGSASGTEFTLTVNRLQP |
| | LE13- | EDSATYYCQQYNSYPLTFGGGTKVDIKTTTPAPRPPTPAPTIASQPLSLRPEACRPAAGG |
| | 05XD | AVHTRGLDFACDIYIWAPLAGTCGVLLLSLVITLYCKRGRKKLLYIFKQPFMRPVQTTQE |
| | R001-E5 | EDGCSCRFPEEEEGGCELRVKFSRSADAPAYKQGQNQLYNELNLGRREEYDVLDKRRGRD |
| | 126168) | PEMGGKPRRKNPQEGLYNELQKDKMAEAYSEIGMKGERRRGKGHDGLYQGLSTATKDTYD |
| | | ALHMQALPPR |
| SEQ ID | M9 (ScFv | QVQLVQSGAEVKKPGASVEVSCKASGYTFTSYYMHWVRQ |
| NO: | domain) | APGQGLEWMGIINPSGGSTGYAQKFQGRVTMTRDTSTSTVHMELSSLRSEDTAVYYCARG |
| 1434 | | GYSSSSDAFDIWGQGTMVTVSSGGGGSGGGGSGGGGSGGGGSDIQMTQSPPSLSASVGDR |
| | | VTITCRASQDISSALAWYQQKPGTPPKLLIYDASSLESGVPSRFSGSGSGTDFTLTISSL |
| | | QPEDFATYYCQQFSSYPLTFGGGTRLEIK |
| SEQ ID | M9 | MALPVTALLLPLALLLHAARPQVQLVQSGAEVKKPGASVEVSCKASGYTFTSYYMHWVRQ |
| NO: | >BE15- | APGQGLEWMGIINPSGGSTGYAQKFQGRVTMTRDTSTSTVHMELSSLRSEDTAVYYCARG |
| 1435 | 00SD | GYSSSSDAFDIWGQGTMVTVSSGGGGSGGGGSGGGGSGGGGSDIQMTQSPPSLSASVGDR |
| | (M9 | VTITCRASQDISSALAWYQQKPGTPPKLLIYDASSLESGVPSRFSGSGSGTDFTLTISSL |
| | BE15- | QPEDFATYYCQQFSSYPLTFGGGTRLEIKTTTPAPRPPTPAPTIASQPLSLRPEACRPAA |
| | 00SD | GGAVHTRGLDFACDIYIWAPLAGTCGVLLLSLVITLYCKRGRKKLLYIFKQPFMRPVQTT |
| | R001-A3 | QEEDGCSCRFPEEEEGGCELRVKFSRSADAPAYKQGQNQLYNELNLGRREEYDVLDKRRG |
| | 126169) | RDPEMGGKPRRKNPQEGLYNELQKDKMAEAYSEIGMKGERRRGKGHDGLYQGLSTATKDT |
| | | YDALHMQALPPR |
| SEQ ID | M10 | QVQLVQSGAEVKKPGASVKVSCKASGYTFTSYGISWVRQ |
| NO: | (ScFv | APGQGLEWMGWISAYNGNTNYAQKLQGRVTMTTDTSTSTAYMELRSLRSDDTAVYYCARV |
| 1436 | domain) | AGGIYYYYGMDVWGQGTTITVSSGGGGSGGGGSGGGGSGGGGSDIVMTQTPDSLAVSLGE |
| | | RATISCKSSHSVLYNRNNKNYLAWYQQKPGQPPKLLFYWASTRKSGVPDRFSGSGSGTDF |
| | | TLTISSLQPEDFATYFCQQTQTFPLTFGQGTRLEIN |
| SEQ ID | M10 | MALPVTALLLPLALLLHAARPQVQLVQSGAEVKKPGASVKVSCKASGYTFTSYGISWVRQ |
| NO: | >RE16- | APGQGLEWMGWISAYNGNTNYAQKLQGRVTMTTDTSTSTAYMELRSLRSDDTAVYYCARV |
| 1437 | 05MD | AGGIYYYYGMDVWGQGTTITVSSGGGGSGGGGSGGGGSGGGGSDIVMTQTPDSLAVSLGE |
| | (M10 | RATISCKSSHSVLYNRNNKNYLAWYQQKPGQPPKLLFYWASTRKSGVPDRFSGSGSGTDF |
| | RE16- | TLTISSLQPEDFATYFCQQTQTFPLTFGQGTRLEINTTTPAPRPPTPAPTIASQPLSLRP |
| | 05MD | EACRPAAGGAVHTRGLDFACDIYIWAPLAGTCGVLLLSLVITLYCKRGRKKLLYIFKQPF |
| | R001- | MRPVQTTQEEDGCSCRFPEEEEGGCELRVKFSRSADAPAYKQGQNQLYNELNLGRREEYD |
| | D10 | VLDKRRGRDPEMGGKPRRKNPQEGLYNELQKDKMAEAYSEIGMKGERRRGKGHDGLYQGL |
| | 126170) | STATKDTYDALHMQALPPR |
| SEQ ID | M11 | QVQLQQSGAEVKKPGASVKVSCKASGYTFTGYYMHWVRQ |
| NO: | (ScFv | APGQGLEWMGWINPNSGGTNYAQNFQGRVTMTRDTSISTAYMELRRLRSDDTAVYYCASG |
| 1438 | domain) | WDFDYWGQGTLVTVSSGGGGSGGGGSGGGGSGGGGSDIRMTQSPSSLSASVGDRVTITCR |
| | | ASQSIRYYLSWYQQKPGKAPKLLIYTASILQNGVPSRFSGSGSGTDFTLTISSLQPEDFA |
| | | TYYCLQTYTTPDFGPGTKVEIK |
| SEQ ID | M11 | MALPVTALLLPLALLLHAARPQVQLQQSGAEVKKPGASVKVSCKASGYTFTGYYMHWVRQ |
| NO: | >NE10- | APGQGLEWMGWINPNSGGTNYAQNFQGRVTMTRDTSISTAYMELRRLRSDDTAVYYCASG |
| 1439 | 19WD | WDFDYWGQGTLVTVSSGGGGSGGGGSGGGGSGGGGSDIRMTQSPSSLSASVGDRVTITCR |
| | (M11 | ASQSIRYYLSWYQQKPGKAPKLLIYTASILQNGVPSRFSGSGSGTDFTLTISSLQPEDFA |
| | NE10- | TYYCLQTYTTPDFGPGTKVEIKTTTPAPRPPTPAPTIASQPLSLRPEACRPAAGGAVHTR |
| | 19WD | GLDFACDIYIWAPLAGTCGVLLLSLVITLYCKRGRKKLLYIFKQPFMRPVQTTQEEDGCS |
| | R001-G2 | CRFPEEEEGGCELRVKFSRSADAPAYKQGQNQLYNELNLGRREEYDVLDKRRGRDPEMGG |
| | 126171) | KPRRKNPQEGLYNELQKDKMAEAYSEIGMKGERRRGKGHDGLYQGLSTATKDTYDALHMQ |
| | | ALPPR |
| SEQ ID | M12 | QVQLVQSGAEVKKPGASVKVSCKASGYTFTGYYMHWVRQ |
| NO: | ScFv | APGQGLEWMGRINPNSGGTNYAQKFQGRVTMTTDTSTSTAYMELRSLRSDDTAVYYCART |
| 1440 | domain) | TTSYAFDIWGQGTMVTVSSGGGGSGGGGSGGGGSGGGGSDIQLTQSPSTLSASVGDRVTI |
| | | TCRASQSISTWLAWYQQKPGKAPNLLIYKASTLESGVPSRFSGSGSGTEFTLTISSLQPD |
| | | DFATYYCQQYNTYSPYTFGQGTKLEIK |
| SEQ ID | M12 | MALPVTALLLPLALLLHAARPQVQLVQSGAEVKKPGASVKVSCKASGYTFTGYYMHWVRQ |
| NO: | >DE12- | APGQGLEWMGRINPNSGGTNYAQKFQGRVTMTTDTSTSTAYMELRSLRSDDTAVYYCART |
| 1441 | 14RD | TTSYAFDIWGQGTMVTVSSGGGGSGGGGSGGGGSGGGGSDIQLTQSPSTLSASVGDRVTI |
| | (M12 | TCRASQSISTWLAWYQQKPGKAPNLLIYKASTLESGVPSRFSGSGSGTEFTLTISSLQPD |
| | DE12- | DFATYYCQQYNTYSPYTFGQGTKLEIKTTTPAPRPPTPAPTIASQPLSLRPEACRPAAGG |
| | 14RD | AVHTRGLDFACDIYIWAPLAGTCGVLLLSLVITLYCKRGRKKLLYIFKQPFMRPVQTTQE |
| | R001-G9 | EDGCSCRFPEEEEGGCELRVKFSRSADAPAYKQGQNQLYNELNLGRREEYDVLDKRRGRD |
| | 126172) | PEMGGKPRRKNPQEGLYNELQKDKMAEAYSEIGMKGERRRGKGHDGLYQGLSTATKDTYD |
| | | ALHMQALPPR |
| SEQ ID | M13 | QVQLVQSGGGLVKPGGSLRLSCEASGFIFSDYYMGWIRQ |
| NO: | (ScFv | APGKGLEWVSYIGRSGSSMYYADSVKGRFTFSRDNAKNSLYLQMNSLRAEDTAVYYCAAS |
| 1442 | domain) | PVVAATEDFQHWGQGTLVTVSSGGGGSGGGGSGGGGSGGGGSDIVMTQTPATLSLSPGER |
| | | ATLSCRASQSVTSNYLAWYQQKPGQAPRLLLFGASTRATGIPDRFSGSGSGTDFTLTINR |
| | | LEPEDFAMYYCQQYGSAPVTFGQGTKLEIK |
| SEQ ID | M13 | MALPVTALLLPLALLLHAARPQVQLVQSGGGLVKPGGSLRLSCEASGFIFSDYYMGWIRQ |
| NO: | >TE13- | APGKGLEWVSYIGRSGSSMYYADSVKGRFTFSRDNAKNSLYLQMNSLRAEDTAVYYCAAS |
| 1443 | 19LD | PVVAATEDFQHWGQGTLVTVSSGGGGSGGGGSGGGGSGGGGSDIVMTQTPATLSLSPGER |
| | (M13 | ATLSCRASQSVTSNYLAWYQQKPGQAPRLLLFGASTRATGIPDRFSGSGSGTDFTLTINR |
| | TE13- | LEPEDFAMYYCQQYGSAPVTFGQGTKLEIKTTTPAPRPPTPAPTIASQPLSLRPEACRPA |
| | 19LD | AGGAVHTRGLDFACDIYIWAPLAGTCGVLLLSLVITLYCKRGRKKLLYIFKQPFMRPVQT |
| | R002-C3 | TQEEDGCSCRFPEEEEGGCELRVKFSRSADAPAYKQGQNQLYNELNLGRREEYDVLDKRR |
| | 126173) | GRDPEMGGKPRRKNPQEGLYNELQKDKMAEAYSEIGMKGERRRGKGHDGLYQGLSTATKD |
| | | TYDALHMQALPPR |
| SEQ ID | M14 | QVQLVQSGAEVRAPGASVKISCKASGFTFRGYYIHWVRQ |
| NO: | (ScFv | APGQGLEWMGIINPSGGSRAYAQKFQGRVTMTRDTSTSTVYMELSSLRSDDTAMYYCART |
| 1444 | domain) | ASCGGDCYYLDYWGQGTLVTVSSGGGGSGGGGSGGGGSGGGGSDIQMTQSPPTLSASVGD |
| | | RVTITCRASENVNIWLAWYQQKPGKAPKLLIYKSSSLASGVPSRFSGSGSGAEFTLTISS |
| | | LQPDDFATYYCQQYQSYPLTFGGGTKVDIK |
| SEQ ID | M14 | MALPVTALLLPLALLLHAARPQVQLVQSGAEVRAPGASVKISCKASGFTFRGYYIHWVRQ |
| NO: | >BS83- | APGQGLEWMGIINPSGGSRAYAQKFQGRVTMTRDTSTSTVYMELSSLRSDDTAMYYCART |
| 1445 | 951D | ASCGGDCYYLDYWGQGTLVTVSSGGGGSGGGGSGGGGSGGGGSDIQMTQSPPTLSASVGD |
| | (M14 | RVTITCRASENVNIWLAWYQQKPGKAPKLLIYKSSSLASGVPSRFSGSGSGAEFTLTISS |
| | BS83- | LQPDDFATYYCQQYQSYPLTFGGGTKVDIKTTTPAPRPPTPAPTIASQPLSLRPEACRPA |
| | 95ID | AGGAVHTRGLDFACDIYIWAPLAGTCGVLLLSLVITLYCKRGRKKLLYIFKQPFMRPVQT |
| | R001-E8 | TQEEDGCSCRFPEEEEGGCELRVKFSRSADAPAYKQGQNQLYNELNLGRREEYDVLDKRR |
| | 126174) | GRDPEMGGKPRRKNPQEGLYNELQKDKMAEAYSEIGMKGERRRGKGHDGLYQGLSTATKD |
| | | TYDALHMQALPPR |
| SEQ ID | M15 | QVQLVQSGGGLVQPGRSLRLSCAASGFTFDDYAMHWVRQ |
| NO: | (ScFv | APGKGLEWVSGISWNSGSIGYADSVKGRFTISRDNAKNSLYLQMNSLRAEDTAVYYCAKD |
| 1446 | domain) | GSSSWSWGYFDYWGQGTLVTVSSGGGGSGGGGSGGGGSSSELTQDPAVSVALGQTVRTTC |
| | | QGDALRSYYASWYQQKPGQAPMLVIYGKNNRPSGIPDRFSGSDSGDTASLTITGAQAEDE |
| | | ADYYCNSRDSSGYPVFGTGTKVTVL |
| SEQ ID | M15 | MALPVTALLLPLALLLHAARPQVQLVQSGGGLVQPGRSLRLSCAASGFTFDDYAMHWVRQ |
| NO: | >HS86- | APGKGLEWVSGISWNSGSIGYADSVKGRFTISRDNAKNSLYLQMNSLRAEDTAVYYCAKD |
| 1447 | 94XD | GSSSWSWGYFDYWGQGTLVTVSSGGGGSGGGGSGGGGSSSELTQDPAVSVALGQTVRTTC |
| | (M15 | QGDALRSYYASWYQQKPGQAPMLVIYGKNNRPSGIPDRFSGSDSGDTASLTITGAQAEDE |
| | HS86- | ADYYCNSRDSSGYPVFGTGTKVTVLTTTPAPRPPTPAPTIASQPLSLRPEACRPAAGGAV |
| | 94XD | HTRGLDFACDIYIWAPLAGTCGVLLLSLVITLYCKRGRKKLLYIFKQPFMRPVQTTQEED |
| | NT | GCSCRFPEEEEGGCELRVKFSRSADAPAYKQGQNQLYNELNLGRREEYDVLDKRRGRDPE |
| | 127553) | MGGKPRRKNPQEGLYNELQKDKMAEAYSEIGMKGERRRGKGHDGLYQGLSTATKDTYDAL |
| | | HMQALPPR |
| SEQ ID | M16 | EVQLVESGGGLVQPGRSLRLSCAASGFTFDDYAMHWVRQ |
| NO: | (ScFv | APGKGLEWVSGISWNSGSTGYADSVKGRFTISRDNAKNSLYLQMNSLRAEDTALYYCAKD |
| 1448 | domain) | SSSWYGGGSAFDIWGQGTMVTVSSGGGGSGGGGSGGGGSSSELTQEPAVSVALGQTVRIT |
| | | CQGDSLRSYYASWYQQKPGQAPVLVIFGRSRRPSGIPDRFSGSSSGNTASLIITGAQAED |
| | | EADYYCNSRDNTANHYVFGTGTKLTVL |
| SEQ ID | M16 | MALPVTALLLPLALLLHAARPEVQLVESGGGLVQPGRSLRLSCAASGFTFDDYAMHWVRQ |
| NO: | >XS87- | APGKGLEWVSGISWNSGSTGYADSVKGRFTISRDNAKNSLYLQMNSLRAEDTALYYCAKD |
| 1449 | 99RD | SSSWYGGGSAFDIWGQGTMVTVSSGGGGSGGGGSGGGGSSSELTQEPAVSVALGQTVRIT |
| | (M16 | CQGDSLRSYYASWYQQKPGQAPVLVIFGRSRRPSGIPDRFSGSSSGNTASLIITGAQAED |
| | XS87- | EADYYCNSRDNTANHYVFGTGTKLTVLTTTPAPRPPTPAPTIASQPLSLRPEACRPAAGG |
| | 99RD | AVHTRGLDFACDIYIWAPLAGTCGVLLLSLVITLYCKRGRKKLLYIFKQPFMRPVQTTQE |
| | NT | EDGCSCRFPEEEEGGCELRVKFSRSADAPAYKQGQNQLYNELNLGRREEYDVLDKRRGRD |
| | 127554) | PEMGGKPRRKNPQEGLYNELQKDKMAEAYSEIGMKGERRRGKGHDGLYQGLSTATKDTYD |
| | | ALHMQALPPR |
| SEQ ID | M17 | EVQLVESGGGLVQPGRSLRLSCAASGFTFDDYAMHWVRQ |
| NO: | (ScFv | APGKGLEWVSGISWNSGSTGYADSVKGRFTISRDNAKNSLYLQMNSLRAEDTALYYCAKD |
| 1450 | domain) | SSSWYGGGSAFDIWGQGTMVTVSSGGGGSGGGGSGGGGSSSELTQDPAVSVALGQTVRIT |
| | | CQGDSLRSYYASWYQQKPGQAPVLVIYGKNNRPSGIPDRFSGSSSGNTASLTITGAQAED |
| | | EADYYCNSRGSSGNHYVFGTGTKVTVL |
| SEQ ID | M17 | MALPVTALLLPLALLLHAARPEVQLVESGGGLVQPGRSLRLSCAASGFTFDDYAMHWVRQ |
| NO: | >NS89- | APGKGLEWVSGISWNSGSTGYADSVKGRFTISRDNAKNSLYLQMNSLRAEDTALYYCAKD |
| 1451 | 94MD | SSSWYGGGSAFDIWGQGTMVTVSSGGGGSGGGGSGGGGSSSELTQDPAVSVALGQTVRIT |
| | (M17 | CQGDSLRSYYASWYQQKPGQAPVLVIYGKNNRPSGIPDRFSGSSSGNTASLTITGAQAED |
| | NS89- | EADYYCNSRGSSGNHYVFGTGTKVTVLTTTPAPRPPTPAPTIASQPLSLRPEACRPAAGG |
| | 94MD | AVHTRGLDFACDIYIWAPLAGTCGVLLLSLVITLYCKRGRKKLLYIFKQPFMRPVQTTQE |
| | NT | EDGCSCRFPEEEEGGCELRVKFSRSADAPAYKQGQNQLYNELNLGRREEYDVLDKRRGRD |
| | 127555) | PEMGGKPRRKNPQEGLYNELQKDKMAEAYSEIGMKGERRRGKGHDGLYQGLSTATKDTYD |
| | | ALHMQALPPR |
| SEQ ID | M18 | QVQLVQSGGGLVQPGGSLRLSCAASGFTFSSYWMHWVRQ |
| NO: | (ScFv | APGKGLVWVSRINSDGSSTSYADSVKGRFTISRDNAKNTLYLQMNSLRAEDTAVYYCVRT |
| 1452 | domain) | GWVGSYYYYMDVWGKGTTVTVSSGGGGSGGGGSGGGGSGGGGSEIVLTQSPGTLSLSPGE |
| | | RATLSCRASQSVSSNYLAWYQQKPGQPPRLLIYDVSTRATGIPARFSGGGSGTDFTLTIS |
| | | SLEPEDFAVYYCQQRSNWPPWTFGQGTKVEIK |
| SEQ ID | M18 | MALPVTALLLPLALLLHAARPQVQLVQSGGGLVQPGGSLRLSCAASGFTFSSYWMHWVRQ |
| NO: | >DS90- | APGKGLVWVSRINSDGSSTSYADSVKGRFTISRDNAKNTLYLQMNSLRAEDTAVYYCVRT |
| 1453 | 09HD | GWVGSYYYYMDVWGKGTTVTVSSGGGGSGGGGSGGGGSGGGGSEIVLTQSPGTLSLSPGE |
| | (M18 | RATLSCRASQSVSSNYLAWYQQKPGQPPRLLIYDVSTRATGIPARFSGGGSGTDFTLTIS |
| | DS90- | SLEPEDFAVYYCQQRSNWPPWTFGQGTKVEIKTTTPAPRPPTPAPTIASQPLSLRPEACR |
| | 09HD | PAAGGAVHTRGLDFACDIYIWAPLAGTCGVLLLSLVITLYCKRGRKKLLYIFKQPFMRPV |
| | R003- | QTTQEEDGCSCRFPEEEEGGCELRVKFSRSADAPAYKQGQNQLYNELNLGRREEYDVLDK |
| | A05 | RRGRDPEMGGKPRRKNPQEGLYNELQKDKMAEAYSEIGMKGERRRGKGHDGLYQGLSTAT |
| | 127556) | KDTYDALHMQALPPR |
| SEQ ID | M19 | QVQLVQSGGGVVQPGRSLRLSCAASGFTFSSYGMHWVRQ |
| NO: | (ScFv | APGKGLEWVAVISYDGSNKYYADSVKGRFTISRDNSKNTLYLQMNSLRAEDTAVYYCAKG |
| 1454 | domain) | YSRYYYYGMDVWGQGTTVTVSSGGGGSGGGGSGGGGSGGGGSEIVMTQSPATLSLSPGER |
| | | AILSCRASQSVYTKYLGWYQQKPGQAPRLLIYDASTRATGIPDRFSGSGSGTDFTLTINR |
| | | LEPEDFAVYYCQHYGGSPLITFGQGTKVDIK |
| SEQ ID | M19 | MALPVTALLLPLALLLHAARPQVQLVQSGGGVVQPGRSLRLSCAASGFTFSSYGMHWVRQ |
| NO: | >TS92- | APGKGLEWVAVISYDGSNKYYADSVKGRFTISRDNSKNTLYLQMNSLRAEDTAVYYCAKG |
| 1455 | 04BD | YSRYYYYGMDVWGQGTTVTVSSGGGGSGGGGSGGGGSGGGGSEIVMTQSPATLSLSPGER |
| | (M19 | AILSCRASQSVYTKYLGWYQQKPGQAPRLLIYDASTRATGIPDRFSGSGSGTDFTLTINR |
| | TS92- | LEPEDFAVYYCQHYGGSPLITFGQGTKVDIKTTTPAPRPPTPAPTIASQPLSLRPEACRP |
| | 04BD | AAGGAVHTRGLDFACDIYIWAPLAGTCGVLLLSLVITLYCKRGRKKLLYIFKQPFMRPVQ |
| | R003- | TTQEEDGCSCRFPEEEEGGCELRVKFSRSADAPAYKQGQNQLYNELNLGRREEYDVLDKR |
| | C06 | RGRDPEMGGKPRRKNPQEGLYNELQKDKMAEAYSEIGMKGERRRGKGHDGLYQGLSTATK |
| | 127557) | DTYDALHMQALPPR |
| SEQ ID | M20 | QVQLVQSGGGLVQPGGSLRLSCAASGFTFSSYAMSWVRQ |
| NO: | (ScFv | APGKGLEWVSAISGSGGSTYYADSVKGRFTISRDNSKNTLYLQMNSLRAEDTAVYYCAKR |
| 1456 | domain) | EAAAGHDWYFDLWGRGTLVTVSSGGGGSGGGGSGGGGSGGGGSDIRVTQSPSSLSASVGD |
| | | RVTITCRASQSISSYLNWYQQKPGKAPKLLIYAASSLQSGVPSRFSGSGSGTDFTLTISS |
| | | LQPEDFATYYCQQSYSIPLTFGQGTKVEIK |
| SEQ ID | M20 (full) | MALPVTALLLPLALLLHAARPQVQLVQSGGGLVQPGGSLRLSCAASGFTFSSYAMSWVRQ |
| NO: | >JS93- | APGKGLEWVSAISGSGGSTYYADSVKGRFTISRDNSKNTLYLQMNSLRAEDTAVYYCAKR |
| 1457 | 08WD | EAAAGHDWYFDLWGRGTLVTVSSGGGGSGGGGSGGGGSGGGGSDIRVTQSPSSLSASVGD |
| | (M20 | RVTITCRASQSISSYLNWYQQKPGKAPKLLIYAASSLQSGVPSRFSGSGSGTDFTLTISS |
| | JS93- | LQPEDFATYYCQQSYSIPLTFGQGTKVEIKTTTPAPRPPTPAPTIASQPLSLRPEACRPA |
| | 08WD | AGGAVHTRGLDFACDIYIWAPLAGTCGVLLLSLVITLYCKRGRKKLLYIFKQPFMRPVQT |
| | R003- | TQEEDGCSCRFPEEEEGGCELRVKFSRSADAPAYKQGQNQLYNELNLGRREEYDVLDKRR |
| | E07 | GRDPEMGGKPRRKNPQEGLYNELQKDKMAEAYSEIGMKGERRRGKGHDGLYQGLSTATKD |
| | 127558) | TYDALHMQALPPR |
| SEQ ID | M21 | QVQLVQSWAEVKKPGASVKVSCKASGYTFTSYYMHWVRQAPGQGLEWMGIINPSGGSTSY |
| NO: | (ScFv | AQKFQGRVTMTRDTSTSTVYMELSNLRSEDTAVYYCARSPRVTTGYFDYWGQGTLVTVSS |
| 1458 | domain) | GGGGSGGGGSGGGGSGGGGSDIQLTQSPSTLSASVGDRVTITCRASQSISSWLAWYQQKP |
| | | GKAPKLLIYKASSLESGVPSRFSGSGSGTEFTLTISSLQPDDFATYYCQQYSSYPLTFGG |
| | | GTRLEIK |
| SEQ ID | M21 (full | MALPVTALLLPLALLLHAARPQVQLVQSWAEVKKPGASVKVSCKASGYTFTSYYMHWVRQ |
| NO: | CAR) | APGQGLEWMGIINPSGGSTSYAQKFQGRVTMTRDTSTSTVYMELSNLRSEDTAVYYCARS |
| 1459 | | PRVTTGYFDYWGQGTLVTVSSGGGGSGGGGSGGGGSGGGGSDIQLTQSPSTLSASVGDRV |
| | | TITCRASQSISSWLAWYQQKPGKAPKLLIYKASSLESGVPSRFSGSGSGTEFTLTISSLQ |
| | | PDDFATYYCQQYSSYPLTFGGGTRLEIKTTTPAPRPPTPAPTIASQPLSLRPEACRPA |
| | | AGGAVHTRGLDFACDIYIWAPLAGTCGVLLLSLVITLYCKRGRKKLLYIFKQPFMRPVQT |
| | | TQEEDGCSCRFPEEEEGGCELRVKFSRSADAPAYKQGQNQLYNELNLGRREEYDVLDKRR |
| | | GRDPEMGGKPRRKNPQEGLYNELQKDKMAEAYSEIGMKGERRRGKGHDGLYQGLSTATKD |
| | | TYDALHMQALPPR |
| SEQ ID | M22 | QVQLVQSGAEVRRPGASVKISCRASGDTSTRHYIHWLRQAPGQGPEWMGVINPTTGPATG |
| NO: | (ScFv | SPAYAQMLQGRVTMTRDTSTRTVYMELRSLRFEDTAVYYCARSVVGRSAPYYFDYWGQGT |
| 1460 | domain) | LVTVSSGGGGSGGGGSGGGGSGGGGSDIQMTQSPSSLSASVGDRVTITCRASQGISDYSA |
| | | WYQQKPGKAPKLLIYAASTLQSGVPSRFSGSGSGTDFTLTISYLQSEDFATYYCQQYYSY |
| | | PLTFGGGTKVDIK |
| SEQ ID | M22 (full | MALPVTALLLPLALLLHAARPQVQLVQSGAEVRRPGASVKISCRASGDTSTRHYIHWLRQ |
| NO: | CAR) | APGQGPEWMGVINPTTGPATGSPAYAQMLQGRVTMTRDTSTRTVYMELRSLRFEDTAVYY |
| 1461 | | CARSVVGRSAPYYFDYWGQGTLVTVSSGGGGSGGGGSGGGGSGGGGSDIQMTQSPSSLSA |
| | | SVGDRVTITCRASQGISDYSAWYQQKPGKAPKLLIYAASTLQSGVPSRFSGSGSGTDFTL |
| | | TISYLQSEDFATYYCQQYYSYPLTFGGGTKVDIKTTTPAPRPPTPAPTIASQPLSLRPEA |
| | | CRPA |
| | | AGGAVHTRGLDFACDIYIWAPLAGTCGVLLLSLVITLYCKRGRKKLLYIFKQPFMRPVQT |
| | | TQEEDGCSCREPEEEEGGCELRVKFSRSADAPAYKQGQNQLYNELNLGRREEYDVLDKRR |
| | | GRDPEMGGKPRRKNPQEGLYNELQKDKMAEAYSEIGMKGERRRGKGHDGLYQGLSTATKD |
| | | TYDALHMQALPPR |
| SEQ ID | M23 | QVQLQQSGAEVKKPGASVKVSCKASGYTFTNYYMHWVRQAPGQGLEWMGIINPSGGYTTY |
| NO: | (ScFv | AQKFQGRLTMTRDTSTSTVYMELSSLRSEDTAVYYCARIRSCGGDCYYFDNWGQGTLVTV |
| 1462 | domain) | SSGGGGSGGGGSGGGGSGGGGSDIQLTQSPSTLSASVGDRVTITCRASENVNIWLAWYQQ |
| | | KPGKAPKLLIYKSSSLASGVPSRFSGSGSGAEFTLTISSLQPDDFATYYCQQYQSYPLTF |
| | | GGGTKVDIK |
| SEQ ID | M23 (full | MALPVTALLLPLALLLHAARPQVQLQQSGAEVKKPGASVKVSCKASGYTFTNYYMHWVRQ |
| NO: | CAR) | APGQGLEWMGIINPSGGYTTYAQKFQGRLTMTRDTSTSTVYMELSSLRSEDTAVYYCARI |
| 1463 | | RSCGGDCYYFDNWGQGTLVTVSSGGGGSGGGGSGGGGSGGGGSDIQLTQSPSTLSASVGD |
| | | RVTITCRASENVNIWLAWYQQKPGKAPKLLIYKSSSLASGVPSRFSGSGSGAEFTLTISS |
| | | LQPDDFATYYCQQYQSYPLTEGGGTKVDIKTTTPAPRPPTPAPTIASQPLSLRPEACRPA |
| | | AGGAVHTRGLDFACDIYIWAPLAGTCGVLLLSLVITLYCKRGRKKLLYIFKQPFMRPVQT |
| | | TQEEDGCSCREPEEEEGGCELRVKFSRSADAPAYKQGQNQLYNELNLGRREEYDVLDKRR |
| | | GRDPEMGGKPRRKNPQEGLYNELQKDKMAEAYSEIGMKGERRRGKGHDGLYQGLSTATKD |
| | | TYDALHMQALPPR |
| SEQ ID | M24 | QITLKESGPALVKPTQTLTLTCTFSGFSLSTAGVHVGWIRQPPGKALEWLALISWADDKR |
| NO: | (ScFv | YRPSLRSRLDITRVTSKDQVVLSMTNMQPEDTATYYCALQGFDGYEANWGPGTLVTVSSG |
| 1464 | domain) | GGGSGGGGSGGGGSGGGGSDIVMTQSPSSLSASAGDRVTITCRASRGISSALAWYQQKPG |
| | | KPPKLLIYDASSLESGVPSRFSGSGSGTDFTLTIDSLEPEDFATYYCQQSYSTPWTFGQG |
| | | TKVDIK |
| SEQ ID | M24 (full | MALPVTALLLPLALLLHAARPQITLKESGPALVKPTQTLTLTCTFSGFSLSTAGVHVGWI |
| NO: | CAR) | RQPPGKALEWLALISWADDKRYRPSLRSRLDITRVTSKDQVVLSMTNMQPEDTATYYCAL |
| 1465 | | QGFDGYEANWGPGTLVTVSSGGGGSGGGGSGGGGSGGGGSDIVMTQSPSSLSASAGDRVT |
| | | ITCRASRGISSALAWYQQKPGKPPKLLIYDASSLESGVPSRFSGSGSGTDFTLTIDSLEP |
| | | EDFATYYCQQSYSTPWTFGQGTKVDIKTTTPAPRPPTPAPTIASQPLSLRPEACRPA |
| | | AGGAVHTRGLDFACDIYIWAPLAGTCGVLLLSLVITLYCKRGRKKLLYIFKQPFMRPVQT |
| | | TQEEDGCSCRFPEEEEGGCELRVKFSRSADAPAYKQGQNQLYNELNLGRREEYDVLDKRR |
| | | GRDPEMGGKPRRKNPQEGLYNELQKDKMAEAYSEIGMKGERRRGKGHDGLYQGLSTATKD |
| | | TYDALHMQALPPR |
| SEQ ID | Ss1 (scFv | QVQLQQSGPELEKPGASVKISCKASGYSFTGYTMNWVKQSHGKSLEWIGLITPYNGASS |
| NO: | domain) | YNQKFRGKATLTVDKSSSTAYMDLLSLTSEDSAVYFCARGGYDGRGFDYWGQGTTVTVS |
| 1466 | | SGGGGSGGGGSGGGGSDIELTQSPAIMSASPGEKVTMTCSASSSVSYMHWYQQKSGTSP |
| | | KRWIYDTSKLASGVPGRFSGSGSGNSYSLTISSVEAEDDATYYCQQWSGYPLTFGAGTK |
| | | LEI |
| SEQ ID | Ss1 (full | MALPVTALLLPLALLLHAARPQVQLQQSGPELEKPGASVKISCKASGYSFTGYTMNWVK |
| NO: | CAR) | QSHGKSLEWIGLITPYNGASSYNQKFRGKATLTVDKSSSTAYMDLLSLTSEDSAVYFCA |
| 1467 | | RGGYDGRGFDYWGQGTTVTVSSGGGGSGGGGSGGGGSDIELTQSPAIMSASPGEKVTMT |
| | | CSASSSVSYMHWYQQKSGTSPKRWIYDTSKLASGVPGRFSGSGSGNSYSLTISSVEAED |
| | | DATYYCQQWSGYPLTFGAGTKLEITTTPAPRPPTPAPTIASQPLSLRPEACRPAAGGAV |
| | | HTRGLDFACDIYIWAPLAGTCGVLLLSLVITLYCKRGRKKLLYIFKQPFMRPVQTTQEE |
| | | DGCSCRFPEEEEGGCELRVKFSRSADAPA |

### RNA Transfection

Disclosed herein are methods for producing an in vitro transcribed RNA CAR IL-15R/IL-15. The present invention also includes a CAR IL-15R/IL-15 encoding RNA construct that can be directly transfected into a cell. A method for generating mRNA for use in transfection can involve in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-2000 bases (SEQ ID NO: 1468) in length. RNA so produced can efficiently transfect different kinds of cells. In one aspect, the template includes sequences for the CAR.

In one aspect the CAR IL-15R/IL-15 is encoded by a messenger RNA (mRNA). In one aspect the mRNA encoding the CAR IL-15R/IL-15 is introduced into an immune effector cell, e.g., a T cell or a NK cell, for production of a CAR IL-15R/IL-15-expressing cell (e.g., CAR IL-15R/IL-15 T cell or CAR IL-15R/IL-15-expressing NK cell).

In one embodiment, the in vitro transcribed RNA CAR IL-15R/IL-15 can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired temple for in vitro transcription is a CAR of the present invention. For example, the template for the RNA CAR comprises an extracellular region comprising a single chain variable domain of an anti-tumor antibody; a hinge region, a transmembrane domain (e.g., a transmembrane domain of CD8a); and a cytoplasmic region that includes an intracellular signaling domain, e.g., comprising the signaling domain of CD3-zeta and the signaling domain of 4-1BB.

In one embodiment, the DNA to be used for PCR contains an open reading frame. The DNA can be from a naturally occurring DNA sequence from the genome of an organism. In one embodiment, the nucleic acid can include some or all of the 5′ and/or 3′ untranslated regions (UTRs). The nucleic acid can include exons and introns. In one embodiment, the DNA to be used for PCR is a human nucleic acid sequence. In another embodiment, the DNA to be used for PCR is a human nucleic acid sequence including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

PCR is used to generate a template for in vitro transcription of mRNA which is used for transfection. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a nucleic acid that is normally transcribed in cells (the open reading frame), including 5′ and 3′ UTRs. The primers can also be designed to amplify a portion of a nucleic acid that encodes a particular domain of interest. In one embodiment, the primers are designed to amplify the coding region of a human cDNA, including all or portions of the 5′ and 3′ UTRs. Primers useful for PCR can be generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Any DNA polymerase useful for PCR can be used in the methods disclosed herein. The reagents and polymerase are commercially available from a number of sources.

Chemical structures with the ability to promote stability and/or translation efficiency may also be used. The RNA preferably has 5′ and 3′ UTRs. In one embodiment, the 5′ UTR is between one and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the nucleic acid of interest. Alternatively, UTR sequences that are not endogenous to the nucleic acid of interest can be added by incorporating the UTR sequences into the forward and reverse primers or by any other modifications of the template. The use of UTR sequences that are not endogenous to the nucleic acid of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous nucleic acid. Alternatively, when a 5′ UTR that is not endogenous to the nucleic acid of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be 5′UTR of an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one preferred embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In a preferred embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100T tail (SEQ ID NO: 1469) (size can be 50-5000 T (SEQ ID NO: 1470)), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines (SEQ ID NO: 1471).

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides (SEQ ID NO: 1472) results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps on also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001).

### Non-Viral Delivery Methods

In some aspects, non-viral methods can be used to deliver a nucleic acid encoding a CAR IL-15R/IL-15 described herein into a cell or tissue or a subject.

In some embodiments, the non-viral method includes the use of a transposon (also called a transposable element). In some embodiments, a transposon is a piece of DNA that can insert itself at a location in a genome, for example, a piece of DNA that is capable of self-replicating and inserting its copy into a genome, or a piece of DNA that can be spliced out of a longer nucleic acid and inserted into another place in a genome. For example, a transposon comprises a DNA sequence made up of inverted repeats flanking genes for transposition.

Exemplary methods of nucleic acid delivery using a transposon include a Sleeping Beauty transposon system (SBTS) and a piggyBac (PB) transposon system. See, e.g., Aronovich et al. Hum. Mol. Genet. 20.R1(2011):R14-20; Singh et al. Cancer Res. 15 (2008):2961-2971; Huang et al. Mol. Ther. 16 (2008):580-589; Grabundzija et al. Mol. Ther. 18 (2010):1200-1209; Kebriaei et al. Blood. 122.21 (2013):166; Williams. Molecular Therapy 16.9 (2008):1515-16; Bell et al. Nat. Protoc. 2.12 (2007):3153-65; and Ding et al. Cell. 122.3 (2005):473-83, all of which are incorporated herein by reference.

The SBTS includes two components: 1) a transposon containing a transgene and 2) a source of transposase enzyme. The transposase can transpose the transposon from a carrier plasmid (or other donor DNA) to a target DNA, such as a host cell chromosome/genome. For example, the transposase binds to the carrier plasmid/donor DNA, cuts the transposon (including transgene(s)) out of the plasmid, and inserts it into the genome of the host cell. See, e.g., Aronovich et al. supra.

Exemplary transposons include a pT2-based transposon. See, e.g., Grabundzija et al. Nucleic Acids Res. 41.3 (2013):1829-47; and Singh et al. Cancer Res. 68.8 (2008):2961-2971, all of which are incorporated herein by reference. Exemplary transposases include a Tc1/mariner-type transposase, e.g., the SB10 transposase or the SB11 transposase (a hyperactive transposase which can be expressed, e.g., from a cytomegalovirus promoter). See, e.g., Aronovich et al.; Kebriaei et al.; and Grabundzija et al., all of which are incorporated herein by reference.

Use of the SBTS permits efficient integration and expression of a transgene, e.g., a nucleic acid encoding a CAR described herein. Provided herein are methods of generating a cell, e.g., T cell or NK cell, that stably expresses a CAR described herein, e.g., using a transposon system such as SBTS.

In accordance with methods described herein, in some embodiments, one or more nucleic acids, e.g., plasmids, containing the SBTS components are delivered to a cell (e.g., T or NK cell). For example, the nucleic acid(s) are delivered by standard methods of nucleic acid (e.g., plasmid DNA) delivery, e.g., methods described herein, e.g., electroporation, transfection, or lipofection. In some embodiments, the nucleic acid contains a transposon comprising a transgene, e.g., a nucleic acid encoding a CAR described herein. In some embodiments, the nucleic acid contains a transposon comprising a transgene (e.g., a nucleic acid encoding a CAR described herein) as well as a nucleic acid sequence encoding a transposase enzyme. In other embodiments, a system with two nucleic acids is provided, e.g., a dual-plasmid system, e.g., where a first plasmid contains a transposon comprising a transgene, and a second plasmid contains a nucleic acid sequence encoding a transposase enzyme. For example, the first and the second nucleic acids are co-delivered into a host cell.

In some embodiments, cells, e.g., T or NK cells, are generated that express a CAR described herein by using a combination of gene insertion using the SBTS and genetic editing using a nuclease (e.g., Zinc finger nucleases (ZFNs), Transcription Activator-Like Effector Nucleases (TALENs), the CRISPR/Cas system, or engineered meganuclease re-engineered homing endonucleases).

In some embodiments, use of a non-viral method of delivery permits reprogramming of cells, e.g., T or NK cells, and direct infusion of the cells into a subject. Advantages of non-viral vectors include but are not limited to the ease and relatively low cost of producing sufficient amounts required to meet a patient population, stability during storage, and lack of immunogenicity.

### Nucleic Acid Constructs Encoding a CAR

The present invention also provides nucleic acid molecules encoding one or more CAR IL-15R/IL-15 constructs described herein. In one aspect, the nucleic acid molecule is provided as a messenger RNA transcript. In one aspect, the nucleic acid molecule is provided as a DNA construct.

Accordingly, in one aspect, the invention pertains to an isolated nucleic acid molecule encoding a chimeric antigen receptor (CAR), an IL-15R molecule and an IL-15 molecule, wherein the CAR comprises an antigen binding domain, a transmembrane domain, and an intracellular signaling domain comprising a stimulatory domain, e.g., a costimulatory signaling domain and/or a primary signaling domain, e.g., zeta chain.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The present invention also provides vectors in which a DNA of the present invention is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A retroviral vector may also be, e.g., a gammaretroviral vector. A gammaretroviral vector may include, e.g., a promoter, a packaging signal (ψ), a primer binding site (PBS), one or more (e.g., two) long terminal repeats (LTR), and a transgene of interest, e.g., a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., “Gammaretroviral Vectors: Biology, Technology and Application” Viruses. 2011 June; 3(6): 677-713.

In another embodiment, the vector comprising the nucleic acid encoding the desired CAR of the invention is an adenoviral vector (A5/35). In another embodiment, the expression of nucleic acids encoding CAR IL-15R/IL-15 can be accomplished using of transposons such as sleeping beauty, CRISPR, CAS9, and zinc finger nucleases. See below June et al. 2009 Nature Reviews Immunology 9.10:704-716, is incorporated herein by reference.

In brief summary, the expression of natural or synthetic nucleic acids encoding CAR IL-15R/IL-15 is typically achieved by operably linking a nucleic acid encoding the CAR IL-15R/IL-15 polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The expression constructs of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

An example of a promoter that is capable of expressing a CAR IL-15R/IL-15 transgene in a mammalian T cell is the EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving CAR IL-15R/IL-15 expression from transgenes cloned into a lentiviral vector. See, e.g., Milone et al., Mol. Ther. 17(8): 1453-1464 (2009).

Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1α promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Another example of a promoter is the phosphoglycerate kinase (PGK) promoter. In embodiments, a truncated PGK promoter (e.g., a PGK promoter with one or more, e.g., 1, 2, 5, 10, 100, 200, 300, or 400, nucleotide deletions when compared to the wild-type PGK promoter sequence) may be desired. The nucleotide sequences of exemplary PGK promoters are provided below.

WT PGK Promoter
(SEQ ID NO: 1473)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACGAGGGACCGCGACAGGCAGACGCTCCCATG
ATCACTCTGCACGCCGAAGGCAAATAGTGCAGGCCGTGCGGCGCTTGGCG
TTCCTTGGAAGGGCTGAATCCCCGCCTCGTCCTTCGCAGCGGCCCCCCGG
GTGTTCCCATCGCCGCTTCTAGGCCCACTGCGACGCTTGCCTGCACTTCT
TACACGCTCTGGGTCCCAGCCGCGGCGACGCAAAGGGCCTTGGTGCGGGT
CTCGTCGGCGCAGGGACGCGTTTGGGTCCCGACGGAACCTTTTCCGCGTT
GGGGTTGGGGCACCATAAGCT
Exemplary truncated PGK Promoters:
PGK100:
(SEQ ID NO: 1474)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTG
PGK200:
(SEQ ID NO: 1475)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACG
PGK300:
(SEQ ID NO: 1476)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACGAGGGACCGCGACAGGCAGACGCTCCCATG
ATCACTCTGCACGCCGAAGGCAAATAGTGCAGGCCGTGCGGCGCTTGGCG
TTCCTTGGAAGGGCTGAATCCCCG
PGK400:
(SEQ ID NO: 1477)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACGAGGGACCGCGACAGGCAGACGCTCCCATG
ATCACTCTGCACGCCGAAGGCAAATAGTGCAGGCCGTGCGGCGCTTGGCG
TTCCTTGGAAGGGCTGAATCCCCGCCTCGTCCTTCGCAGCGGCCCCCCGG
GTGTTCCCATCGCCGCTTCTAGGCCCACTGCGACGCTTGCCTGCACTTCT
TACACGCTCTGGGTCCCAGCCG

A vector may also include, e.g., a signal sequence to facilitate secretion, a polyadenylation signal and transcription terminator (e.g., from Bovine Growth Hormone (BGH) gene), an element allowing episomal replication and replication in prokaryotes (e.g. SV40 origin and ColEl or others known in the art) and/or elements to allow selection (e.g., ampicillin resistance gene and/or zeocin marker).

In order to assess the expression of a CAR IL-15R/IL-15 polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479:79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In one embodiment, the vector can further comprise a nucleic acid encoding a second CAR. In one embodiment, the second CAR includes an antigen binding domain to a target expressed on acute myeloid leukemia cells, such as, e.g., CD123, CD34, CLL-1, folate receptor beta, or FLT3; or a target expressed on a B cell, e.g., CD10, CD19, CD20, CD22, CD34, CD123, FLT-3, ROR1, CD79b, CD179b, or CD79a. In one embodiment, the vector comprises a nucleic acid sequence encoding a first CAR that specifically binds a first antigen and includes an intracellular signaling domain having a costimulatory signaling domain but not a primary signaling domain, and a nucleic acid encoding a second CAR that specifically binds a second, different, antigen and includes an intracellular signaling domain having a primary signaling domain but not a costimulatory signaling domain.

In one embodiment, the vector comprises a nucleic acid encoding a CAR IL-15R/IL-15 described herein and a nucleic acid encoding an inhibitory CAR. In one embodiment, the inhibitory CAR comprises an antigen binding domain that binds an antigen found on normal cells but not cancer cells. In one embodiment, the inhibitory CAR comprises the antigen binding domain, a transmembrane domain and an intracellular domain of an inhibitory molecule. For example, the intracellular domain of the inhibitory CAR can be an intracellular domain of PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta.

In embodiments, the vector may comprise two or more nucleic acid sequences encoding a CAR IL-15R/IL-15, e.g., a CAR IL-15R/IL-15 described herein and a second CAR, e.g., an inhibitory CAR or a CAR that specifically binds to a different antigen. In such embodiments, the two or more nucleic acid sequences encoding the CAR IL-15R/IL-15 are encoded by a single nucleic molecule in the same frame and as a single polypeptide chain. In this aspect, the two or more CARs, can, e.g., be separated by one or more peptide cleavage sites. (e.g., an auto-cleavage site or a substrate for an intracellular protease). Examples of peptide cleavage sites include the following, wherein the GSG residues are optional:

T2A:
(SEQ ID NO: 1478)
(GSG) E G R G S L L T C G D V E E N P G P
P2A:
(SEQ ID NO: 1479)
(GSG) A T N F S L L K Q A G D V E E N P G P
E2A:
(SEQ ID NO: 1480)
(GSG) Q C T N Y A L L K L A G D V E S N P G P
F2A:
(SEQ ID NO: 1481)
(GSG) V K Q T L N F D L L K L A G D V E S N P G P

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY). A preferred method for the introduction of a polynucleotide into a host cell is calcium phosphate transfection

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5:505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

The present invention further provides a vector comprising a CAR IL-15R/IL-15 encoding nucleic acid molecule. In one aspect, a CAR IL-15R/IL-15 vector can be directly transduced into a cell, e.g., a T cell or NK cell. In one aspect, the vector is a cloning or expression vector, e.g., a vector including, but not limited to, one or more plasmids (e.g., expression plasmids, cloning vectors, minicircles, minivectors, double minute chromosomes), retroviral and lentiviral vector constructs. In some embodiments, the vector is a multicistronic vector. In one aspect, the vector is capable of expressing the CAR IL-15R/IL-15 construct in mammalian T cells or NK cells. In one aspect, the mammalian T cell is a human T cell. In one aspect, the mammalian NK cell is a human NK cell.

### Sources of Cells

Prior to expansion and genetic modification, a source of cells, e.g., immune effector cells (e.g., T cells or NK cells), is obtained from a subject. The term “subject” is intended to include living organisms in which an immune response can be elicited (e.g., mammals). Examples of subjects include humans, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors.

In certain aspects of the present invention, any number of immune effector cell (e.g., T cell or NK cell) lines available in the art, may be used. In certain aspects of the present invention, T cells can be obtained from a unit of blood collected from a subject using any number of techniques known to the skilled artisan, such as Ficoll™ separation. In one preferred aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and to place the cells in an appropriate buffer or media for subsequent processing steps. In one aspect of the invention, the cells are washed with phosphate buffered saline (PBS). In an alternative aspect, the wash solution lacks calcium and may lack magnesium or may lack many if not all divalent cations.

Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

It is recognized that the methods of the application can utilize culture media conditions comprising 5% or less, for example 2%, human AB serum, and employ known culture media conditions and compositions, for example those described in Smith et al., “Ex vivo expansion of human T cells for adoptive immunotherapy using the novel Xeno-free CTS Immune Cell Serum Replacement” Clinical & Translational Immunology (2015) 4, e31; doi: 10.1038/cti.2014.31.

In one aspect, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD4+, CD8+, CD45RA+, and/or CD45RO+T cells, can be further isolated by positive or negative selection techniques. For example, in one aspect, T cells are isolated by incubation with anti-CD3/anti-CD28 (e.g., 3×28)-conjugated beads, such as DYNABEADS® M-450 CD3/CD28 T, for a time period sufficient for positive selection of the desired T cells. In one aspect, the time period is about 30 minutes. In a further aspect, the time period ranges from 30 minutes to 36 hours or longer and all integer values there between. In a further aspect, the time period is at least 1, 2, 3, 4, 5, or 6 hours. In yet another preferred aspect, the time period is 10 to 24 hours. In one aspect, the incubation time period is 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In certain aspects, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain aspects, it may be desirable to enrich for or positively select for regulatory T cells which typically express CD4+, CD25+, CD62Lhi, GITR+, and FoxP3+. In certain aspects, it may be desirable to enrich for cells that are CD127low. Alternatively, in certain aspects, T regulatory cells are depleted by anti-C25 conjugated beads or other similar method of selection.

The methods described herein can include, e.g., selection of a specific subpopulation of immune effector cells, e.g., T cells, that are a T regulatory cell-depleted population, CD25+ depleted cells, using, e.g., a negative selection technique, e.g., described herein. Preferably, the population of T regulatory depleted cells contains less than 30%, 25%, 20%, 15%, 10%, 5%, 4%, 3%, 2%, 1% of CD25+ cells.

In one embodiment, T regulatory cells, e.g., CD25+ T cells, are removed from the population using an anti-CD25 antibody, or fragment thereof, or a CD25-binding ligand, IL-2. In one embodiment, the anti-CD25 antibody, or fragment thereof, or CD25-binding ligand is conjugated to a substrate, e.g., a bead, or is otherwise coated on a substrate, e.g., a bead. In one embodiment, the anti-CD25 antibody, or fragment thereof, is conjugated to a substrate as described herein.

In one embodiment, the T regulatory cells, e.g., CD25+ T cells, are removed from the population using CD25 depletion reagent from Miltenyi™. In one embodiment, the ratio of cells to CD25 depletion reagent is 1e7 cells to 20 uL, or 1e7 cells to15 uL, or 1e7 cells to 10 uL, or 1e7 cells to 5 uL, or 1e7 cells to 2.5 uL, or 1e7 cells to 1.25 uL. In one embodiment, e.g., for T regulatory cells, e.g., CD25+ depletion, greater than 500 million cells/ml is used. In a further aspect, a concentration of cells of 600, 700, 800, or 900 million cells/ml is used.

In one embodiment, the population of immune effector cells to be depleted includes about 6×10⁹ CD25+ T cells. In other aspects, the population of immune effector cells to be depleted include about 1×10⁹ to 1×10¹⁰ CD25+ T cell, and any integer value in between. In one embodiment, the resulting population T regulatory depleted cells has 2×10⁹ T regulatory cells, e.g., CD25+ cells, or less (e.g., 1×10⁹, 5×10⁸, 1×10⁸, 5×10⁷, 1×10⁷, or less CD25+ cells).

In one embodiment, the T regulatory cells, e.g., CD25+ cells, are removed from the population using the CliniMAC system with a depletion tubing set, such as, e.g., tubing 162-01. In one embodiment, the CliniMAC system is run on a depletion setting such as, e.g., DEPLETION2.1.

Without wishing to be bound by a particular theory, decreasing the level of negative regulators of immune cells (e.g., decreasing the number of unwanted immune cells, e.g., TREG cells), in a subject prior to apheresis or during manufacturing of a CAR-expressing cell product can reduce the risk of subject relapse. For example, methods of depleting TREG cells are known in the art. Methods of decreasing TREG cells include, but are not limited to, cyclophosphamide, anti-GITR antibody (an anti-GITR antibody described herein), CD25-depletion, and combinations thereof.

In some embodiments, the manufacturing methods comprise reducing the number of (e.g., depleting) TREG cells prior to manufacturing of the CAR-expressing cell. For example, manufacturing methods comprise contacting the sample, e.g., the apheresis sample, with an anti-GITR antibody and/or an anti-CD25 antibody (or fragment thereof, or a CD25-binding ligand), e.g., to deplete TREG cells prior to manufacturing of the CAR-expressing cell (e.g., T cell, NK cell) product.

In an embodiment, a subject is pre-treated with one or more therapies that reduce TREG cells prior to collection of cells for CAR-expressing cell product manufacturing, thereby reducing the risk of subject relapse to CAR-expressing cell treatment. In an embodiment, methods of decreasing TREG cells include, but are not limited to, administration to the subject of one or more of cyclophosphamide, anti-GITR antibody, CD25-depletion, or a combination thereof. Administration of one or more of cyclophosphamide, anti-GITR antibody, CD25-depletion, or a combination thereof, can occur before, during or after an infusion of the CAR-expressing cell product.

In an embodiment, a subject is pre-treated with cyclophosphamide prior to collection of cells for CAR IL-15R/IL-15-expressing cell product manufacturing, thereby reducing the risk of subject relapse to CAR IL-15R/IL-15-expressing cell treatment. In an embodiment, a subject is pre-treated with an anti-GITR antibody prior to collection of cells for CAR IL-15R/IL-15-expressing cell product manufacturing, thereby reducing the risk of subject relapse to CAR IL-15R/IL-15-expressing cell treatment.

In one embodiment, the population of cells to be removed are neither the regulatory T cells or tumor cells, but cells that otherwise negatively affect the expansion and/or function of CAR IL-15R/IL-15 T cells, e.g. cells expressing CD14, CD11b, CD33, CD15, or other markers expressed by potentially immune suppressive cells. In one embodiment, such cells are envisioned to be removed concurrently with regulatory T cells and/or tumor cells, or following said depletion, or in another order.

The methods described herein can include more than one selection step, e.g., more than one depletion step. Enrichment of a T cell population by negative selection can be accomplished, e.g., with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail can include antibodies to CD14, CD20, CD11b, CD16, HLA-DR, and CD8.

The methods described herein can further include removing cells from the population which express a tumor antigen, e.g., a tumor antigen that does not comprise CD25, e.g., CD19, CD30, CD38, CD123, CD20, CD14 or CD11b, to thereby provide a population of T regulatory depleted, e.g., CD25+ depleted, and tumor antigen depleted cells that are suitable for expression of a CAR, e.g., a CAR described herein. In one embodiment, tumor antigen expressing cells are removed simultaneously with the T regulatory, e.g., CD25+ cells. For example, an anti-CD25 antibody, or fragment thereof, and an anti-tumor antigen antibody, or fragment thereof, can be attached to the same substrate, e.g., bead, which can be used to remove the cells or an anti-CD25 antibody, or fragment thereof, or the anti-tumor antigen antibody, or fragment thereof, can be attached to separate beads, a mixture of which can be used to remove the cells. In other embodiments, the removal of T regulatory cells, e.g., CD25+ cells, and the removal of the tumor antigen expressing cells is sequential, and can occur, e.g., in either order.

Also provided are methods that include removing cells from the population which express a check point inhibitor, e.g., a check point inhibitor described herein, e.g., one or more of PD1+ cells, LAG3+ cells, and TIM3+ cells, to thereby provide a population of T regulatory depleted, e.g., CD25+ depleted cells, and check point inhibitor depleted cells, e.g., PD1+, LAG3+ and/or TIM3+ depleted cells. Exemplary check point inhibitors include PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta. In embodiments, the checkpoint inhibitor is PD1 or PD-L1. In one embodiment, check point inhibitor expressing cells are removed simultaneously with the T regulatory, e.g., CD25+ cells. For example, an anti-CD25 antibody, or fragment thereof, and an anti-check point inhibitor antibody, or fragment thereof, can be attached to the same bead which can be used to remove the cells, or an anti-CD25 antibody, or fragment thereof, and the anti-check point inhibitor antibody, or fragment there, can be attached to separate beads, a mixture of which can be used to remove the cells. In other embodiments, the removal of T regulatory cells, e.g., CD25+ cells, and the removal of the check point inhibitor expressing cells is sequential, and can occur, e.g., in either order.

In one embodiment, a T cell population can be selected that expresses one or more of IFN-γ, TNFα, IL-17A, IL-2, IL-3, IL-4, GM-CSF, IL-10, IL-13, granzyme B, and perforin, or other appropriate molecules, e.g., other cytokines. Methods for screening for cell expression can be determined, e.g., by the methods described in PCT Publication No.: WO 2013/126712.

For isolation of a desired population of cells by positive or negative selection, the concentration of cells and surface (e.g., particles such as beads) can be varied. In certain aspects, it may be desirable to significantly decrease the volume in which beads and cells are mixed together (e.g., increase the concentration of cells), to ensure maximum contact of cells and beads. For example, in one aspect, a concentration of 2 billion cells/ml is used. In one aspect, a concentration of 1 billion cells/ml is used. In a further aspect, greater than 100 million cells/ml is used. In a further aspect, a concentration of cells of 10, 15, 20, 25, 30, 35, 40, 45, or 50 million cells/ml is used. In yet one aspect, a concentration of cells from 75, 80, 85, 90, 95, or 100 million cells/ml is used. In further aspects, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T cells that normally have weaker CD28 expression. In a related aspect, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one aspect, the concentration of cells used is 5×10e6/ml. In other aspects, the concentration used can be from about 1×10⁵/ml to 1×10⁶/ml, and any integer value in between.

In other aspects, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain aspects, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as immune effector cells, e.g., T cells or NK cells, isolated and frozen for later use in cell therapy, e.g., T cell therapy, for any number of diseases or conditions that would benefit from cell therapy, e.g., T cell therapy, such as those described herein. In one aspect a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use. In certain aspects, the immune effector cells (e.g., T cells or NK cells) may be expanded, frozen, and used at a later time. In certain aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In a further aspect of the present invention, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

In one embodiment, the immune effector cells expressing a CAR IL-15R/IL-15 molecule, e.g., a CAR IL-15R/IL-15 molecule described herein, are obtained from a subject that has received a low, immune enhancing dose of an mTOR inhibitor. In an embodiment, the population of immune effector cells, e.g., T cells, to be engineered to express a CAR IL-15R/IL-15, are harvested after a sufficient time, or after sufficient dosing of the low, immune enhancing, dose of an mTOR inhibitor, such that the level of PD1 negative immune effector cells, e.g., T cells, or the ratio of PD1 negative immune effector cells, e.g., T cells/PD1 positive immune effector cells, e.g., T cells, in the subject or harvested from the subject has been, at least transiently, increased.

In other embodiments, population of immune effector cells, e.g., T cells, which have, or will be engineered to express a CAR IL-15R/IL-15, can be treated ex vivo by contact with an amount of an mTOR inhibitor that increases the number of PD1 negative immune effector cells, e.g., T cells or increases the ratio of PD1 negative immune effector cells, e.g., T cells/PD1 positive immune effector cells, e.g., T cells.

In one embodiment, a T cell population is diaglycerol kinase (DGK)-deficient. DGK-deficient cells include cells that do not express DGK RNA or protein, or have reduced or inhibited DGK activity. DGK-deficient cells can be generated by genetic approaches, e.g., administering RNA-interfering agents, e.g., siRNA, shRNA, miRNA, to reduce or prevent DGK expression. Alternatively, DGK-deficient cells can be generated by treatment with DGK inhibitors described herein.

In one embodiment, a T cell population is Ikaros-deficient. Ikaros-deficient cells include cells that do not express Ikaros RNA or protein, or have reduced or inhibited Ikaros activity, Ikaros-deficient cells can be generated by genetic approaches, e.g., administering RNA-interfering agents, e.g., siRNA, shRNA, miRNA, to reduce or prevent Ikaros expression. Alternatively, Ikaros-deficient cells can be generated by treatment with Ikaros inhibitors, e.g., lenalidomide.

In embodiments, a T cell population is DGK-deficient and Ikaros-deficient, e.g., does not express DGK and Ikaros, or has reduced or inhibited DGK and Ikaros activity. Such DGK and Ikaros-deficient cells can be generated by any of the methods described herein.

In an embodiment, the NK cells are obtained from the subject. In another embodiment, the NK cells are an NK cell line, e.g., NK-92 cell line (Conkwest).

### Modifications of CAR Cells, Including Allogeneic CAR Cells

In embodiments described herein, the immune effector cell can be an allogeneic immune effector cell, e.g., T cell or NK cell. For example, the cell can be an allogeneic T cell, e.g., an allogeneic T cell lacking expression of a functional T cell receptor (TCR) and/or human leukocyte antigen (HLA), e.g., HLA class I and/or HLA class II, and/or beta-2 microglobulin (B2m). Compositions of allogeneic CAR and methods thereof have been described in, e.g., pages 227-237 of WO 2016/014565, incorporated herein by reference in its entirety.

In some embodiments, a cell, e.g., a T cell or a NK cell, is modified to reduce the expression of a TCR, and/or HLA, and/or B2m, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), using, e.g., a method described herein, e.g., siRNA, shRNA, clustered regularly interspaced short palindromic repeats (CRISPR) transcription-activator like effector nuclease (TALEN), or zinc finger endonuclease (ZFN).

In some embodiments, a cell, e.g., a T cell or a NK cell is engineered to express a telomerase subunit, e.g., the catalytic subunit of telomerase, e.g., TERT, e.g., hTERT. In one embodiment, such modification improves persistence of the cell in a patient.

### Activation and Expansion of T Cells

T cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T cells of the invention may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besançon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In certain aspects, the primary stimulatory signal and the costimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one aspect, the agent providing the costimulatory signal is bound to a cell surface and the agent providing the primary activation signal is in solution or coupled to a surface. In certain aspects, both agents can be in solution. In one aspect, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In one aspect, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the costimulatory signal is an anti-CD28 antibody or antigen-binding fragment thereof; and both agents are co-immobilized to the same bead in equivalent molecular amounts. In one aspect, a 1:1 ratio of each antibody bound to the beads for CD4+ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1. In one particular aspect an increase of from about 1 to about 3 fold is observed as compared to the expansion observed using a ratio of 1:1. In one aspect, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e., the ratio of CD3:CD28 is less than one. In certain aspects of the invention, the ratio of anti CD28 antibody to anti CD3 antibody bound to the beads is greater than 2:1. In one particular aspect, a 1:100 CD3:CD28 ratio of antibody bound to beads is used. In one aspect, a 1:75 CD3:CD28 ratio of antibody bound to beads is used. In a further aspect, a 1:50 CD3:CD28 ratio of antibody bound to beads is used. In one aspect, a 1:30 CD3:CD28 ratio of antibody bound to beads is used. In one preferred aspect, a 1:10 CD3:CD28 ratio of antibody bound to beads is used. In one aspect, a 1:3 CD3:CD28 ratio of antibody bound to the beads is used. In yet one aspect, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain aspects the ratio of cells to particles ranges from 1:100 to 100:1 and any integer values in-between and in further aspects the ratio comprises 1:9 to 9:1 and any integer values in between, can also be used to stimulate T cells. The ratio of anti-CD3- and anti-CD28-coupled particles to T cells that result in T cell stimulation can vary as noted above, however certain preferred values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one preferred ratio being at least 1:1 particles per T cell. In one aspect, a ratio of particles to cells of 1:1 or less is used. In one particular aspect, a preferred particle:cell ratio is 1:5. In further aspects, the ratio of particles to cells can be varied depending on the day of stimulation. For example, in one aspect, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular aspect, the ratio of particles to cells is 1:1 on the first day of stimulation and adjusted to 1:5 on the third and fifth days of stimulation. In one aspect, particles are added on a daily or every other day basis to a final ratio of 1:1 on the first day, and 1:5 on the third and fifth days of stimulation. In one aspect, the ratio of particles to cells is 2:1 on the first day of stimulation and adjusted to 1:10 on the third and fifth days of stimulation. In one aspect, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type. In one aspect, the most typical ratios for use are in the neighborhood of 1:1, 2:1 and 3:1 on the first day.

In further aspects of the present invention, the cells, such as T cells, are combined with agent-coated beads, the beads and the cells are subsequently separated, and then the cells are cultured. In an alternative aspect, prior to culture, the agent-coated beads and cells are not separated but are cultured together. In a further aspect, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one aspect the cells (for example, 10⁴ to 10⁹ T cells) and beads (for example, DYNABEADS® M-450 CD3/CD28 T paramagnetic beads at a ratio of 1:1) are combined in a buffer, for example PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In certain aspects, it may be desirable to significantly decrease the volume in which particles and cells are mixed together (i.e., increase the concentration of cells), to ensure maximum contact of cells and particles. For example, in one aspect, a concentration of about 10 billion cells/ml, 9 billion/ml, 8 billion/ml, 7 billion/ml, 6 billion/ml, 5 billion/ml, or 2 billion cells/ml is used. In one aspect, greater than 100 million cells/ml is used. In a further aspect, a concentration of cells of 10, 15, 20, 25, 30, 35, 40, 45, or 50 million cells/ml is used. In yet one aspect, a concentration of cells from 75, 80, 85, 90, 95, or 100 million cells/ml is used. In further aspects, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain aspects. For example, using high concentration of cells allows more efficient selection of CD8+ T cells that normally have weaker CD28 expression.

In one embodiment, cells transduced with a nucleic acid encoding a CAR IL-15R/IL-15, e.g., a CAR IL-15R/IL-15 described herein, are expanded, e.g., by a method described herein. In one embodiment, the cells are expanded in culture for a period of several hours (e.g., about 2, 3, 4, 5, 6, 7, 8, 9, 10, 15, 18, 21 hours) to about 14 days (e.g., 1, 2, 3, 4, 5, 6, 7, 8, 9, 10, 11, 12, 13 or 14 days). In one embodiment, the cells are expanded for a period of 4 to 9 days. In one embodiment, the cells are expanded for a period of 8 days or less, e.g., 7, 6 or 5 days. In one embodiment, the cells, e.g., a CAR IL-15R/IL-15 expressing cell described herein, are expanded in culture for 5 days, and the resulting cells are more potent than the same cells expanded in culture for 9 days under the same culture conditions. Potency can be defined, e.g., by various T cell functions, e.g. proliferation, target cell killing, cytokine production, activation, migration, or combinations thereof. In one embodiment, the cells, e.g., a CAR IL-15R/IL-15 expressing cell described herein, expanded for 5 days show at least a one, two, three or four fold increase in cells doublings upon antigen stimulation as compared to the same cells expanded in culture for 9 days under the same culture conditions. In one embodiment, the cells, e.g., the cells expressing a CAR IL-15R/IL-15 described herein, are expanded in culture for 5 days, and the resulting cells exhibit higher proinflammatory cytokine production, e.g., IFN-γ and/or GM-CSF levels, as compared to the same cells expanded in culture for 9 days under the same culture conditions. In one embodiment, the cells, e.g., a CAR IL-15R/IL-15 expressing cell described herein, expanded for 5 days show at least a one, two, three, four, five, ten fold or more increase in pg/ml of proinflammatory cytokine production, e.g., IFN-γ and/or GM-CSF levels, as compared to the same cells expanded in culture for 9 days under the same culture conditions.

In one aspect of the present invention, the mixture may be cultured for several hours (about 3 hours) to about 14 days or any hourly integer value in between. In one aspect, the mixture may be cultured for 21 days. In one aspect of the invention the beads and the T cells are cultured together for about eight days. In one aspect, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

In one embodiment, the cells are expanded in an appropriate media (e.g., media described herein) that includes one or more interleukin that result in at least a 200-fold (e.g., 200-fold, 250-fold, 300-fold, 350-fold) increase in cells over a 14 day expansion period, e.g., as measured by a method described herein such as flow cytometry. In one embodiment, the cells are expanded in the presence of IL-15 and/or IL-7 (e.g., IL-15 and IL-7).

In embodiments, methods described herein, e.g., CAR IL-15R/IL-15-expressing cell manufacturing methods, comprise removing T regulatory cells, e.g., CD25+ T cells, from a cell population, e.g., using an anti-CD25 antibody, or fragment thereof, or a CD25-binding ligand, IL-2. Methods of removing T regulatory cells, e.g., CD25+ T cells, from a cell population are described herein. In embodiments, the methods, e.g., manufacturing methods, further comprise contacting a cell population (e.g., a cell population in which T regulatory cells, such as CD25+ T cells, have been depleted; or a cell population that has previously contacted an anti-CD25 antibody, fragment thereof, or CD25-binding ligand) with IL-15 and/or IL-7. For example, the cell population (e.g., that has previously contacted an anti-CD25 antibody, fragment thereof, or CD25-binding ligand) is expanded in the presence of IL-15 and/or IL-7.

In some embodiments a CAR-expressing cell described herein is contacted with a composition comprising a interleukin-15 (IL-15) polypeptide, a interleukin-15 receptor alpha (IL-15Ra) polypeptide, or a combination of both a IL-15 polypeptide and a IL-15Ra polypeptide e.g., hetIL-15, during the manufacturing of the CAR-expressing cell, e.g., ex vivo. In embodiments, a CAR-expressing cell described herein is contacted with a composition comprising a IL-15 polypeptide during the manufacturing of the CAR-expressing cell, e.g., ex vivo. In embodiments, a CAR-expressing cell described herein is contacted with a composition comprising a combination of both a IL-15 polypeptide and a IL-15 Ra polypeptide during the manufacturing of the CAR-expressing cell, e.g., ex vivo. In embodiments, a CAR-expressing cell described herein is contacted with a composition comprising hetIL-15 during the manufacturing of the CAR-expressing cell, e.g., ex vivo.

In one embodiment the CAR-expressing cell described herein is contacted with a composition comprising hetIL-15 during ex vivo expansion. In an embodiment, the CAR-expressing cell described herein is contacted with a composition comprising an IL-15 polypeptide during ex vivo expansion. In an embodiment, the CAR-expressing cell described herein is contacted with a composition comprising both an IL-15 polypeptide and an IL-15Ra polypeptide during ex vivo expansion. In one embodiment the contacting results in the survival and proliferation of a lymphocyte subpopulation, e.g., CD8+ T cells.

T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population. Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Once a CAR IL-15R/IL-15 is constructed, various assays can be used to evaluate the activity of the molecule, such as but not limited to, the ability to expand T cells following antigen stimulation, sustain T cell expansion in the absence of re-stimulation, and anti-cancer activities in appropriate in vitro and animal models. Assays to evaluate the effects of a CAR IL-15R/IL-15 are described in further detail below.

Western blot analysis of CAR expression in primary T cells can be used to detect the presence of monomers and dimers. See, e.g., Milone et al., Molecular Therapy 17(8): 1453-1464 (2009). Very briefly, T cells (1:1 mixture of CD4⁺ and CD8⁺ T cells) expressing the CARs are expanded in vitro for more than 10 days followed by lysis and SDS-PAGE under reducing conditions. CARs containing the full length TCR-ζ cytoplasmic domain and the endogenous TCR-ζ chain are detected by western blotting using an antibody to the TCR-ζ chain. The same T cell subsets are used for SDS-PAGE analysis under non-reducing conditions to permit evaluation of covalent dimer formation.

In vitro expansion of CAR⁺ IL-15R/IL-15 T cells following antigen stimulation can be measured by flow cytometry. For example, a mixture of CD4⁺ and CD8⁺ T cells are stimulated with αCD3/αCD28 aAPCs followed by transduction with lentiviral vectors expressing GFP under the control of the promoters to be analyzed. Exemplary promoters include the CMV IE gene, EF-1α, ubiquitin C, or phosphoglycerokinase (PGK) promoters. GFP fluorescence is evaluated on day 6 of culture in the CD4⁺ and/or CD8⁺ T cell subsets by flow cytometry. See, e.g., Milone et al., Molecular Therapy 17(8): 1453-1464 (2009). Alternatively, a mixture of CD4⁺ and CD8⁺ T cells are stimulated with αCD3/αCD28 coated magnetic beads on day 0, and transduced with CAR IL-15R/IL-15 on day 1 using a multicistronic lentiviral vector expressing CAR IL-15R/IL-15 along with eGFP using a 2A ribosomal skipping sequence. Cultures are re-stimulated with antigen-expressing cells, such as multiple myeloma cell lines or K562 expressing the antigen, following washing. Exogenous IL-2 is added to the cultures every other day at 100 IU/ml. GFP⁺ T cells are enumerated by flow cytometry using bead-based counting. See, e.g., Milone et al., Molecular Therapy 17(8): 1453-1464 (2009).

Sustained CAR⁺ T cell expansion in the absence of re-stimulation can also be measured. See, e.g., Milone et al., Molecular Therapy 17(8): 1453-1464 (2009). Briefly, mean T cell volume (fl) is measured on day 8 of culture using a Coulter Multisizer III particle counter, a Nexcelom Cellometer Vision or Millipore Scepter, following stimulation with αCD3/αCD28 coated magnetic beads on day 0, and transduction with the indicated CAR IL-15R/IL-15 on day 1.

Animal models can also be used to measure a CART activity. For example, xenograft model using human antigen-specific CAR⁺ T cells to treat a primary human multiple myeloma in immunodeficient mice can be used. See, e.g., Milone et al., Molecular Therapy 17(8): 1453-1464 (2009). Very briefly, after establishment of MM, mice are randomized as to treatment groups. Different numbers of CAR IL-15R/IL-15 T cells can be injected into immunodeficient mice bearing MM. Animals are assessed for disease progression and tumor burden at weekly intervals. Survival curves for the groups are compared using the log-rank test. In addition, absolute peripheral blood CD4⁺ and CD8⁺ T cell counts 4 weeks following T cell injection in the immunodeficient mice can also be analyzed. Mice are injected with multiple myeloma cells and 3 weeks later are injected with T cells engineered to express CAR IL-15R/IL-15, e.g., by a multicistronic lentiviral vector that encodes the CAR IL-15R/IL-15 linked to eGFP. T cells are normalized to 45-50% input GFP⁺ T cells by mixing with mock-transduced cells prior to injection, and confirmed by flow cytometry. Animals are assessed for leukemia at 1-week intervals. Survival curves for the CAR⁺ IL-15R/IL-15 T cell groups are compared using the log-rank test.

Assessment of cell proliferation and cytokine production has been previously described, e.g., at Milone et al., Molecular Therapy 17(8): 1453-1464 (2009). Briefly, assessment of CAR IL-15R/IL-15-mediated proliferation is performed in microtiter plates by mixing washed T cells with K562 cells expressing the antigen or other antigen-expressing myeloma cells are irradiated with gamma-radiation prior to use. Anti-CD3 (clone OKT3) and anti-CD28 (clone 9.3) monoclonal antibodies are added to cultures with KT32-BBL cells to serve as a positive control for stimulating T-cell proliferation since these signals support long-term CD8⁺ T cell expansion ex vivo. T cells are enumerated in cultures using CountBright™ fluorescent beads (Invitrogen, Carlsbad, CA) and flow cytometry as described by the manufacturer. CAR⁺ IL-15R/IL-15 T cells are identified by GFP expression using T cells that are engineered with eGFP-2A linked CAR-expressing lentiviral vectors. For CAR+IL-15R/IL-15 T cells not expressing GFP, the CAR⁺ IL-15R/IL-15 T cells are detected with biotinylated recombinant antigen protein and a secondary avidin-PE conjugate. CD4+ and CD8⁺ expression on T cells are also simultaneously detected with specific monoclonal antibodies (BD Biosciences). Cytokine measurements are performed on supernatants collected 24 hours following re-stimulation using the human TH1/TH2 cytokine cytometric bead array kit (BD Biosciences, San Diego, CA) according the manufacturer's instructions. Fluorescence is assessed using a FACScalibur flow cytometer, and data is analyzed according to the manufacturer's instructions.

Cytotoxicity can be assessed by a standard 51Cr-release assay. See, e.g., Milone et al., Molecular Therapy 17(8): 1453-1464 (2009). Briefly, target cells (e.g., K562 lines expressing the antigen and primary multiple myeloma cells) are loaded with 51Cr (as NaCrO4, New England Nuclear, Boston, MA) at 37° C. for 2 hours with frequent agitation, washed twice in complete RPMI and plated into microtiter plates. Effector T cells are mixed with target cells in the wells in complete RPMI at varying ratios of effector cell:target cell (E:T). Additional wells containing media only (spontaneous release, SR) or a 1% solution of triton-X 100 detergent (total release, TR) are also prepared. After 4 hours of incubation at 37° C., supernatant from each well is harvested. Released 51Cr is then measured using a gamma particle counter (Packard Instrument Co., Waltham, MA). Each condition is performed in at least triplicate, and the percentage of lysis is calculated using the formula: % Lysis=(ER−SR)/(TR−SR), where ER represents the average 51Cr released for each experimental condition. Alternatively, cytotoxicity can also be assessed using a Bright-Glo™ Luciferase Assay.

Imaging technologies can be used to evaluate specific trafficking and proliferation of CAR IL-15R/IL-15 expressing cells in tumor-bearing animal models. Such assays have been described, for example, in Barrett et al., Human Gene Therapy 22:1575-1586 (2011). Briefly, NOD/SCID/γc^{-/-} (NSG) mice or other immunodeficient are injected IV with multiple myeloma cells followed 7 days later with CART cells 4 hour after electroporation with the CAR IL-15R/IL-15 constructs. The T cells are stably transfected with a lentiviral construct to express firefly luciferase, and mice are imaged for bioluminescence. Alternatively, therapeutic efficacy and specificity of a single injection of CAR⁺ IL-15R/IL-15 T cells in a multiple myeloma xenograft model can be measured as the following: NSG mice are injected with multiple myeloma cells transduced to stably express firefly luciferase, followed by a single tail-vein injection of T cells electroporated with CAR construct days later. Animals are imaged at various time points post injection. For example, photon-density heat maps of firefly luciferasepositive tumors in representative mice at day 5 (2 days before treatment) and day 8 (24 hr post CAR⁺ PBLs) can be generated.

Alternatively, or in combination to the methods disclosed herein, methods and compositions for one or more of: detection and/or quantification of CAR IL-15R/IL-15-expressing cells (e.g., in vitro or in vivo (e.g., clinical monitoring)); immune cell expansion and/or activation; and/or CAR-specific selection, that involve the use of a CAR ligand, are disclosed. In one exemplary embodiment, the CAR ligand is an antibody that binds to the CAR molecule, e.g., binds to the extracellular antigen binding domain of CAR (e.g., an antibody that binds to the antigen binding domain, e.g., an anti-idiotypic antibody; or an antibody that binds to a constant region of the extracellular binding domain). In other embodiments, the CAR ligand is a CAR antigen molecule (e.g., a CAR antigen molecule as described herein).

In one aspect, a method for detecting and/or quantifying CAR IL-15R/IL-15 expressing cells is disclosed. For example, the CAR ligand can be used to detect and/or quantify CAR IL-15R/IL-15-expressing cells in vitro or in vivo (e.g., clinical monitoring of CAR-expressing cells in a patient, or dosing a patient). The method includes:
providing the CAR ligand (optionally, a labelled CAR ligand, e.g., a CAR ligand that includes a tag, a bead, a radioactive or fluorescent label);
acquiring the CAR IL-15R/IL-15-expressing cell (e.g., acquiring a sample containing CAR IL-15R/IL-15-expressing cells, such as a manufacturing sample or a clinical sample);
contacting the CAR IL-15R/IL-15-expressing cell with the CAR ligand under conditions where binding occurs, thereby detecting the level (e.g., amount) of the CAR-expressing cells present. Binding of the CAR IL-15R/IL-15-expressing cell with the CAR ligand can be detected using standard techniques such as FACS, ELISA and the like.

In another aspect, a method of expanding and/or activating cells (e.g., immune effector cells) is disclosed. The method includes:
providing a CAR IL-15R/IL-15-expressing cell (e.g., a first CAR IL-15R/IL-15-expressing cell or a transiently expressing CAR cell);
contacting said CAR IL-15R/IL-15-expressing cell with a CAR ligand, e.g., a CAR ligand as described herein), under conditions where immune cell expansion and/or proliferation occurs, thereby producing the activated and/or expanded cell population.

In certain embodiments, the CAR ligand is present on (e.g., is immobilized or attached to a substrate, e.g., a non-naturally occurring substrate). In some embodiments, the substrate is a non-cellular substrate. The non-cellular substrate can be a solid support chosen from, e.g., a plate (e.g., a microtiter plate), a membrane (e.g., a nitrocellulose membrane), a matrix, a chip or a bead. In embodiments, the CAR ligand is present in the substrate (e.g., on the substrate surface). The CAR ligand can be immobilized, attached, or associated covalently or non-covalently (e.g., cross-linked) to the substrate. In one embodiment, the CAR ligand is attached (e.g., covalently attached) to a bead. In the aforesaid embodiments, the immune cell population can be expanded in vitro or ex vivo. The method can further include culturing the population of immune cells in the presence of the ligand of the CAR molecule, e.g., using any of the methods described herein.

In other embodiments, the method of expanding and/or activating the cells further comprises addition of a second stimulatory molecule, e.g., CD28. For example, the CAR ligand and the second stimulatory molecule can be immobilized to a substrate, e.g., one or more beads, thereby providing increased cell expansion and/or activation.

In yet another aspect, a method for selecting or enriching for a CAR IL-15R/IL-15 expressing cell is provided. The method includes contacting the CAR IL-15R/IL-15 expressing cell with a CAR ligand as described herein; and selecting the cell on the basis of binding of the CAR ligand.

In yet other embodiments, a method for depleting, reducing and/or killing a CAR expressing cell is provided. The method includes contacting the CAR IL-15R/IL-15 expressing cell with a CAR ligand as described herein; and targeting the cell on the basis of binding of the CAR ligand, thereby reducing the number, and/or killing, the CAR IL-15R/IL-15-expressing cell. In one embodiment, the CAR ligand is coupled to a toxic agent (e.g., a toxin or a cell ablative drug). In another embodiment, the anti-idiotypic antibody can cause effector cell activity, e.g., ADCC or ADC activities.

Exemplary anti-CAR antibodies that can be used in the methods disclosed herein are described, e.g., in WO 2014/190273 and by Jena et al., “Chimeric Antigen Receptor (CAR)-Specific Monoclonal Antibody to Detect CD19-Specific T cells in Clinical Trials”, PLOS March 2013 8:3 e57838, the contents of which are incorporated by reference. In one embodiment, the anti-idiotypic antibody molecule recognizes an anti-CD19 antibody molecule, e.g., an anti-CD19 scFv. For instance, the anti-idiotypic antibody molecule can compete for binding with the CD19-specific CAR mAb clone no. 136.20.1 described in Jena et al., PLOS March 2013 8:3 e57838; may have the same CDRs (e.g., one or more of, e.g., all of, VH CDR1, VH CDR2, CH CDR3, VL CDR1, VL CDR2, and VL CDR3, using the Kabat definition, the Chothia definition, or a combination of tthe Kabat and Chothia definitions) as the CD19-specific CAR mAb clone no. 136.20.1; may have one or more (e.g., 2) variable regions as the CD19-specific CAR mAb clone no. 136.20.1, or may comprise the CD19-specific CAR mAb clone no. 136.20.1. In some embodiments, the anti-idiotypic antibody was made according to a method described in Jena et al. In another embodiment, the anti-idiotypic antibody molecule is an anti-idiotypic antibody molecule described in WO 2014/190273. In some embodiments, the anti-idiotypic antibody molecule has the same CDRs (e.g., one or more of, e.g., all of, VH CDR1, VH CDR2, CH CDR3, VL CDR1, VL CDR2, and VL CDR3) as an antibody molecule of WO 2014/190273 such as 136.20.1; may have one or more (e.g., 2) variable regions of an antibody molecule of WO 2014/190273, or may comprise an antibody molecule of WO 2014/190273 such as 136.20.1. In other embodiments, the anti-CAR antibody binds to a constant region of the extracellular binding domain of the CAR molecule, e.g., as described in WO 2014/190273. In some embodiments, the anti-CAR antibody binds to a constant region of the extracellular binding domain of the CAR molecule, e.g., a heavy chain constant region (e.g., a CH2-CH3 hinge region) or light chain constant region. For instance, in some embodiments the anti-CAR antibody competes for binding with the 2D3 monoclonal antibody described in WO 2014/190273, has the same CDRs (e.g., one or more of, e.g., all of, VH CDR1, VH CDR2, CH CDR3, VL CDR1, VL CDR2, and VL CDR3) as 2D3, or has one or more (e.g., 2) variable regions of 2D3, or comprises 2D3 as described in WO 2014/190273.

In some aspects and embodiments, the compositions and methods herein are optimized for a specific subset of T cells, e.g., as described in U.S. Ser. No. 62/031,699 filed Jul. 31, 2014, the contents of which are incorporated herein by reference in their entirety. In some embodiments, the optimized subsets of T cells display an enhanced persistence compared to a control T cell, e.g., a T cell of a different type (e.g., CD8⁺ or CD4⁺) expressing the same construct.

In some embodiments, a CD4⁺ T cell comprises a CAR IL-15R/IL-15 described herein, which CAR IL-15R/IL-15 comprises an intracellular signaling domain suitable for (e.g., optimized for, e.g., leading to enhanced persistence in) a CD4⁺ T cell, e.g., an ICOS domain. In some embodiments, a CD8⁺ T cell comprises a CAR IL-15R/IL-15 described herein, which CAR IL-15R/IL-15 comprises an intracellular signaling domain suitable for (e.g., optimized for, e.g., leading to enhanced persistence of) a CD8⁺ T cell, e.g., a 4-1BB domain, a CD28 domain, or another costimulatory domain other than an ICOS domain.

In an aspect, described herein is a method of treating a subject, e.g., a subject having cancer. The method includes administering to said subject, an effective amount of:
1) a CD4⁺ T cell comprising a CAR IL-15R/IL-15 (the CAR^{CD4+})
comprising:
an antigen binding domain, e.g., an antigen binding domain described herein;
a transmembrane domain; and
an intracellular signaling domain, e.g., a first costimulatory domain, e.g., an ICOS domain; and
2) a CD8⁺ T cell comprising a CAR IL-15R/IL-15 (the CAR^{CD8+}) comprising:
an antigen binding domain, e.g., an antigen binding domain described herein;
a transmembrane domain; and
an intracellular signaling domain, e.g., a second costimulatory domain, e.g., a 4-1BB domain, a CD28 domain, or another costimulatory domain other than an ICOS domain;
wherein the CAR^{CD4+} and the CAR^{CD8+} differ from one another.

Optionally, the method further includes administering:
3) a second CD8+ T cell comprising a CAR IL-15R/IL-15 (the second CAR^{CD8+}) comprising:
   an antigen binding domain, e.g., an antigen binding domain described herein;
   a transmembrane domain; and
      an intracellular signaling domain, wherein the second CAR^{CD8+} comprises an intracellular signaling domain, e.g., a costimulatory signaling domain, not present on the CAR^{CD8+}, and, optionally, does not comprise an ICOS signaling domain.

Other assays, including those that are known in the art can also be used to evaluate the CAR IL-15R/IL-15 constructs of the invention.

### IL-15 and IL-15 Receptor Molecules

In embodiments of any of the compositions or methods disclosed herein, the cytokine molecule is an IL-15 molecule, e.g., a full length, a fragment or a variant of IL-15, e.g., human IL-15. In embodiments, the IL-15 molecule is a wild-type, human IL-15, e.g., having the amino acid sequence of SEQ ID NO: 1007. In other embodiments, the IL-15 molecule is a variant of human IL-15, e.g., having one or more amino acid modifications compared to SEQ ID NO: 1007. In some embodiments, the IL-15 molecule comprises a mutation, e.g., an N72D point mutation as disclosed in Zhu X. et al., (2009) Journal of Immunology 183(6): 3598, the entire contents of which are hereby incorporated by reference.

Human Interleukin 15
(SEQ ID NO: 1007)

| | |
|---|---|
| 1 | MVLGTIDLCS CFSAGLPKTE ANWVNVISDL KKIEDLIQSM |
| | HIDATLYTES DVHPSCKVTA |
| 61 | MKCFLLELQV ISLESGDASI HDTVENLIIL ANNSLSSNGN |
| | VTESGCKECE ELEEKNIKEF |
| 121 | LQSFVHIVQM FINTS |

In embodiments of any of the compositions or methods disclosed herein, the IL-15 receptor molecule is an IL-15 receptor alpha (IL-15Ra), e.g., a full length, a fragment or a variant of IL-15Ra, e.g., human IL-15Ra. In embodiments, the IL-15Ra molecule is a wild-type, human IL-15Ra, e.g., having the amino acid sequence of SEQ ID NO: 1008. In other embodiments, the IL-15Ra molecule is a variant of human IL-15Ra, e.g., having one or more amino acid modifications compared to SEQ ID NO: 1008.

Human Interleukin 15 receptor alpha
(SEQ ID NO: 1008)

| | |
|---|---|
| 1 | MAPRRARGCR TLGLPALLLL LLLRPPATRG ITCPPPMSVE |
| | HADIWVKSYS LYSRERYICN |
| 61 | SGFKRKAGTS SLTECVLNKA TNVAHWTTPS LKCIRDPALV |
| | HQRPAPPSTV TTAGVTPQPE |
| 121 | SLSPSGKEPA ASSPSSNNTA ATTAAIVPGS QLMPSKSPST |
| | GTTEISSHES SHGTPSQTTA |
| 181 | KNWELTASAS HQPPGVYPQG HSDTTVAIST STVLLCGLSA |
| | VSLLACYLKS RQTPPLASVE |
| 241 | MEAMEALPVT WGTSSRDEDL ENCSHHL |

### IL-15/IL-15Ra Complexes

In certain embodiments, a combination described herein comprises an IL-15/IL-15Ra complex. In some embodiments, the IL-15/IL-15Ra complex is chosen from NIZ985 (Novartis), ATL-803 (Altor) or CYP0150 (Cytune). In some embodiments, the IL-15/IL-15RA complex is NIZ985. Without wishing to be bound by theory, it is believed that in some embodiments, IL-15 potentiates, e.g., enhances, Natural Killer cells to eliminate, e.g., kill, pancreatic cancer cells. In an embodiment, response, e.g., therapeutic response, to a combination described herein, e.g., a combination comprising an IL-15/IL15Ra complex, in, e.g., an animal model of colorectal cancer is associated with Natural Killer cell infiltration.

### Exemplary IL-15/IL-15Ra Complexes

In one embodiment, the IL-15/IL-15Ra complex comprises human IL-15 complexed with a soluble form of human IL-15Ra. The complex may comprise IL-15 covalently or noncovalently bound to a soluble form of IL-15Ra. In a particular embodiment, the human IL-15 is noncovalently bonded to a soluble form of IL-15Ra. In a particular embodiment, the human IL-15 of the composition comprises an amino acid sequence of SEQ ID NO: 1001 in Table 11 and the soluble form of human IL-15Ra comprises an amino acid sequence of SEQ ID NO:1002 in Table 11, as described in WO 2014/066527, incorporated by reference in its entirety. The molecules described herein can be made by vectors, host cells, and methods described in WO 2007/084342, incorporated by reference in its entirety.

TABLE 11
Amino acid and nucleotide sequences of
exemplary IL-15/IL-15Ra complexes
NIZ985

| | | |
|---|---|---|
| SEQ ID | Human | NWVNVISDLKKIEDLIQSMHIDATLYTESDVHPSC |
| NO: 1001 | IL-15 | KVTAMKCFLLELQVISLESGDASIHDTVENLIILA |
| | | NNSLSSNGNVTESGCKECEELEEKNIKEFLQSFVH |
| | | IVQMFINTS |
| SEQ ID | Human | ITCPPPMSVEHADIWVKSYSLYSRERYICNSGFKR |
| NO: 1002 | Soluble | KAGTSSLTECVLNKATNVAHWTTPSLKCIRDPALV |
| | IL-15Ra | HQRPAPPSTVTTAGVTPQPESLSPSGKEPAASSPS |
| | | SNNTAATTAAIVPGSQLMPSKSPSTGTTEISSHES |
| | | SHGTPSQTTAKNWELTASASHQPPGVYPQG |

Without wishing to be bound by theory, it is believed that in microsatellite stable CRCs with low T cell infiltration, IL-15 may promote, e.g., increase, T cell priming (e.g., as described in Lou, K. J. SciBX 7 (16); 10.1038/SCIBX.2014.449). In some embodiments, the combination promotes, e.g., increases T cell priming. Without wishing to be bound by theory, it is further believed that IL-15 may induce NK cell infiltration. In some embodiments, response to an IL-15/IL-15RA complex can result in NK cell infiltration.

### Other Exemplary IL-15/IL-15Ra Complexes

In one embodiment, the IL-15/IL-15Ra complex is ALT-803, an IL-15/IL-15Ra Fc fusion protein (IL-15N72D:IL-15RaSu/Fc soluble complex). ALT-803 is disclosed in WO 2008/143794, incorporated by reference in its entirety. In one embodiment, the IL-15/IL-15Ra Fc fusion protein comprises the sequences as disclosed in Table 12.

In one embodiment, the IL-15/IL-15Ra complex comprises IL-15 fused to the sushi domain of IL-15Ra (CYP0150, Cytune). The sushi domain of IL-15Ra refers to a domain beginning at the first cysteine residue after the signal peptide of IL-15Ra, and ending at the fourth cysteine residue after said signal peptide. The complex of IL-15 fused to the sushi domain of IL-15Ra is disclosed in WO 2007/04606 and WO 2012/175222, incorporated by reference in their entirety. In one embodiment, the IL-15/IL-15Ra sushi domain fusion comprises the sequences as disclosed in Table 12.

TABLE 12
Amino acid sequences of other exemplary IL-15/IL-15Ra complexes
ALT-803 (Altor)

| | | |
|---|---|---|
| SEQ ID | IL-15N72D | NWVNVISDLKKIEDLIQSMHIDATLYTESDVHPSCKVTA |
| NO: 1003 | | MKCFLLELQVISLESGDASIHDTVENLIILANDSLSSNG |
| | | NVTESGCKECEELEEKNIKEFLQSFVHIVQMFINTS |
| SEQ ID | IL-15RaSu/ | ITCPPPMSVEHADIWVKSYSLYSRERYICNSGFKRKAGT |
| NO: 1004 | Fc | SSLTECVLNKATNVAHWTTPSLKCIREPKSCDKTHTCPP |
| | | CPAPELLGGPSVFLFPPKPKDTLMISRTPEVTCVVVDVS |
| | | HEDPEVKFNWYVDGVEVHNAKTKPREEQYNSTYRVVSVL |
| | | TVLHQDWLNGKEYKCKVSNKALPAPIEKTISKAKGQPRE |
| | | PQVYTLPPSRDELTKNQVSLTCLVKGFYPSDIAVEWESN |
| | | GQPENNYKTTPPVLDSDGSFFLYSKLTVDKSRWQQGNVF |
| | | SCSVMHEALHNHYTQKSLSLSPGK |

IL-15/IL-15Ra sushi domain fusion (Cytune)

| | | |
|---|---|---|
| SEQ ID | Human IL- | NWVNVISDLKKIEDLIQSMHIDATLYTESDVHPSCKVTA |
| NO: 1005 | 15 | MKCFLLELQVISLESGDASIHDTVENLIILANNSLSSNG |
| | | NVTESGCKECEELEXKNIKEFLQSFVHIVQMFINTS |
| | | Where X is E or K |
| SEQ ID | Human IL- | ITCPPPMSVEHADIWVKSYSLYSRERYICNSGFKRKAGT |
| NO: 1006 | 15Ra sushi | SSLTECVLNKATNVAHWTTPSLKCIRDPALVHQRPAPP |
| | and hinge | |
| | domains | |

### Therapeutic Application

### Methods Using Biomarkers for Evaluating CAR-Effectiveness, Subject Suitability, or Sample Suitability

In another aspect, the invention features a method of evaluating or monitoring the effectiveness of a CAR-expressing cell therapy in a subject (e.g., a subject having a cancer). The method includes acquiring a value of effectiveness to the CAR IL-15R/IL-15 therapy, subject suitability, or sample suitability, wherein said value is indicative of the effectiveness or suitability of the CAR-expressing cell therapy.

In some embodiments of any of the methods disclosed herein, the subject is evaluated prior to receiving, during, or after receiving, the CAR IL-15R/IL-15-expressing cell therapy.

In some embodiments of any of the methods disclosed herein, a responder (e.g., a complete responder) has, or is identified as having, a greater level or activity of one, two, or more (all) of GZMK, PPF1BP2, or naïve T cells as compared to a non-responder.

In some embodiments of any of the methods disclosed herein, a non-responder has, or is identified as having, a greater level or activity of one, two, three, four, five, six, seven, or more (e.g., all) of IL22, IL-2RA, IL-21, IRF8, IL8, CCL17, CCL22, effector T cells, or regulatory T cells, as compared to a responder.

In an embodiment, a relapser is a patient having, or who is identified as having, an increased level of expression of one or more of (e.g., 2, 3, 4, or all of) the following genes, compared to non relapsers: MIR199A1, MIR1203, uc021ovp, ITM2C, and HLA-DQB1 and/or a decreased levels of expression of one or more of (e.g., 2, 3, 4, 5, 6, 7, 8, 9, 10, 11, or all of) the following genes, compared to non relapsers: PPIAL4D, TTTY10, TXLNG2P, MIR4650-1, KDM5D, USP9Y, PRKY, RPS4Y2, RPS4Y1, NCRNA00185, SULT1E1, and EIF1AY.

In some embodiments of any of the methods disclosed herein, a non-responder has, or is identified as having, a greater percentage of an immune cell exhaustion marker, e.g., one, two or more immune checkpoint inhibitors (e.g., PD-1, PD-L1, TIM-3 and/or LAG-3). In one embodiment, a non-responder has, or is identified as having, a greater percentage of PD-1, PD-L1, or LAG-3 expressing immune effector cells (e.g., CD4+ T cells and/or CD8+ T cells) (e.g., CAR-expressing CD4+ cells and/or CD8+ T cells) compared to the percentage of PD-1 or LAG-3 expressing immune effector cells from a responder.

In one embodiment, a non-responder has, or is identified as having, a greater percentage of immune cells having an exhausted phenotype, e.g., immune cells that co-express at least two exhaustion markers, e.g., co-expresses PD-1, PD-L1 and/or TIM-3. In other embodiments, a non-responder has, or is identified as having, a greater percentage of immune cells having an exhausted phenotype, e.g., immune cells that co-express at least two exhaustion markers, e.g., co-expresses PD-1 and LAG-3.

In some embodiments of any of the methods disclosed herein, a non-responder has, or is identified as having, a greater percentage of PD-1/PD-L1+/LAG-3+ cells in the CAR IL-15R/IL-15-expressing cell population compared to a responder (e.g., a complete responder) to the CAR-expressing cell therapy.

In some embodiments of any of the methods disclosed herein, a partial responder has, or is identified as having, a higher percentages of PD-1/PD-L1+/LAG-3+ cells, than a responder, in the CAR IL-15R/IL-15-expressing cell population.

In some embodiments of any of the methods disclosed herein, a non-responder has, or is identified as having, an exhausted phenotype of PD1/PD-L1+ CAR+ and co-expression of LAG3 in the CAR IL-15R/IL-15-expressing cell population.

In some embodiments of any of the methods disclosed herein, a non-responder has, or is identified as having, a greater percentage of PD-1/PD-L1+/TIM-3+ cells in the CAR-expressing cell population compared to the responder (e.g., a complete responder).

In some embodiments of any of the methods disclosed herein, a partial responders has, or is identified as having, a higher percentage of PD-1/PD-L1+/TIM-3+ cells, than responders, in the CAR IL-15R/IL-15-expressing cell population.

In some embodiments of any of the methods disclosed herein, the presence of CD8+CD27+CD45RO-T cells in an apheresis sample is a positive predictor of the subject response to a CAR IL-15R/IL-15-expressing cell therapy.

In some embodiments of any of the methods disclosed herein, a high percentage of PD1+ CAR+ and LAG3+ or TIM3+ T cells in an apheresis sample is a poor prognostic predictor of the subject response to a CAR IL-15R/IL-15-expressing cell therapy.

In some embodiments of any of the methods disclosed herein, the responder (e.g., the complete or partial responder) has one, two, three or more (or all) of the following profile:
(i) has a greater number of CD27+ immune effector cells compared to a reference value, e.g., a non-responder number of CD27+ immune effector cells;
(ii) has a greater number of CD8+ T cells compared to a reference value, e.g., a non-responder number of CD8+ T cells;
(iii) has a lower number of immune cells expressing one or more checkpoint inhibitors, e.g., a checkpoint inhibitor chosen from PD-1, PD-L1, LAG-3, TIM-3, or KLRG-1, or a combination, compared to a reference value, e.g., a non-responder number of cells expressing one or more checkpoint inhibitors; or
(iv) has a greater number of one, two, three, four or more (all) of resting TEFF cells, resting TREG cells, naïve CD4 cells, unstimulated memory cells or early memory T cells, or a combination thereof, compared to a reference value, e.g., a non-responder number of resting TEFF cells, resting TREG cells, naïve CD4 cells, unstimulated memory cells or early memory T cells.

In some embodiments of any of the methods disclosed herein, the cytokine level or activity is chosen from one, two, three, four, five, six, seven, eight, or more (or all) of cytokine CCL20/MIP3a, IL17A, IL6, GM-CSF, IFN-γ, IL10, IL13, IL2, IL21, IL4, IL5, IL9 or TNFα, or a combination thereof. The cytokine can be chosen from one, two, three, four or more (all) of IL-17a, CCL20, IL2, IL6, or TNFa. In one embodiment, an increased level or activity of a cytokine is chosen from one or both of IL-17a and CCL20, is indicative of increased responsiveness or decreased relapse.

In embodiments, the responder, a non-responder, a relapser or a non-relapser identified by the methods herein can be further evaluated according to clinical criteria. For example, a complete responder has, or is identified as, a subject having a disease, e.g., a cancer, who exhibits a complete response, e.g., a complete remission, to a treatment. A complete response may be identified, e.g., using the NCCN Guidelines®, or Cheson et al, J Clin Oncol 17:1244 (1999) and Cheson et al., “Revised Response Criteria for Malignant Lymphoma”, J Clin Oncol 25:579-586 (2007) (both of which are incorporated by reference herein in their entireties), as described herein. A partial responder has, or is identified as, a subject having a disease, e.g., a cancer, who exhibits a partial response, e.g., a partial remission, to a treatment. A partial response may be identified, e.g., using the NCCN Guidelines®, or Cheson criteria as described herein. A non-responder has, or is identified as, a subject having a disease, e.g., a cancer, who does not exhibit a response to a treatment, e.g., the patient has stable disease or progressive disease. A non-responder may be identified, e.g., using the NCCN Guidelines®, or Cheson criteria as described herein.

Alternatively, or in combination with the methods disclosed herein, responsive to said value, performing one, two, three four or more of:
administering e.g., to a responder or a non-relapser, a CAR IL-15R/IL-15-expressing cell therapy;
administered an altered dosing of a CAR IL-15R/IL-15-expressing cell therapy;
altering the schedule or time course of a CAR IL-15R/IL-15-expressing cell therapy;
administering, e.g., to a non-responder or a partial responder, an additional agent in combination with a CAR IL-15R/IL-15-expressing cell therapy, e.g., a checkpoint inhibitor, e.g., a checkpoint inhibitor described herein;
administering to a non-responder or partial responder a therapy that increases the number of younger T cells in the subject prior to treatment with a CAR IL-15R/IL-15-expressing cell therapy;
modifying a manufacturing process of a CAR IL-15R/IL-15-expressing cell therapy, e.g., enriching for younger T cells prior to introducing a nucleic acid encoding a CAR, or increasing the transduction efficiency, e.g., for a subject identified as a non-responder or a partial responder;
administering an alternative therapy, e.g., for a non-responder or partial responder or relapser; or
if the subject is, or is identified as, a non-responder or a relapser, decreasing the TREG cell population and/or TREG gene signature, e.g., by one or more of CD25 depletion, administration of cyclophosphamide, anti-GITR antibody, or a combination thereof.

In certain embodiments, the subject is pre-treated with an anti-GITR antibody. In certain embodiment, the subject is treated with an anti-GITR antibody prior to infusion or re-infusion.

### Combination Therapies

A CAR IL-15R/IL-15-expressing cell described herein may be used in combination with other known agents and therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

A CAR IL-15R/IL-15-expressing cell described herein and the at least one additional therapeutic agent can be administered simultaneously, in the same or in separate compositions, or sequentially. For sequential administration, the CAR-expressing cell described herein can be administered first, and the additional agent can be administered second, or the order of administration can be reversed.

The CAR IL-15R/IL-15 therapy and/or other therapeutic agents, procedures or modalities can be administered during periods of active disorder, or during a period of remission or less active disease. The CAR therapy can be administered before the other treatment, concurrently with the treatment, post-treatment, or during remission of the disorder.

When administered in combination, the CAR IL-15R/IL-15 therapy and the additional agent (e.g., second or third agent), or all, can be administered in an amount or dose that is higher, lower or the same than the amount or dosage of each agent used individually, e.g., as a monotherapy. In certain embodiments, the administered amount or dosage of the CAR IL-15R/IL-15 therapy, the additional agent (e.g., second or third agent), or all, is lower (e.g., at least 20%, at least 30%, at least 40%, or at least 50%) than the amount or dosage of each agent used individually, e.g., as a monotherapy. In other embodiments, the amount or dosage of the CAR IL-15R/IL-15 therapy, the additional agent (e.g., second or third agent), or all, that results in a desired effect (e.g., treatment of cancer) is lower (e.g., at least 20%, at least 30%, at least 40%, or at least 50% lower) than the amount or dosage of each agent used individually, e.g., as a monotherapy, required to achieve the same therapeutic effect.

In some embodiments, the invention discloses a combination therapy including a CAR IL-15R/IL-15-expressing cell therapy described herein, an RNA molecule described herein (or a nucleic acid molecule encoding the RNA molecule), and an additional therapeutic agent.

### PD-1 Inhibitor

In some embodiments, the additional therapeutic agent is a PD-1 inhibitor. In some embodiments, the PD-1 inhibitor is chosen from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune).

In one embodiment, the PD-1 inhibitor is an anti-PD-1 antibody molecule. In one embodiment, the PD-1 inhibitor is an anti-PD-1 antibody molecule as described in US 2015/0210769, published on Jul. 30, 2015, entitled “Antibody Molecules to PD-1 and Uses Thereof,” incorporated by reference in its entirety. In one embodiment, the anti-PD-1 antibody molecule comprises the CDRs, variable regions, heavy chains and/or light chains of BAP049-Clone-E or BAP049-Clone-B disclosed in US 2015/0210769. The antibody molecules described herein can be made by vectors, host cells, and methods described in US 2015/0210769, incorporated by reference in its entirety.

In one embodiment, the anti-PD-1 antibody molecule is Nivolumab (Bristol-Myers Squibb), also known as MDX-1106, MDX-1106-04, ONO-4538, BMS-936558, or OPDIVO®. Nivolumab (clone 5C4) and other anti-PD-1 antibodies are disclosed in U.S. Pat. No. 8,008,449 and WO 2006/121168, incorporated by reference in their entirety. In one embodiment, the anti-PD-1 antibody molecule is Pembrolizumab (Merck & Co), also known as Lambrolizumab, MK-3475, MK03475, SCH-900475, or KEYTRUDA®. Pembrolizumab and other anti-PD-1 antibodies are disclosed in Hamid, O. et al. (2013) New England Journal of Medicine 369 (2): 134-44, U.S. Pat. No. 8,354,509, and WO 2009/114335, incorporated by reference in their entirety. In one embodiment, the anti-PD-1 antibody molecule is Pidilizumab (CureTech), also known as CT-011. Pidilizumab and other anti-PD-1 antibodies are disclosed in Rosenblatt, J. et al. (2011) J Immunotherapy 34(5): 409-18, U.S. Pat. Nos. 7,695,715, 7,332,582, and 8,686,119, incorporated by reference in their entirety. In one embodiment, the anti-PD-1 antibody molecule is MEDI0680 (Medimmune), also known as AMP-514. MEDI0680 and other anti-PD-1 antibodies are disclosed in U.S. Pat. No. 9,205,148 and WO 2012/145493, incorporated by reference in their entirety. In one embodiment, the anti-PD-1 antibody molecule is REGN2810 (Regeneron). In one embodiment, the anti-PD-1 antibody molecule is PF-06801591 (Pfizer). In one embodiment, the anti-PD-1 antibody molecule is BGB-A317 or BGB-108 (Beigene). In one embodiment, the anti-PD-1 antibody molecule is INCSHR1210 (Incyte), also known as INCSHR01210 or SHR-1210. In one embodiment, the anti-PD-1 antibody molecule is TSR-042 (Tesaro), also known as ANB011.

Further known anti-PD-1 antibody molecules include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety.

In one embodiment, the PD-1 inhibitor is a peptide that inhibits the PD-1 signaling pathway, e.g., as described in U.S. Pat. No. 8,907,053, incorporated by reference in its entirety. In one embodiment, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In one embodiment, the PD-1 inhibitor is AMP-224 (B7-DCIg (Amplimmune), e.g., disclosed in WO 2010/027827 and WO 2011/066342, incorporated by reference in their entirety).

### PD-L1 Inhibitors

In some embodiments, the additional therapeutic agent is a PD-L1 inhibitor. In some embodiments, the PD-L1 inhibitor is chosen from FAZ053 (Novartis), Atezolizumab (Genentech/Roche), Avelumab (Merck Serono and Pfizer), Durvalumab (MedImmune/AstraZeneca), or BMS-936559 (Bristol-Myers Squibb).

In one embodiment, the PD-L1 inhibitor is an anti-PD-L1 antibody molecule. In one embodiment, the PD-L1 inhibitor is an anti-PD-L1 antibody molecule as disclosed in US 2016/0108123, published on Apr. 21, 2016, entitled “Antibody Molecules to PD-L1 and Uses Thereof,” incorporated by reference in its entirety. In one embodiment, the anti-PD-L1 antibody molecule comprises the CDRs, variable regions, heavy chains and/or light chains of BAP058-Clone O or BAP058-Clone N disclosed in US 2016/0108123.

In one embodiment, the anti-PD-L1 antibody molecule is Atezolizumab (Genentech/Roche), also known as MPDL3280A, RG7446, RO5541267, YW243.55.S70, or TECENTRIQ™. Atezolizumab and other anti-PD-L1 antibodies are disclosed in U.S. Pat. No. 8,217,149, incorporated by reference in its entirety. In one embodiment, the anti-PD-L1 antibody molecule is Avelumab (Merck Serono and Pfizer), also known as MSB0010718C. Avelumab and other anti-PD-L1 antibodies are disclosed in WO 2013/079174, incorporated by reference in its entirety. In one embodiment, the anti-PD-L1 antibody molecule is Durvalumab (MedImmune/AstraZeneca), also known as MEDI4736. Durvalumab and other anti-PD-L1 antibodies are disclosed in U.S. Pat. No. 8,779,108, incorporated by reference in its entirety. In one embodiment, the anti-PD-L1 antibody molecule is BMS-936559 (Bristol-Myers Squibb), also known as MDX-1105 or 12A4. BMS-936559 and other anti-PD-L1 antibodies are disclosed in U.S. Pat. No. 7,943,743 and WO 2015/081158, incorporated by reference in their entirety.

Further known anti-PD-L1 antibodies include those described, e.g., in WO 2015/181342, WO 2014/100079, WO 2016/000619, WO 2014/022758, WO 2014/055897, WO 2015/061668, WO 2013/079174, WO 2012/145493, WO 2015/112805, WO 2015/109124, WO 2015/195163, U.S. Pat. Nos. 8,168,179, 8,552,154, 8,460,927, and 9,175,082, incorporated by reference in their entirety.

### LAG-3 Inhibitors

In some embodiments, the additional therapeutic agent is a LAG-3 inhibitor. In some embodiments, the LAG-3 inhibitor is chosen from LAG525 (Novartis), BMS-986016 (Bristol-Myers Squibb), or TSR-033 (Tesaro).

In one embodiment, the LAG-3 inhibitor is an anti-LAG-3 antibody molecule. In one embodiment, the LAG-3 inhibitor is an anti-LAG-3 antibody molecule as disclosed in US 2015/0259420, published on Sep. 17, 2015, entitled “Antibody Molecules to LAG-3 and Uses Thereof,” incorporated by reference in its entirety. In one embodiment, the anti-LAG-3 antibody molecule comprises the CDRs, variable regions, heavy chains and/or light chains of BAP050-Clone I or BAP050-Clone J disclosed in US 2015/0259420.

In one embodiment, the anti-LAG-3 antibody molecule is BMS-986016 (Bristol-Myers Squibb), also known as BMS986016. BMS-986016 and other anti-LAG-3 antibodies are disclosed in WO 2015/116539 and U.S. Pat. No. 9,505,839, incorporated by reference in their entirety. In one embodiment, the anti-LAG-3 antibody molecule is TSR-033 (Tesaro). In one embodiment, the anti-LAG-3 antibody molecule is IMP731 or GSK2831781 (GSK and Prima BioMed). IMP731 and other anti-LAG-3 antibodies are disclosed in WO 2008/132601 and U.S. Pat. No. 9,244,059, incorporated by reference in their entirety. In one embodiment, the anti-LAG-3 antibody molecule is IMP761 (Prima BioMed).

Further known anti-LAG-3 antibodies include those described, e.g., in WO 2008/132601, WO 2010/019570, WO 2014/140180, WO 2015/116539, WO 2015/200119, WO 2016/028672, U.S. Pat. Nos. 9,244,059, 9,505,839, incorporated by reference in their entirety.

In one embodiment, the anti-LAG-3 inhibitor is a soluble LAG-3 protein, e.g., IMP321 (Prima BioMed), e.g., as disclosed in WO 2009/044273, incorporated by reference in its entirety.

### TIM-3 Inhibitors

In some embodiments, the additional therapeutic agent is a TIM-3 inhibitor. In some embodiments, the TIM-3 inhibitor is MGB453 (Novartis) or TSR-022 (Tesaro).

In one embodiment, the TIM-3 inhibitor is an anti-TIM-3 antibody molecule. In one embodiment, the TIM-3 inhibitor is an anti-TIM-3 antibody molecule as disclosed in US 2015/0218274, published on Aug. 6, 2015, entitled “Antibody Molecules to TIM-3 and Uses Thereof,” incorporated by reference in its entirety. In one embodiment, the anti-TIM-3 antibody molecule comprises the CDRs, variable regions, heavy chains and/or light chains of ABTIM3-hum11 or ABTIM3-hum03 disclosed in US 2015/0218274.

In one embodiment, the anti-TIM-3 antibody molecule is TSR-022 (AnaptysBio/Tesaro). In one embodiment, the anti-TIM-3 antibody molecule comprises one or more of the CDR sequences (or collectively all of the CDR sequences), the heavy chain or light chain variable region sequence, or the heavy chain or light chain sequence of APE5137 or APE5121. APE5137, APE5121, and other anti-TIM-3 antibodies are disclosed in WO 2016/161270, incorporated by reference in its entirety. In one embodiment, the anti-TIM-3 antibody molecule is the antibody clone F38-2E2.

Further known anti-TIM-3 antibodies include those described, e.g., in WO 2016/111947, WO 2016/071448, WO 2016/144803, U.S. Pat. Nos. 8,552,156, 8,841,418, and 9,163,087, incorporated by reference in their entirety.

### Chemotherapeutic Agents

In some embodiments, the additional therapeutic agent is a chemotherapeutic agent. Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, tositumomab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide).

General Chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Exemplary alkylating agents include, without limitation, nitrogen mustards, ethylenimine derivatives, alkyl sulfonates, nitrosoureas and triazenes): uracil mustard (Aminouracil Mustard®), Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Exemplary mTOR inhibitors include, e.g., temsirolimus; ridaforolimus (formally known as deferolimus, (1R,2R,4S)-4-[(2R)-2 [(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.0^{4,9}]hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RAD001); rapamycin (AY22989, Sirolimus®); simapimod (CAS 164301-51-3); emsirolimus, (5-{2,4-Bis[(3S)-3-methylmorpholin-4-yl]pyrido[2,3-d]pyrimidin-7-yl}-2-methoxyphenyl) methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7 (8H)-one (PF04691502, CAS 1013101-36-4); and N²-[1,4-dioxo-4-[[4-(4-oxo-8-phenyl-4H-1-benzopyran-2-yl) morpholinium-4-yl]methoxy]butyl]-L-arginylglycyl-L-α-aspartyIL-serine-inner salt (SEQ ID NO: 1482) (SF1126, CAS 936487-67-1), and XL765.

Exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon γ, CAS 951209-71-5, available from IRX Therapeutics).

Exemplary anthracyclines include, e.g., doxorubicin (Adriamycin® and Rubex®); bleomycin (Lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin.

Exemplary vinca alkaloids include, e.g., vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®).

Exemplary proteosome inhibitors include bortezomib (Velcade®); carfilzomib (PX-171-007, (S)-4-Methyl-N-((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)-pentanamide); marizomib (NPI-0052); ixazomib citrate (MLN-9708); delanzomib (CEP-18770); and O-Methyl-N-[(2-methyl-5-thiazolyl) carbonyl]-L-seryl-O-methyl-N-[(1S)-2-[(2R)-2-methyl-2-oxiranyl]-2-oxo-1-(phenylmethyl)ethyl]-L-serinamide (ONX-0912).

### Biopolymer Delivery Methods

In some embodiments, one or more CAR-expressing cells as disclosed herein can be administered or delivered to the subject via a biopolymer scaffold, e.g., a biopolymer implant. Biopolymer scaffolds can support or enhance the delivery, expansion, and/or dispersion of the CAR-expressing cells described herein. A biopolymer scaffold comprises a biocompatible (e.g., does not substantially induce an inflammatory or immune response) and/or a biodegradable polymer that can be naturally occurring or synthetic.

Examples of suitable biopolymers include, but are not limited to, agar, agarose, alginate, alginate/calcium phosphate cement (CPC), beta-galactosidase (β-GAL), (1,2,3,4,6-pentaacetyl a-D-galactose), cellulose, chitin, chitosan, collagen, elastin, gelatin, hyaluronic acid collagen, hydroxyapatite, poly(3-hydroxybutyrate-co-3-hydroxy-hexanoate) (PHBHHx), poly(lactide), poly(caprolactone) (PCL), poly(lactide-co-glycolide) (PLG), polyethylene oxide (PEO), poly(lactic-co-glycolic acid) (PLGA), polypropylene oxide (PPO), polyvinyl alcohol) (PVA), silk, soy protein, and soy protein isolate, alone or in combination with any other polymer composition, in any concentration and in any ratio. The biopolymer can be augmented or modified with adhesion- or migration-promoting molecules, e.g., collagen-mimetic peptides that bind to the collagen receptor of lymphocytes, and/or stimulatory molecules to enhance the delivery, expansion, or function, e.g., anti-cancer activity, of the cells to be delivered. The biopolymer scaffold can be an injectable, e.g., a gel or a semi-solid, or a solid composition.

In some embodiments, CAR-expressing cells described herein are seeded onto the biopolymer scaffold prior to delivery to the subject. In embodiments, the biopolymer scaffold further comprises one or more additional therapeutic agents described herein (e.g., another CAR-expressing cell, an antibody, or a small molecule) or agents that enhance the activity of a CAR-expressing cell, e.g., incorporated or conjugated to the biopolymers of the scaffold. In embodiments, the biopolymer scaffold is injected, e.g., intratumorally, or surgically implanted at the tumor or within a proximity of the tumor sufficient to mediate an anti-tumor effect. Additional examples of biopolymer compositions and methods for their delivery are described in Stephan et al., Nature Biotechnology, 2015, 33:97-101; and WO2014/110591.

### Pharmaceutical Compositions and Treatments

Pharmaceutical compositions of the present invention may comprise a CAR-expressing cell, e.g., a plurality of CAR-expressing cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are in one aspect formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

In one embodiment, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In one embodiment, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

When “an immunologically effective amount,” “an anti-tumor effective amount,” “a tumor-inhibiting effective amount,” or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988).

In certain aspects, it may be desired to administer activated T cells to a subject and then subsequently redraw blood (or have an apheresis performed), activate T cells therefrom according to the present invention, and reinfuse the patient with these activated and expanded T cells. This process can be carried out multiple times every few weeks. In certain aspects, T cells can be activated from blood draws of from 10 cc to 400 cc. In certain aspects, T cells are activated from blood draws of 20 cc, 30 cc, 40 cc, 50 cc, 60 cc, 70 cc, 80 cc, 90 cc, or 100 cc.

The administration of the subject compositions may be carried out in any convenient manner, including by aerosol inhalation, injection, ingestion, transfusion, implantation or transplantation. The compositions described herein may be administered to a patient trans arterially, subcutaneously, intradermally, intratumorally, intranodally, intramedullary, intramuscularly, by intravenous (i.v.) injection, or intraperitoneally. In one aspect, the T cell compositions of the present invention are administered to a patient by intradermal or subcutaneous injection. In one aspect, the CAR-expressing cell (e.g., T cell or NK cell) compositions of the present invention are administered by i.v. injection. The compositions of CAR-expressing cells (e.g., T cells or NK cells) may be injected directly into a tumor, lymph node, or site of infection.

In a particular exemplary aspect, subjects may undergo leukapheresis, wherein leukocytes are collected, enriched, or depleted ex vivo to select and/or isolate the cells of interest, e.g., immune effector cells (e.g., T cells or NK cells). These immune effector cell (e.g., T cell or NK cell) isolates may be expanded by methods known in the art and treated such that one or more CAR constructs of the invention may be introduced, thereby creating a CAR-expressing cell (e.g., CAR T cell or CAR-expressing NK cell) of the invention. Subjects in need thereof may subsequently undergo standard treatment with high dose chemotherapy followed by peripheral blood stem cell transplantation. In certain aspects, following or concurrent with the transplant, subjects receive an infusion of the expanded CAR-expressing cells (e.g., CAR T cells or NK cells) of the present invention. In an additional aspect, expanded cells are administered before or following surgery.

In embodiments, lymphodepletion is performed on a subject, e.g., prior to administering one or more cells that express a CAR described herein. In embodiments, the lymphodepletion comprises administering one or more of melphalan, cytoxan, cyclophosphamide, and fludarabine.

The dosage of the above treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

In one embodiment, the CAR is introduced into immune effector cells (e.g., T cells or NK cells), e.g., using in vitro transcription, and the subject (e.g., human) receives an initial administration of CAR immune effector cells (e.g., T cells or NK cells) of the invention, and one or more subsequent administrations of the CAR immune effector cells (e.g., T cells or NK cells) of the invention, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of the CAR immune effector cells (e.g., T cells or NK cells) of the invention are administered to the subject (e.g., human) per week, e.g., 2, 3, or 4 administrations of the CAR immune effector cells (e.g., T cells or NK cells) of the invention are administered per week. In one embodiment, the subject (e.g., human subject) receives more than one administration of the CAR immune effector cells (e.g., T cells or NK cells) per week (e.g., 2, 3 or 4 administrations per week) (also referred to herein as a cycle), followed by a week of no CAR immune effector cells (e.g., T cells or NK cells) administrations, and then one or more additional administration of the CAR immune effector cells (e.g., T cells or NK cells) (e.g., more than one administration of the CAR immune effector cells (e.g., T cells or NK cells) per week) is administered to the subject. In another embodiment, the subject (e.g., human subject) receives more than one cycle of CAR immune effector cells (e.g., T cells or NK cells), and the time between each cycle is less than 10, 9, 8, 7, 6, 5, 4, or 3 days. In one embodiment, the CAR immune effector cells (e.g., T cells or NK cells) are administered every other day for 3 administrations per week. In one embodiment, the CAR immune effector cells (e.g., T cells or NK cells) of the invention are administered for at least two, three, four, five, six, seven, eight or more weeks.

In one aspect, CAR-expressing cells (e.g., CARTs or CAR-expressing NK cells) are generated using lentiviral viral vectors, such as lentivirus. CAR-expressing cells (e.g., CARTs or CAR-expressing NK cells) generated that way will have stable CAR expression.

In one aspect, CAR-expressing cells, e.g., CARTs, are generated using a viral vector such as a gammaretroviral vector, e.g., a gammaretroviral vector described herein. CARTs generated using these vectors can have stable CAR expression.

In one aspect, CAR-expressing cells (e.g., CARTs or CAR-expressing NK cells) transiently express CAR vectors for 4, 5, 6, 7, 8, 9, 10, 11, 12, 13, 14, 15 days after transduction. Transient expression of CARs can be effected by RNA CAR vector delivery. In one aspect, the CAR RNA is transduced into the cell, e.g., T cell or NK cell, by electroporation.

A potential issue that can arise in patients being treated using transiently expressing CAR-expressing cells (e.g., CARTs or CAR-expressing NK cells) (particularly with murine scFv bearing CAR-expressing cells (e.g., CARTs or CAR-expressing NK cells)) is anaphylaxis after multiple treatments.

Without being bound by this theory, it is believed that such an anaphylactic response might be caused by a patient developing humoral anti-CAR response, i.e., anti-CAR antibodies having an anti-IgE isotype. It is thought that a patient's antibody producing cells undergo a class switch from IgG isotype (that does not cause anaphylaxis) to IgE isotype when there is a ten to fourteen day break in exposure to antigen.

If a patient is at high risk of generating an anti-CAR antibody response during the course of transient CAR therapy (such as those generated by RNA transductions), CAR-expressing cell (e.g., CART or CAR-expressing NK cell) infusion breaks should not last more than ten to fourteen days.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compositions of the present invention and practice the claimed methods. The following working examples specifically point out various aspects of the present invention, and are not to be construed as limiting in any way the remainder of the disclosure.

### Example 1: Long Term Solid Tumor Control with CAR T Cells Co-Expressing IL-15R/IL-15

### Abstract

CAR19 T cells, chimeric antigen receptor T cells directed against CD19, have been demonstrated to induce long term disease remission in relapsed/refractory patients with B-cell malignancies and have recently received FDA approval for the treatment of pediatric and young adult patients with acute lymphoblastic leukemia (ALL). The ability of CAR T cells to expand and persist in the patient is correlated with response, thus increasing CAR T cell survival and persistence should enhance therapeutic efficacy. Such strategies are of particular relevance in CAR T cell therapy for the treatment of solid tumors, where CAR T cells need to maintain function in the hostile tumor microenvironment characterized by suppressive signals, nutrient deprivation, and low oxygen.

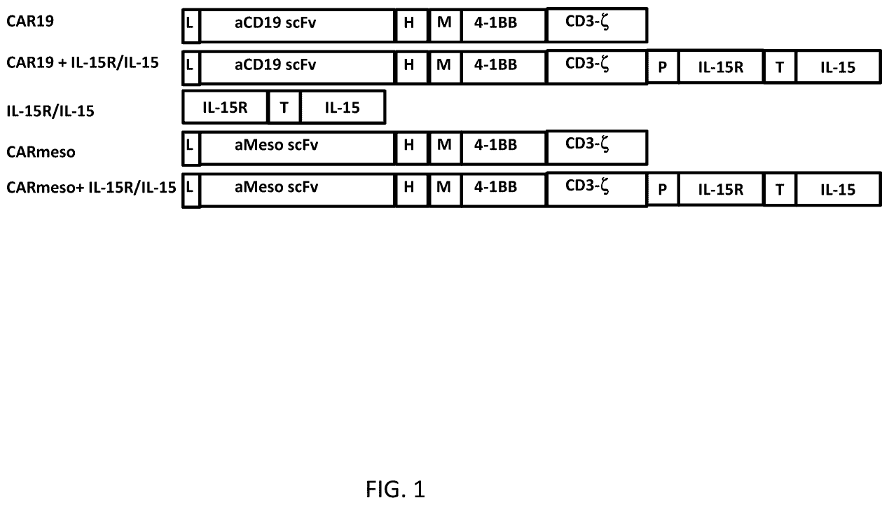

To this goal, we have generated CAR IL-15R/IL-15 constructs (FIG. 1). IL-15 signaling is known to generate survival signals to maintain naïve and memory CD8 T cells in the absence of antigen stimulation, and reduce apoptotic signaling.

During four rounds of re-stimulation over 4 weeks, CAR T cells co-expressing IL-15R/IL-15 proliferated similarly to IL-2 supplemented cultures. After the fourth re-stimulation, CAR T cells co-expressing IL-15R/IL-15 were maintained in culture without further cytokine supplements or antigen. These cultures persisted over an additional 10 weeks without expansion of CAR T cell numbers, and maintained antigen specific response, degranulation, IFN-γ release, and similar mitochondrial activity as week 4. Addition of IL-21 during 4 weeks of re-stimulation, improved long term survival for IL-15R/IL-15 CAR T cells. Additionally, persisting CAR T cells co-expressing IL-15R/IL-15 cultured without antigen stimulation for 4 weeks exhibited a greater naïve (CCR7+, CD45RO−) phenotype.

CAR T cells expressing IL-15R/IL-15 displayed enhanced long term anti-tumor efficacy in vivo. After 30 days post CAR T cell infusion, conventional CARmeso T cells were no longer able to control AsPC1 tumor, but CARmeso T cells co-expressing IL-15R/IL-15 maintained tumor control for over 70 days.

In summary, CAR T cells co-expressing IL-15R/IL-15 exhibited enhanced survival, performance and phenotypic profile, suggesting that this platform improvement holds promise for increasing the efficacy of CAR T cell therapy in tumors, e.g., solid tumors.

### Results:

Lentiviral constructs were designed to express chimeric antigen receptors (CAR) targeting huCD19 (CAR19) or hu mesothelin (CARmeso) with a CD8 leader (L), CD8 hinge (H), transmembrane domain (M), and intracellular domains (ICD) CD137 (4-1BB) and CD3 zeta (CD3-z) as shown in FIG. 1. IL-15Ra and IL-15 were co-expressed using the T2A system (T) which was co-expressed with the CAR using the P2A system (P) (FIG. 1). The CAR19 construct comprised a chimeric antigen receptor (CAR) targeting huCD19 with a CD8 leader (L), CD8 hinge (H), transmembrane domain (M), and intracellular domains (ICD) CD137 (4-1BB) and CD3 zeta (CD3-z).

The CAR19+IL-15R/-IL15 construct comprised the CAR19 construct and a P2A system, IL-15R, a T2A system and IL-15. The IL-15R/-IL15 construct comprised IL-15R, a T2A system and IL-15. The CARmeso+IL-15R/-IL15 construct comprised the CARmeso construct and a P2A system, IL-15R, a T2A system and IL-15. The CARmeso construct comprised a chimeric antigen receptor (CAR) targeting human mesothelin, with a CD8 leader (L), CD8 hinge (H), transmembrane domain (M), and intracellular domains (ICD) CD137 (4-1BB) and CD3 zeta (CD3-z). Lentiviral constructs were pseudotyped with VSV/G.

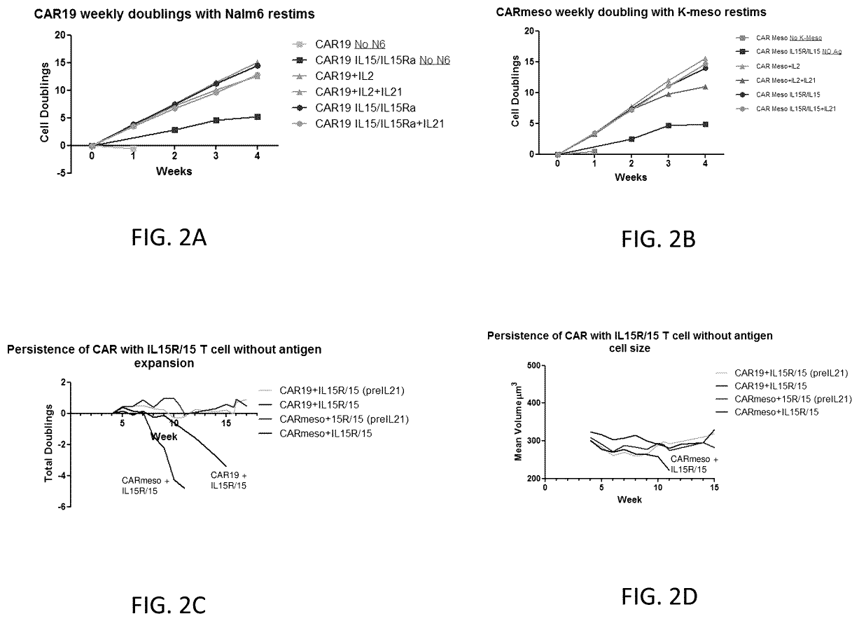

Co-expression of IL-15R/IL-15 with CAR expression was found to promote survival and persistence in long term cultures, with and without antigen stimulation. As shown in FIGS. 2A-2D, CAR19 and CARmeso T cells, were expanded through four rounds of re-stimulation with Nalm6 (N6) or K562-meso cells. Some groups were cultured with no antigen (No N6, No K-meso, or No Ag), while others were supplemented with 100 U/ml IL-2 and/or 10 ug/ml IL-21. After 4 weeks of re-stimulation, T cells were cultured alone re-placing half media volume every 3-4 days without any cytokine supplements for up to an additional 13 weeks (FIGS. 2C and 2D).

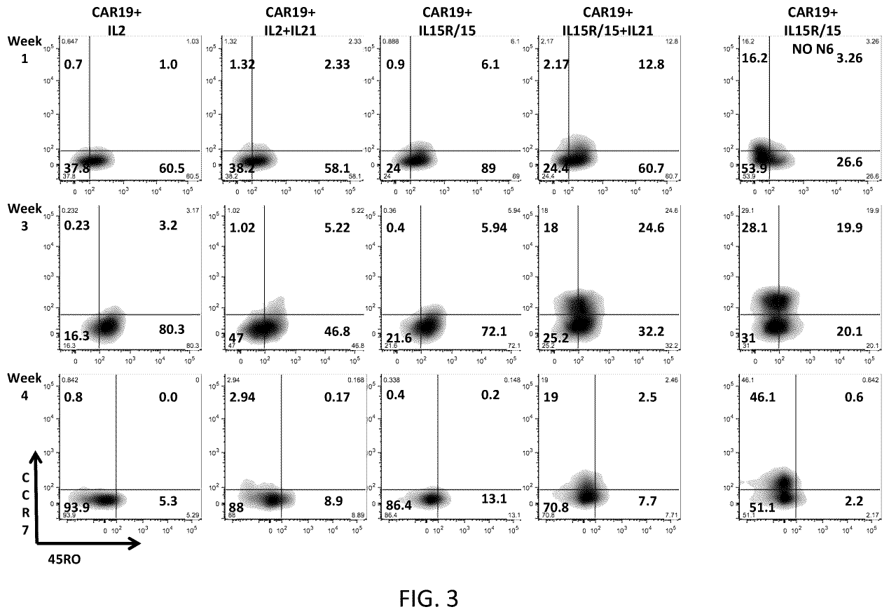

After surviving more than 60 days in culture after initial expansion without antigen, CAR T cells co-expressing IL-15R/IL-15 showed a naïve (CCR7+, CD45RO−) phenotype. It was observed that CAR19 T cells co-expressing IL-15R/IL-15 show enhanced CCR7 expression in non stimulated cells and in activated cells with addition of IL-21 (FIG. 3). CAR19 T cells cultures at the end of weeks 1, 3 and 4 were stained for CCR7 and CD45 expression and the T cell subsets were defined as follows: naïve (CCR7+/CD45RO−), Central memory (CCR7+/CD45RO+), Effector memory (CCR7−/CD45RO+) and terminal effectors (CCR7−/CD45RO−). T cell cultures at the end of week 4 primarily consisted of CD8+ T cells, e.g., >90% of CD8+ T cells. CAR T cells expressing IL-15R/IL-15 from initial expansions co-cultured with four rounds of re-stimulation with antigen-expressing tumor cells, displayed a less differentiated phenotype (CCR7+/−, CD45RO+) than CAR T cells alone, and reverted to a more naïve (CCR7+, CD45RO−) population as targeted antigen was eliminated.

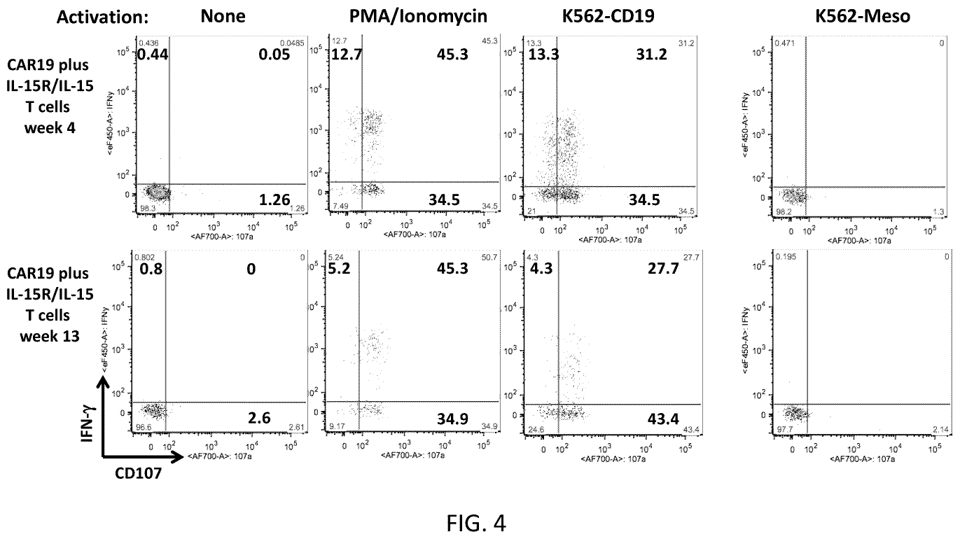

CAR19 T cells co-expressing IL-15R/IL-15 were shown to maintain functional response to antigen after persisting 10 weeks in antigen free cultures. Effector functions of CAR19 T cells were assessed. T cell populations at week 4 and week 13 were co-cultured with or without either K562 cells expressing CD19 (K562-CD19) or mesothelin (K562-Meso), or PMA and ionomycin and incubated for 6 hours with CD107a, monesin and Brefeldin A. The resulting T cell populations were fluorescently stained for live/dead, surface expression of CD3, CD4, and CD8, followed by intracellular staining to detect IFN-γ. Upon activation in co-cultures with antigen-expressing tumor cells, CAR T cells expressing IL-15R/IL-15 secreted cytokines, maintained cytolytic function and proliferative capacity (FIG. 4).

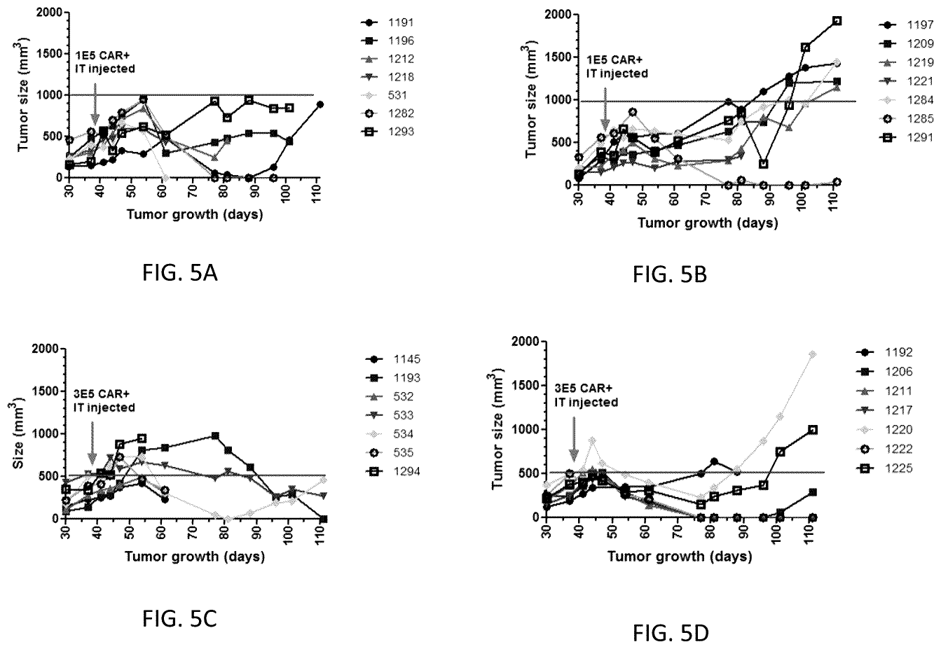

CAR T cells expressing IL-15R/IL-15 displayed enhanced long-term anti-tumor efficacy in vivo. CARmeso+IL-15R/IL-15 T cells exhibited superior long-term tumor control compared to CARmeso T cells alone in the AsPC1 pancreatic cancer xenograft NSG mouse model. CARmeso T cells co-expressing IL-15R/IL-15, and CARmeso T cells were evaluated in the AsPC1 NSG mouse model. One million AsPC1 cells were injected subcutaneously into the right flanks of NSG mice to establish tumors. Tumor growth was monitored by caliper measurements. On day 38 (tumors averaged 250 mm³) either 1×10⁵ or 3×10⁵ CAR+ T cells were injected intratumorally (IT) and tumors were monitored twice weekly. Initially, both CARmeso and CARmeso T cells co-expressing IL15R/15 T cells exhibited similar anti-tumor efficacy in the AsPC1 pancreatic cancer xenograft models. However, after 30 days post CAR T cell infusion only CARmeso T cells co-expressing IL-15R/IL-15 T cells displayed long term tumor control, while CARmeso T cells were no longer able to control AsPC1 tumor (FIGS. 5A-5D). This difference was more pronounced at the higher T cell dose. CARmeso T cells co-expressing IL-15R/IL-15 maintained tumor control for over 70 days.

### CONCLUSION

Continuous expression of IL-15R/IL-15 was shown to endow CAR T cells with, e.g., enhanced survival and persistence in long term cultures, both with and without antigen stimulation. CAR T cells co-expressing IL-15R/IL-15 also proliferated similarly to IL-2 supplemented cultures. Additionally, CAR T cells co-expressing IL-15R/IL-15 persisted over 10 weeks without antigen, exhibited a more naïve and memory phenotype (CCR7+), and maintained antigen specific degranulation and IFN-g production. Addition of IL-21 during 4 weeks of re-stimulation resulted in improved long-term survival of CAR+IL-15R/IL-15 T cells cultured without antigen. Finally, the data showed that CAR T cells co-expressing IL-15R/IL-15 exhibited enhanced anti-tumor efficacy in the AsPC1 pancreatic cancer xenograft models. In summary, CAR T cells co-expressing IL-15R/IL-15 exhibited enhanced survival, performance and phenotypic profiles, suggesting that, e.g., this platform improvement holds promise for increasing the efficacy of CAR T cell therapy in tumors, e.g., solid tumors.

### EQUIVALENTS

The disclosures of each and every patent, patent application, and publication cited herein are hereby incorporated herein by reference in their entirety. While this invention has been disclosed with reference to specific aspects, it is apparent that other aspects and variations of this invention may be devised by others skilled in the art without departing from the true spirit and scope of the invention. The appended claims are intended to be construed to include all such aspects and equivalent variations.

1. A nucleic acid molecule, comprising:
(i) a first nucleic acid sequence encoding a chimeric antigen receptor (CAR) molecule that binds to an antigen;
(ii) a second nucleic acid sequence encoding an IL-15 receptor (IL-15R) molecule; and
(iii) a third nucleic acid sequence encoding an IL-15 molecule,
wherein the first nucleic acid sequence, second nucleic acid sequence and third nucleic acid sequence are disposed on a single nucleic acid molecule.

2. (canceled)

3. The nucleic acid molecule of claim 1, wherein the single nucleic acid molecule has the following arrangement in an N- to C-terminal orientation:
(i) the first nucleic acid sequence-a first linker-the second nucleic acid sequence-a second linker-the third nucleic acid sequence;
(ii) the first nucleic acid sequence-a first linker-the third nucleic acid sequence-a second linker-the second nucleic acid sequence;
(iii) the second nucleic acid sequence-a first linker-the third nucleic acid sequence-a second linker-the first nucleic acid sequence; or
(iv) the third nucleic acid sequence-a first linker-the second nucleic acid sequence-a second linker-the first nucleic acid sequence,
wherein the first and second linkers are different.

4. (canceled)

5. (canceled)

6. The nucleic acid molecule of claim 3, wherein the first linker encodes a P2A site and the second linker encodes a T2A site, wherein:
(i) the first linker comprises the nucleotide sequence of SEQ ID NO: 23, or a sequence at least about 85%, 90%, 95%, 99% or more identical thereto, and/or having one, two, three, or more substitutions, insertions, deletions, or modifications, and/or
(ii) the second linker comprises a nucleotide sequence encoding SEQ ID NO: 1478, or a sequence at least about 85%, 90%, 95%, 99% or more identical thereto, and/or having one, two, three, or more substitutions, insertions, deletions, or modifications.

7. The nucleic acid molecule of claim 1, wherein the CAR molecule comprises, in an N- to C-terminal orientation, an antigen binding domain that binds to the antigen, a transmembrane domain, and an intracellular signaling domain.

8. (canceled)

9. The nucleic acid molecule of claim 1, wherein:
(i) the third nucleic acid sequence encodes an amino acid comprising the sequence of SEQ ID NO: 1002, a sequence at least about 85%, 90%, 95%, 99% or more identical to SEQ ID NO: 1002, or a sequence having one, two, three or more substitutions, insertions, deletions, or modifications compared to SEQ ID NO: 1002; and/or
(ii) the second nucleic acid sequence encodes an amino acid comprising the sequence of SEQ ID NO: 1001, a sequence at least about 85%, 90%, 95%, 99% or more identical to SEQ ID NO: 1001, or a sequence having one, two, three or more substitutions, insertions, deletions, or modifications compared to SEQ ID NO: 1001.

10. (canceled)

11. The nucleic acid molecule of claim 1, wherein:
(a) the antigen is selected from: CD19; BCMA; CD123; CD22; CD30; CD171; CS-1; C-type lectin-like molecule-1, CD33; epidermal growth factor receptor variant III (EGFRvIII); ganglioside G2 (GD2); ganglioside GD3; TNF receptor family member; B-cell maturation antigen; Tn antigen ((Tn Ag) or (GalNAcα-Ser/Thr)); Tn-O-Glycopeptides, sTn-O-Glycopeptides; prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2; Mesothelin; Interleukin 11 receptor alpha (IL-11Ra); prostate stem cell antigen (PSCA); Protease Serine 21; vascular endothelial growth factor receptor 2 (VEGFR2); Lewis (Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene polypeptide consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3; transglutaminase 5 (TGS5); high molecular weight-melanoma-associated antigen (HMWMAA); 0-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1, melanoma antigen recognized by T cells 1; Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like, Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); IL-13Ra2; bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); or immunoglobulin lambda-like polypeptide 1 (IGLL1);
(b) the transmembrane domain comprises:
(i) a transmembrane domain of a protein chosen from the alpha, beta, or zeta chain of the T-cell receptor, CD28, CD3 epsilon, CD45, CD4, CD5, CD8, CD9, CD16, CD22, CD33, CD37, CD64, CD80, CD86, CD123, CD134, CD137, or CD154;
(ii) the amino acid sequence of SEQ ID NO: 1026, or a sequence at least about 85%, 90%, 95%, 99% or more identical thereto, and/or having one, two, three, or more substitutions, insertions, or deletions;
(c) the intracellular signaling domain comprises a primary signaling domain, wherein the primary signaling domain comprises:
(i) a functional signaling domain derived from CD3 zeta, TCR zeta, FcR gamma, FcR beta, CD3 gamma, CD3 delta, CD3 epsilon, CD5, CD22, CD79a, CD79b, CD278 (ICOS), FcεRI, DAP10, DAP12, or CD66d; or
(ii) the amino acid sequence of SEQ ID NO: 1034 or 1037 or a sequence at least about 85%, 90%, 95%, 99% or more identical thereto, and/or having one, two, three, or more substitutions, insertions, or deletions; and/or
(d) the intracellular signaling domain comprises a costimulatory domain, wherein:
(i) the costimulatory domain comprises a functional signaling domain derived from MHC class I molecule, TNF receptor protein, Immunoglobulin-like protein, cytokine receptor, integrin, signalling lymphocytic activation molecule (SLAM), activating NK cell receptor, BTLA, a Toll ligand receptor, OX40, CD2, CD7, CD27, CD28, CD30, CD40, CDS, ICAM-1, 4-1BB (CD137), B7-H3, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD28-OX40, CD28-4-1BB, or a ligand that specifically binds with CD83; or
(ii) the costimulatory domain comprises the amino acid sequence of SEQ ID NO: 1029, or a sequence at least about 85%, 90%, 95%, 99% or more identical thereto, and/or having one, two, three or more substitutions, insertions, or deletions.

12.-17. (canceled)

18. A CAR IL-15/IL15R polypeptide comprising:
(i) a chimeric antigen receptor (CAR) molecule that binds to an antigen;
(ii) an IL-15 receptor (IL-15R) molecule; and
(iii) an IL-15 molecule,
wherein (i)-(iii) are expressed in the same frame on a single polypeptide chain.

19. The polypeptide of claim 18, wherein the polypeptide has the following arrangement in an N- to C-terminal orientation:
(i) CAR molecule-a first linker-IL-15R molecule-a second linker-IL-15 molecule;
(ii) CAR molecule-a first linker-IL-15 molecule-a second linker-IL-15R molecule;
(iii) IL-15R molecule-a first linker-IL-15 molecule-a second linker-CAR molecule; or
(iv) IL-15 molecule-a first linker-IL-15R molecule-a second linker-CAR molecule, wherein the first and second linkers are different.

20. (canceled)

21. (canceled)

22. The polypeptide of claim 19, wherein the first linker comprises a P2A site and the second linker comprises a T2A site, wherein:
(i) the first linker comprises the amino acid sequence of SEQ ID NO: 1479, or a sequence at least about 85%, 90%, 95%, 99% or more identical thereto, and/or having one, two, three, or more substitutions, insertions, deletions, or modifications, and/or
(ii) the second linker comprises the amino acid sequence of SEQ ID NO: 1478, or a sequence at least about 85%, 90%, 95%, 99% or more identical thereto, and/or having one, two, three, or more substitutions, insertions, deletions, or modifications.

23. The polypeptide of claim 18, wherein the CAR molecule comprises, in an N- to C-terminal orientation, an antigen binding domain that binds to the antigen, a transmembrane domain, and an intracellular signaling domain, optionally wherein the antigen binding domain is connected to the transmembrane domain by a hinge domain.

24. The polypeptide of claim 18, comprising:
(i) the amino acid sequence of SEQ ID NO: 1002, a sequence at least about 85%, 90%, 95%, 99% or more identical to SEQ ID NO: 1002, or a sequence having one, two, three, or more substitutions, insertions, deletions, or modifications compared to SEQ ID NO: 1002; or
(ii) the amino acid sequence of SEQ ID NO: 1001, a sequence at least about 85%, 90%, 95%, 99% or more identical to SEQ ID NO:1001, or a sequence having one, two, three or more substitutions, insertions, deletions, or modifications compared to SEQ ID NO: 1001.

25. (canceled)

26. A cell, comprising the nucleic acid molecule of claim 1.

27. A vector, comprising the nucleic acid molecule of claim 1.

28. A method of making a population of immune effector cells expressing Chimeric Antigen Receptor (CAR) molecule, IL-15R molecule and IL-15 molecule (“CAR IL-15/IL-15R expressing cells”), comprising:
a) providing a population of immune effector cells;
b) contacting the population of immune effector cells with a nucleic acid molecule of claim 1; and
c) maintaining the cells under conditions that allow expression of the CAR polypeptide, thereby making a population of CAR IL-15/IL-15R expressing immune effector cells.

29. (canceled)

30. (canceled)

31. The method of claim 28, further comprising contacting the population of cells with a ligand that binds to the CAR molecule, thereby stimulating the population of cells, and/or with an IL-21 molecule.

32.-41. (canceled)

42. The method of claim 28, comprising:
(i) expanding the population of cells, e.g., for at least 1, 2, 3, 4, 5, 6, 7, 8, 9, 10, 11, 12, 13, 14, 15, 16, 17, 18, 19, 20, 21, 22, 23, 24, 25, 26, 27, or 28 days or for 1-7, 7-14, 14-21, or 14-28 days; or
(ii) expanding the population of cells by at least, 5, 10, 20, 30, 40, 50, 60, 70, 80, 90, or 100-fold change in cell number or more under growth conditions of Example 1.

43. (canceled)

44. A method of evaluating a population of CAR IL-15/IL-15R expressing cells, comprising measuring the level of CD45RO and CCR7 in a sample of the population of CAR IL-15/IL-15R expressing cells, wherein:
a low level of CD45RO expression CD45RO− and a high level of CCR7 expression CCR7+ is indicative that the sample is suitable for treatment; and
a high level of CD45RO expression D45RO+ and a low level of CCR7 expression CCR7− is indicative that the sample is not suitable for treatment, thereby evaluating the population of CAR IL-15/IL-15R expressing cells.

45.-49. (canceled)

50. A method of treating a subject having a disease associated with expression of an antigen, comprising administering to the subject an effective number of a population of cells of claim 26.

51. A method of providing an anti-cancer immune response in a subject having a disease associated with expression of an antigen, comprising administering to the subject an effective number of a population of cells of claim 26.

52. The method of claim 50, wherein the disease associated with expression of an antigen, is a cancer.

53. (canceled)

54. The method of claim 52, wherein the cancer is:
(a) a solid tumor selected from the group consisting of mesothelioma; lung cancer; pancreatic cancer; esophageal cancer, ovarian cancer, breast cancer, colorectal cancer, bladder cancer, glioblastoma, prostate cancer, cervical cancer, skin cancer, melanoma, renal cancer, liver cancer, brain cancer, thymoma, sarcoma, carcinoma, uterine cancer, kidney cancer, gastrointestinal cancer, urothelial cancer, pharynx cancer, head and neck cancer, rectal cancer, or any combination thereof; and/or
(b) a hematological cancer that is a leukemia or lymphoma selected from the group consisting of chronic lymphocytic leukemia (CLL), mantle cell lymphoma (MCL), multiple myeloma, acute lymphoblastic leukemia (ALL), Hodgkin lymphoma, B-cell acute lymphoblastic leukemia (BALL), T-cell acute lymphoblastic leukemia (TALL), small lymphocytic lymphoma (SLL), B cell prolymphocytic leukemia, blastic plasmacytoid dendritic cell neoplasm, Burkitt's lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+ large B-cell lymphoma, large B-cell lymphoma arising in HHV8-associated multicentric Castleman disease, primary effusion lymphoma, B-cell lymphoma, acute myeloid leukemia (AML), and unclassifiable lymphoma.

55.-57. (canceled)

58. The method of claim 50, further comprising administering an additional therapeutic agent.

59. (canceled)

60. A method of evaluating or predicting a subject's responsiveness to a CAR IL-15/IL-15R-expressing cell therapy, comprising acquiring a value for the activity or expression level of CD45RO and CCR7 in a population of cells isolated from the subject, wherein:
a low level of CD45RO expression CD45RO− and a high level of CCR7 expression CCR7+ is indicative or predictive of increased responsiveness of the subject to the CAR IL-15/IL-15R-expressing cell therapy; and
a high level of CD45RO expression CD45RO+ and a low level of CCR7 expression CCR7− is indicative or predictive of decreased responsiveness of the subject to the CAR IL-15/IL-15R-expressing cell therapy,
thereby evaluating or predicting the subject's responsiveness to the CAR IL-15/IL-15R-expressing cell therapy.
